SEQUENCE LISTING (23 sequences)

SEQ ID NO: 1 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 2 - RNA, 6255 nt, Homo sapiens
  source 1..6255: mol_type = mRNA
actctaacaagtgactgcgcggcccgcgcccggggcggtgactgcggcaagccccctgggtccccgcgcggcgcatcccagcctgggcgggacgctcggccgcggcgaggcgggcaagcctggcagggcagagggagccccggctccgaggttgctcttcgcacccgaggatcagtcttggccccaaagcgcgacgcacaaatccacataacctgaggaccatggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaaagccctcccgcgcccccaccccagaccccgagccaccccaggaaaagcacaaggactgccgccttctcgctcccgccagcagcatagactggactggaccagacaatgttgtgtttggatttgtaactgttttttgttttttgtttgagttggttgattggggtttgatttgcttttgaaaagatttttatttttagaggcagggctgcattgggagcatccagaactgctaccttcctagatgtttccccagaccgctggctgagattccctcacctgtcgcttcctagaatccccttctccaaacgattagtctaaattttcagagagaaatagataaaacacgccacagcctgggaaggagcgtgctctaccctgtgctaagcacggggttcgcgcaccaggtgtctttttccagtccccagaagcagagagcacagcccctgctgtgtgggtctgcaggtgagcagacaggacaggtgtgccgccacccaagtgccaagacacagcagggccaacaacctgtgcccaggccagcttcgagctacatgcatctagggcggagaggctgcacttgtgagagaaaatactatttcaagtcatattctgcgtaggaaaatgaattggttggggaaagtcgtgtctgtcagactgccctgggtggagggagacgccgggctagagcctttgggatcgtcctggattcactggctttgcggaggctgctcagatggcctgagcctcccgaggcttgctgccccgtaggaggagactgtcttcccgtgggcatatctggggagccctgttccccgctttttcactcccatacctttaatggcccccaaaatctgtcactacaatttaaacaccagtcccgaaatttggatcttctttctttttgaatctctcaaacggcaacattcctcagaaaccaaagctttatttcaaatctcttccttccctggctggttccatctagtaccagaggcctcttttcctgaagaaatccaatcctagccctcattttaattatgtacatctgtttgtagccacaagcctgaatttctcagtgttggtaagtttctttacctaccctcactatatattattctcgttttaaaacccataaaggagtgatttagaacagtcattaattttcaactcaatgaaatatgtgaagcccagcatctctgttgctaacacacagagctcacctgtttgaaaccaagctttcaaacatgttgaagctctttactgtaaaggcaagccagcatgtgtgtccacacatacataggatggctggctctgcacctgtaggatattggaatgcacagggcaattgagggactgagccagaccttcggagagtaatgccaccagatcccctaggaaagaggaggcaaatggcactgcaggtgagaaccccgcccatccgtgctatgacatggaggcactgaagcccgaggaaggtgtgtggagattctaatcccaacaagcaagggtctccttcaagattaatgctatcaatcattaaggtcattactctcaaccacctaggcaatgaagaatataccatttcaaatatttacagtacttgtcttcaccaacactgtcccaaggtgaaatgaagcaacagagaggaaattgtacataagtacctcagcatttaatccaaacaggggttcttagtctcagcactatgacattttgggctgactacttatttgttaggcgggagctctcctgtgcattgtaggataattagcagtatccctggtggctacccaatagacgccagtagcaccccgaattgacaacccaaactctccagacatcaccaactgtcccctgcgaggagaaatcactcctgggggagaaccactgacccaaatgaattctaaaccaatcaaatgtctgggaagccctccaagaaaaaaaatagaaaagcacttgaagaatattcccaatattcccggtcagcagtatcaaggctgacttgtgttcatgtggagtcattataaattctataaatcaattattccccttcggtcttaaaaatatatttcctcataaacatttgagttttgttgaaaagatggagtttacaaagataccattcttgagtcatggatttctctgctcacagaagggtgtggcatttggaaacgggaataaacaaaattgctgcaccaatgcactgagtgaaggaagagagacagaggatcaagggctttagacagcactccttcaatatgcaatcacagagaaagatgcgccttatccaagttaatatctctaaggtgagagccttcttagagtcagtttgttgcaaatttcacctactctgttcttttccatccatccccctgagtcagttggttgaagggagttattttttcaagtggaattcaaacaaagctcaaaccagaactgtaaatagtgattgcaggaattcttttctaaactgctttgccctttcctctcactgccttttatagccaatataaatgtctctttgcacaccttttgttgtggttttatattgtaacaccatttttctttgaaactattgtatttaaagtaaggtttcatattatgtcagcaagtaattaacttatgtttaaaaggtggccatatcatgtaccaaaagttgctgaagtttctcttctagctggtaaagtaggagtttgcatgacttcacactttttttgcgtagtttcttctgttgtatgatggcgtgagtgtgtgtcttgggtaccgctgtgtactactgtgtgcctagattccatgcactctcgttgtgtttgaagtaaatattggagaccggagggtaacaggttggcctgttgattacagctagtaatcgctgtgtcttgttccgccccctccctgacaccccagcttcccaggatgtggaaagcctggatctcagctccttgccccatatcccttctgtaatttgtacctaaagagtgtgattatcctaattcaagagtcactaaaactcatcacattatcattgcatatcagcaaagggtaaagtcctagcaccaattgcttcacataccagcatgttccatttccaatttagaattagccacataataaaatcttagaatcttccttgagaaagagctgcctgagatgtagttttgttatatggttccccaccgaccatttttgtgcttttttcttgttttgttttgttttgactgcactgtgagttttgtagtgtcctcttcttgccaaaacaaacgcgagatgaactggacttatgtagacaaatcgtgatgccagtgtatccttcctttcttcagttccagcaataatgaatggtcaacttttttaaaatctagatctctctcattcatttcaatgtatttttactttaagatgaaccaaaattattagacttatttaagatgtacaggcatcagaaaaaagaagcacataatgcttttggtgcgatggcactcactgtgaacatgtgtaaccacatattaatatgcaatattgtttccaatactttctaatacagttttttataatgttgtgtgtggtgattgttcaggtcgaatctgttgtatccagtacagctttaggtcttcagctgcccttctggcgagtacatgcacaggattgtaaatgagaaatgcagtcatatttccagtctgcctctatgatgatgttaaattattgctgtttagctgtgaacaagggatgtaccactggaggaatagagtatccttttgtacacattttgaaatgcttcttctgtagtgatagaacaaataaatgcaacgaatactctgtctgccctatcccgtgaagtccacactggcgtaagagaaggcccagcagagcaggaatctgcctagactttctcccaatgagatcccaatatgagagggagaagagatgggcctcaggacagctgcaataccacttgggaacacatgtggtgtcttgatgtggccagcgcagcagttcagcacaacgtacctcccatctacaacagtgctggacgtgggaattctaagtcccagtcttgagggtgggtggagatggagggcaacaagagatacatttccagttctccactgcagcatgcttcagtcattctgtgagtggccgggcccagggccctcacaatttcactaccttgtcttttacatagtcataagaattatcctcaacatagccttttgacgctgtaaatcttgagtattcatttacccttttctgatctcctggaaacagctgcctgcctgcattgcacttctcttcccgaggagtggggtaaatttaaaagtcaagttatagtttggatgttagtatagaattttgaaattgggaattaaaaatcaggactggggactgggagaccaaaaatttctgatcccatttctgatggatgtgtcacaccttttctgtcaaaataaaatgtcttggaggttatgactccttggtgaaa

SEQ ID NO: 3 - RNA, 1560 nt, Homo sapiens
  source 1..1560: mol_type = other RNA
atggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaa

SEQ ID NO: 4 - protein, 477 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 5 - RNA, 6129 nt, Homo sapiens
  source 1..6129: mol_type = mRNA
cctttctctgcaccctggtggagtgtgaaggcagcagaggaaccttttggaggaggaagaggacacagaggccctgtagccaggcaccaagatccctcccaggtggctgggtctgaggggaactccgagcagccctaggtcctcaaagtctggatttgtgtggaaaaggcagctctcacttggccttggcgaggcctcggttggttgataacctgaggaccatggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaaagccctcccgcgcccccaccccagaccccgagccaccccaggaaaagcacaaggactgccgccttctcgctcccgccagcagcatagactggactggaccagacaatgttgtgtttggatttgtaactgttttttgttttttgtttgagttggttgattggggtttgatttgcttttgaaaagatttttatttttagaggcagggctgcattgggagcatccagaactgctaccttcctagatgtttccccagaccgctggctgagattccctcacctgtcgcttcctagaatccccttctccaaacgattagtctaaattttcagagagaaatagataaaacacgccacagcctgggaaggagcgtgctctaccctgtgctaagcacggggttcgcgcaccaggtgtctttttccagtccccagaagcagagagcacagcccctgctgtgtgggtctgcaggtgagcagacaggacaggtgtgccgccacccaagtgccaagacacagcagggccaacaacctgtgcccaggccagcttcgagctacatgcatctagggcggagaggctgcacttgtgagagaaaatactatttcaagtcatattctgcgtaggaaaatgaattggttggggaaagtcgtgtctgtcagactgccctgggtggagggagacgccgggctagagcctttgggatcgtcctggattcactggctttgcggaggctgctcagatggcctgagcctcccgaggcttgctgccccgtaggaggagactgtcttcccgtgggcatatctggggagccctgttccccgctttttcactcccatacctttaatggcccccaaaatctgtcactacaatttaaacaccagtcccgaaatttggatcttctttctttttgaatctctcaaacggcaacattcctcagaaaccaaagctttatttcaaatctcttccttccctggctggttccatctagtaccagaggcctcttttcctgaagaaatccaatcctagccctcattttaattatgtacatctgtttgtagccacaagcctgaatttctcagtgttggtaagtttctttacctaccctcactatatattattctcgttttaaaacccataaaggagtgatttagaacagtcattaattttcaactcaatgaaatatgtgaagcccagcatctctgttgctaacacacagagctcacctgtttgaaaccaagctttcaaacatgttgaagctctttactgtaaaggcaagccagcatgtgtgtccacacatacataggatggctggctctgcacctgtaggatattggaatgcacagggcaattgagggactgagccagaccttcggagagtaatgccaccagatcccctaggaaagaggaggcaaatggcactgcaggtgagaaccccgcccatccgtgctatgacatggaggcactgaagcccgaggaaggtgtgtggagattctaatcccaacaagcaagggtctccttcaagattaatgctatcaatcattaaggtcattactctcaaccacctaggcaatgaagaatataccatttcaaatatttacagtacttgtcttcaccaacactgtcccaaggtgaaatgaagcaacagagaggaaattgtacataagtacctcagcatttaatccaaacaggggttcttagtctcagcactatgacattttgggctgactacttatttgttaggcgggagctctcctgtgcattgtaggataattagcagtatccctggtggctacccaatagacgccagtagcaccccgaattgacaacccaaactctccagacatcaccaactgtcccctgcgaggagaaatcactcctgggggagaaccactgacccaaatgaattctaaaccaatcaaatgtctgggaagccctccaagaaaaaaaatagaaaagcacttgaagaatattcccaatattcccggtcagcagtatcaaggctgacttgtgttcatgtggagtcattataaattctataaatcaattattccccttcggtcttaaaaatatatttcctcataaacatttgagttttgttgaaaagatggagtttacaaagataccattcttgagtcatggatttctctgctcacagaagggtgtggcatttggaaacgggaataaacaaaattgctgcaccaatgcactgagtgaaggaagagagacagaggatcaagggctttagacagcactccttcaatatgcaatcacagagaaagatgcgccttatccaagttaatatctctaaggtgagagccttcttagagtcagtttgttgcaaatttcacctactctgttcttttccatccatccccctgagtcagttggttgaagggagttattttttcaagtggaattcaaacaaagctcaaaccagaactgtaaatagtgattgcaggaattcttttctaaactgctttgccctttcctctcactgccttttatagccaatataaatgtctctttgcacaccttttgttgtggttttatattgtaacaccatttttctttgaaactattgtatttaaagtaaggtttcatattatgtcagcaagtaattaacttatgtttaaaaggtggccatatcatgtaccaaaagttgctgaagtttctcttctagctggtaaagtaggagtttgcatgacttcacactttttttgcgtagtttcttctgttgtatgatggcgtgagtgtgtgtcttgggtaccgctgtgtactactgtgtgcctagattccatgcactctcgttgtgtttgaagtaaatattggagaccggagggtaacaggttggcctgttgattacagctagtaatcgctgtgtcttgttccgccccctccctgacaccccagcttcccaggatgtggaaagcctggatctcagctccttgccccatatcccttctgtaatttgtacctaaagagtgtgattatcctaattcaagagtcactaaaactcatcacattatcattgcatatcagcaaagggtaaagtcctagcaccaattgcttcacataccagcatgttccatttccaatttagaattagccacataataaaatcttagaatcttccttgagaaagagctgcctgagatgtagttttgttatatggttccccaccgaccatttttgtgcttttttcttgttttgttttgttttgactgcactgtgagttttgtagtgtcctcttcttgccaaaacaaacgcgagatgaactggacttatgtagacaaatcgtgatgccagtgtatccttcctttcttcagttccagcaataatgaatggtcaacttttttaaaatctagatctctctcattcatttcaatgtatttttactttaagatgaaccaaaattattagacttatttaagatgtacaggcatcagaaaaaagaagcacataatgcttttggtgcgatggcactcactgtgaacatgtgtaaccacatattaatatgcaatattgtttccaatactttctaatacagttttttataatgttgtgtgtggtgattgttcaggtcgaatctgttgtatccagtacagctttaggtcttcagctgcccttctggcgagtacatgcacaggattgtaaatgagaaatgcagtcatatttccagtctgcctctatgatgatgttaaattattgctgtttagctgtgaacaagggatgtaccactggaggaatagagtatccttttgtacacattttgaaatgcttcttctgtagtgatagaacaaataaatgcaacgaatactctgtctgccctatcccgtgaagtccacactggcgtaagagaaggcccagcagagcaggaatctgcctagactttctcccaatgagatcccaatatgagagggagaagagatgggcctcaggacagctgcaataccacttgggaacacatgtggtgtcttgatgtggccagcgcagcagttcagcacaacgtacctcccatctacaacagtgctggacgtgggaattctaagtcccagtcttgagggtgggtggagatggagggcaacaagagatacatttccagttctccactgcagcatgcttcagtcattctgtgagtggccgggcccagggccctcacaatttcactaccttgtcttttacatagtcataagaattatcctcaacatagccttttgacgctgtaaatcttgagtattcatttacccttttctgatctcctggaaacagctgcctgcctgcattgcacttctcttcccgaggagtggggtaaatttaaaagtcaagttatagtttggatgttagtatagaattttgaaattgggaattaaaaatcaggactggggactgggagaccaaaaatttctgatcccatttctgatggatgtgtcacaccttttctgtcaaaataaaatgtcttggaggttatgactccttggtgaaa

SEQ ID NO: 6 - RNA, 6129 nt, Homo sapiens; identical to SEQ ID NO: 5
  source 1..6129: mol_type = other RNA

SEQ ID NO: 7 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 8 - RNA, 1320 nt, Homo sapiens
  source 1..1320: mol_type = mRNA
ctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtagcacggtttctattga

SEQ ID NO: 9 - RNA, 885 nt, Homo sapiens
  source 1..885: mol_type = other RNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 10 - protein, 265 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKGGSLPKVEAKFINYVKNCFRMTDQEAIQDLWQWRKSL

SEQ ID NO: 11 - RNA, 1233 nt, Homo sapiens
  source 1..1233: mol_type = mRNA
ctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtagcacggtttctattga

SEQ ID NO: 12 - RNA, 798 nt, Homo sapiens
  source 1..798: mol_type = other RNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 13 - protein, 751 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 14 - RNA, 2654 nt, Homo sapiens
  source 1..2654: mol_type = mRNA
gtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 15 - RNA, 2256 nt, Homo sapiens
  source 1..2256: mol_type = other RNA
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 16 - protein, 707 aa, Homo sapiens
MGQTGKKSEKGPVCWRKRVKSEYMRLRQLKRFRRADEVKSMFSSNRQKILERTEILNQEWKQRRIQPVHILTSVSSLRGTREVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 17 - RNA, 2522 nt, Homo sapiens
  source 1..2522: mol_type = mRNA
gtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggaggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 18 - RNA, 2124 nt, Homo sapiens
  source 1..2124: mol_type = other RNA
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggaggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttga

SEQ ID NO: 19 - RNA, 48 nt, synthetic construct
  source 1..48: mol_type = other RNA
gccgaagctataaacagcgaactcgagttcgctgtttatagcttcggc

SEQ ID NO: 20 - protein, 767 aa, Homo sapiens
MGRRSRGRRLQQQQRPEDAEDGAEGGGKRGEAGWEGGYPEIVKENKLFEHYYQELKIVPEGEWGQFMDALREPLPATLRITGYKSHAKEILHCLKNKYFKELEDLEVDGQKVEVPQPLSWYPEELAWHTNLSRKILRKSPHLEKFHQFLVSETESGNISRQEAVSMIPPLLLNVRPHHKILDMCAAPGSKTTQLIEMLHADMNVPFPEGFVIANDVDNKRCYLLVHQAKRLSSPCIMVVNHDASSIPRLQIDVDGRKEILFYDRILCDVPCSGDGTMRKNIDVWKKWTTLNSLQLHGLQLRIATRGAEQLAEGGRMVYSTCSLNPIEDEAVIASLLEKSEGALELADVSNELPGLKWMPGITQWKVMTKDGQWFTDWDAVPHSRHTQIRPTMFPPKDPEKLQAMHLERCLRILPHHQNTGGFFVAVLVKKSSMPWNKRQPKLQGKSAETRESTQLSPADLTEGKPTDPSKLESPSFTGTGDTEIAHATEDLENNGSKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKILLTQENPFFRKLSSETYSQAKDLAKGSIVLKYEPDSANPDALQCPIVLCGWRGKASIRTFVPKNERLHYLRMMGLEVLGEKKKEGVILTNESAASTGQPDNDVTEGQRAGEPNSPDAEEANSPDVTAGCDPAGVHPPR

SEQ ID NO: 21 - RNA, 2304 nt, Homo sapiens
  source 1..2304: mol_type = other RNA
atggggcggcggtcgcggggtcggcggctccagcaacagcagcggccggaggacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtga

SEQ ID NO: 22 - protein, 732 aa, Homo sapiens
MGRRSRGRRLQQQQRPEDAEDGAEGGGKRGEAGWEGGYPEIVKENKLFEHYYQELKIVPEGEWGQFMDALREPLPATLRITGYKRYPEELAWHTNLSRKILRKSPHLEKFHQFLVSETESGNISRQEAVSMIPPLLLNVRPHHKILDMCAAPGSKTTQLIEMLHADMNVPFPEGFVIANDVDNKRCYLLVHQAKRLSSPCIMVVNHDASSIPRLQIDVDGRKEILFYDRILCDVPCSGDGTMRKNIDVWKKWTTLNSLQLHGLQLRIATRGAEQLAEGGRMVYSTCSLNPIEDEAVIASLLEKSEGALELADVSNELPGLKWMPGITQWKVMTKDGQWFTDWDAVPHSRHTQIRPTMFPPKDPEKLQAMHLERCLRILPHHQNTGGFFVAVLVKKSSMPWNKRQPKLQGKSAETRESTQLSPADLTEGKPTDPSKLESPSFTGTGDTEIAHATEDLENNGSKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKILLTQENPFFRKLSSETYSQAKDLAKGSIVLKYEPDSANPDALQCPIVLCGWRGKASIRTFVPKNERLHYLRMMGLEVLGEKKKEGVILTNESAASTGQPDNDVTEGQRAGEPNSPDAEEANSPDVTAGCDPAGVHPPR

SEQ ID NO: 23 - RNA, 2199 nt, Homo sapiens
  source 1..2199: mol_type = other RNA
atggggcggcggtcgcggggtcggcggctccagcaacagcagcggccggaggacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaaggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtga

(19) United States
(12) Patent Application Publication
(10) Pub. No.: US 2026/0335308 A1
(43) Pub. Date: Oct. 29, 2026

(54) METHODS AND COMPOSITIONS FOR TREATMENT OF DISEASE

(71) Applicant: THE UNIVERSITY OF CHICAGO, Chicago, IL (US)

(72) Inventors: Chuan HE, Chicago, IL (US); Boyang GAO, Chicago, IL (US); Wenlong LI, Chicago, IL (US)

(21) Appl. No.: 19/642,983

(22) Filed: Apr. 9, 2026

Related U.S. Application Data
(60) Provisional application No. 63/786,029, filed on Apr. 9, 2025.

(51) Int. Cl. A61K 31/496; A61K 31/225; A61K 31/404; A61K 31/405; A61K 31/4155; A61K 31/427; A61K 31/4545; A61K 31/4725; A61K 31/506; A61K 31/5377; A61K 31/706; A61K 45/06; A61K 47/55; A61P 35/02; C07D 209/12; C07D 209/18; C07D 401/14; C07D 403/06; C07D 405/06; C07D 409/06; C07D 417/06; C07D 487/04; C12N 15/113; C12N 15/86

(52) U.S. Cl. CPC A61K 31/496; A61K 31/225; A61K 31/404; A61K 31/405; A61K 31/4155; A61K 31/427; A61K 31/4545; A61K 31/4725; A61K 31/506; A61K 31/5377; A61K 31/706; A61K 45/06; A61K 47/55; A61P 35/02; C07D 209/12; C07D 209/18; C07D 401/14; C07D 403/06; C07D 405/06; C07D 409/06; C07D 417/06; C07D 487/04; C12N 15/113; C12N 15/86; C12N 2310/14; C12N 2310/314; C12N 2310/315; C12N 2310/321; C12N 2310/531; C12N 2740/15043

(57) ABSTRACT

Aspects of the present disclosure are directed to at least methods and compositions for diagnosis and/or treatment of diseases, which may comprise cancer and/or pre-cancerous cells. Treatment of a disease may comprises administration of one or more inhibitors of IKZF1, EZH2, and/or NPM1. Also provided herein are methods of treatment of a disease in an individual comprising administering one or more inhibitors of IKZF1, EZH2, and/or NPM1 to the individual as a co-therapy with 5-azaC.

### CROSS REFERENCE TO RELATED APPLICATIONS

This application claims the benefit of the U.S. Provisional Patent Application No. 63/786,029 filed Apr. 9, 2025 the disclosure of which is herein incorporated by reference in their entirety.

### SEQUENCE LISTING

The instant application contains a Sequence Listing which has been submitted in ST26 format and is hereby incorporated by reference in its entirety. Said ST26 copy, created on Apr. 8, 2026, is named ARCD_P0873WO_Sequence_Listing.xml and is 63,399 bytes in size.

### BACKGROUND

### I. Field

Aspects of this invention relate to at least the field of molecular biology and medicine, including at least compositions and methods for therapeutic purposes.

### II. Background

The gene Ten-eleven translocation enzyme 2 (TET2) and TET2 protein pathway associated genes are frequently disrupted in various diseases, such as human cancers, and have been shown to drive myeloid malignancy initiation and progression. TET2 deficiency has been shown to result in globally opened chromatin and activation of genes contributing to aberrant hematopoietic stem cell self-renewal. However, the open chromatin consistently observed in TET2-deficient mouse embryonic stem cells, leukemic cells, and hematopoietic progenitor and stem cells is inconsistent with TET2's described role of DNA 5mC oxidation. Additionally, suitable biomarkers for determining efficacy of treatment options associated with diseases comprising TET2 mutations and/or TET pathway associated mutations are lacking. There exists a need for methods and compositions for treating TET2 and/or TET2 pathway deficient diseases, such as cancer and/or pre-cancers.

5-azacytidine (5-azaC) is a DNA hypomethylating agent clinically used to improve outcomes in myeloid malignancies. However, 5-azaC treatment causes gene dysregulation inconsistent with DNA hypomethylation changes (1-4). Some patients that have benefited from 5-azaC treatment did not display notable DNA demethylation (5). These observations suggest alternative mechanisms of action by 5-azaC. As a ribonucleoside analogue, 5-azaC is more readily incorporated into nascent RNA. There exists a need for identification of biomarkers associated with 5-azaC treatment efficacy and/or targets for combination therapy.

### SUMMARY

The inventors demonstrate herein that RNA 5-methylcytosine (m⁵C) depletion by 5-azaC treatment, particularly at early time points, induced leukemia cell death, causing transcriptional repression in contrast to 5-azaC DNA hypomethylating functions. While not being bound by theory, mechanistically, in contrast to its DNA demethylation function, the RNA-dependent process by 5-azaC causes transcriptional repression, disrupting genes involved in cell cycle regulation and DNA repair. The results provided herein demonstrate how 5-azaC impairs two specific m⁵C-mediated transcriptional regulatory pathways. First, depletion of m⁵C in chromatin-associated RNA (caRNA) disrupts the MBD6-mediated H2AK119ub removal. In parallel, this also impairs SRSF2 recruitment and the downstream H3K27ac deposition by p300. Loss of the caRNA methyltransferase NSUN2 caused prolonged cell cycle, defective DNA repair, and shifted hematopoietic lineage commitment toward erythropoiesis, mirroring the effects of 5-azaC treatment. Furthermore, the inventors have performed leukemia cell line screens and identified that TET2, Ikaros family zinc finger protein 1 (IKZF1), Enhancer of zeste homolog 2 (EZH2), and/or nucleophosmin 1 (NPM1) depletion can sensitize cells to 5-azaC treatment, consistent with the observed RNA-dependent cytotoxicity of 5-azaC in leukemia cells. The findings provided herein have unveiled the cytotoxicity of 5-azaC through RNA m⁵C depletion, identified biomarkers for prediction of 5-azaC treatment efficacy, and provided future directions for therapy developments based on 5-azaC.

Incorporated herein by reference are the contents of the inventors co-pending application PCT/US2024/049478 filed Oct. 1, 2024.

The present disclosure addresses certain needs outlined above by providing at least methods, compositions, and kits for treatment of diseases associated with chromatin state misregulation, in particular, chromatin state misregulation associated with leukemia and/or mutations in TET2 and/or TET2 pathway associated genes.

In some aspects, provided herein are methods of treating a disease in an individual, comprising the step of administering one or more inhibitors of Ikaros family zinc finger protein 1 (IKZF1), Enhancer of zeste homolog 2 (EZH2), and/or nucleophosmin 1 (NPM1), to an individual in need thereof. In some aspects, methods comprise the step of administering one or more inhibitors of IKZF1. In some aspects, methods comprise the step of administering one or more inhibitors of EZH2. In some aspects, methods comprise the step of administering one or more inhibitors of NPM1. In some aspects, an individual is administered an additional therapy, optionally wherein the additional therapy comprises administration of 5-AzaC.

In some aspects, a disease comprises cancer of the blood, lung, brain, breast, skin, pancreas, liver, colon, head and neck, kidney, thyroid, stomach, spleen, gallbladder, bone, ovary, testes, endometrium, prostate, rectum, anus, and/or cervix. In some aspects, the disease comprises clonal hematopoiesis of indeterminate potential (CHIP). In some aspects, the disease is characterized by atherosclerosis, myocardial fibrosis, and/or heart failure. In some aspects, the cancer comprises a blood cancer. In some aspects, the blood cancer comprises a leukemia. In some aspects, the blood cancer comprises a myeloid malignancy. In some aspects, the myeloid malignancy comprises acute myeloid leukemia. In some aspects, the myeloid malignancy comprises chronic myelomonocytic leukemia. In some aspects, the myeloid malignancy comprises myelodysplastic syndrome. In some aspects, the cancer comprises a glioma. In some aspects, the glioma comprises glioblastoma. In some aspects, the method comprises reducing proliferation of a cancer and/or pre-cancerous cell.

In some aspects, a disease is associated with diseased cells comprising one or more mutations in one or more genes encoding a ten-eleven translocation (tet) methylcytosine dioxygenase 2 (TET2), ASXL transcriptional regulator 1 (ASXL1), isocitrate dehydrogenase 1 (IDH1), isocitrate dehydrogenase 2 (IDH2), tumor protein p53 (p53), DNA (cytosine-5-)-methyltransferase 3A (DNMT3A), Janus kinase 2 (JAK2), Protein Phosphatase Mn2+/Mg2+-Dependent 1D (PPM1D), Spliceosome Factor 3b1 (SF3B1), and/or Serine and Arginine Rich Splicing Factor 2 (SRSF2). In some aspects, a disease is associated with diseased cells comprising a rearrangement of the lysine methyltransferase 2A (KMT2A)/mixed-lineage leukemia (MLL) gene.

In some aspects, the disease is associated with diseased cells with one or more mutations in one or more genes encoding components of a canonical and/or non-canonical Polycomb Repressive Complex (PRC). In some aspects, the one or more mutations in one or more genes encoding components of PRC comprises one or more loss of function mutations. In some aspects, the one or more mutations in one or more genes encoding components of PRC comprises one or more mutations in E3 Ubiquitin Ligase RING1A/B, Polycomb Group Ring Finger 1 (PCGF1), Polycomb Group Ring Finger 2 (PCGF2), Polycomb Group Ring Finger 3 (PCGF3), Polycomb Group Ring Finger 4 (PCGF4), Polycomb Group Ring Finger 5 (PCGF5), and/or Polycomb Group Ring Finger 6 (PCGF6). In some aspects, the disease is associated with diseased cells with one or more mutations in one or more genes encoding Polycomb Repressive-Deubiquitinase (PR-DUB) complex associated components O-linked N-acetylglucosamine Transferase (OGT), Lysine Demethylase 1B (KDM1B), Forkhead Box K1 (FOXK1), Forkhead Box K2 (FOXK2), BRCA1 Associated Protein 1 (BAP1), ASXL Transcriptional Regulator 1 (ASXL1), ASXL Transcriptional Regulator 2 (ASXL2), ASXL Transcription Regulator 3 (ASXL3), and/or Host Cell Factor C1 (HCFC1). In some aspects, the one or more mutations in PR-DUB complex associated components OGT, KDM1B, FOXK1, FOXK2, BAP1, ASXL1, ASXL2, ASXL3, and/or HCFC1 comprises one or more gain of function mutations.

In some aspects, a disease is associated with diseased cells with one or more mutations in a TET2 encoding gene. In some aspects, the one or more mutations in a TET2 encoding gene comprises one or more loss of function mutations. In some aspects, the disease is associated with diseased cells comprising one or more mutations in one or more genes encoding SRSF2, ASXL1, p53, DNMT3A, JAK2, PPM1D, and/or SF3B1.

In some aspects, methods provided herein comprise co-therapy with 5-azaC administration. In some aspects, an individual is administered an additional therapy, optionally wherein the additional therapy comprises administration of 5-azaC. In some aspects, an individual is administered an additional therapy, wherein the additional therapy comprises administration of 5-azaC. In some aspects, the administration of 5-azaC occurs before, concurrently, and/or during administration of an inhibitor of IKZF1, EZH2, and/or NPM1. In some aspects, the additional therapy comprises surgery, radiation, chemotherapy, hormone therapy, and/or immunotherapy. In some aspects, the additional therapy comprises administration of an inhibitor of TET2. In some aspects, the inhibitor of TET2 comprises C35 and/or TETi76. In some aspects, the additional therapy comprises administration of an inhibitor of NSUN1 and/or NSUN2. In some aspects, the additional therapy comprises administration of an inhibitor of NSUN2. In some aspects, the inhibitor of NSUN2 comprises a compound of Formula I, as provided herein. In various aspects, the inhibitor of NSUN2 may comprise one or more of Compounds 1 to 57 as provided herein. In various aspects, the inhibitor of NSUN2 may comprise Compound 48, Compound 1, Compound 12, Compound 18, Compound 19, Compound 24 or any pharmaceutically appropriate salt thereof. In various aspects, the inhibitor of NSUN2 may comprise Compound 48 or a pharmaceutically appropriate salt thereof. In some aspects, the additional therapy comprises administering an inhibitor of MBD6.

In some aspects, methods provided herein comprise a step of diagnosing the disease in the individual. In some aspects, the disease is associated with diseased cells characterized as comprising an open chromatin state relative to non-diseased cells of the same developmental lineage. In some aspects, administering the one or more IKZF1, EZH2, and/or NPM1 inhibitors results in promotion of a closed chromatin state in one or more diseased cells in the individual. In some aspects, the disease is characterized as pro-inflammatory. In some aspects, the methods comprise inhibition of diseased cell proliferation.

In some aspects, the administering the one or more IKZF1, EZH2, and/or NPM1 inhibitors decreases m⁵C levels in one or more chromatin associated RNA (caRNA) and/or decreases association of one or more caRNA with a PR-DUB complex in one or more diseased cells in the individual. In some aspects, the administering the one or more IKZF1, EZH2, and/or NPM1 inhibitors increases oxidation of m⁵C in the one or more caRNA and/or inhibits installation of m⁵C in the one or more caRNA in one or more diseased cells in the individual. In some aspects, histone ubiquitination is increased in one or more diseased cells after administration of the one or more IKZF1, EZH2, and/or NPM1 inhibitors. In some aspects, the histone ubiquitination comprises or consists of ubiquitination on H2A. In some aspects, the histone ubiquitination occurs at a site comprising or consisting essentially of H2AK119. In some aspects, the administering the one or more IKZF1, EZH2, and/or NPM1 inhibitors modifies histone methylation in one or more diseased cells in the individual. In some aspects, the histone that comprises modified methylation comprises or consists essentially of H3. In some aspects, the histone comprises modified methylation on a methylation site comprising or consisting essentially of H3K27me3. In some aspects, the histone that comprises modified methylation is localized near or at a genetic locus overlapping with a histone that comprises modified histone ubiquitination.

In some aspects, the one or more inhibitors of IKZF1, EZH2, and/or NPM1 comprise a polynucleotide at least partially complementary to a gene encoding IKZF1, EZH2, and/or NPM1. In some aspects, the polynucleotide comprises a short hairpin RNA and/or small interfering RNA. In some aspects, the polynucleotide comprises a sequence at least 80% complementary to at least 15, 20, 25, or more than 25 contiguous nucleotides of any one or more of SEQ ID NOs: 2-3, 5-6, 8-9, 11-12, 14-15, or 17-19. In some aspects, the polynucleotide comprises modified RNA phosphoramidites. In some aspects, the polynucleotide comprises RNA with one or more 2′-O-Methyl (2′-OMe) or 2′-O-Methoxyethyl (2′-MOE) modifications. In some aspects, the polynucleotide comprises RNA with every nucleotide comprising a 2′-MOE modification. In some aspects, the polynucleotide comprises RNA with one or more phosphorothioate bonds. In some aspects, the polynucleotide is comprised within a lentiviral particle and/or nanoparticle. In some aspects, the inhibitor of IKZF1, EZH2, and/or NPM1 comprises more than one polynucleotide. In some aspects, the one or more inhibitors of IKZF1, EZH2, and/or NPM1 comprise a proteolysis targeting chimera.

Also provided herein are methods of treating a disease in an individual, wherein the disease is characterized by cells comprising one or more mutations in IKZF1, EZH2, NPM1, TET2, ASXL1, IDH1, IDH2, p53, DNMT3A, JAK2, PPM1D, SF3B1, and/or SRSF2 encoding genes and/or a rearrangement of the lysine methyltransferase 2A (KMT2AY mixed-lineage leukemia (MLL) gene, the method comprising administering one or more inhibitors of IKZF1, EZH2, NPM1, MBD6, TET2, and/or NSUN2 to the individual in need thereof. In some aspects, the disease is characterized by cells comprising a TET2 mutation and the method comprises administering an inhibitor of NSUN2 to the subject. In some aspects, the disease is characterized by cells comprising a rearrangement of the lysine methyltransferase 2A (KMT2AY mixed-lineage leukemia (MLL) gene and the method comprises administering an inhibitor of NSUN2 to the subject. In various aspects, the inhibitor of NSUN2 may comprise a compound of Formula I as provided herein. In various aspects, the inhibitor of NSUN2 may comprise one or more of Compounds 1 to 57 as provided herein. In various aspects, the inhibitor of NSUN2 may comprise Compound 48, Compound 1, Compound 12, Compound 18, Compound 19, Compound 24 or any pharmaceutically appropriate salt thereof. In various aspects, the inhibitor of NSUN2 may comprise Compound 48 or a pharmaceutically appropriate salt thereof

Also provided herein are methods of treating a disease in an individual, comprising administering 5-AzaC and one or more inhibitors IKZF1, EZH2, and/or NPM1, to an individual in need thereof.

Also provided herein are methods of treating a disease in an individual, comprising determining the gene expression levels and/or protein activity levels of one or more of IKZF1, EZH2, and/or NPM1 in diseased cells in the individual, and administering 5-AzaC to the individual if the expression levels and/or protein activity levels of IKZF1, EZH2, and/or NPM1 are reduced relative to expression levels in a non-diseased cell of the same developmental lineage in the individual.

Also provided herein are methods of treating a disease in an individual, comprising administering one or more inhibitors of NSUN2 to the individual in need thereof. In various aspects, the inhibitor of NSUN2 comprises a compound of Formula I as provided herein, or a pharmaceutically appropriate salt thereof. In various aspects, the inhibitor of NSUN2 may comprise one or more of Compounds 1 to 57 as provided herein, or any pharmaceutically appropriate salt thereof. In various aspects, the inhibitor of NSUN2 may comprise Compound 48, Compound 1, Compound 12, Compound 18, Compound 19, Compound 24 or any pharmaceutically appropriate salt thereof. In various aspects, the inhibitor of NSUN2 may comprise Compound 48 or a pharmaceutically appropriate salt thereof.

In some aspects, the disease is characterized by cells comprising one or more mutations in IKZF1, EZH2, NPM1, TET2, ASXL1, IDH1, IDH2, p53, DNMT3A, JAK2, PPM1D, SF3B1, and/or SRSF2 encoding genes, and/or a rearrangement of a lysine methyltransferase 2A (KMT2A)/mixed-lineage leukemia (MLL) gene. In various aspects, the disease is characterized by cells comprising a mutation in a TET2 encoding gene. In various aspects, the disease is characterized by cells comprising a rearrangement of a lysine methyltransferase 2A (KMT2A)/mixed-lineage leukemia (MLL) gene. In some aspects, the disease comprises cancer of the blood, lung, brain, breast, skin, pancreas, liver, colon, head and neck, kidney, thyroid, stomach, spleen, gallbladder, bone, ovary, testes, endometrium, prostate, rectum, anus, and/or cervix. In some aspects, the disease comprises clonal hematopoiesis of indeterminate potential (CHIP). In some aspects, the disease is characterized by atherosclerosis, myocardial fibrosis, and/or heart failure. In some aspects, the cancer comprises a blood cancer. In some aspects, the blood cancer comprises a leukemia. In some aspects, the blood cancer comprises a myeloid malignancy. In some aspects, the myeloid malignancy comprises acute myeloid leukemia. In some aspects, the myeloid malignancy comprises chronic myelomonocytic leukemia. In some aspects, the myeloid malignancy comprises myelodysplastic syndrome. In some aspects, the cancer comprises a glioma. In some aspects, the glioma comprises glioblastoma. In some aspects, the one or more inhibitors of EZH2 comprises Tazemetostat, CPI-1205, GSK2816126, PF-06821497, SHR2554, and/or DS3201b. In some aspects, the one or more inhibitors of IKZF1 comprises CC-92480.

Also provided herein are manufactured articles for use in performing any of the methods described herein. Also provided herein are means for generating a manufactured article for diagnosing and/or treating an individual. Also provided herein are kits comprising means for performing any of the methods described herein.

In some aspects, provided herein are compounds of Formula (I), or a pharmaceutically appropriate salt thereof, wherein Formula (I) is Ar-L-B, Ar may be a heteroaryl moiety or an aryl moiety substituted with a heteroaryl substituent, wherein Ar is optionally substituted with 1 to 3 Ry substituents, each Ry independently selected from halogen, cyano, nitro, alkyl, haloalkyl, alkoxy, haloalkoxy, alkoxycarbonyl, carboxy(C1-C6)alkoxy, aryl, heteroaryl, aryloxy, and aralkoxy, wherein the heteroaryl moiety or the heteroaryl substituent comprises at least one ring heteroatom selected from nitrogen, oxygen, and sulfur, and when the heteroaryl moiety or the heteroaryl substituent comprises a ring nitrogen atom, the ring nitrogen atom is unsubstituted or substituted with RN, wherein RN may be selected from SO2RX, C(═O)RX, and C(═O)ORX, and wherein RX may be selected from substituted or unsubstituted C1-C6 alkyl, substituted or unsubstituted aryl, substituted or unsubstituted heteroaryl, and substituted or unsubstituted heterocyclyl; L is —CH═, wherein the carbon atom of L is singly bonded to Ar and doubly bonded to a carbon atom of B; and B may be selected from a barbiturate moiety, a thiobarbiturate moiety, a Meldrum's acid moiety, a thiazolidinedione moiety, a pyrazolone moiety, an indane-1,3-dione moiety, a cyanoacrylic acid moiety, a cyanoacrylate moiety, and an aryl-substituted acrylonitrile moiety.

In some aspects, B may be a barbiturate moiety or a thiobarbiturate moiety.

In some aspects, B may be a pyrimidine-2,4,6(1H,3H,5H)-trione moiety or a 2-thioxodihydropyrimidine-4,6(1H,5H)-dione moiety.

In some aspects, B may be

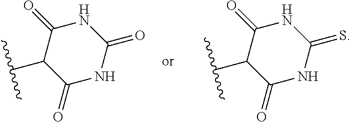 [SMILES: *C1C(=O)NC(=O)NC1=O ; *C1C(=O)NC(=S)NC1=O]

In some aspects, B may be a Meldrum's acid moiety, a thiazolidinedione moiety, a pyrazolone moiety, an indane-1,3-dione moiety, a cyanoacrylic acid moiety, a cyanoacrylate moiety, or an aryl-substituted acrylonitrile moiety.

In some aspects, B may be

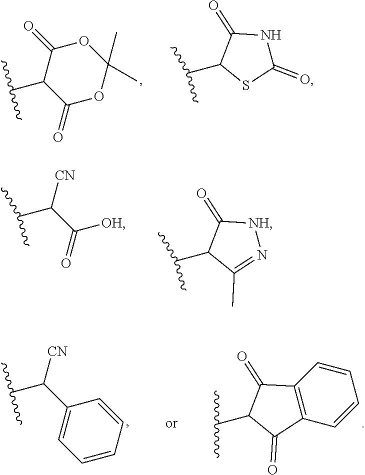

In some aspects, Ar may be a heteroaryl moiety. In some aspects, Ar may be a fused heteroaryl moiety comprising nitrogen. In some aspects, Ar may be an indolyl moiety or a pyrrolopyridinyl moiety. In some aspects, Ar may be an indol-3-yl moiety, an indol-2-yl moiety, an indol-4-yl moiety, an indol-5-yl moiety or an indol-6-yl moiety. In some aspects, Ar may be a fused heteroaryl moiety comprising sulfur. In some aspects, Ar may be a benzothiophenyl moiety. In some aspects, Ar may be a fused heteroaryl moiety comprising oxygen. In some aspects, Ar may be a dioxolo-fused indolyl moiety. In some aspects, Ar may be a pyrrolyl moiety or a thiazolyl moiety. In some aspects, Ar may be an aryl moiety substituted with a heteroaryl substituent. In some aspects, Ar may be a phenyl moiety substituted with a thiazolyl moiety.

In some aspects, the R_{N} substituent may be —SO2R_{X}. In some aspects, the R_{N} substituent may be C(═O)R_{X}. In some aspects, the R_{N} substituent may be C(═O)OR_{X}.

In some aspects, R_{X} may be aryl, heteroaryl, or heterocyclyl. In some aspects, the aryl may be phenyl or naphthyl. In some aspects, R_{X} may be benzo[d][1,3]dioxol-5-yl. In some aspects, the heterocyclyl may be a nitrogen-containing heterocyclyl. In some aspects, the heterocyclyl may be substituted on a ring nitrogen atom with tert-butoxycarbonyl. In some aspects, the heterocyclyl may be piperidinyl. In some aspects, R_{X} may be substituted with 1 to 3 substituents independently selected from halogen, cyano, alkyl, alkoxy, haloalkyl, haloalkoxy, and aryloxy. In some aspects, R_{X} may be substituted or unsubstituted C1-C6 alkyl. In some aspects, Rx may be tert-butyl.

In some aspects, at least one R_{y} substituent may be selected from trifluoromethyl, trifluoromethoxy, methoxycarbonyl, benzyloxy, nitro, phenyl, furyl, methoxy, chloro, cyano, methyl, and phenoxy.

In some aspects, the compound may comprise a structure selected from:

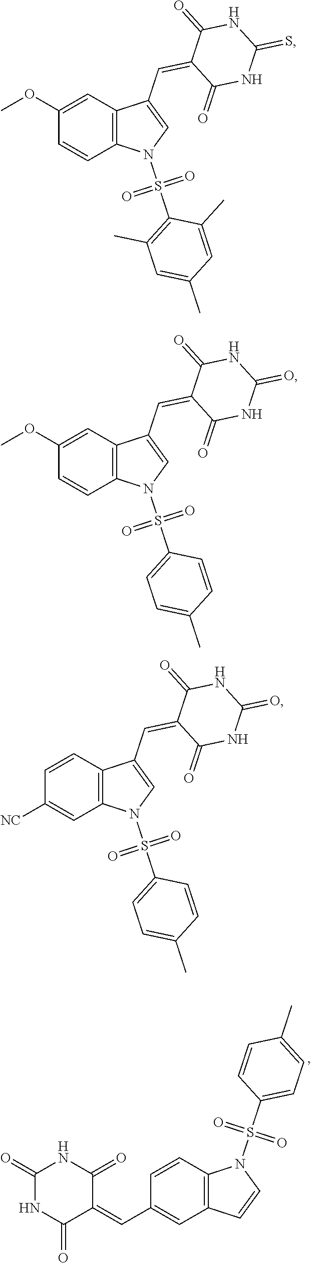

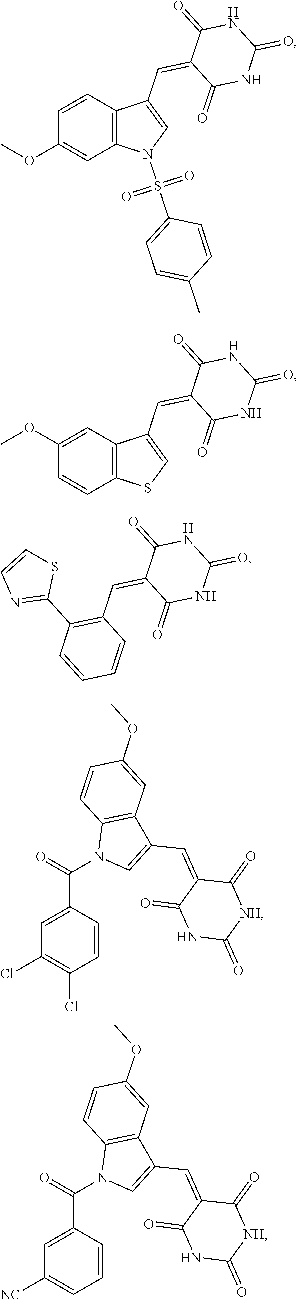

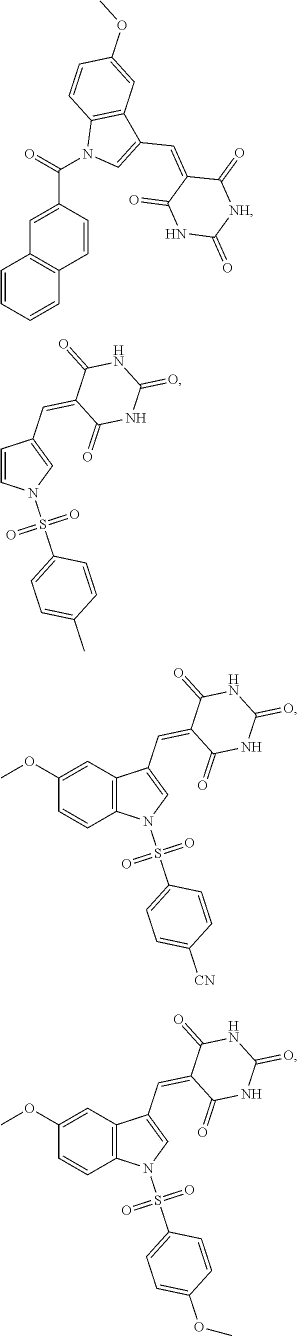

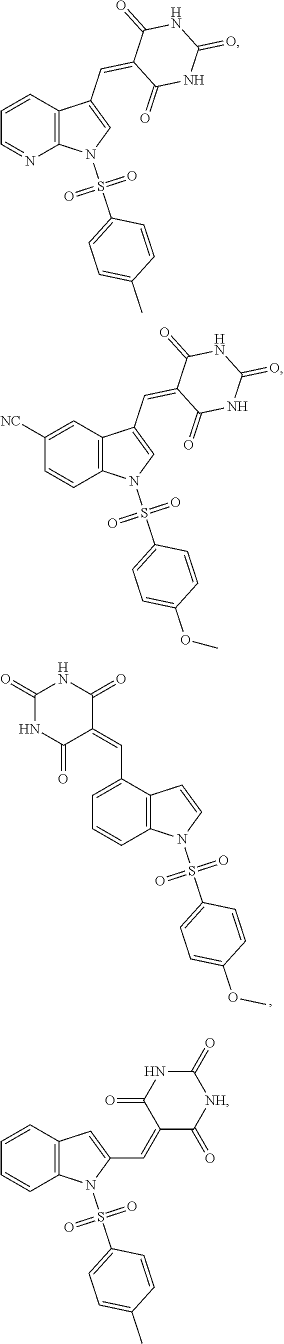

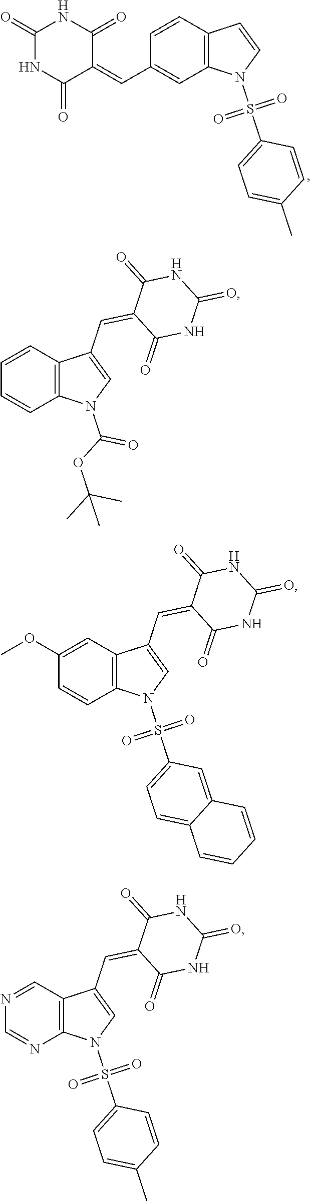 [SMILES: Cc1ccc(S(=O)(=O)n2ccc3ccc(C=C4C(=O)NC(=O)NC4=O)cc32)cc1 ; CC(C)(C)OC(=O)n1cc(C=C2C(=O)NC(=O)NC2=O)c2ccccc21 ; COc1ccc2c(c1)c(C=C1C(=O)NC(=O)NC1=O)cn2S(=O)(=O)c1ccc2ccccc2c1 ; Cc1ccc(S(=O)(=O)n2cc(C=C3C(=O)NC(=O)NC3=O)c3cncnc32)cc1]

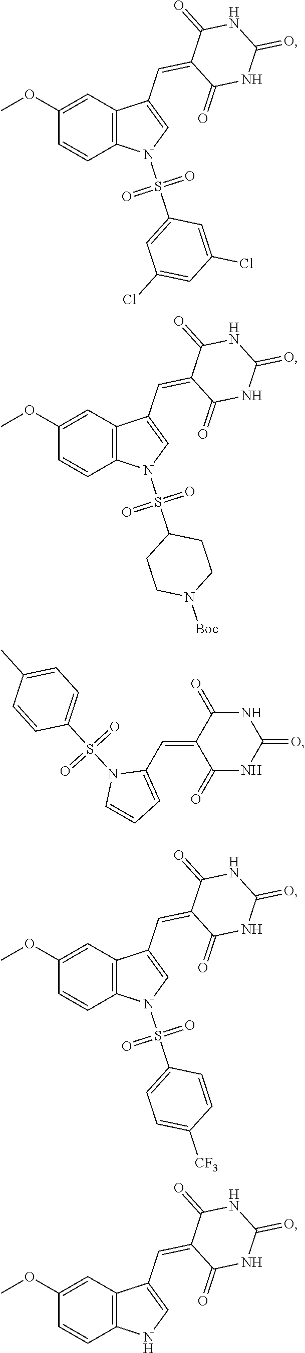

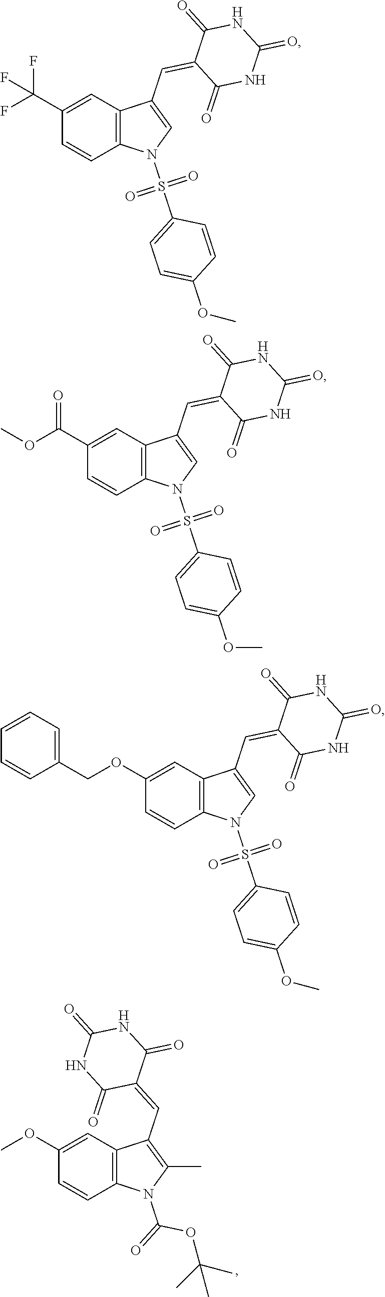

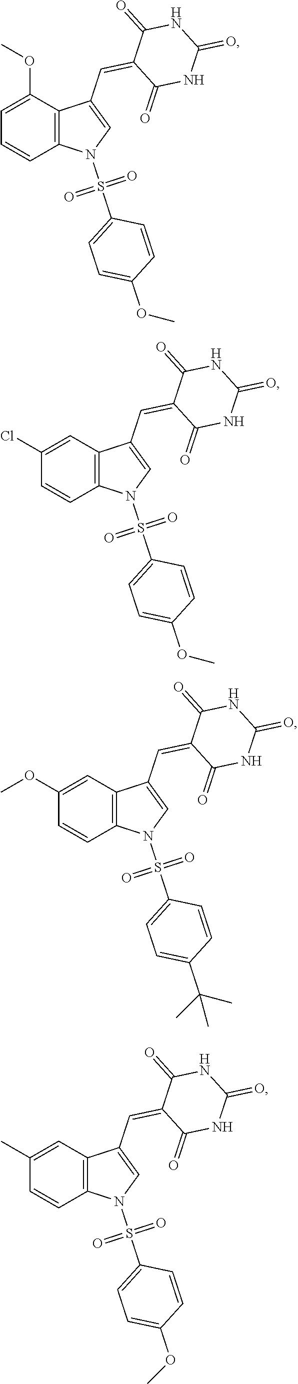

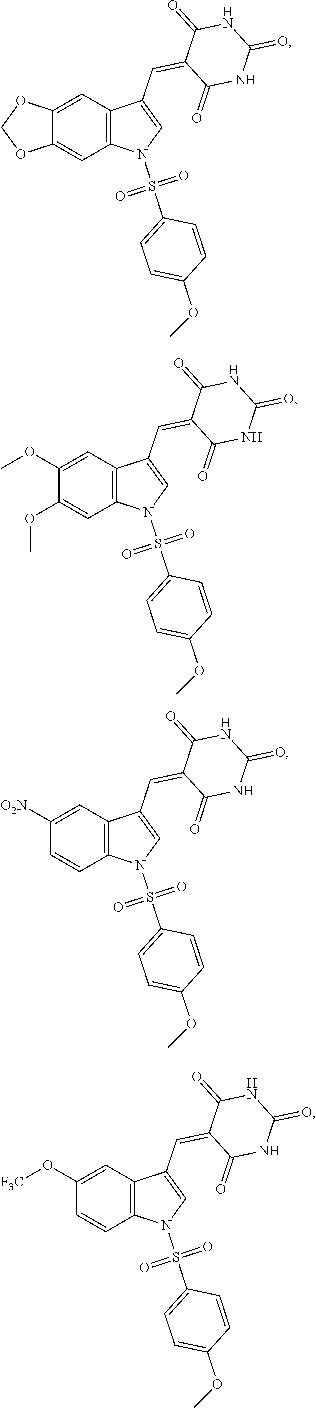

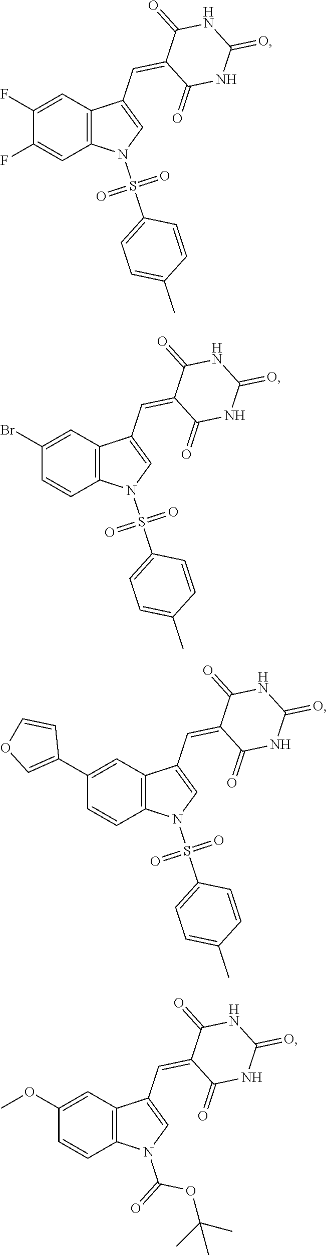

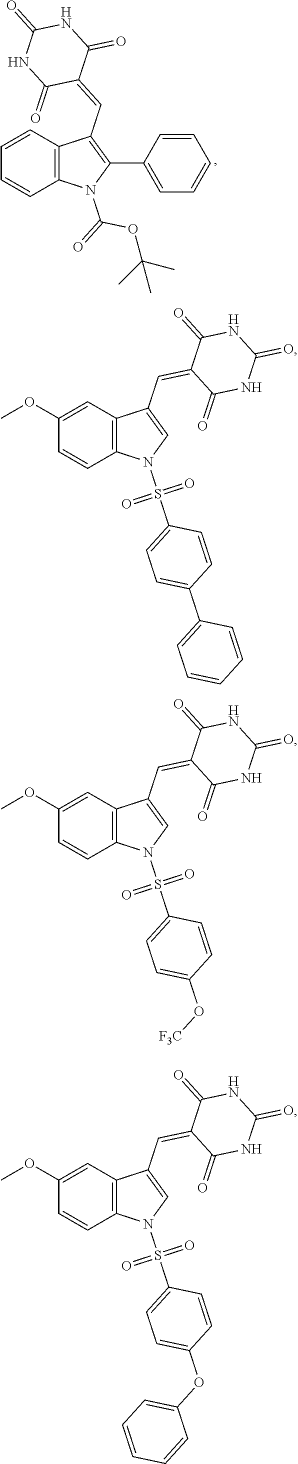

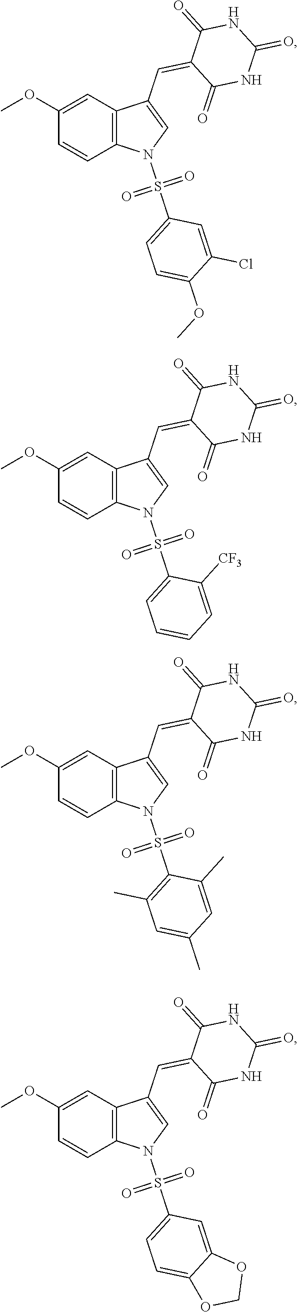

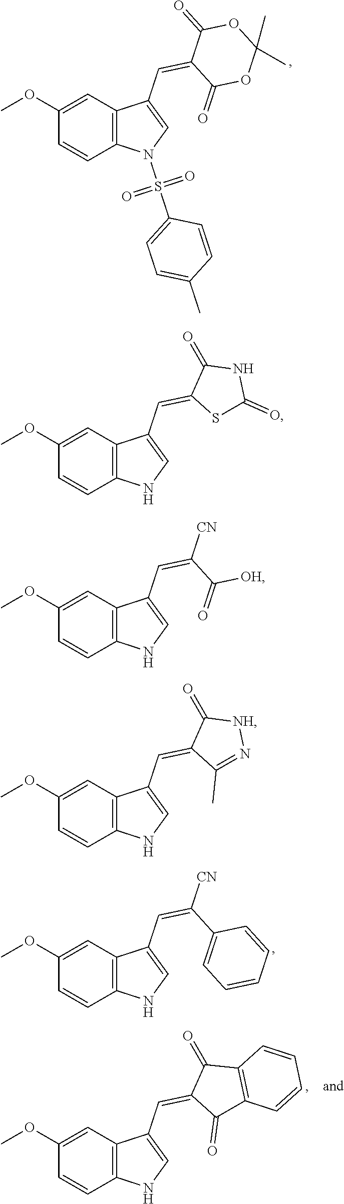

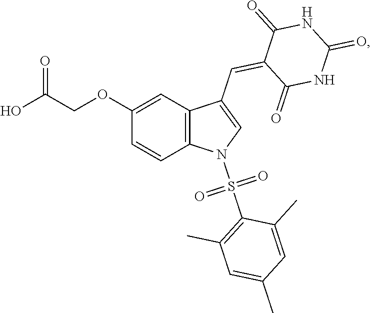 [SMILES: Cc1cc(C)c(S(=O)(=O)n2cc(C=C3C(=O)NC(=O)NC3=O)c3cc(OCC(=O)O)ccc32)c(C)c1]

or a pharmaceutically appropriate salt thereof.

In some aspects, a compound is provided having a structure selected from:

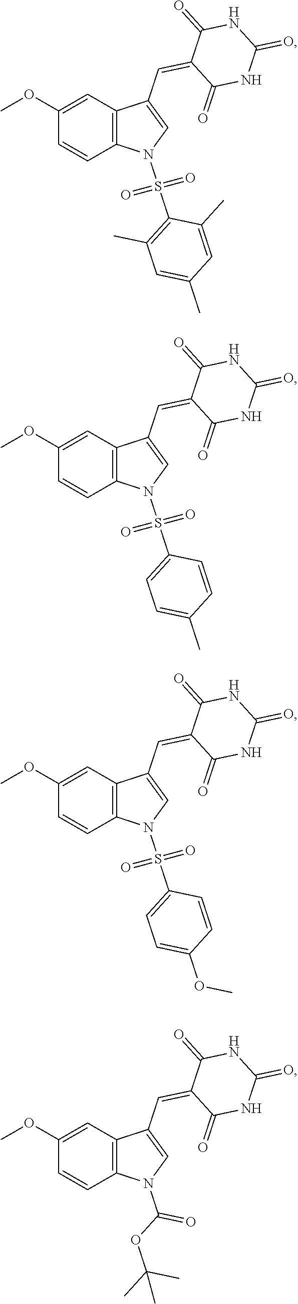

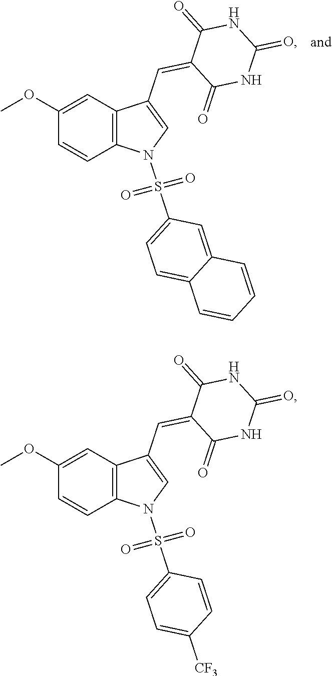 [SMILES: COc1ccc2c(c1)c(C=C1C(=O)NC(=O)NC1=O)cn2S(=O)(=O)c1ccc2ccccc2c1 ; COc1ccc2c(c1)c(C=C1C(=O)NC(=O)NC1=O)cn2S(=O)(=O)c1ccc(C(F)(F)F)cc1]

or a pharmaceutically appropriate salt thereof.

In some aspects, a compound is provided having a structure of

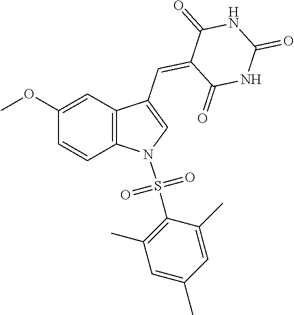 [SMILES: COc1ccc2c(c1)c(C=C1C(=O)NC(=O)NC1=O)cn2S(=O)(=O)c1c(C)cc(C)cc1C]

or a pharmaceutically appropriate salt thereof.

In some aspects, provided herein is a pharmaceutical composition comprising a compound of Formula (I) as described herein and at least one pharmaceutically appropriate carrier or excipient.

Certain aspects of the present disclosure are characterized through the following enumerated aspects.

Aspect 1 includes a method of treating a disease in an individual, comprising the step of administering one or more inhibitors of Ikaros family zinc finger protein 1 (IKZF1), Enhancer of zeste homolog 2 (EZH2), and/or nucleophosmin 1 (NPM1), to an individual in need thereof.

Aspect 2 depends upon Aspect 1, comprising the step of administering one or more inhibitors of IKZF1.

Aspect 3 depends upon Aspect 1, comprising the step of administering one or more inhibitors of EZH2.

Aspect 4 depends upon Aspect 1, comprising the step of administering one or more inhibitors of NPM1.

Aspect 5 depends upon any one of Aspects 1 to 4, wherein the disease comprises cancer of the blood, lung, brain, breast, skin, pancreas, liver, colon, head and neck, kidney, thyroid, stomach, spleen, gallbladder, bone, ovary, testes, endometrium, prostate, rectum, anus, and/or cervix.

Aspect 6 depends upon any one of Aspects 1 to 5, wherein the disease comprises clonal hematopoiesis of indeterminate potential (CHIP).

Aspect 7 depends upon Aspect 6, wherein the disease is characterized by atherosclerosis, myocardial fibrosis, and/or heart failure.

Aspect 8 depends upon Aspect 5, wherein the cancer comprises a blood cancer.

Aspect 9 depends upon Aspect 8, wherein the blood cancer comprises a leukemia.

Aspect 10 depends upon Aspect 8, wherein the blood cancer comprises a myeloid malignancy.

Aspect 11 depends upon Aspect 10, wherein the myeloid malignancy comprises acute myeloid leukemia.

Aspect 12 depends upon Aspect 10, wherein the myeloid malignancy comprises chronic myelomonocytic leukemia.

Aspect 13 depends upon Aspect 10, wherein the myeloid malignancy comprises myelodysplastic syndrome.

Aspect 14 depends upon Aspect 5, wherein the cancer comprises a glioma.

Aspect 15 depends upon Aspect 14, wherein the glioma comprises glioblastoma.

Aspect 16 depends upon any one of Aspects 1 to 15, comprising reducing proliferation of a cancer and/or pre-cancerous cell.

Aspect 17 depends upon any one of Aspects 1 to 16, wherein the disease is associated with diseased cells comprising one or more mutations in one or more genes encoding a ten-eleven translocation (tet) methylcytosine dioxygenase 2 (TET2), ASXL transcriptional regulator 1 (ASXL1), isocitrate dehydrogenase 1 (IDH1), isocitrate dehydrogenase 2 (IDH2), tumor protein p53 (p53), DNA (cytosine-5-)-methyltransferase 3A (DNMT3A), Janus kinase 2 (JAK2), Protein Phosphatase Mn2+/Mg2+-Dependent 1D (PPM1D), Spliceosome Factor 3b1 (SF3B1), and/or Serine and Arginine Rich Splicing Factor 2 (SRSF2).

Aspect 18 depends upon any one of Aspects 1 to 17, wherein the disease is associated with diseased cells comprising a rearrangement of the lysine methyltransferase 2A (KMT2A)/mixed-lineage leukemia (MLL) gene.

Aspect 19 depends upon any one of Aspects 1 to 18, wherein the disease is associated with diseased cells with one or more mutations in one or more genes encoding components of a canonical and/or non-canonical Polycomb Repressive Complex (PRC).

Aspect 20 depends upon Aspect 19, wherein the one or more mutations in one or more genes encoding components of PRC comprises one or more loss of function mutations.

Aspect 21 depends upon Aspect 19, wherein the one or more mutations in one or more genes encoding components of PRC comprises one or more mutations in E3 Ubiquitin Ligase RING1A/B, Polycomb Group Ring Finger 1 (PCGF1), Polycomb Group Ring Finger 2 (PCGF2), Polycomb Group Ring Finger 3 (PCGF3), Polycomb Group Ring Finger 4 (PCGF4), Polycomb Group Ring Finger 5 (PCGF5), and/or Polycomb Group Ring Finger 6 (PCGF6).

Aspect 22 depends upon any one of Aspects 1 to 21, wherein the disease is associated with diseased cells with one or more mutations in one or more genes encoding Polycomb Repressive-Deubiquitinase (PR-DUB) complex associated components O-linked N-acetylglucosamine Transferase (OGT), Lysine Demethylase 1B (KDM1B), Forkhead Box K1 (FOXK1), Forkhead Box K2 (FOXK2), BRCA1 Associated Protein 1 (BAP1), ASXL Transcriptional Regulator 1 (ASXL1), ASXL Transcriptional Regulator 2 (ASXL2), ASXL Transcription Regulator 3 (ASXL3), and/or Host Cell Factor C1 (HCFC1).

Aspect 23 depends upon Aspect 22, wherein the one or more mutations in PR-DUB complex associated components OGT, KDM1B, FOXK1, FOXK2, BAP1, ASXL1, ASXL2, ASXL3, and/or HCFC1 comprises one or more gain of function mutations.

Aspect 24 depends upon Aspect 17, wherein the disease is associated with diseased cells with one or more mutations in a TET2 encoding gene.

Aspect 25 depends upon Aspect 24, wherein the one or more mutations in a TET2 encoding gene comprises one or more loss of function mutations.

Aspect 26 depends upon Aspect 25, wherein the disease is associated with diseased cells comprising one or more mutations in one or more genes encoding SRSF2, ASXL1, p53, DNMT3A, JAK2, PPM1D, and/or SF3B1.

Aspect 27 depends upon any one of Aspects 1 to 26, wherein the individual is administered an additional therapy, optionally wherein the additional therapy comprises administration of 5-AzaC.

Aspect 28 depends upon Aspect 27, wherein the additional therapy comprises administration of 5-AzaC.

Aspect 29 depends upon Aspect 27, wherein the additional therapy comprises surgery, radiation, chemotherapy, hormone therapy, and/or immunotherapy.

Aspect 30 depends upon Aspect 27, wherein the additional therapy comprises administration of an inhibitor of TET2.

Aspect 31 depends upon Aspect 30, wherein the inhibitor of TET2 comprises C35 and/or TETi76.

Aspect 32 depends upon Aspect 27, wherein the additional therapy comprises administration of an inhibitor of NSUN1 and/or NSUN2.

Aspect 33 depends upon Aspect 32, wherein the additional therapy comprises administration of an inhibitor of NSUN2.

Aspect 34 depends upon Aspect 33, wherein the inhibitor of NSUN2 comprises a compound of any one of Aspects 94 to 126.

Aspect 35 depends upon Aspect 33, wherein the inhibitor of NSUN2 comprises a compound of Aspect 125.

Aspect 36 depends upon Aspect 33, wherein the inhibitor of NSUN2 comprises a compound of Aspect 126.

Aspect 37 depends upon any one of Aspects 27 to 36, further comprising administering an inhibitor of MBD6.

Aspect 38 depends upon any one of Aspects 1 to 37, further comprising a step of diagnosing the disease in the individual.

Aspect 39 depends upon any one of Aspects 1 to 38, wherein the disease is associated with diseased cells characterized as comprising an open chromatin state relative to non-diseased cells of the same developmental lineage.

Aspect 40 depends upon any one of Aspects 1 to 39, wherein administering the one or more IKZF1, EZH2, and/or NPM1 inhibitors results in promotion of a closed chromatin state in one or more diseased cells in the individual.

Aspect 41 depends upon any one of Aspects 1 to 40, wherein the disease is characterized as pro-inflammatory.

Aspect 42 depends upon any one of Aspects 1 to 41, comprising inhibition of diseased cell proliferation.

Aspect 43 depends upon any one of Aspects 1 to 42, wherein administering the one or more IKZF1, EZH2, and/or NPM1 inhibitors decreases m⁵C levels in one or more chromatin associated RNA (caRNA) and/or decreases association of one or more caRNA with a PR-DUB complex in one or more diseased cells in the individual.

Aspect 44 depends upon Aspect 43, wherein administering the one or more IKZF1, EZH2, and/or NPM1 inhibitors increases oxidation of m⁵C in the one or more caRNA and/or inhibits installation of m⁵C in the one or more caRNA in one or more diseased cells in the individual.

Aspect 45 depends upon any one of Aspects 1 to 44, wherein histone ubiquitination is increased in one or more diseased cells after administration of the one or more IKZF1, EZH2, and/or NPM1 inhibitors.

Aspect 46 depends upon Aspect 45, wherein the histone ubiquitination comprises or consists of ubiquitination on H2A.

Aspect 47 depends upon Aspect 46, wherein the histone ubiquitination occurs at a site comprising or consisting essentially of H2AK119.

Aspect 48 depends upon any one of Aspects 1 to 47, wherein administering the one or more IKZF1, EZH2, and/or NPM1 inhibitors modifies histone methylation in one or more diseased cells in the individual.

Aspect 49 depends upon Aspect 48, wherein a histone that comprises modified methylation comprises or consists essentially of H3.

Aspect 50 depends upon Aspect 49, wherein the histone comprises modified methylation on a methylation site comprising or consisting essentially of H3K27me3.

Aspect 51 depends upon Aspect 48, wherein the histone that comprises modified methylation is localized near or at a genetic locus overlapping with a histone that comprises modified histone ubiquitination.

Aspect 52 depends upon any one of Aspects 1 to 51, wherein the one or more inhibitors of IKZF1, EZH2, and/or NPM1 comprise a polynucleotide at least partially complementary to a gene encoding IKZF1, EZH2, and/or NPM1.

Aspect 53 depends upon Aspect 52, wherein the polynucleotide comprises a short hairpin RNA and/or small interfering RNA.

Aspect 54 depends upon any one of Aspects 52 to 53, wherein the polynucleotide comprises a sequence at least 80% complementary to at least 15, 20, 25, or more than 25 contiguous nucleotides of any one or more of SEQ ID NOs: 2-3, 5-6, 8-9, 11-12, 14-15, or 17-19.

Aspect 55 depends upon any one of Aspects 52 to 54, wherein the polynucleotide comprises modified RNA phosphoramidites.

Aspect 56 depends upon any one of Aspects 52 to 55, wherein the polynucleotide comprises RNA with one or more 2′-O-Methyl (2′-OMe) or 2′-O-Methoxyethyl (2′-MOE) modifications.

Aspect 57 depends upon Aspect 56, wherein the polynucleotide comprises RNA with every nucleotide comprising a 2′-MOE modification.

Aspect 58 depends upon any one of Aspects 52 to 57, wherein the polynucleotide comprises RNA with one or more phosphorothioate bonds.

Aspect 59 depends upon any one of Aspects 52 to 58, wherein the polynucleotide is comprised within a lentiviral particle and/or nanoparticle.

Aspect 60 depends upon any one of Aspects 52 to 59, wherein the inhibitor of IKZF1, EZH2, and/or NPM1 comprises more than one polynucleotide.

Aspect 61 depends upon any one of Aspects 1 to 60, wherein the one or more inhibitors of IKZF1, EZH2, and/or NPM1 comprise a proteolysis targeting chimera.

Aspect 62 includes a method of treating a disease in an individual, wherein the disease is characterized by cells comprising one or more mutations in IKZF1, EZH2, NPM1, TET2, ASXL1, IDH1, IDH2, p53, DNMT3A, JAK2, PPM1D, SF3B1, and/or SRSF2 encoding genes and/or a rearrangement of the lysine methyltransferase 2A (KMT2A)/mixed-lineage leukemia (MLL) gene, the method comprising administering one or more inhibitors of IKZF1, EZH2, NPM1, MBD6, TET2, and/or NSUN2 to the individual in need thereof.

Aspect 63 depends upon Aspect 62, wherein the disease is characterized by cells comprising a TET2 mutation and the method comprises administering an inhibitor of NSUN2 to the subject.

Aspect 64 depends upon Aspect 62, wherein the disease is characterized by cells comprising a rearrangement of the lysine methyltransferase 2A (KMT2A)/mixed-lineage leukemia (MLL) gene and the method comprises administering an inhibitor of NSUN2 to the subject.

Aspect 65 depends upon Aspect 63 or 64, wherein the inhibitor of NSUN2 comprises a compound of any one of Aspects 94 to 126.

Aspect 66 depends upon Aspect 63 or 64, wherein the inhibitor of NSUN2 comprises a compound of Aspect 124.

Aspect 67 depends upon Aspect 63 or 64, wherein the inhibitor of NSUN2 comprises a compound of Aspect 125.

Aspect 68 depends upon Aspect 63 or 64, wherein the inhibitor of NSUN2 comprises a compound of Aspect 126.

Aspect 69 includes a method of treating a disease in an individual, comprising administering 5-AzaC and one or more inhibitors IKZF1, EZH2, and/or NPM1, to an individual in need thereof.

Aspect 70 includes a method of treating a disease in an individual, comprising determining the gene expression levels and/or protein activity levels of one or more of IKZF1, EZH2, and/or NPM1 in diseased cells in the individual, and administering 5-AzaC to the individual if the expression levels and/or protein activity levels of IKZF1, EZH2, and/or NPM1 are reduced relative to expression levels in a non-diseased cell of the same developmental lineage in the individual.

Aspect 71 includes a method of treating a disease in an individual, comprising administering one or more inhibitors of NSUN2 to the individual in need thereof.

Aspect 72 depends upon Aspect 71, wherein the inhibitor of NSUN2 comprises a compound of any one of Aspects 94 to 126.

Aspect 73 depends upon Aspect 71, wherein the inhibitor of NSUN2 comprises a compound of Aspect 124.

Aspect 74 depends upon Aspect 71, wherein the inhibitor of NSUN2 comprises a compound of Aspect 125.

Aspect 75 depends upon Aspect 71, wherein the inhibitor of NSUN2 comprises a compound of Aspect 126.

Aspect 76 depends upon any one of Aspects 58 to 75, wherein the disease is characterized by cells comprising one or more mutations in IKZF1, EZH2, NPM1, TET2, ASXL1, IDH1, IDH2, p53, DNMT3A, JAK2, PPM1D, SF3B1, and/or SRSF2 encoding genes; and/or a rearrangement of a lysine methyltransferase 2A (KMT2A)/mixed-lineage leukemia (MLL) gene.

Aspect 77 depends upon Aspect 76, wherein the disease is characterized by cells comprising a mutation in a TET2 encoding gene.

Aspect 78 depends upon Aspect 76, wherein the disease is characterized by cells comprising a rearrangement of a lysine methyltransferase 2A (KMT2A)/mixed-lineage leukemia (MLL) gene.

Aspect 79 depends upon any one of Aspects 62 to 78, wherein the disease comprises cancer of the blood, lung, brain, breast, skin, pancreas, liver, colon, head and neck, kidney, thyroid, stomach, spleen, gallbladder, bone, ovary, testes, endometrium, prostate, rectum, anus, and/or cervix.

Aspect 80 depends upon any one of Aspects 62 to 79, wherein the disease comprises clonal hematopoiesis of indeterminate potential (CHIP).

Aspect 81 depends upon any one of Aspects 62 to 80, wherein the disease is characterized by atherosclerosis, myocardial fibrosis, and/or heart failure.

Aspect 82 depends upon any one of Aspects 62 to 81, wherein the cancer comprises a blood cancer.

Aspect 83 depends upon Aspect 82, wherein the blood cancer comprises a leukemia.

Aspect 84 depends upon Aspect 82, wherein the blood cancer comprises a myeloid malignancy.

Aspect 85 depends upon Aspect 84, wherein the myeloid malignancy comprises acute myeloid leukemia.

Aspect 86 depends upon Aspect 84, wherein the myeloid malignancy comprises chronic myelomonocytic leukemia.

Aspect 87 depends upon Aspect 84, wherein the myeloid malignancy comprises myelodysplastic syndrome.

Aspect 88 depends upon Aspect 79, wherein the cancer comprises a glioma.

Aspect 89 depends upon Aspect 88, wherein the glioma comprises glioblastoma.

Aspect 90 depends upon any one of Aspects 1 to 88, wherein the one or more inhibitors of EZH2 comprises Tazemetostat, CPI-1205, GSK2816126, PF-06821497, SHR2554, and/or DS3201b.

Aspect 91 depends upon any one of Aspects 1 to 88, wherein the one or more inhibitors of IKZF1 comprises CC-92480.

Aspect 92 includes a manufactured article for use in performing the method of any one of Aspects 1 to 91.

Aspect 93 includes a kit comprising means for performing the method according to any one of Aspects 1 to 92.

Aspect 94 includes a compound of Formula (I) or pharmaceutical appropriate salt thereof, wherein Formula (I) is Ar-L-B, Ar is a heteroaryl moiety or an aryl moiety substituted with a heteroaryl substituent, wherein Ar is optionally substituted with 1 to 3 R_{y} substituents, each R_{y} independently selected from halogen, cyano, nitro, alkyl, haloalkyl, alkoxy, haloalkoxy, alkoxycarbonyl, carboxy(C1-C6)alkoxy, aryl, heteroaryl, aryloxy, and aralkoxy, wherein the heteroaryl moiety or the heteroaryl substituent comprises at least one ring heteroatom selected from nitrogen, oxygen, and sulfur, and when the heteroaryl moiety or the heteroaryl substituent comprises a ring nitrogen atom, the ring nitrogen atom is unsubstituted or substituted with R_{N}, wherein R_{N} is selected from SO2R_{X}, C(═O)R_{X}, and C(═O)OR_{X}, and wherein R_{X} is selected from substituted or unsubstituted C1-C6 alkyl, substituted or unsubstituted aryl, substituted or unsubstituted heteroaryl, and substituted or unsubstituted heterocyclyl; L is —CH═, wherein the carbon atom of L is singly bonded to Ar and doubly bonded to a carbon atom of B; and B is selected from a barbiturate moiety, a thiobarbiturate moiety, a Meldrum's acid moiety, a thiazolidinedione moiety, a pyrazolone moiety, an indane-1,3-dione moiety, a cyanoacrylic acid moiety, a cyanoacrylate moiety, and an aryl-substituted acrylonitrile moiety.

Aspect 95 depends upon Aspect 94, wherein B is a barbiturate moiety or a thiobarbiturate moiety.

Aspect 96 depends upon Aspect 95, wherein B is a pyrimidine-2,4,6(1H,3H,5H)-trione moiety or a 2-thioxodihydropyrimidine-4,6(1H,5H)-dione moiety.

Aspect 97 depends upon Aspect 96, wherein B is

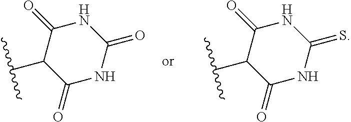 [SMILES: *C1C(=O)NC(=O)NC1=O ; *C1C(=O)NC(=S)NC1=O]

Aspect 98 depends upon Aspect 94, wherein B is a Meldrum's acid moiety, a thiazolidinedione moiety, a pyrazolone moiety, an indane-1,3-dione moiety, a cyanoacrylic acid moiety, a cyanoacrylate moiety, or an aryl-substituted acrylonitrile moiety.

Aspect 99 depends upon Aspect 98, wherein B is

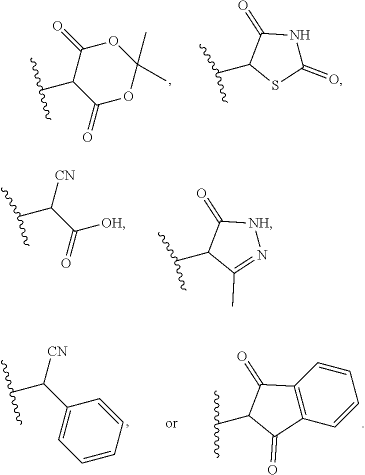

Aspect 100 depends upon any one of Aspects 94 to 99, wherein Ar is a heteroaryl moiety.

Aspect 101 depends upon Aspect 100, wherein Ar is a fused heteroaryl moiety comprising nitrogen.

Aspect 102 depends upon Aspect 101, wherein Ar is an indolyl moiety or a pyrrolopyridinyl moiety.

Aspect 103 depends upon Aspect 102, wherein Ar is an indol-3-yl moiety, an indol-2-yl moiety, an indol-4-yl moiety, an indol-5-yl moiety or an indol-6-yl moiety.

Aspect 104 depends upon Aspect 99, wherein Ar is a fused heteroaryl moiety comprising sulfur.

Aspect 105 depends upon Aspect 104, wherein Ar is a benzothiophenyl moiety.

Aspect 106 depends upon Aspect 99, wherein Ar is a fused heteroaryl moiety comprising oxygen.

Aspect 107 depends upon Aspect 106, wherein Ar is a dioxolo-fused indolyl moiety.

Aspect 108 depends upon Aspect 99, wherein Ar is a pyrrolyl moiety or a thiazolyl moiety.

Aspect 109 depends upon any one of Aspects 94 to 99, wherein Ar is an aryl moiety substituted with a heteroaryl substituent.

Aspect 110 depends upon Aspect 109, wherein Ar is a phenyl moiety substituted with a thiazolyl moiety.

Aspect 111 depends upon any one of Aspects 94 to 110, wherein the R_{N} substituent is —SO₂R_{X}.

Aspect 112 depends upon any one of Aspects 94 to 110, wherein the R_{N} substituent is C(═O)R_{X}.

Aspect 113 depends upon any one of Aspects 94 to 110, wherein the R_{N} substituent is C(═O)OR_{X}.

Aspect 114 depends upon any one of Aspects 111 to 113, wherein R_{X} is aryl, heteroaryl, or heterocyclyl.

Aspect 115 depends upon Aspect 114, wherein the aryl is phenyl or naphthyl.

Aspect 116 depends upon Aspect 114, wherein R_{X} is benzo[d][1,3]dioxol-5-yl.

Aspect 117 depends upon Aspect 114, wherein the heterocyclyl is a nitrogen-containing heterocyclyl.

Aspect 118 depends upon Aspect 117, wherein the heterocyclyl is substituted on a ring nitrogen atom with tert-butoxycarbonyl.

Aspect 119 depends upon Aspect 117 or 118, wherein the heterocyclyl is piperidinyl.

Aspect 120 depends upon Aspect 114, wherein R_{X} is substituted with 1 to 3 substituents independently selected from halogen, cyano, alkyl, alkoxy, haloalkyl, haloalkoxy, and aryloxy.

Aspect 121 depends upon any one of Aspects 111 to 113, wherein R_{X} is substituted or unsubstituted C1-C6 alkyl.

Aspect 122 depends upon Aspect 121, wherein Rx is tert-butyl.

Aspect 123 depends upon any one of Aspects 94 to 122, wherein at least one Ry substituent is selected from trifluoromethyl, trifluoromethoxy, methoxycarbonyl, benzyloxy, nitro, phenyl, furyl, methoxy, chloro, cyano, methyl, and phenoxy.

Aspect 124 depends upon any one of Aspects 94 to 123, wherein the compound comprises a structure selected from:

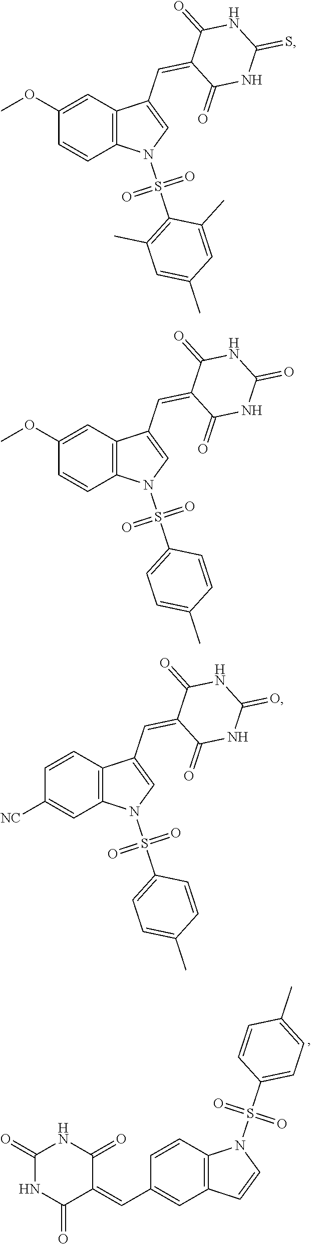

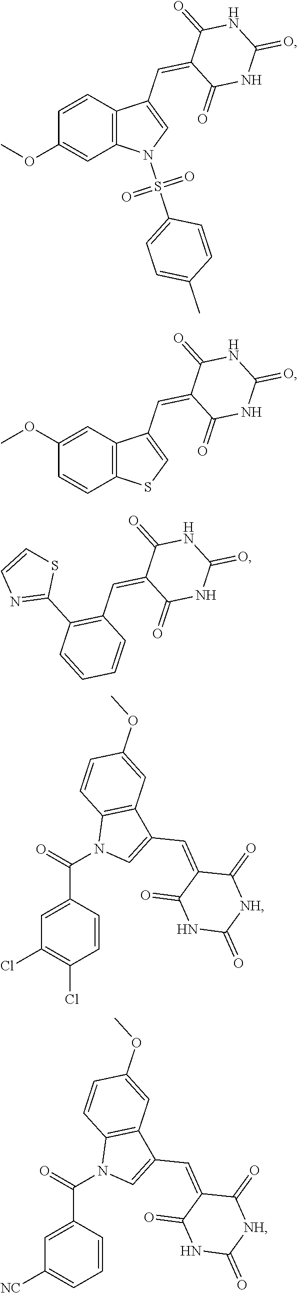

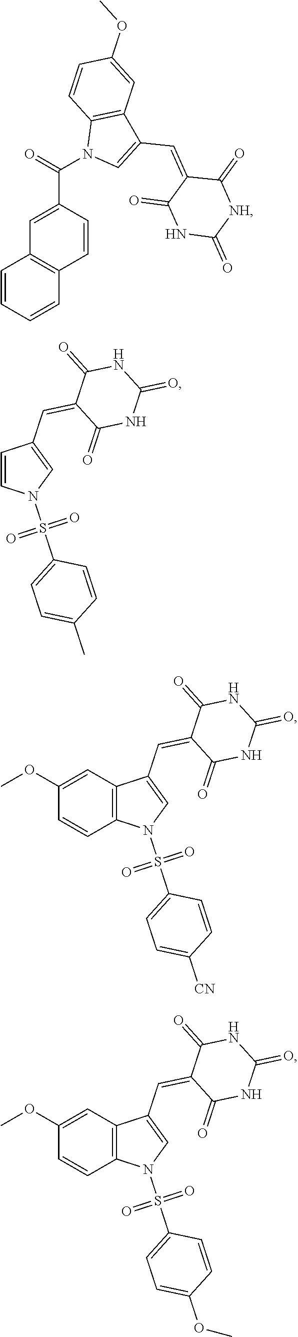

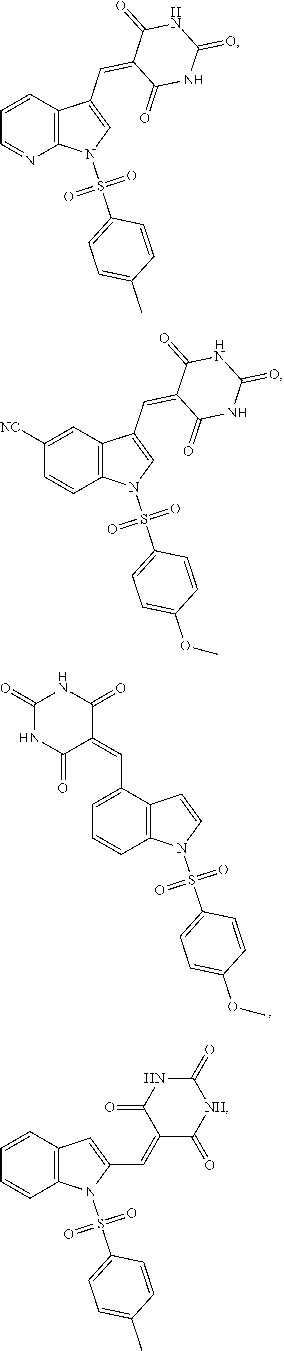

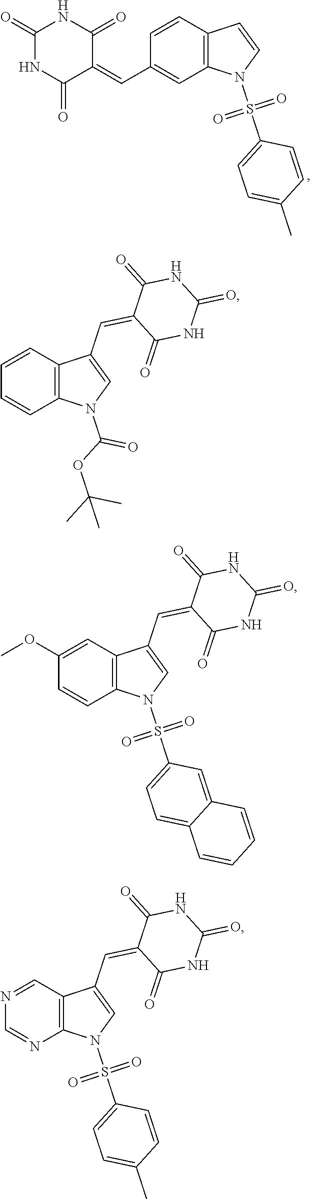 [SMILES: Cc1ccc(S(=O)(=O)n2ccc3ccc(C=C4C(=O)NC(=O)NC4=O)cc32)cc1 ; CC(C)(C)OC(=O)n1cc(C=C2C(=O)NC(=O)NC2=O)c2ccccc21 ; COc1ccc2c(c1)c(C=C1C(=O)NC(=O)NC1=O)cn2S(=O)(=O)c1ccc2ccccc2c1 ; Cc1ccc(S(=O)(=O)n2cc(C=C3C(=O)NC(=O)NC3=O)c3cncnc32)cc1]

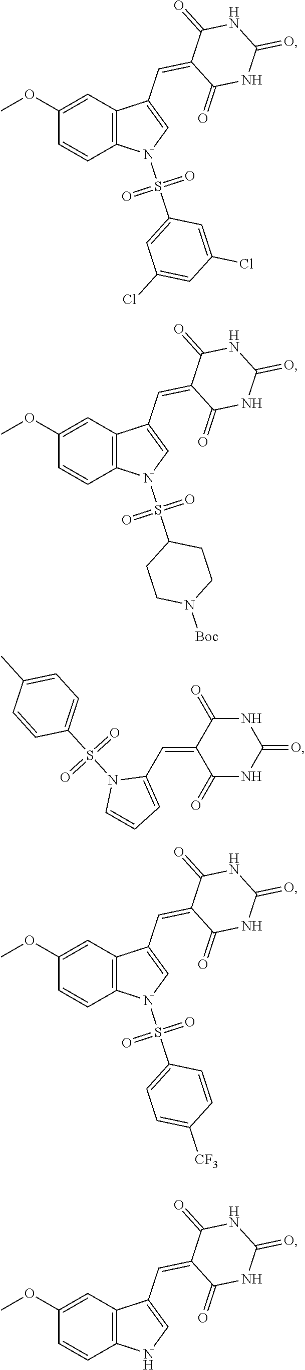 [SMILES: COc1ccc2c(c1)c(C=C1C(=O)NC(=O)NC1=O)cn2S(=O)(=O)c1cc(Cl)cc(Cl)c1 ; COc1ccc2c(c1)c(C=C1C(=O)NC(=O)NC1=O)cn2S(=O)(=O)C1CCN(C(=O)OC(C)(C)C)CC1 ; Cc1ccc(S(=O)(=O)n2cccc2C=C2C(=O)NC(=O)NC2=O)cc1 ; COc1ccc2c(c1)c(C=C1C(=O)NC(=O)NC1=O)cn2S(=O)(=O)c1ccc(C(F)(F)F)cc1 ; COc1ccc2[nH]cc(C=C3C(=O)NC(=O)NC3=O)c2c1]

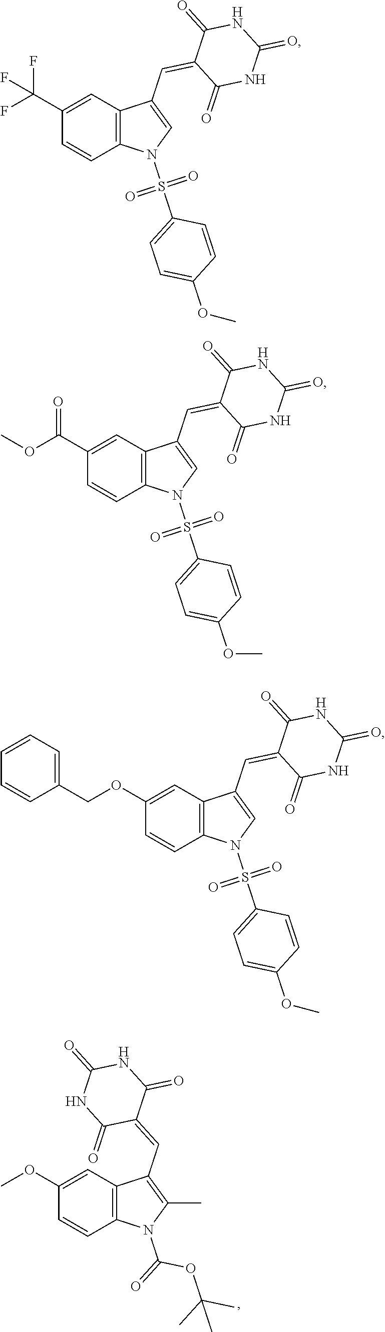

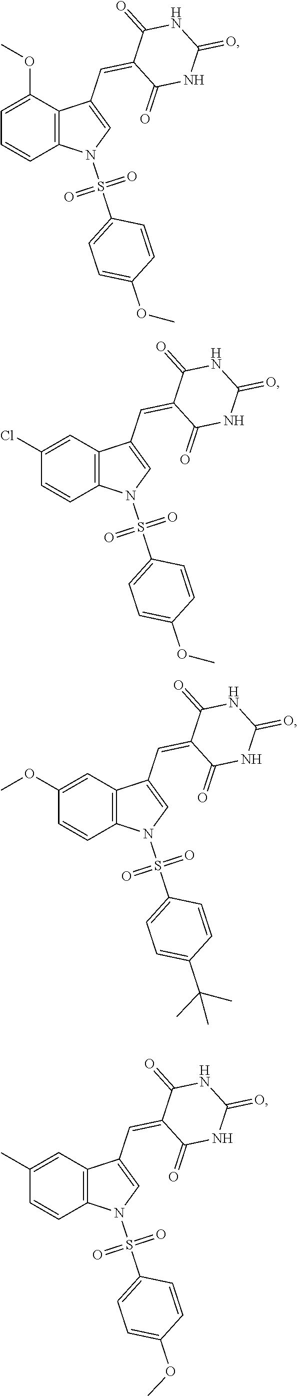

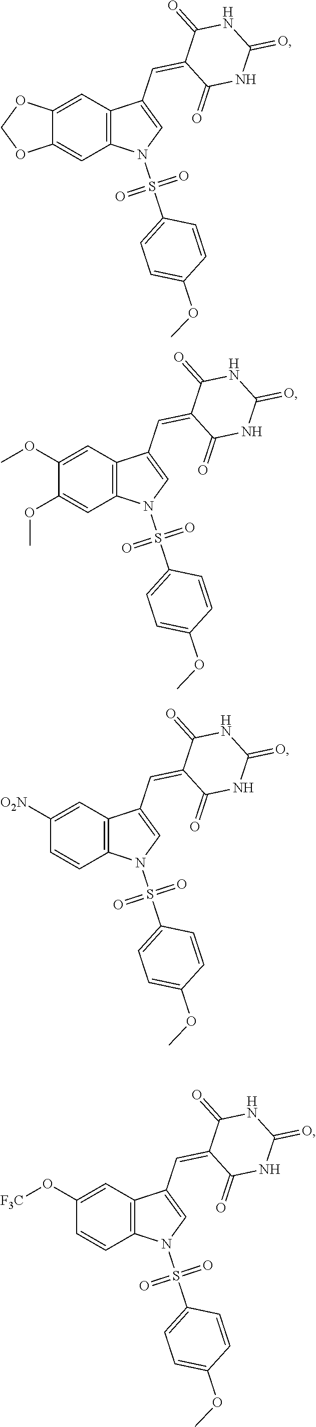

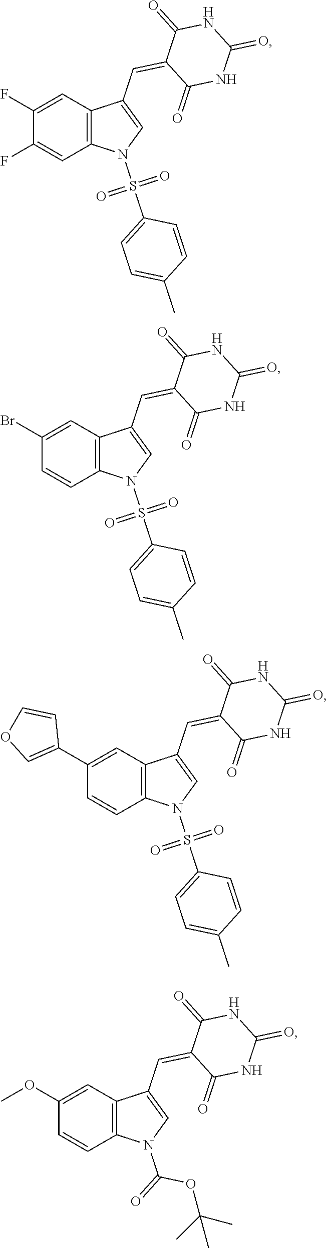

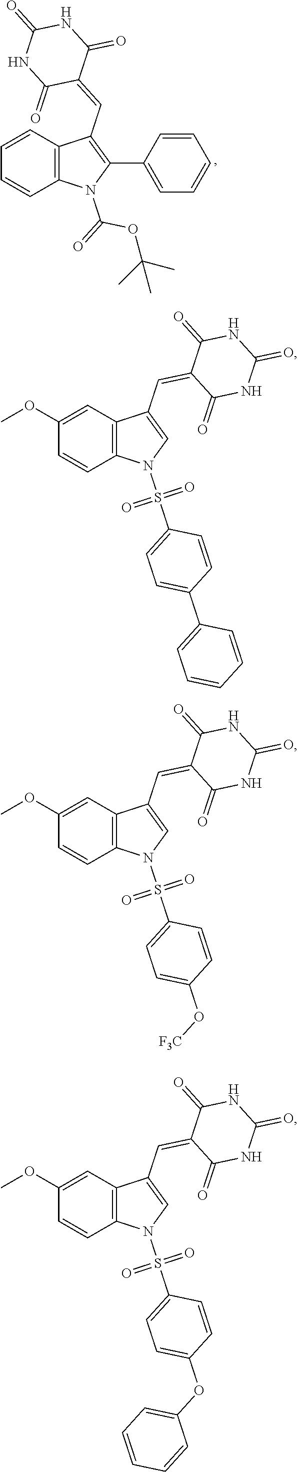

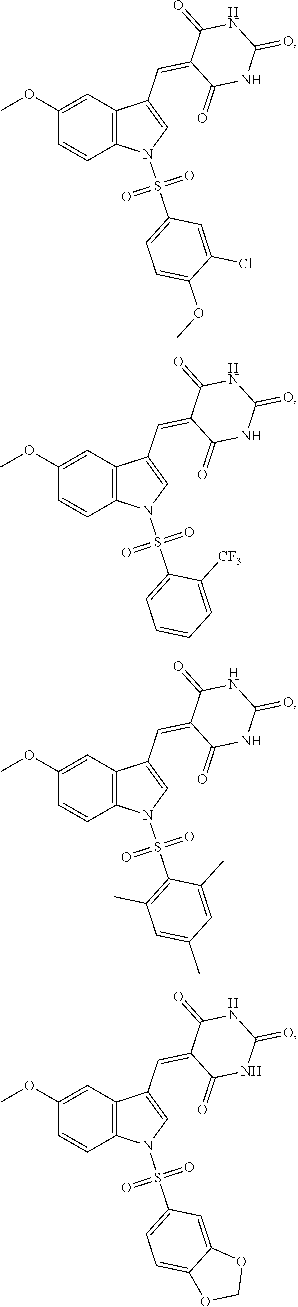

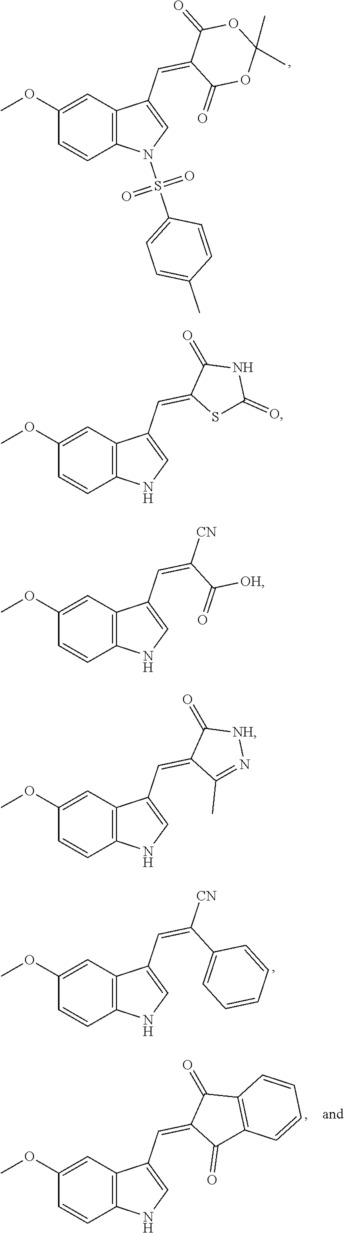

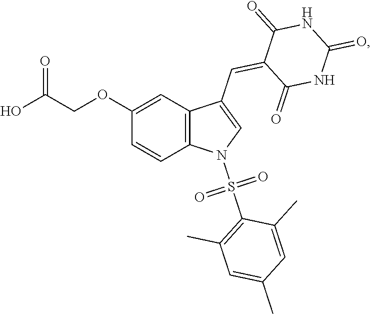 [SMILES: Cc1cc(C)c(S(=O)(=O)n2cc(C=C3C(=O)NC(=O)NC3=O)c3cc(OCC(=O)O)ccc32)c(C)c1]

or a pharmaceutically appropriate salt thereof.

Aspect 125 includes a compound having a structure selected from:

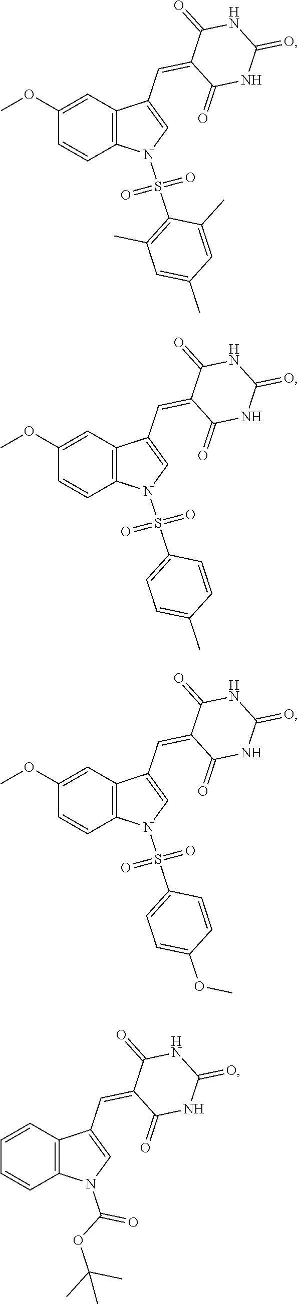

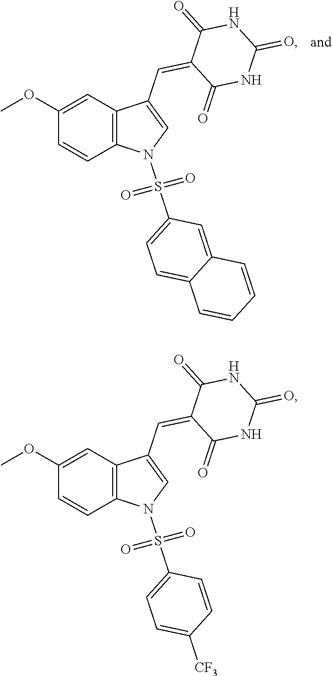 [SMILES: COc1ccc2c(c1)c(C=C1C(=O)NC(=O)NC1=O)cn2S(=O)(=O)c1ccc2ccccc2c1 ; COc1ccc2c(c1)c(C=C1C(=O)NC(=O)NC1=O)cn2S(=O)(=O)c1ccc(C(F)(F)F)cc1]

or a pharmaceutically appropriate salt thereof.

Aspect 126 includes a compound comprising the structure of

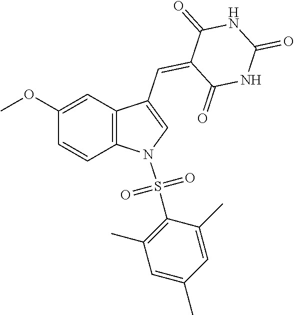 [SMILES: COc1ccc2c(c1)c(C=C1C(=O)NC(=O)NC1=O)cn2S(=O)(=O)c1c(C)cc(C)cc1C]

or a pharmaceutically appropriate salt thereof.

Aspect 127 includes a pharmaceutical composition comprising a compound of any one of Aspects 94 to 126 and at least one pharmaceutically appropriate carrier or excipient.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. Inventions described herein may be better understood by reference to one or more of these drawings in combination with the detailed description of specific aspects presented herein.

FIGS. 1A-1M, 5-azaC induced RNA m⁵C depletion and leukemia cell death at early time points. FIG. 1A Changes in m⁵C/A levels in caRNA and tRNA, and 5mC/A levels in gDNA after 3 μM 5-azaC treatment in MOLM13. Modification levels were normalized to controls. FIGS. 1B-1E Cell proliferation of MOLM13 (1B), SKM-1 (1C), THP1 (1D) and K562 (1E) after 5-azaC or decitabine treatment, measured using CellTiter-Glo®. FIG. 1F GSEA of MSigDB cell cycle gene set in 5-azaC-treated MOLM13. FIG. 1G Cell cycle distribution of MOLM13 cells after 16 hours of 5-azaC or decitabine treatment. FIG. 1H GSEA of MSigDB DNA repair gene set in 5-azaC-treated MOLM13. FIG. 1I EdU incorporation during DNA damage repair after UV irradiation in MOLM13 treated with 3 μM 5-azaC or decitabine for 16 hours. Only G1-phase populations are shown. Blk, no irradiation. FIG. 1J GSEA of MSigDB apoptosis gene set in 5-azaC-treated MOLM13. FIG. 1K TUNEL fluorescent signals in MOLM13 cells following 3 μM 5-azaC or decitabine treatment. FIG. 1L Cell proliferation of MOLM13 after 3 μM 5-azaC or decitabine treatment in combination with 0.2 nM venetoclax for 24 hours. HMA, hypomethylating agent. FIG. 1M GSEA of myeloid cell differentiation gene set in 5-azaC-treated LK cells.

FIGS. 2A-2H, 5-azaC induced transcriptional repression through caRNA m⁵C depletion. FIG. 2A EU fluorescent signals in MOLM13 cells after 3 μM 5-azaC treatment. FIG. 2B EU fluorescent signals in AML patient samples after 3 μM 5-azaC treatment. FIG. 2C Nascent RNA-seq of MOLM13 cells after 3 μM 5-azaC treatment. DEGs are defined as |log 2 fold change (5-azaC/Ctrl)|>1 and adj.p-value <0.05. DEG numbers are annotated on the plot. Adj.p-value, adjusted p-value. FIGS. 2D-2E GSEA of transcriptional changes in cell cycle (2D) and DNA repair (2E) gene sets after 16 hours of 5-azaC treatment in MOLM13. FIG. 2F m⁵C enrichment across repeat families in control MOLM13. Circle sizes represent normalized read counts from MeRIP-seq. The dashed line indicates a value of 1. FIG. 2G Differential m⁵C enrichment across repeat families in MOLM13 after 16 hours of 3 μM 5-azaC treatment. Circle sizes represent normalized MeRIP-seq read counts in untreated samples. The dashed line indicates a value of −0.58. FIG. 2H Differential levels of caRNA transcripts from genes adjacent to m⁵C sites (m⁵C) versus other genes (Others).

FIGS. 3A-3F, 5-azaC induced transcriptional repression through m⁵C-dependent H2AK119ub regulation. FIG. 3A Immunoblotting of H2AK119ub changes after 16-hour 3 μM 5-azaC treatment in MOLM13. FIG. 3B Integrative Genome Viewer (IGV) tracks showing m⁵C MeRIP-seq, H2AK119ub CUT&Tag and MBD6 CLIP-seq signals at the TINAGL1 locus after 16-hour 3 μM 5-azaC treatment. Track scales are indicated in brackets. FIG. 3C Metaplot and heatmap of H2AK119ub level changes at m⁵C sites after 5-azaC treatment. FIG. 3D H2AK119ub changes across repeat families. Circle sizes represent normalized CUT&Tag read counts in control samples. The dashed line indicates a value of 0.58. FIG. 3E Immunoblotting of H2AK119ub changes following doxycycline (Dox)-induced NSUN2 depletion for 5 days in MOLM13. FIG. 3F MOLM13 proliferation following 5-azaC treatment after 48-hour shRNA-mediated MBD6 depletion. Time points indicate hours after 5-azaC treatment.

FIGS. 4A-4E, 5-azaC induced transcriptional repression through H3K27ac downregulation. FIG. 4A Immunoblotting of H3K27ac changes in SKM1 after 16-hour 3 μM 5-azaC treatment. FIG. 4B Metaplot and heatmap of H3K27ac changes across all annotated genes after 16-hour 3 μM 5-azaC treatment in SKM1. FIG. 4C Metaplot of H3K27ac changes in cell cycle and DNA repair gene sets in SKM1. FIG. 4D H3K27ac (left) and nascent RNA-seq (right) changes in genes grouped into four quantiles based on fold changes of nearby H3K27ac peaks in SKM1. Q1 represents the quantile with the most negative fold change. FIG. 4E Immunoblotting of H3K27ac changes after 5-day Dox-induced depletion of NSUN2 in SKM1.

FIGS. 5A-5G, 5-azaC downregulated H3K27ac through SRSF2-mediated p300 recruitment. FIG. 5A Immunoblotting of H3K27ac changes after 48-hour shRNA-mediated SRSF2 depletion in SKM1. FIG. 5B Co-immunofluorescence of SRSF2 and p300 in SKM1. Scale bar: 5 μm. FIG. 5C Quantification of SRSF2-p300 colocalization along the marked line in (5B). FIG. 5D Proximity ligation assay (PLA) of SRSF2 and p300 in SKM1. Scale bar: 5 μm. FIG. 5E Metaplot and heatmap of SRSF2 and H3K27ac changes at SRSF2 target sites after 5-azaC treatment. FIG. 5F Metaplot and heatmap of SRSF2 CUT&Tag level changes at downregulated SRSF2 peaks after 5-azaC treatment. Samples were treated with Dox for 5-day NSUN2 depletion. FIG. 5G IGV tracks showing m⁵C MeRIP-seq, SRSF2 CUT&Tag, and H3K27ac CUT&Tag signals at genomic locus near PEG10 after 16-hour 5-azaC treatment or Dox-induced NSUN2 depletion. Track scales are indicated in brackets.

FIGS. 6A-6L, NSUN2 regulated leukemia proliferation and hematopoietic lineage commitment. FIG. 6A Cell proliferation of MOLM13 cells after Dox-induced NSUN2 depletion, measured using CellTiter-Glo®. FIG. 6B GSEA of MSigDB cell cycle gene set in MOLM13 after 5-day Dox-induced NSUN2 depletion. FIG. 6C Cell cycle distribution of MOLM13 cells after 5-day Dox-induced NSUN2 depletion. FIG. 6D GSEA of MSigDB DNA repair gene set in MOLM13 after 5-day Dox-induced NSUN2 depletion. FIG. 6E Immunoblotting of DNA repair genes after 5-day Dox-induced NSUN2 depletion. FIG. 6F Immunoblotting of γH2A.X 5-day Dox-induced NSUN2 depletion followed by 50 J/m² UVC irradiation. FIG. 6G EdU incorporation during DNA damage repair after UVC irradiation in MOLM13 after 5-day Dox-induced NSUN2 depletion. Only G0/G1-phase populations are shown. Blk, no irradiation. FIG. 6H Flow cytometry analysis of Lin⁻Sca1⁺c-Kit⁺ (LSK), Lin⁻Sca1⁻c-Kit⁺ (LKS⁻), granulocyte-monocyte progenitor (GMP), megakaryocyte-erythroid progenitor (MEP), and common myeloid progenitor (CMP) populations in bone marrow (BM) from 6-month-old wild-type (WT) and Nsun2−/− mice (left), and quantification of LSK, CMP and GMP populations in the total BM cells (right). N=5 mice/genotype. FIG. 6I Flow cytometry analysis of erythroid cells in the spleen of 6-month-old WT and Nsun2−/− mice (left), and quantification of the Ter119⁺ erythroblasts (right). R1: pro-, R2: basophilic-, R3: late basophilic- and polychromatophilic-, and R4: orthochromatophilic-erythroblasts. FIG. 6J CFU-C assay of BM cells from 6-month-old WT and Nsun2−/− mice. FIG. 6K Representative GEMM colonies formed by BM cells from 6-month-old WT and Nsun2−/− mice. Scale bar, 200 μm. FIG. 6L Quantification of Ter119⁺ erythroid cells percentage at different time points during in vitro erythroid differentiation using the hematopoietic stem and progenitor LK cells derived from mouse BM cells (n=3).

FIGS. 7A-7G, Screening of leukemia cell lines identified mutations associated with 5-azaC sensitivity. FIG. 7A Proliferation of 26 leukemia cell lines after 24-hour treatment with 5-azaC (left) or decitabine (middle), along with their reported mutations from public databases (right). FIG. 7B Proliferation of THP1 WT and TET2 KO cells after 5-azaC treatment. FIG. 7C Proliferation of K562 cells treated with 5-azaC after Dox-induced IKZF1 depletion (GCCGAAGCTATAAACAGCGAACTCGAGTTCGCTGTTTATAGCTTCGGC—shRNA, SEQ ID NO: 19). Time points indicate hours after 5-azaC treatment. Proliferation was measured by CellTiter-Glo®. FIG. 7D Proliferation of NB4 cells treated with 5-azaC after Dox-induced IKZF1 depletion (GCCGAAGCTATAAACAGCGAACTCGAGTTCGCTGTTTATAGCTTCGGC—shRNA, SEQ ID NO: 19). Time points indicate hours after 5-azaC treatment. Proliferation was measured by CellTiter-Glo®. FIG. 7E PCA of RNA-seq of K562 cells with Dox-induced IKZF1 depletion and/or a 24-hour 5-azaC (3 μM) treatment FIG. 7F Differential expression of Sens-up, Sens-down and other genes (Others) in K562 cells with 5-azaC (3 μM) treatment without IKZF1 depletion. The lilac circle represents value of mean in each group of genes. FIG. 7G Differential expression of Sens-up, Sens-down and other genes (Others) in K562 cells with Dox-induced IKZF1 depletion without 5-azaC treatment. Circles represent value of mean in each group of genes.

FIGS. 8A-8J, 5-azaC modulated cell cycle, DNA repair and apoptosis mediated by RNA m⁵C. FIG. 8A Volcano plot of RNA-seq from MOLM13 cells after 24-hour 5-azaC treatment. DEGs are defined as |log₂ fold change|>0.58 and adj.p-value <0.05. DEG numbers are annotated on the plot. Adj.p-value, adjusted p-value. FIG. 8B GO analysis of the top enriched biological processes in DEGs after 5-azaC treatment in MOLM13. The top 20 GO terms enriched in downregulated genes and all GO terms enriched in upregulated genes with FDR<0.05 were plotted. FIG. 8C Volcano plot of RNA-seq from SKM1 cells after 24-hour 5-azaC treatment. DEGs are defined as |log₂ fold change|>0.58 and adj.p-value <0.05. DEG numbers were annotated on the plot. Adj.p-value, adjusted p-value. FIG. 8D GO analysis of the top enriched biological processes in DEGs after 5-azaC treatment in SKM1. The top 20 GO terms enriched in downregulated genes and the top 9 GO terms enriched in upregulated genes are plotted. FIG. 8E Cell cycle distribution of SKM1 cells after 16 hours of 5-azaC or decitabine treatment. FIG. 8F Immunoblotting of DNA repair genes after 3 μM 5-azaC treatment. FIG. 8G Immunoblotting of γH2A.X after 3 μM 5-azaC treatment followed by 50 J/m² UVC irradiation. FIG. 8H EdU incorporation during DNA damage repair after UVC irradiation in MOLM13 treated with 3 μM 5-azaC or decitabine for 16 hours. Only G2/M-phase populations are shown. Blk, no irradiation. FIG. 8I GSEA of MSigDB cell cycle gene set in 5-azaC-treated Lin⁻c-Kit⁺ (LK) cells. FIG. 8J GSEA of MSigDB DNA repair gene set in 5-azaC-treated LK cells.

FIGS. 9A-90, 5-azaC induced transcriptional repression through caRNA m⁵C depletion. FIGS. 9A-9E EU fluorescent signals in SKM1 (9A), HEL (9B), OCI-AML3 (9C), HL60 (9D), and THP1 (9E) cells after 3 μM 5-azaC treatment. FIG. 9F Nascent RNA-seq of SKM1 cells after 3 μM 5-azaC treatment. DEGs are defined as |log₂ fold change (5-azaC/Ctrl)|>1 and adj.p-value <0.05. DEG numbers are annotated on the plot. Adj.p-value, adjusted p-value. FIGS. 9G-9H GSEA of transcriptional changes in cell cycle (9G) and DNA repair (9H) gene sets after 16 hours of 5-azaC treatment in SKM1. FIGS. 9I-9J EdU incorporation during DNA damage repair after UV irradiation in MOLM13 treated with 3 μM 5-azaC for 16 hours. Samples annotated with ActD were treated with 1 μg/mL ActD for 2 hours prior to irradiation. Only G0/G1- (9I) and G2/M- (9J) phase populations are shown. Blk, no irradiation. FIG. 9K Metaplot of m⁵C MeRIP-seq signal changes at m⁵C sites from 5-azaC treated and non-treated MOLM13. FIG. 9L Percentage of sequencing reads mapped to repetitive elements (Repeats) or not (Others) in input and MeRIP samples from control MOLM13 cells. The percentage is the average of three replicates. FIG. 9M Metaplot of m⁵C MeRIP-seq signal changes at m⁵C sites from 5-azaC treated and non-treated SKM1. FIG. 9N Percentage of sequencing reads mapped to repetitive elements (Repeats) or not (Others) in input and MeRIP samples from control SKM1 cells. The percentage is the average of three replicates. FIG. 9O Differential levels of caRNA repeat subfamilies in MOLM13 after 16-hour 5-azaC treatment.

FIGS. 10A-10E, 5-azaC induced transcriptional repression through m⁵C-dependent H2AK119ub regulation. FIGS. 10A-10B Metaplot of H2AK119ub changes across all annotated genes (10A) or specific gene sets (10B) after 16-hour 5-azaC treatment in MOLM13. FIG. 10C Metaplot and heatmap of MBD6 CLIP-seq signal changes at m⁵C sites that are downregulated after 16-hour 5-azaC treatment in MOLM13. FIG. 10D Knockdown efficiency of m⁵C methyltransferases and DNMT1 after 5-day Dox-induced depletion of the respective gene. Expression levels were first normalized to ACTB, followed by normalization to the respective controls. FIG. 10E Immunoblotting of H2AK119ub changes following Dox-induced depletion of m⁵C methyltransferases and DNMT1 for 5 days in MOLM13.

FIGS. 11A-11K, 5-azaC induced transcriptional repression through H3K27ac downregulation. FIG. 11A Immunoblotting of H3K27ac changes in MOLM13 treated with 3 μM 5-azaC treatment. FIG. 11B Immunoblotting of H3K27ac changes in SKM1 treated with 1 μM 5-azaC treatment, with 5-azaC replenished every 24 hours. FIG. 11C Metaplot and heatmap of H3K27ac level changes across all annotated genes after 16-hour 3 μM 5-azaC treatment in MOLM13. FIG. 11D Metaplot of H3K27ac changes in cell cycle and DNA repair gene sets in MOLM13. FIG. 11E H3K27ac (left) and nascent RNA-seq (right) changes in genes grouped into four quantiles based on fold changes of nearby H3K27ac peaks in MOLM13. Q1 represents the quantile with the most negative fold change. FIGS. 11F-11G H3K27ac changes across repeat families in SKM1 (11F) and MOLM13 (11G). Circle sizes represent normalized CUT&Tag read counts in control samples. The dashed line indicates a value of −0.58. FIGS. 11H-11I Metaplot of H3K27ac level changes at m⁵C sites from the corresponding cell lines after 5-azaC treatment in SKM1 (11H) and MOLM13 (11I). FIG. 11J Immunoblotting of H3K27ac changes following Dox-induced depletion of RNA m⁵C methyltransferases and DNMT1 in MOLM13. FIG. 11K Metaplot of H3K27ac level changes at m⁵C sites after 5-day Dox-induced NSUN2 depletion in SKM1.

FIGS. 12A-12H, 5-azaC downregulated H3K27ac through SRSF2-mediated p300 recruitment. FIG. 12A Immunoblotting of H3K27ac changes after 48-hour shRNA-mediated SRSF2 depletion in MOLM13. FIG. 12B Immunoblotting of SRSF2 levels across different cell lines. The samples were loaded with the same cell number. FIG. 12C Immunoprecipitation of SRSF2 with different antibodies: Abc (SRSF2 antibody ab204916), Ptg (SRSF2 antibody 20371-1-AP), Sig (SRSF2 antibody S4045). FIG. 12D Immunoblotting of SRSF2-Myc overexpression in K562 (upper) and Myc-tag CLIP followed by end ligation of pCp-AF488 fluorophore (lower). FIG. 12E Metaplot of SRSF2 CUT&Tag signal changes in cell cycle (upper) and DNA repair (lower) gene sets in SKM1. FIG. 12F Metaplot and heatmap of SRSF2 CUT&Tag signal changes at m⁵C sites after 16-hour 3 μM 5-azaC treatment in SKM1. FIGS. 12G-12H Metaplot of SRSF2 CUT&Tag signal changes across all annotated genes (12G) or specific gene sets (12H) following 5-day Dox-induced NSUN2 depletion in SKM1.

FIGS. 13A-13N, NSUN2 regulated leukemia proliferation and hematopoietic lineage commitment. FIG. 13A Cell cycle distribution of SKM1 cells after 5-day Dox-induced NSUN2 depletion. FIG. 13B EdU incorporation during DNA damage repair after UVC irradiation in MOLM13 after 5-day Dox-induced NSUN2 depletion. Only G2/M-phase populations are shown. Blk, no irradiation. FIG. 13C Cell proliferation of HEL with overexpression of NSUN2 followed by 24-hour 5-azaC treatment. FIG. 13D Deletion efficiency of Nsun2−/− mice. FIG. 13E Flow cytometry analysis of Lin⁻c-Kit⁺ (LK) cell population in progeny cells from WT and Nsun2−/− CFU-C assays shown in FIG. 6D. FIG. 13F Flow cytometry analysis of erythroid cell population in progeny cells from WT and Nsun2−/− CFU-C assays shown in FIG. 6D. FIG. 13G Quantification of CD45.2⁺ cells in the PB of recipient mice at different time points after transplantation. FIG. 13H Quantification of CD45.2⁺ cells in the BM of recipient mice at 32 weeks after transplantation. FIG. 13I Quantification of donor-derived GMP population in the BM of recipient mice at 32 weeks after transplantation. FIG. 13J Percentages of donor-derived lineage population in the PB of recipient mice at 32 weeks after transplantation, including myeloid (Gr-1⁺/CD11b⁺) cells, erythroid (Ter119⁺) cells, T (CD4⁺ and CD8⁺) cells and B (B220⁺) cells. FIG. 13K Percentages of donor-derived lineage population in the BM of recipient mice at 32 weeks after transplantation, including myeloid (Gr-1⁺/CD11b⁺) cells, erythroid (Ter119⁺) cells, T (CD4⁺ and CD8⁺) cells and B (B220⁺) cells. FIG. 13L-N Flow cytometry analyses of Ter119⁺ erythroid cells during in vitro erythroid differentiation (upper left), quantification of Ter119⁺ erythroid cells (upper right), and the representative images of the cell morphology stained by Wright-Giemsa following cytospin (lower). Scale bar, 20 μm. FIG. 13L shows flow cytometry analyses of Ter119⁺ erythroid cells at day 2. FIG. 13M shows flow cytometry analyses of Ter119⁺ erythroid cells at day 4. FIG. 13N shows flow cytometry analyses of Ter119⁺ erythroid cells at day 17.

FIGS. 14A-14I, Gene network inference identified mutations associated with 5-azaC sensitivity. FIG. 14A KD efficiency of IKZF1 and NPM1 after a 5-day Dox-induced depletion of the respective gene. Expression levels were first normalized to ACTB, followed by normalization to the respective controls. FIGS. 14B-14C Cell cycle distribution measured by EdU incorporation in cells following 5-day Dox-induced IKZF1 depletion, followed by a 16-hour 5-azaC (3 μM) treatment. FIG. 14B shows cell cycle distribution for K562 cells. FIG. 14C shows cell cycle distribution for NB4 cells. FIG. 14D Metaplot of IKZF1 ChIP-seq in K562 cells at caRNA m⁵C sites. FIG. 14E Schematic representation of the identification of genes determining 5-azaC sensitivity through gene network-based inference using NetBID2. FIG. 14F Expression of frequently mutated genes in leukemia that correlate with 5-azaC sensitivity and their target genes in the network. FIG. 14G GSEA of positive and negative targets of frequently mutated genes in leukemia related to 5-azaC sensitivity. Log₂ FC refers to log₂ fold change gene expression (resistant/sensitive). FIG. 14H Cell proliferation of THP1 following 24-hour 5-azaC treatment after Dox-induced depletion of NPM1. FIG. 14I Cell proliferation of HEL following 24-hour 5-azaC treatment after 48-hour 10 μM Tazemetostat treatment.

FIG. 15, Selective enrichment of NSUN2 and NSUN6 by biotinylated small-molecule pull-down. Shown are representative Western blots for NSUN1-4.

FIG. 16, Covalent inhibition of NSUN2 by NS71 assessed by washout assay. Shown is a depiction of NSUN2 protein activity after incubation with NS71 or DMSO at room temperature for 0.5 h.

FIG. 17, Quantification of tRNA m⁵C levels by UHPLC—MS/MS. Shown are tRNA m³C levels in the presence or absence of increasing concentrations of an illustrative NSUN2 inhibitor (NS-71).

FIG. 18, Pharmacokinetic profile of an illustrative NSUN2 inhibitor (NS-33) in male ICR mice following intraperitoneal administration (10 mg/kg). Shown are plasma concentrations of NS-33 in ICR mice after intraperitoneal administration.

FIG. 19, Pharmacokinetic profile of an illustrative NSUN2 inhibitor (NS-71) in male ICR mice following intraperitoneal administration (10 mg/kg). Shown are plasma concentrations of NS-71 in ICR mice after intraperitoneal administration.

### DETAILED DESCRIPTION

### I. Terminology

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or. It is specifically contemplated that A, B, or C may be specifically excluded from an aspect.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term sequence as used herein in reference to a polynucleotide refers to the nucleotide sequence such as “A” for adenosine, “G” for guanine, “C” for cytosine, “T” for thymine, “U” for uracil, “I” for inosine, and “N” for “A”/“C”/“U”/“T”/“G”/“I”.

As used herein, the terms “individual,” “subject,” and “patient” are used interchangeably and can refer to a human or non-human.

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed. The terms described above may be used interchangeably. A “modified protein” or “modified polypeptide” or “engineered protein” or “engineered polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as catalytic activity, RNA-binding activity, etc.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid phase peptide synthesis (SPPS) or other in vitro methods. In particular aspects, there are isolated nucleic acid segments and recombinant vectors incorporating nucleic acid sequences that encode a polypeptide (e.g., an enzymatic domain, such as a deaminase domain, or a fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 410, 415, 420, 425, 430, 435, 440, 445, 450, 455, 460, 465, 470, 475, 480, 485, 490, 495, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting, localization, linking, etc.).

In certain aspects, nucleic acid sequences can exist in a variety of instances such as: isolated segments and recombinant vectors of incorporated sequences or recombinant polynucleotides encoding an enzyme, or a fragment, derivative, or variant thereof, polynucleotides sufficient for targeting a complex to specific loci, polynucleotides sufficient to mediate caRNA methylation (e.g., m⁵C) level modifications, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, ancillary components of CRISPR/Cas systems, functional oligonucleotides, donor constructs, rescue constructs, and complementary sequences of the foregoing described herein. Nucleic acids encoding fusion proteins that include the proteins/polypeptides described herein are also contemplated. The nucleic acids can be single-stranded or double-stranded and can comprise RNA and/or DNA nucleotides and artificial variants thereof (e.g., peptide nucleic acids, etc.).

The term “polynucleotide” refers to a nucleic acid molecule that either is recombinant or has been isolated from total genomic nucleic acid. Included within the term “polynucleotide” are oligonucleotides (e.g., nucleic acids typically 200 residues or less, or 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In certain respects, the term “gene,” “polynucleotide,” or “nucleic acid” is used to refer to a nucleic acid that encodes a protein, polypeptide, or peptide (including any sequences required for proper transcription, post-translational modification, or localization), or a functional RNA species, such as but not limited to, CRISPR/Cas system ancillary components, linking elements, targeting RNAs, etc. As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, mutants, and functional RNA species. A nucleic acid encoding all or part of a polypeptide and/or functional RNA species may contain a contiguous nucleic acid sequence encoding all or a portion of such a polypeptide and/or functional RNA species. It also is contemplated that a particular polypeptide and/or functional RNA species may be encoded by nucleic acids containing variations having slightly different nucleic acid sequences but, nonetheless, encode the same or substantially similar protein and/or RNA species.

In certain aspects, there are polynucleotide variants having substantial identity to the sequences disclosed herein; those comprising at least 70%, 75%, 80%, 85%, 90%, 91%, 92%, 93%, 94%, 95%, 96%, 97%, 98%, or 99% or higher sequence identity, including all values and ranges there between, compared to a polynucleotide sequence provided herein using the methods known in the art and/or described herein (e.g., BLAST analysis using standard parameters). In certain aspects, the isolated polynucleotide will comprise a nucleotide sequence encoding a polypeptide and/or functional RNA species that has at least 90%, 95%, or above, identity to an amino acid sequence and/or RNA sequence described herein, over the entire length of the sequence; or a nucleotide sequence complementary to said isolated polynucleotide.

Nucleic acid segments, regardless of the length of the coding sequence itself, may be combined with other nucleic acid sequences, such as promoters, polyadenylation signals, enhancers, destabilization sites, restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

The term “near or at”, particularly when used in the context of chromosomal distance, as used herein generally refers to a distance of less than, 4.5-5.5 kb (e.g., less than 5.5 kb, less than 5 kb, less than 4kb, less than 4.5 kb, less than 3 kb, less than 2 kb, or less than 1 kb, or any range derivable therein) between two locations.

A “diseased cell” as used herein generally refers to a cell associated with a disease or disorder comprising one or more genetic mutations relative to a healthy cell in the same individual. The one or more genetic mutations (including but not limited to, large or small truncations, point mutations, indels, complex fusions, chromosomal translocations, and gene rearrangements, etc.) result in an abnormal phenotype relative to cells that do not comprise the one or more mutations (e.g. “healthy cells”). Abnormal phenotypes may include reduced or increased fitness, such as phenotypes associated with cancerous cells. In certain aspects, a diseased cell comprises one or more mutations in one or more oncogenes and/or tumor suppressor genes. In some aspects, a diseased cell may comprise one or more mutations in one or more genes encoding ten-eleven translocation (tet) methylcytosine dioxygenase 2 (TET2), ASXL transcriptional regulator 1 (ASXL1), ASXL Transcriptional Regulator 2 (ASXL2), ASXL Transcription Regulator 3 (ASXL3), isocitrate dehydrogenase 1 (IDH1), isocitrate dehydrogenase 2 (IDH2), tumor protein p53 (p53), DNA (cytosine-5-)-methyltransferase 3A (DNMT3A), Janus kinase 2 (JAK2), Protein Phosphatase Mn2+/Mg2+-Dependent 1D (PPM1D), Spliceosome Factor 3b1 (SF3B1), Serine and Arginine Rich Splicing Factor 2 (SRSF2), a component of a canonical and/or non-canonical Polycomb Repressive Complex (PRC) (e.g., E3 Ubiquitin Ligase RING1A/B, Polycomb Group Ring Finger 1 (PCGF1), Polycomb Group Ring Finger 2 (PCGF2), Polycomb Group Ring Finger 3 (PCGF3), Polycomb Group Ring Finger 4 (PCGF4), Polycomb Group Ring Finger 5 (PCGF5), and/or Polycomb Group Ring Finger 6 (PCGF6)), Polycomb Repressive-Deubiquitinase (PR-DUB) complex associated components O-linked N-acetylglucosamine Transferase (OGT), Lysine Demethylase 1B (KDM1B), Forkhead Box K1 (FOXK1), Forkhead Box K2 (FOXK2), BRCA1 Associated Protein 1 (BAP1), and/or Host Cell Factor C1 (HCFC1). In some aspects, a diseased cell may comprise one or more chromosomal rearrangements or gene fusions resulting in expression of an oncogenic fusion protein. In some aspects, a diseased cell may comprise a rearrangement of the lysine methyltransferase 2A (KMT2A; also known as mixed-lineage leukemia (MLL)) gene, including but not limited to fusions of KMT2A with one or more partner genes (e.g., AFF1 (AF4), MLLT3 (AF9), MLLT1 (ENL), MLLT10 (AF10), ELL, and/or AFDN (AF6)), wherein said rearrangement results in expression of an oncogenic KMT2A fusion protein (KMT2A rearrangement; KMT2A-r).

The term “proteolysis targeting chimera” or “PROTAC” as used herein refers to a chimeric molecule comprising two protein binding molecules: one capable of engaging an E3 ubiquitin ligase and another that binds to a target protein meant for degradation. Recruitment of the E3 ligase to the target protein results in ubiquitination and subsequent degradation of the target protein via the proteosome. In some aspects, a PROTAC disclosed herein may target an IKZF1 protein, an NPM1 protein, a TET2 protein, an EZH2 protein, or any combination thereof. In some aspects, a PROTAC disclosed herein may target an IKZF1 protein. In some aspects, a PROTAC disclosed herein may target an NPM1 protein. In some aspects, a PROTAC disclosed herein may target an EZH2 protein.

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987, all of which are incorporated herein by reference in their entirety.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain aspects, as used herein, “aliphatic” is used to indicate those aliphatic groups (cyclic, acyclic, substituted, unsubstituted, branched or unbranched) having 1-20 carbon atoms (C1-C20 aliphatic). In certain aspects, the aliphatic group has 1-10 carbon atoms (C1-C10 aliphatic). In certain aspects, the aliphatic group has 1-6 carbon atoms (C1-C6 aliphatic). In certain aspects, the aliphatic group has 1-5 carbon atoms (C1-C5 aliphatic). In certain aspects, the aliphatic group has 1-4 carbon atoms (C1-C4 aliphatic). In certain aspects, the aliphatic group has 1-3 carbon atoms (C1-C3 aliphatic). In certain aspects, the aliphatic group has 1-2 carbon atoms (C1-C2 aliphatic). Aliphatic group substituents include, but are not limited to, any of the substituents described herein, that result in the formation of a stable moiety.

The term “alkyl,” as used herein, refers to saturated, straight- or branched-chain hydrocarbon radicals derived from a hydrocarbon moiety containing between one and twenty carbon atoms by removal of a single hydrogen atom. In some aspects, the alkyl group employed in the invention contains 1-20 carbon atoms (C1-C20 alkyl). In another aspect, the alkyl group employed contains 1-15 carbon atoms (C1-C15 alkyl). In another aspect, the alkyl group employed contains 1-10 carbon atoms (C1-C10 alkyl). In another aspect, the alkyl group employed contains 1-8 carbon atoms (C1-C8 alkyl). In another aspect, the alkyl group employed contains 1-6 carbon atoms (C1-C6 alkyl). In another aspect, the alkyl group employed contains 1-5 carbon atoms (C1-C5 alkyl). In another aspect, the alkyl group employed contains 1-4 carbon atoms (C1-C4 alkyl). In another aspect, the alkyl group employed contains 1-3 carbon atoms (C1-C3 alkyl). In another aspect, the alkyl group employed contains 1-2 carbon atoms (C1-C2 alkyl). Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents. Alkyl group substituents include, but are not limited to, any of the substituents described herein, that result in the formation of a stable moiety. The term “alkylene,” as used herein, refers to a biradical derived from an alkyl group, as defined herein, by removal of two hydrogen atoms. Alkylene groups may be cyclic or acyclic, branched or unbranched, substituted or unsubstituted. Alkylene group substituents include, but are not limited to, any of the substituents described herein, that result in the formation of a stable moiety.

The term “alkenyl,” as used herein, denotes a monovalent group derived from a straight- or branched-chain hydrocarbon moiety having at least one carbon-carbon double bond by the removal of a single hydrogen atom. In certain aspects, the alkenyl group employed in the invention contains 2-20 carbon atoms (C2-C20 alkenyl). In some aspects, the alkenyl group employed in the invention contains 2-15 carbon atoms (C2-C15 alkenyl). In another aspect, the alkenyl group employed contains 2-10 carbon atoms (C2-C10C alkenyl). In still other aspects, the alkenyl group contains 2-8 carbon atoms (C2-C8 alkenyl). In yet other aspects, the alkenyl group contains 2-6 carbons (C2-C6 alkenyl). In yet other aspects, the alkenyl group contains 2-5 carbons (C2-C5 alkenyl). In yet other aspects, the alkenyl group contains 2-4 carbons (C2-C4 alkenyl). In yet other aspects, the alkenyl group contains 2-3 carbons (C2-C3 alkenyl). In yet other aspects, the alkenyl group contains 2 carbons (C2 alkenyl). Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety. The term “alkenylene,” as used herein, refers to a biradical derived from an alkenyl group, as defined herein, by removal of two hydrogen atoms. Alkenylene groups may be cyclic or acyclic, branched or unbranched, substituted or unsubstituted. Alkenylene group substituents include, but are not limited to, any of the substituents described herein, that result in the formation of a stable moiety.

The term “alkynyl,” as used herein, refers to a monovalent group derived from a straight- or branched-chain hydrocarbon having at least one carbon-carbon triple bond by the removal of a single hydrogen atom. In certain aspects, the alkynyl group employed in the invention contains 2-20 carbon atoms (C2-C20 alkynyl). In some aspects, the alkynyl group employed in the invention contains 2-15 carbon atoms (C2-C15 alkynyl). In another aspect, the alkynyl group employed contains 2-10 carbon atoms (C2-C10 alkynyl). In still other aspects, the alkynyl group contains 2-8 carbon atoms (C2-C8 alkynyl). In still other aspects, the alkynyl group contains 2-6 carbon atoms (C2-C6 alkynyl). In still other aspects, the alkynyl group contains 2-5 carbon atoms (C2-C5 alkynyl). In still other aspects, the alkynyl group contains 2-4 carbon atoms (C2-C4 alkynyl). In still other aspects, the alkynyl group contains 2-3 carbon atoms (C2-C3 alkynyl). In still other aspects, the alkynyl group contains 2 carbon atoms (C2 alkynyl). Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety. The term “alkynylene,” as used herein, refers to a biradical derived from an alkynylene group, as defined herein, by removal of two hydrogen atoms. Alkynylene groups may be cyclic or acyclic, branched or unbranched, substituted or unsubstituted. Alkynylene group substituents include, but are not limited to, any of the substituents described herein, that result in the formation of a stable moiety.

The term “carbocyclic” or “carbocyclyl” as used herein, refers to an as used herein, refers to a cyclic aliphatic group containing 3-10 carbon ring atoms (C3-C10 carbocyclic). Carbocyclic group substituents include, but are not limited to, any of the substituents described herein, that result in the formation of a stable moiety.

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that further contains one or more heteroatoms (e.g., oxygen, sulfur, nitrogen, phosphorus, or silicon atoms) between carbon atoms. In certain aspects, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl,” “heteroalkynyl,” and the like. Furthermore, as used herein, the terms “heteroalkyl,” “heteroalkenyl,” “heteroalkynyl,” and the like encompass both substituted and unsubstituted groups. In certain aspects, as used herein, “heteroaliphatic” is used to indicate those heteroaliphatic groups (cyclic, acyclic, substituted, unsubstituted, branched or unbranched) having 1-20 carbon atoms and 1-6 heteroatoms (C1-C20 heteroaliphatic). In certain aspects, the heteroaliphatic group contains 1-10 carbon atoms and 1-4 heteroatoms (C1-C10 heteroaliphatic). In certain aspects, the heteroaliphatic group contains 1-6 carbon atoms and 1-3 heteroatoms (C1-C6 heteroaliphatic). In certain aspects, the heteroaliphatic group contains 1-5 carbon atoms and 1-3 heteroatoms (C1-C5 heteroaliphatic). In certain aspects, the heteroaliphatic group contains 1-4 carbon atoms and 1-2 heteroatoms (C1-C4 heteroaliphatic). In certain aspects, the heteroaliphatic group contains 1-3 carbon atoms and 1 heteroatom (C1-C3 heteroaliphatic). In certain aspects, the heteroaliphatic group contains 1-2 carbon atoms and 1 heteroatom (C1-C2 heteroaliphatic). Heteroaliphatic group substituents include, but are not limited to, any of the substituents described herein, that result in the formation of a stable moiety.

The term “heteroalkyl,” as used herein, refers to an alkyl moiety, as defined herein, which contain one or more heteroatoms (e.g., oxygen, sulfur, nitrogen, phosphorus, or silicon atoms) in between carbon atoms. In certain aspects, the heteroalkyl group contains 1-20 carbon atoms and 1-6 heteroatoms (C1-C20 heteroalkyl). In certain aspects, the heteroalkyl group contains 1-10 carbon atoms and 1-4 heteroatoms (C1-C10 heteroalkyl). In certain aspects, the heteroalkyl group contains 1-6 carbon atoms and 1-3 heteroatoms (C1-C6 heteroalkyl). In certain aspects, the heteroalkyl group contains 1-5 carbon atoms and 1-3 heteroatoms (C1-C5 heteroalkyl). In certain aspects, the heteroalkyl group contains 1-4 carbon atoms and 1-2 heteroatoms (C1-C4 heteroalkyl). In certain aspects, the heteroalkyl group contains 1-3 carbon atoms and 1 heteroatom (C1-C3 heteroalkyl). In certain aspects, the heteroalkyl group contains 1-2 carbon atoms and 1 heteroatom (C1-2 heteroalkyl). The term “heteroalkylene,” as used herein, refers to a biradical derived from an heteroalkyl group, as defined herein, by removal of two hydrogen atoms. Heteroalkylene groups may be cyclic or acyclic, branched or unbranched, substituted or unsubstituted. Heteroalkylene group substituents include, but are not limited to, any of the substituents described herein, that result in the formation of a stable moiety.

The term “heteroalkenyl,” as used herein, refers to an alkenyl moiety, as defined herein, which further contains one or more heteroatoms (e.g., oxygen, sulfur, nitrogen, phosphorus, or silicon atoms) in between carbon atoms. In certain aspects, the heteroalkenyl group contains 2-20 carbon atoms and 1-6 heteroatoms (C2-C20 heteroalkenyl). In certain aspects, the heteroalkenyl group contains 2-10 carbon atoms and 1-4 heteroatoms (C2-C10 heteroalkenyl). In certain aspects, the heteroalkenyl group contains 2-6 carbon atoms and 1-3 heteroatoms (C2-C6 heteroalkenyl). In certain aspects, the heteroalkenyl group contains 2-5 carbon atoms and 1-3 heteroatoms (C2-C5 heteroalkenyl). In certain aspects, the heteroalkenyl group contains 2-4 carbon atoms and 1-2 heteroatoms (C2-C4 heteroalkenyl). In certain aspects, the heteroalkenyl group contains 2-3 carbon atoms and 1 heteroatom (C2-C3 heteroalkenyl). The term “heteroalkenylene,” as used herein, refers to a biradical derived from an heteroalkenyl group, as defined herein, by removal of two hydrogen atoms. Heteroalkenylene groups may be cyclic or acyclic, branched or unbranched, substituted or unsubstituted.

The term “heteroalkynyl,” as used herein, refers to an alkynyl moiety, as defined herein, which further contains one or more heteroatoms (e.g., oxygen, sulfur, nitrogen, phosphorus, or silicon atoms) in between carbon atoms. In certain aspects, the heteroalkynyl group contains 2-20 carbon atoms and 1-6 heteroatoms (C2-C20 heteroalkynyl). In certain aspects, the heteroalkynyl group contains 2-10 carbon atoms and 1-4 heteroatoms (C2-C10 heteroalkynyl). In certain aspects, the heteroalkynyl group contains 2-6 carbon atoms and 1-3 heteroatoms (C2-C6 heteroalkynyl). In certain aspects, the heteroalkynyl group contains 2-5 carbon atoms and 1-3 heteroatoms (C2-C5 heteroalkynyl). In certain aspects, the heteroalkynyl group contains 2-4 carbon atoms and 1-2 heteroatoms (C2-C4 heteroalkynyl). In certain aspects, the heteroalkynyl group contains 2-3 carbon atoms and 1 heteroatom (C2-C3 heteroalkynyl). The term “heteroalkynylene,” as used herein, refers to a biradical derived from an heteroalkynyl group, as defined herein, by removal of two hydrogen atoms. Heteroalkynylene groups may be cyclic or acyclic, branched or unbranched, substituted or unsubstituted.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain aspects, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropyranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety. As used herein, a “nitrogen-containing heterocyclyl” refers to a heterocyclyl group, as defined herein, comprising at least one ring nitrogen atom. Exemplary nitrogen-containing heterocyclyl groups include, but are not limited to, piperidinyl, piperazinyl, morpholinyl, pyrrolidinyl, azetidinyl, azepanyl, diazepanyl, imidazolidinyl, oxazolidinyl, and the like, each of which may be unsubstituted or optionally substituted, including substitution on a ring nitrogen atom (e.g., with alkyl, acyl, alkoxycarbonyl, or other substituents described herein).

The term “aryl,” as used herein, refers to an aromatic mono- or polycyclic ring system having 3-20 ring atoms, of which all the ring atoms are carbon, and which may be substituted or unsubstituted. In certain aspects of the present invention, “aryl” refers to a mono, bi, or tricyclic C4-C20 aromatic ring system having one, two, or three aromatic rings which include, but are not limited to, phenyl, biphenyl, naphthyl, and the like, which may bear one or more substituents. The term “aryl” expressly encompasses fused polycyclic carbocyclic aromatic ring systems, including but not limited to naphthalenyl (naphthyl), anthracenyl, phenanthrenyl, fluorenyl, indenyl, and acenaphthenyl, in addition to monocyclic aryl groups such as phenyl. Aryl substituents include, but are not limited to, any of the substituents described herein, that result in the formation of a stable moiety. The term “arylene,” as used herein refers to an aryl biradical derived from an aryl group, as defined herein, by removal of two hydrogen atoms. Arylene groups may be substituted or unsubstituted. Arylene group substituents include, but are not limited to, any of the substituents described herein, that result in the formation of a stable moiety. Additionally, arylene groups may be incorporated as a linker group into an alkylene, alkenylene, alkynylene, heteroalkylene, heteroalkenylene, or heteroalkynylene group, as defined herein.

The term “heteroaryl,” as used herein, refers to an aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. The term “heteroaryl” expressly encompasses fused bicyclic and tricyclic heteroaromatic ring systems in which a heteroaromatic ring is fused to one or more additional aromatic carbocyclic rings or additional heteroaromatic rings. Examples of heteroaryls include, but are not limited to pyrrolyl, pyrazolyl, imidazolyl, pyridinyl, pyrimidinyl, pyrazinyl, pyridazinyl, triazinyl, tetrazinyl, pyrrolizinyl, indolyl, isoindolyl, indazolyl, quinolinyl, isoquinolinyl, quinolizinyl, cinnolinyl, quinazolinyl, phthalazinyl, naphthyridinyl, quinoxalinyl, thiophenyl, benzothiophenyl (thianaphthenyl), furanyl, benzofuranyl, isobenzofuranyl, benzothiazolyl, thiazolyl, isothiazolyl, thiadiazolyl, oxazolyl, isoxazolyl, oxadiazolyl, benzimidazolyl, pyrrolopyridinyl (including 1H-pyrrolo[2,3-b]pyridinyl, 1H-pyrrolo[2,3-c]pyridinyl, 1H-pyrrolo[3,2-b]pyridinyl, and related isomers), and dioxolo-fused indolyl ring systems (including ring systems in which an indolyl group is fused with a 1,3-dioxole ring, such as those derived from benzo[d][1,3]dioxol-5-yl substituents), and the like, which may bear one or more substituents. Heteroaryl substituents include, but are not limited to, any of the substituents described herein, that result in the formation of a stable moiety. The term “heteroarylene,” as used herein, refers to a biradical derived from a heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety.

Aryls, heteroaryls and cyclic moieties may be fused to form a fused “bicyclic” compound. The term “bicyclic” refers to a compound with two conjoined rings. The two conjoined rings can be conjoined in a fused, bridged, or spiro arrangement. A fused bicyclic compound includes two rings that share two atoms. In some aspects a fused bicyclic compound herein is referred to as a “fused aryl”, “fused heteroaryl”, “fused cycle”, “fused heterocycle” or “fused aryl heterocycle” depending on whether one or both of the two conjoined rings are aromatic or aliphatic as defined above. A bridged bicyclic compound includes two rings that share three or more atoms and a bridge that contains at least one atom, where the bridge atom is covalently bound to the shared ring atoms. A spiro bicyclic compound includes two rings that are joined at a common atom.

The term “acyl,” as used herein, is a subset of a substituted alkyl group, and refers to a group having the general formula —C(═O)RA, —C(═O)ORA, —C(═O)—O—C(═O)RA, —C(═O)SRA, —C(═O)N(RA)2, —C(═S)RA, —C(═S)N(RA)2, and —C(═S)S(RA), —C(═NRA)RA, —C(═NRA)ORA, —C(═NRA)SRA, and —C(═NRA)N(RA)2, wherein RA is hydrogen; halogen; substituted or unsubstituted hydroxyl; substituted or unsubstituted thiol; substituted or unsubstituted amino; acyl; optionally substituted aliphatic; optionally substituted heteroaliphatic; optionally substituted alkyl; optionally substituted alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di heteroarylamino; or two RA groups taken together form a 5- to 6-membered heterocyclic ring. Exemplary acyl groups include aldehydes (—CHO), carboxylic acids (—CO2H), ketones, acyl halides, esters, amides, imines, carbonates, carbamates, and ureas. Acyl substituents include, but are not limited to, any of the substituents described herein, that result in the formation of a stable moiety.

The term “acylene,” as used herein, is a subset of a substituted alkylene, substituted alkenylene, substituted alkynylene, substituted heteroalkylene, substituted heteroalkenylene, or substituted heteroalkynylene group, and refers to an acyl group having the general formulae: —R0-(C═X1)-R0-, —R—X2(C═X1)-R0-, or —R0-X2(C═X1)X3-R0-, where X1, X2, and X3 is, independently, oxygen, sulfur, or NRr, wherein Rr is hydrogen or optionally substituted aliphatic, and R0 is an optionally substituted alkylene, alkenylene, alkynylene, heteroalkylene, heteroalkenylene, or heteroalkynylene group, as defined herein. Exemplary acylene groups wherein R0 is alkylene includes —(CH2)T-O(C═O)—(CH2)T-; —(CH2)T-NRr(C═O)—(CH2)T-; —(CH2)T-O(C═NRr)-(CH2)T-; —(CH2)T-NRr(C═NRr)-(CH2)T-; —(CH2)T-(C═O)—(CH2)T-; —(CH2)T-(C═NRr)-(CH2)T-; —(CH2)T-S(C═S)—(CH2)T-; —(CH2)T-NRr(C═S)—(CH2)-; —(CH2)T-S(C═NRr)-(CH2)T-; —(CH2)T-O(C═S)—(CH2)T-; —(CH2)T-(C═S)—(CH2)T-; or —(CH2)T-S(C═O)—(CH2)T-, and the like, which may bear one or more substituents; and wherein each instance of T is, independently, an integer between 0 to 20. Acylene substituents include, but are not limited to, any of the substituents described herein, that result in the formation of a stable moiety.

The term “amine” or “amino,” as used herein, refers to a group of the formula (—NH2). A “substituted amino” refers either to a mono-substituted amine (—NHRh) of a disubstituted amine (—NRh2), wherein the Rh substituent is any substituent as described herein that results in the formation of a stable moiety (e.g., aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain aspects, the Rh substituents of the di-substituted amino group (—NRh2) form a 5- to 6-membered heterocyclic ring.

The term “hydroxy” or “hydroxyl,” as used herein, refers to a group of the formula (—OH). The term “alkoxy,” as used herein, refers to a group of the formula (—ORi), wherein Ri can be any substituent which results in a stable moiety (e.g., aliphatic, alkyl, alkenyl, alkynyl, heteroaliphatic, heterocyclic, aryl, heteroaryl, acyl, nitro, alkylaryl, arylalkyl, and the like, each of which may or may not be further substituted). In certain aspects, “alkoxy” refers to a saturated (C1-C20)alkoxy group, wherein the alkyl portion is a straight, branched, or cyclic alkyl group as defined herein. In certain aspects, the alkoxy group has 1-6 carbon atoms (C1-C6 alkoxy). In certain aspects, the alkoxy group has 1-4 carbon atoms (C1-C4 alkoxy). Exemplary alkoxy groups include, but are not limited to, methoxy (—OCH3), ethoxy (—OCH2CH3), n-propoxy, isopropoxy, n-butoxy, sec-butoxy, tert-butoxy, n-pentyloxy, n-hexyloxy, and cycloalkyloxy groups such as cyclopropylmethoxy. Alkoxy group substituents include, but are not limited to, any of the substituents described herein, that result in the formation of a stable moiety.

The term “thio” or “thiol,” as used herein, refers to a group of the formula (—SH). A “substituted thiol” refers to a group of the formula (—SRr), wherein Rr can be any substituent that results in the formation of a stable moiety (e.g., a thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “imino,” as used herein, refers to a group of the formula (═NRr), wherein Rr corresponds to hydrogen or any substituent as described herein, that results in the formation of a stable moiety (for example, an amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted).

The term “azide” or “azido,” as used herein, refers to a group of the formula (—N3).

The terms “halo” and “halogen,” as used herein, refer to an atom selected from fluorine (fluoro, —F), chlorine (chloro, —Cl), bromine (bromo, —Br), and iodine (iodo, —I).

The term “cyano,” as used herein, refers to a group of the formula (—C≡N). The term “cyano” may be used interchangeably with “nitrile” when referring to a substituent attached to a carbon skeleton.

The term “nitro,” as used herein, refers to a group of the formula (—NO2).

The term “haloalkyl,” as used herein, refers to an alkyl group, as defined herein, substituted with one or more halogen atoms, as defined herein. The haloalkyl group may be mono-, di-, tri-, or perhalogenated. In certain aspects, haloalkyl contains 1-6 carbon atoms (C1-C6 haloalkyl). In certain aspects, haloalkyl contains 1-4 carbon atoms (C1-C4 haloalkyl). Exemplary haloalkyl groups include, but are not limited to, fluoromethyl, difluoromethyl, trifluoromethyl (—CF3), chloromethyl, bromomethyl, dichloromethyl, trichloromethyl, 2-fluoroethyl, 2,2,2-trifluoroethyl, pentafluoroethyl, and 3,3,3-trifluoropropyl. The term “haloalkyl” expressly encompasses perfluoroalkyl groups, such as trifluoromethyl (—CF3) and pentafluoroethyl (—C2F5). Haloalkyl group substituents include, but are not limited to, any of the substituents described herein, that result in the formation of a stable moiety.

The term “haloalkoxy,” as used herein, refers to an alkoxy group, as defined herein, in which the alkyl portion is substituted with one or more halogen atoms, as defined herein. The haloalkoxy group may be mono-, di-, tri-, or perhalogenated. In certain aspects, haloalkoxy contains 1-6 carbon atoms in the alkyl portion (C1-C6 haloalkoxy). In certain aspects, haloalkoxy contains 1-4 carbon atoms (C1-C4 haloalkoxy). Exemplary haloalkoxy groups include, but are not limited to, fluoromethoxy, difluoromethoxy, trifluoromethoxy (—OCF3), chloromethoxy, 2-fluoroethoxy, 2,2,2-trifluoroethoxy, and pentafluoroethoxy. The term “haloalkoxy” expressly encompasses perfluoroalkoxy groups, such as trifluoromethoxy (—OCF3). Haloalkoxy group substituents include, but are not limited to, any of the substituents described herein, that result in the formation of a stable moiety.

The term “alkoxycarbonyl,” as used herein, refers to a group of the formula —C(═O)OR, wherein R is an alkyl group, as defined herein. In certain aspects, alkoxycarbonyl refers to a (C1-C6)alkoxycarbonyl group. In certain aspects, alkoxycarbonyl refers to a (C1-C4)alkoxycarbonyl group. Exemplary alkoxycarbonyl groups include, but are not limited to, methoxycarbonyl (—C(═O)OCH3), ethoxycarbonyl (—C(═O)OEt), n-propoxycarbonyl, isopropoxycarbonyl, and tert-butoxycarbonyl (—C(═O)OC(CH3)3; also referred to as Boc). Alkoxycarbonyl group substituents include, but are not limited to, any of the substituents described herein, that result in the formation of a stable moiety.

The term “aryloxy,” as used herein, refers to a group of the formula —ORa, wherein Ra is an aryl group, as defined herein, which may be substituted or unsubstituted. Exemplary aryloxy groups include, but are not limited to, phenoxy, naphthyloxy, biphenyloxy, and the like, which may bear one or more substituents. Aryloxy group substituents include, but are not limited to, any of the substituents described herein, that result in the formation of a stable moiety.

The term “aralkoxy,” as used herein, refers to a group of the formula —ORb, wherein Rb is an arylalkyl (aralkyl) group comprising an aryl group, as defined herein, appended through an alkylene chain, as defined herein. In certain aspects, the alkylene portion of the aralkoxy group contains 1-6 carbon atoms (C1-C6 alkylene). In certain aspects, the alkylene portion contains 1-3 carbon atoms. Exemplary aralkoxy groups include, but are not limited to, benzyloxy (—OCH2Ph), 2-phenylethoxy, 3-phenylpropoxy, naphthalenylmethoxy, and the like. Aralkoxy group substituents include, but are not limited to, any of the substituents described herein, that result in the formation of a stable moiety.

The term “carboxy(C1-C6)alkoxy,” as used herein, refers to a group of the formula —O—(C1-C6 alkylene)-C(═O)OH, i.e., an alkoxy group, as defined herein, in which the alkyl chain contains 1 to 6 carbon atoms and terminates in a free carboxylic acid group (—COOH). Exemplary carboxy(C1-C6)alkoxy groups include, but are not limited to, carboxymethoxy (—OCH2COOH), 2-carboxyethoxy (—OCH2CH2COOH), 3-carboxypropoxy (—O(CH2)3COOH), and 4-carboxybutoxy (—O(CH2)4COOH). Carboxy(C1-C6)alkoxy group substituents include, but are not limited to, any of the substituents described herein, that result in the formation of a stable moiety.

The compounds disclosed herein may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

The term “barbiturate moiety,” as used herein, refers to a pyrimidine-2,4,6(1H,3H,5H)-trione ring system, i.e., a six-membered heterocyclic ring comprising nitrogen atoms at positions 1 and 3 and carbonyl groups at positions 2, 4, and 6. The C5 position of the barbiturate ring system is the site through which the compounds of Formula (I) are connected via the L linker (—CH═) to form the exocyclic double bond. The term “barbiturate moiety” encompasses unsubstituted barbituric acid-derived rings as well as N1- and/or N3-substituted variants thereof. An exemplary barbiturate moiety is the ring system of barbituric acid (pyrimidine-2,4,6(1H,3H,5H)-trione).

The term “thiobarbiturate moiety,” as used herein, refers to a 2-thioxodihydropyrimidine-4,6(1H,5H)-dione ring system, i.e., the ring system corresponding to barbituric acid in which the C2 carbonyl group (C2=O) is replaced by a thione (C2=S), while the C4 and C6 carbonyl groups are retained. As with the barbiturate moiety, the C5 position is the site of connection to the L linker in compounds of Formula (I). The term “thiobarbiturate moiety” encompasses unsubstituted 2-thiobarbituric acid-derived rings as well as N1- and/or N3-substituted variants thereof. An exemplary thiobarbiturate moiety is the ring system of 2-thiobarbituric acid (2-thioxodihydropyrimidine-4,6(1H,5H)-dione).

The term “Meldrum's acid moiety,” as used herein, refers to a 1,3-dioxane-4,6-dione ring system. Meldrum's acid is 2,2-dimethyl-1,3-dioxane-4,6-dione. As used in the compounds of the present disclosure, a “Meldrum's acid moiety” refers to the 1,3-dioxane-4,6-dione ring system that provides a reactive carbon at the C5 position, through which the compounds of Formula (I) are connected via the L linker to form the exocyclic double bond. The term encompasses the parent Meldrum's acid ring system (2,2-dimethyl-1,3-dioxane-4,6-dione) as well as C2-substituted variants thereof.

The term “thiazolidinedione moiety,” as used herein, refers to a thiazolidine-2,4-dione ring system, i.e., a five-membered heterocyclic ring comprising a sulfur atom at position 1, a nitrogen atom at position 3, and carbonyl groups at positions 2 and 4. The C5 position of the thiazolidinedione ring is the reactive methylene site through which the compounds of Formula (I) are connected via the L linker (—CH═) to form the exocyclic double bond. The term “thiazolidinedione moiety” encompasses unsubstituted thiazolidine-2,4-dione rings as well as N3-substituted variants thereof.

The term “pyrazolone moiety,” as used herein, refers to a pyrazolin-5-one ring system, i.e., a five-membered ring containing two adjacent nitrogen atoms and a carbonyl group. The term encompasses the various tautomeric forms of the pyrazolone ring system, including 1H-pyrazol-5(4H)-one (5-pyrazolone) and 1H-pyrazol-3(2H)-one (3-pyrazolone) tautomers, as well as N1- and/or N2-substituted variants thereof. The C4 position of the pyrazolone ring is the reactive methylene site through which the compounds of Formula (I) are connected via the L linker (—CH═) to form the exocyclic double bond.

The term “indane-1,3-dione moiety,” as used herein, refers to a 1H-indene-1,3(2H)-dione ring system (also known as 1,3-indandione), i.e., a bicyclic ring system in which a benzene ring is fused to a five-membered 1,3-cyclopentanedione ring. The C2 methylene position of the indane-1,3-dione ring is the reactive site through which the compounds of Formula (I) are connected via the L linker (—CH═) to form the exocyclic double bond. The benzene ring of the indane-1,3-dione moiety may be unsubstituted or optionally substituted with one or more substituents as described herein.

The term “cyanoacrylic acid moiety,” as used herein, refers to a —C(C≡N)(COOH) fragment that, in combination with the L linker (—CH═) in compounds of Formula (I), provides the structural motif —CH═C(C≡N)(COOH), corresponding to an α-cyanoacrylic acid (2-cyanoacrylic acid) functionality. An exemplary compound bearing a cyanoacrylic acid moiety is 2-cyanoacrylic acid (CH2=C(CN)(COOH)).

The term “cyanoacrylate moiety,” as used herein, refers to a —C(C≡N)(C(═O)OR) fragment, wherein R is an alkyl, aryl, or heteroaryl group as defined herein, that in combination with the L linker (—CH═) in compounds of Formula (I), provides the structural motif —CH═C(C≡N)(C(═O)OR), corresponding to an α-cyanoacrylate ester functionality. Exemplary cyanoacrylate moieties include methyl α-cyanoacrylate (—C(C≡N)(C(═O)OCH3)) and ethyl α-cyanoacrylate (—C(C≡N)(C(═O)OEt)) moieties.

The term “aryl-substituted acrylonitrile moiety,” as used herein, refers to a —C(C N)(Ar) fragment, wherein Ar is an aryl or heteroaryl group as defined herein, that in combination with the L linker (—CH═) in compounds of Formula (I), provides the structural motif —CH═C(C≡N)(Ar), corresponding to a 2-aryl acrylonitrile (α-aryl acrylonitrile) functionality. The aryl or heteroaryl substituent may be unsubstituted or substituted with one or more substituents as described herein. An exemplary aryl-substituted acrylonitrile compound of Formula (I) is (E)-3-(indol-3-yl)-2-phenylacrylonitrile.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

A variety of aspects are discussed throughout this application. Any aspect discussed with respect to one aspect applies to other aspects as well and vice versa. Each aspect described herein is understood to be aspects that are applicable to all aspects. It is contemplated that any aspect discussed herein can be implemented with respect to any method or composition, and vice versa. Furthermore, compositions and kits can be used to achieve methods disclosed herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

### II. Overview

Gene expression is regulated through multiple interconnected layers, including chromatin organization, transcriptional control, RNA processing, and translation. While DNA methylation and histone modifications have long been recognized as key regulators of chromatin state and transcription, it has become increasingly evident that chemical modifications on RNA (epitranscriptomic modifications) represent an additional and essential regulatory layer governing gene expression programs. More than one hundred distinct RNA modifications have been identified, including N6-methyladenosine (m⁶A), pseudouridine (Ψ), and 5-methylcytosine (m³C). These modifications influence diverse aspects of RNA metabolism, including RNA stability, localization, translation efficiency, and interactions with RNA-binding proteins. In addition to their established roles in post-transcriptional regulation, emerging evidence indicates that RNA modifications can also directly influence transcriptional processes and chromatin states through interactions with chromatin-associated RNA and protein complexes. 5-azaC is the first-line therapy for high-risk myelodysplastic syndromes (MDS) (6) and acute myeloid leukemia (AML) in older adults not eligible for intensive therapies (7). However, the response rate of 5-azaC varies around 50% (8,9), with frequent resistance development further limiting its efficacy. Currently, there are no reliable biomarkers of 5-azaC efficacy prior to administration, indicating a lack of understanding of its mechanism of action.

Among RNA modifications, 5-methylcytosine (m³C) in RNA has emerged as a key regulator of cellular function and gene expression. RNA m³C is catalyzed by RNA methyltransferases of the NSUN family, including NSUN2 and NSUN6. NSUN2 is a broadly acting methyltransferase that modifies multiple RNA substrates, including transfer RNA (tRNA), messenger RNA (mRNA), and noncoding RNAs. In contrast, NSUN6 exhibits more substrate specificity, primarily targeting defined tRNA substrates. As a ribonucleoside analog of cytidine, 5-azaC can be incorporated into nucleic acids, with 10%-20% integrated into DNA and 80%-90% into RNA (10). Interestingly, previous studies predominantly focused on the DNA-dependent cytotoxicity of 5-azaC, highlighting its role as a hypomethylation agent that covalently inhibits DNA methyltransferases (11-13), thereby disrupting DNA 5mC deposition and epigenetic regulation. However, DNA demethylation targets do not align with the differentially expressed genes following 5-azaC treatment (1-4), suggesting alternative regulatory mechanisms. In addition to DNA demethylation, 5-azaC also impairs the activity of RNA m⁵C methyltransferases (14,15), whose effect and underlying pathway on leukemia cytotoxicity have been largely overlooked. The deoxy form of 5-azaC, decitabine, which exclusively incorporates into DNA, exhibits notably weaker effects on MDS and AML treatment (16-18). These observations suggest a possibly significant contribution of the RNA-dependent cytotoxicity for 5-azaC. Additionally, patients that have benefited from 5-azaC administration did not necessarily exhibit DNA hypomethylation (5). Moreover, it has been indicated that sensitivity to 5-azaC can be associated with expression levels and regulatory roles of RNA m⁵C methyltransferases (19). The combination of these findings suggest a potentially important role of RNA m⁵C depletion in 5-azaC treatment, yet detailed mechanisms connecting RNA m⁵C depletion with cytotoxicity of 5-azaC are still lacking.

RNA m⁵C regulates multiple cellular processes (20,21). In rRNA, m⁵C modifications influence ribosomal biogenesis (22), while in tRNA, they impact RNA stability (23), both contributing to translational regulation. In mRNA, m⁵C may affect splicing (24), nuclear export (25), and RNA stability (26,27), likely mediated by the m⁵C-binding proteins SRSF2, ALYREF, and/or YBX1, respectively. Recent studies have demonstrated that RNA m⁵C can play a role in linking RNA to chromatin regulation and transcriptional control. Specifically, m⁵C-modified chromatin-associated RNA (caRNA) may be recognized by reader proteins, such as MBD6, which recruit chromatin-modifying complexes, including the BAP1 complex, to genomic loci. The inventors group have recently discovered a chromatin and transcriptional regulatory role of m⁵C in caRNA (28). Those data showed that caRNA m⁵C can be recognized by MBD6 (28) and MBD5 (29), which then recruit the BAP1 complex (PR-DUB) for removal of histone H2AK119ub to confer transcription activation. Oxidation of caRNA m⁵C by the TET2 enzyme suppresses this gene activation pathway (28).Loss or mutation of TET2 has been associated with increased chromatin accessibility and aberrant transcriptional activation, which contribute to leukemogenesis, further highlighting the functional importance of RNA m⁵C-mediated chromatin regulation.

Consistent with its role in chromatin regulation, RNA m⁵C has also been implicated in modulating cellular responses to therapeutic agents. Nucleoside analog-based chemotherapeutics such as 5-azacytidine can be incorporated into RNA and perturb RNA cytosine methylation, including m⁵C marks on chromatin-associated RNA. Recent studies suggest that these early alterations in RNA methylation are, at least in part, mediated through disruption of NSUN2-dependent methylation activity, thereby linking RNA m⁵C pathways to changes in chromatin-associated RNA function and downstream cellular responses. In addition to its roles in cellular gene regulation, RNA m⁵C has also been identified in viral RNAs, where it contributes to the regulation of viral RNA stability, gene expression, and replication. Collectively, these findings establish RNA m⁵C as an important regulator at the interface of RNA metabolism, chromatin regulation, transcriptional control, and therapeutic response. In this study, the inventors have characterized the RNA-dependent cytotoxicity of 5-azaC by capturing its cellular responses at early time points. The results provided herein show that the RNA-dependent pathways were sufficient to induce leukemia cell death, disrupting cell cycle regulation and DNA repair, and promoting apoptosis. Contrary to the conventional view of 5-azaC as a DNA hypomethylating agent, its RNA-dependent effects caused transcriptional repression, contributing to the cellular dysregulation. Mechanistically, 5-azaC depleted m⁵C on repeat RNA, reducing MBD6 binding and subsequent recruitment of the BAP1 deubiquitylase complex for histone H2AK119ub removal. In parallel, caRNA m⁵C depletion also disrupted SRSF2 binding to chromatin, impairing p300 recruitment and H3K27ac deposition. These transcriptional effects were primarily mediated through 5-azaC-induced inhibition of NSUN2, an RNA methyltransferase responsible for caRNA m⁵C deposition. Indeed, NSUN2 depletion recapitulated the cellular dysregulation observed with 5-azaC treatment at early time points. Through the study of NSUN2, the inventors have also uncovered that the 5-azaC-induced hematopoietic commitment towards the erythroid lineage is RNA-dependent. Finally, the results provided herein, including the leukemia cell line screen, have identified TET2, IKZF1, EZH2, and/or NPM1 mutations as regulators of 5-azaC sensitivity. These findings highlight the significance of RNA-dependent mechanisms in 5-azaC treatment, and provide new insights into efficacy prediction and novel therapy development based on 5-azaC. Further exploration into new inhibitors of NSUN2 provide new means to pharmacologically target RNA m³C methyltransferases, a clinical unmet need.

Mutation of TET2 has been described as driving myeloid malignancy initiation and progression. TET2 deficiency has also been described as leading to a globally opened chromatin state (e.g., euchromatin) and aberrant activation of genes, contributing to aberrant cell self-renewal (e.g., aberrant hematopoietic stem cell (HSC) self-renewal). However, the open chromatin consistently observed in TET2-deficient mouse embryonic stem cells, leukemic cells, and hematopoietic progenitor and stem cells (HPSCs) is inconsistent with TET2's described role of DNA 5mC oxidation. As described herein, the inventors have shown herein and/or previously (see e.g., Zou et al., 2024 (28), Guo et al., 2024 (29), and PCT/US2024/049478 filed Oct. 1, 2024, each of which are incorporated herein in their entirety), that chromatin-associated RNA (caRNA, e.g., chromatin-associated retrotransposon RNA) 5-methylcytosine (m⁵C) can be recognized by the methyl-CpG-binding domain protein MBD6, which can guide deubiquitylation of nearby histone H2AK119ub to promote an open chromatin state. TET2 can oxidize m⁵C and antagonize this caRNA m⁵C associated MBD6-dependent H2AK119ub deubiquitylation. Thereby, TET2 depletion can lead to globally decreased H2AK119ub, more open chromatin, and increased/aberrant transcription in cells, such as stem cells.

As such, in certain aspects, provided herein are methods of treatment of diseases associated with diseased cells, such as human leukemias, comprising inhibition of TET2, IKZF1, EZH2, and/or NPM1, wherein said inhibition can result in a synergistic and/or synthetic lethal effect upon diseased cells (e.g., cancer cells). In certain aspects, inhibiting IKZF1, EZH2, and/or NPM1 protein activity (including silencing expression of IKZF1, EZH2, and/or NPM1) can selectively inhibit proliferation of TET2 mutant cells. In some aspects, inhibiting IKZF1, EZH2, and/or NPM1 protein activity (including silencing expression of IKZF1, EZH2, and/or NPM1) can selectively inhibit proliferation of TET2 mutant leukemic cells in vitro and in vivo. In certain aspects, inhibiting IKZF1, EZH2, and/or NPM1 protein activity (including silencing expression of IKZF1, EZH2, and/or NPM1) can selectively inhibit proliferation of cells that comprise functional TET2. In some aspects, inhibiting IKZF1, EZH2, and/or NPM1 protein activity (including silencing expression of IKZF1, EZH2, and/or NPM1) can selectively inhibit proliferation of cells that comprise functional TET2 in vitro and in vivo.

In some aspects, inhibiting IKZF1, EZH2, and/or NPM1 protein activity (including silencing expression of IKZF1, EZH2, and/or NPM1) in combination with 5-azaC treatment can result in improved and/or synergistic inhibition of disease cell (such as leukemic cell) proliferation in vitro and in vivo. In certain aspects, inhibiting IKZF1, EZH2, and/or NPM1 protein activity (including silencing expression of IKZF1, EZH2, and/or NPM1) in combination with 5-azaC treatment can result in selective inhibition of proliferation of diseased cells that comprise functional TET2. In some aspects, inhibiting IKZF1, EZH2, and/or NPM1 protein activity (including silencing expression of IKZF1, EZH2, and/or NPM1) in combination with 5-azaC treatment can inhibit proliferation of cells that comprise functional TET2 in vitro and in vivo. In certain aspects, inhibiting IKZF1, EZH2, and/or NPM1 protein activity (including silencing expression of IKZF1, EZH2, and/or NPM1) in combination with 5-azaC treatment can result in selective inhibition proliferation of diseased cells that comprise mutant TET2. In some aspects, inhibiting IKZF1, EZH2, and/or NPM1 protein activity (including silencing expression of IKZF1, EZH2, and/or NPM1) in combination with 5-azaC treatment can inhibit proliferation of cells that comprise mutant TET2 in vitro and in vivo.

In some aspects, provided herein are methods of treating a disease in a subject, such as but not limited to cancers such as leukemia, comprising measuring the gene and/or protein activity of IKZF1, EZH2, and/or NPM1, and if the level of gene and/or protein activity is reduced relative to controls and/or anticipated levels, treating the subject with 5-azaC. In some aspects, provided herein are methods of treating a disease in a subject, such as but not limited to cancers such as leukemia, comprising measuring the gene and/or protein activity of IKZF1, EZH2, and/or NPM1, and if the level of gene and/or protein activity are increased, similar, and/or not significantly reduced relative to controls and/or anticipated levels, not treating the subject with solely 5-azaC but instead treating with one or more alternative therapies and/or one or more combination therapies (such as but not limited to, inhibition of TET2, IKZF1, EZH2, and/or NPM1).

In some aspects, an inhibitor of IKZF1 comprises, consists essentially of, or consists of CC-92480. In some aspects, an inhibitor of EZH2 comprises, consists essentially of, or consists of CPI-1205, GSK2816126, PF-06821497, SHR2554, and/or DS3201b.

In certain aspects, provided herein are methods of treatment of diseases associated with diseased cells, such as human leukemias, comprising inhibition of NSUN2, alone or in combination with inhibition of TET2, IKZF1, EZH2, and/or NPM1 as provided herein above, wherein said inhibition can result in a synergistic and/or synthetic lethal effect upon diseased cells (e.g., cancer cells). In certain aspects, inhibiting NSUN2 protein activity (including silencing expression of NSUN2 and/or administering a small molecule inhibitor of NSUN2 provided herein) can selectively inhibit proliferation of TET2 mutant cells. In certain aspects, inhibiting NSUN2 protein activity (including silencing expression of NSUN2 and/or administering a small molecule inhibitor of NSUN2 provided herein) can selectively inhibit proliferation of mutant cells comprising a rearrangement of the lysine methyltransferase 2A (KMT2A; also known as mixed-lineage leukemia (MLL)) gene.

In some aspects inhibiting NSUN2 protein activity (including silencing expression of NSUN2 and/or administering a small molecule inhibitor of NSUN2 provided herein) can selectively inhibit proliferation of TET2 mutant leukemic cells in vitro and in vivo. In some aspects inhibiting NSUN2 protein activity (including silencing expression of NSUN2 and/or administering a small molecule inhibitor of NSUN2 provided herein) can selectively inhibit proliferation of mutant leukemic cells comprising a rearrangement of the lysine methyltransferase 2A (KMT2A; also known as mixed-lineage leukemia (MLL)) gene in vitro and in vivo.

In certain aspects, inhibiting NSUN2 protein activity (including silencing expression of NSUN2 and/or administering a small molecule inhibitor of NSUN2 provided herein) can selectively inhibit proliferation of cells that comprise functional TET2. In some aspects, inhibiting NSUN2 protein activity (including silencing expression of NSUN2 and/or administering a small molecule inhibitor of NSUN2 provided herein) can selectively inhibit proliferation of cells that comprise functional TET2 in vitro and in vivo.

In certain aspects, inhibiting NSUN2 protein activity (including silencing expression of NSUN2 and/or administering a small molecule inhibitor of NSUN2 provided herein) can selectively inhibit proliferation of cells that comprise a rearrangement of the lysine methyltransferase 2A (KMT2A; also known as mixed-lineage leukemia (MLL)) gene. In some aspects, inhibiting NSUN2 protein activity (including silencing expression of NSUN2 and/or administering a small molecule inhibitor of NSUN2 provided herein) can selectively inhibit proliferation of cells that comprise a rearrangement of the lysine methyltransferase 2A (KMT2A; also known as mixed-lineage leukemia (MLL)) gene in vitro and in vivo.

In some aspects, inhibiting NSUN2 protein activity (including silencing expression of NSUN2 and/or administering a small molecule inhibitor of NSUN2 provided herein) in combination with 5-azaC treatment can result in improved and/or synergistic inhibition of disease cell (such as leukemic cell) proliferation in vitro and in vivo. In certain aspects, inhibiting NSUN2 protein activity (including silencing expression of NSUN2 and/or administering a small molecule inhibitor of NSUN2 provided herein) in combination with 5-azaC treatment can result in selective inhibition of proliferation of diseased cells that comprise functional TET2. In some aspects, inhibiting NSUN2 protein activity (including silencing expression of NSUN2 and/or administering a small molecule inhibitor of NSUN2 provided herein) in combination with 5-azaC treatment can inhibit proliferation of cells that comprise functional TET2 in vitro and in vivo. In certain aspects, inhibiting NSUN2 protein activity (including silencing expression of NSUN2 and/or administering a small molecule inhibitor of NSUN2 provided herein) in combination with 5-azaC treatment can result in selective inhibition proliferation of diseased cells that comprise mutant TET2. In some aspects, inhibiting NSUN2 protein activity (including silencing expression of NSUN2 and/or administering a small molecule inhibitor of NSUN2 provided herein) in combination with 5-azaC treatment can inhibit proliferation of cells that comprise mutant TET2 in vitro and in vivo. In some aspects, inhibiting NSUN2 protein activity (including silencing expression of NSUN2 and/or administering a small molecule inhibitor of NSUN2 provided herein) in combination with 5-azaC treatment can inhibit proliferation of cells that comprise a rearrangement of the lysine methyltransferase 2A (KMT2A; also known as mixed-lineage leukemia (MLL)) gene in vitro and in vivo.

In some aspects, the current disclosure provides technologies including at least polynucleotides, proteins, polypeptides, and/or vectors, and further provides methods and/or compositions comprising any one or more of the aforementioned components. In certain aspects, polynucleotides may encode sequences comprising RNA m⁵C modifying catalytic domains (including isolated domains, polypeptides, and/or proteins), engineered small nucleolar RNA (snoRNA), CRISPR/Cas proteins/polypeptides, and/or ancillary RNA components (e.g., CRISPR RNA (crRNA), trans-activating CRISPR RNA (tracrRNA), single guide RNA (sgRNA), etc.), antisense oligonucleotides (ASO), etc. In certain aspects, one or more of the polynucleotides and/or polypeptides can be engineered.

In certain aspects, polynucleotides, proteins, polypeptides, and/or peptide sequences for wild type or mutant versions of various genes, such as site-specific target genes, have been previously disclosed, and may be found in the recognized computerized databases. In certain aspects, polynucleotides, proteins, polypeptides, and/or peptide sequences for wild type versions of various effector proteins and/or RNA molecules, have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there can be between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

It is contemplated that in compositions of the disclosure, there can be between about 0.001 mg and about 10 mg of total polynucleotide, shRNA, RNA, siRNA, etc. per ml. The concentration of nucleic acids in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

In certain aspects, provided herein are methods, compositions, polynucleotides, polypeptides, and/or vectors comprising engineered RNA m⁵C modifying (e.g., installing, reading, or erasing) catalytic domains (e.g., including complete enzymes and/or components of enzymes), engineered Cas proteins, and/or guide oligonucleotides

The oligonucleotides, polypeptides, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, exactly, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 or more contiguous amino acids or nucleic acids, or any range derivable therein, of SEQ ID NOs: 1-23. In specific aspects, the nucleic acid encoding the peptide or polypeptide is codon optimized for expression in a mammal. In certain aspects, the peptide or polypeptide is not naturally occurring and/or is in a combination of peptides or polypeptides.

The polypeptides of the disclosure may include at least, at most, or exactly 1, 2, 3, 4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422 substitutions (or any range derivable therein). In some aspects, the substitution is with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the polypeptide comprises one or more substitutions at one or more amino acid positions in any one of the proteins or polynucleotides encoding the same identified in SEQ ID NOs: 1-23, wherein each substitution is independently chosen from an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; and wherein the polypeptide or polynucleotide encoding the same is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range or value therein) sequence identity to one of SEQ ID NOs: 1-23.

In some aspects, the protein or polypeptide, or polynucleotide encoding the same may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92,93,94,95,96,97,98,99, 100,101, 102,103, 104, 105,106, 107, 108,109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304,305,306,307,308,309,310,311,312,313,314,315,316,317,318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399,400, 401,402, 403,404, 405, 406,407, 408, 409,410, 411,412, 413, 414,415, 416, 417, 418,419, 420,421, 422,423, 424, 425,426, 427, 428,429, 430,431, 432, 433, 434, 435, 436, 437, 438, 439, 440, 441, 442, 443, 444, 445, 446, 447, 448, 449, 450, 451, 452, 453, 454, 455, 456,457, 458, 459, 460,461, 462, 463,464, 465, 466,467, 468,469, 470, 471,472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704,705,706,707,708,709,710,711,712,713,714,715,716,717,718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, or 2060 of SEQ ID NOs: 1-23, or any other amino acid or nucleotide noted therein, and have or have at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 1-23.

In some aspects, the protein, polypeptide, or nucleic acid may comprise, comprise at least, or comprise at most 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402,403, 404,405, 406,407, 408, 409,410, 411, 412,413, 414,415, 416, 417,418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000, or more (or any derivable range therein) contiguous amino acids or nucleic acids of SEQ ID NOs: 1-23.

In some aspects, the polypeptide, protein, or nucleic acid may comprise at least, at most, or exactly 1to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309,310,311,312,313,314,315,316,317,318,319,320,321,322,323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000, or more (or any derivable range therein) contiguous amino acids or nucleic acids of SEQ ID NOs: 1-23 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOs: 1-23.

In some aspects there is a nucleic acid molecule or polypeptide starting at position 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422, or any other nucleotide or amino acid of any of SEQ ID NOs: 1-23 and comprising at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000, or greater than 2000 (or any derivable range therein) contiguous amino acids or nucleic acids of any of SEQ ID NOs: 1-23.

### III. Leukemia, TET2 Pathway, Mutations and Diseases Associated Therewith

Aspects of the present disclosure are directed to compositions and methods for treatment of leukemia and/or diseases associated with abnormal m⁵C RNA feature levels.

Somatic loss-of-function mutations in the ten-eleven translocation 2 (TET2) gene occur in a significant proportion of patients with myeloid malignancies. Genetic studies of patients with myeloid malignancies have identified recurrent somatic alterations in the majority of patients with myeloproliferative neoplasms (MPNs), myelodysplastic syndromes (MDSs), and acute myeloid leukemia (AML). In some aspects, provided herein are methods of treating myeloproliferative neoplasms (MPNs), myelodysplastic syndromes (MDSs), and/or acute myeloid leukemia (AML). Several studies have identified recurrent mutations of known and putative epigenetic modifiers in patients with myeloid malignancies. These include somatic mutations in chromatin-modifying enzymes and in DNA methyltransferases. In addition, biologic studies of recurrent chromosomal translocations, including MLL fusions, have shown that leukemogenic fusion proteins alter epigenetic regulation in hematopoietic cells, resulting in changes in chromatin state at specific loci. In certain aspects, provided herein are methods of treating MLL. Taken together, these data suggest that somatic alterations in genes that regulate the epigenetic state of hematopoietic cells are a common pathogenetic event in leukemogenesis. In certain aspects, provided herein are methods of treating diseases associated with somatic alterations in genes that regulate the epigenetic state of hematopoietic cells, such as but not limited to hematopoietic stem cells.

Somatic deletions and loss-of-function mutations in the ten-eleven translocation 2 (TET2) gene were identified in 10%-20% of patients with MDS and MPN; subsequent studies identified recurrent TET2 mutations in patients with chronic myelomonocytic leukemia (CMML) and AML, and demonstrated that TET2 mutations were associated with adverse outcome in intermediate-risk AML. Within the TET family of proteins, TET1, TET2, and TET3 have been shown to modify DNA by hydroxylating 5-methylcytosine (5mC), and the TET2 mutant proteins observed in these patients with myeloid malignancies have been shown to be deficient in this enzymatic function. Mutations that impact 5-hydroxymethylation represent a mechanism of transformation in myeloid malignancies (see e.g., Kelly Moran-Crusio et al., Tet2 Loss Leads to Increased Hematopoietic Stem Cell Self-Renewal and Myeloid Transformation. Cancer Cell 2011; which is incorporated herein by reference in its entirety for the purposes described herein).

Additionally, inactivating mutations in Tet methylcytosine dioxygenase 2 (TET2) have been detected in peripheral blood cells of a remarkable 5%-10% of adults greater than 65 years of age. The TET2 inactivating mutations have been reported to impart a hematopoietic stem cell advantage and resultant clonal hematopoiesis of indeterminate potential (CHIP) with skewed myelomonocytic differentiation. CHIP has been reported to be associated with an overall increased risk of transformation to a hematological malignancy, especially myeloproliferative and myelodysplastic neoplasms (MPN, MDS) and acute myeloid leukemia (AML), of approximately 0.5% to 1% per year. However, it is becoming increasingly possible to identify individuals at greatest risk, based on CHIP mutational characteristics. CHIP, and particularly TET2-mutant CHIP, has also been reported to be a significant risk factor for cardiovascular diseases, related in part to hyper-inflammatory progeny macrophages that carry TET2 inactivating mutations. Therefore, somatic TET2 mutations, and mutations in TET2 pathways, can contribute to myeloid expansion and innate immune dysregulation with age and can contribute to prevalent diseases in the developed world (e.g., cancer and/or cardiovascular disease).

Chromosomal rearrangements involving the lysine methyltransferase 2A (KMT2A; also known as mixed-lineage leukemia (MLL)) gene (GRCh38.p14 Chromosome 11, location NC_000011.10 (118307205 . . . 118397539; 11q23.3)) occur in approximately 5%-10% of all acute leukemias and represent one of the most frequent cytogenetic abnormalities in AML, acute lymphoblastic leukemia (ALL), and therapy-related leukemias. The KMT2A gene encodes a histone H3 lysine 4 (H3K4) methyltransferase that functions as a master regulator of hematopoietic gene expression programs, including expression of HOX genes essential for normal hematopoietic development. KMT2A rearrangements result in the in-frame fusion of KMT2A with one of more than 80 reported partner genes; the most frequent fusions involve AFF1 (AF4), MLLT3 (AF9), MLLT1 (ENL), MLLT10 (AF10), ELL, and AFDN (AF6). The resulting KMT2A fusion oncoproteins aberrantly recruit transcriptional elongation complexes, including the super elongation complex (SEC) and the DOT1L complex, leading to aberrant activation of KMT2A target genes and dysregulation of hematopoietic differentiation. KMT2A-rearranged (KMT2A-r) leukemias are characterized by a global dysregulation of epigenetic regulation in hematopoietic cells, resulting in changes in chromatin state at specific loci and a block in normal hematopoietic differentiation. In certain aspects, provided herein are methods of treating KMT2A-rearranged leukemias and/or diseases associated with dysregulated epigenetic regulation of hematopoietic gene expression resulting from KMT2A rearrangement. In some aspects, KMT2A-r leukemia cells exhibit increased sensitivity to inhibition of NSUN2, IKZF1, and/or other targets described herein, consistent with the role of these targets in regulating the epigenetic state of hematopoietic cells. Additional exemplary KMT2A fusion partner genes and information related thereto can be found in the NCBI database under Gene ID: 4297.

Ikaros family zinc finger 1 (IKZF1; also known as IK1; LYF1; LyF-1; CVID13; IKAROS; PPP1R92; PRO0758; ZNFN1A1; Hs.54452) gene (GRCh38.p14, Chromosome 7, location NC_000007.14 (50303455 . . . 50405101; 7p12.2)) encodes a transcription factor that belongs to the family of zinc-finger DNA-binding proteins associated with chromatin remodeling. Generally expression of this protein is restricted to the fetal and adult hemo-lymphopoietic system, and it functions as a regulator of lymphocyte differentiation. Several alternatively spliced transcript variants encoding different isoforms have been described for this gene. Most isoforms share a common C-terminal domain, which contains two zinc finger motifs that are required for hetero- or homo-dimerization, and for interactions with other proteins. The isoforms, however, differ in the number of N-terminal zinc finger motifs that bind DNA and in nuclear localization signal presence, resulting in members with and without DNA-binding properties. Only a few isoforms contain the requisite three or more N-terminal zinc motifs that confer high affinity binding to a specific core DNA sequence element in the promoters of target genes. The non-DNA-binding isoforms are largely found in the cytoplasm, and are thought to function as dominant-negative factors. Overexpression of some dominant-negative isoforms have been associated with B-cell malignancies, such as acute lymphoblastic leukemia (ALL). Exemplary human wildtype IKZF1 protein isoforms 1 and 2 are provided as SEQ ID NOs: 1-6. Additional exemplary human wildtype IKZF1 protein isoforms and encoding genes, and information related thereto, including but not limited to isoforms 3, 4, 5, 6, 7, 8, ik-6, ik-7, ik-7(del), ik-8, ik-8(del), 15, 16, 17, etc. can be found in the NCBI database under Gene ID: 10320. In some aspects, methods disclosed herein comprise, consist essentially of, or consist of inhibition of IKZF1. In some aspects, methods disclosed herein comprise measuring the expression levels of IKZF1. In some aspects, methods disclosed herein comprise measuring the expression levels of IKZF1 as a biomarker for disease susceptibility to 5-azaC. In some aspects, methods disclosed herein comprise administering one or more inhibitors of IKZF1. The one or more inhibitors of IKZF1 may, in some aspects, comprise a polynucleotide at least partially complementary to a gene encoding IKZF1 (e.g., a short hairpin RNA and/or small interfering RNA). In some aspects, the one or more inhibitors of IKZF1 comprise a proteolysis targeting chimera (PROTAC) targeting IKZF1. In some aspects, methods disclosed herein comprise inhibiting and/or targeting all isoforms of IKZF1 as reported on NCBI. In some aspects, methods disclosed herein comprise inhibiting and/or targeting most of the isoforms of IKZF1 as reported on NCBI.

Nucleophosmin 1 (NPM1; also known as B23; NPM) gene (GRCh38.p14 Chromosome 5, location NC_000005.10 (171387116 . . . 171410900; 5q35.1)) encodes protein involved in several cellular processes, including centrosome duplication, protein chaperoning, and cell proliferation. The encoded phosphoprotein shuttles between the nucleolus, nucleus, and cytoplasm, chaperoning ribosomal proteins and core histones from the nucleus to the cytoplasm. This protein is also known to sequester the tumor suppressor ARF in the nucleolus, protecting it from degradation until it is needed. Mutations in this gene are associated with acute myeloid leukemia. Dozens of pseudogenes of this gene have been identified. Exemplary human wildtype NPM1 protein isoforms and encoding genes are provided as SEQ ID NOs: 7-12. Additional exemplary human wildtype NPM1 protein isoforms and encoding genes, and information related thereto, such as but not limited to isoforms 3, 4, 5, 6, etc. can be found in the NCBI database under Gene ID: 4869. In some aspects, methods disclosed herein comprise, consist essentially of, or consist of inhibition of NPM1. In some aspects, methods disclosed herein comprise measuring the expression levels of NPM1. In some aspects, methods disclosed herein comprise measuring the expression levels of NPM1 as a biomarker for disease susceptibility to 5-azaC. In some aspects, methods disclosed herein comprise administering one or more inhibitors of NPM1. The one or more inhibitors of NPM1 may, in some aspects, comprise a polynucleotide at least partially complementary to a gene encoding NPM1 (e.g., a short hairpin RNA and/or small interfering RNA). In some aspects, the one or more inhibitors of NPM1 comprise a proteolysis targeting chimera (PROTAC) targeting NPM1.

Enhancer of zeste 2 polycomb repressive complex 2 subunit (EZH2; also known as WVS; ENX1; KMT6; WVS2; ENX-1; EZH2b; KMT6A) gene (GRCh38.p14 Chromosome 7, location NC_000007.14 (148807383 . . . 148884291, complement; 7q36.1)) encodes protein a member of the Polycomb-group (PcG) family. PcG family members form multimeric protein complexes, which are involved in maintaining the transcriptional repressive state of genes over successive cell generations. This protein associates with the embryonic ectoderm development protein, the VAV1 oncoprotein, and the X-linked nuclear protein. This protein may play a role in the hematopoietic and central nervous systems. Multiple alternatively spliced transcript variants encoding distinct isoforms have been identified for this gene.

Exemplary human wildtype EZH2 protein isoforms and encoding genes are provided as SEQ ID NOs: 13-18. Additional exemplary human wildtype EZH2 protein isoforms and encoding genes, and information related thereto, such as but not limited to isoforms c, d, and etc. can be found in the NCBI database under Gene ID: 2146. In some aspects, methods disclosed herein comprise, consist essentially of, or consist of inhibition of EZH2. In some aspects, methods disclosed herein comprise measuring the expression levels of EZH2. In some aspects, methods disclosed herein comprise measuring the expression levels of EZH2 as a biomarker for disease susceptibility to 5-azaC. In some aspects, methods disclosed herein comprise administering one or more inhibitors of EZH2. The one or more inhibitors of EZH2 may, in some aspects, comprise a polynucleotide at least partially complementary to a gene encoding EZH2 (e.g., a short hairpin RNA and/or small interfering RNA). In some aspects, the one or more inhibitors of EZH2 comprise a proteolysis targeting chimera (PROTAC) targeting EZH2.

NSUN2 (NOP2/Sun RNA methyltransferase 2) is a methyltransferase that has been reported to catalyze the methylation of cytosine to 5-methylcytosine (m⁵C) in tRNAs, such as at position 34 of intron-containing tRNA(Leu)(CAA) precursors. The m⁵C modification in tRNA(Leu)(CAA) is necessary to stabilize the anticodon-codon pairing and correctly translate target mRNA. Alternatively spliced transcript variants encoding different isoforms have been noted for this gene. In some aspects, NSUN2 functions as an m⁵C RNA writer protein and/or writer complex component. Exemplary human wildtype NSUN2 protein isoforms and encoding genes are provided as SEQ ID NOs: 21-23. Additional exemplary human wildtype NSUN1 protein isoforms and encoding genes, and information related thereto, can be found in the NCBI database under Gene ID: 54888. In some aspects, methods disclosed herein comprise, consist essentially of, or consist of inhibition of NSUN2. In some aspects, methods disclosed herein comprise administering one or more inhibitors of NSUN2. The one or more inhibitors of NSUN2 may, in some aspects, comprise a polynucleotide at least partially complementary to a gene encoding NSUN2 (e.g., a short hairpin RNA and/or small interfering RNA). In some aspects, the one or more inhibitors of NSUN2 comprise a proteolysis targeting chimera (PROTAC) targeting NSUN2. In some aspects, the one or more inhibitors of NSUN2 comprise a small molecule inhibitor.

In some aspects, the one or more inhibitors of NSUN2 comprise a compound of Formula I, as described in Section VII below.

In some aspects, a protein comprises, consists essentially of, or consists of an amino acid sequence or is encoded by a polynucleotide sequence, with about, exactly, or at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage derivable therein, identity to any one of SEQ ID NOs: 1-23.

In some aspects, an inhibitor of one or more proteins (e.g., 1, 2, 3, 4, 5, 6, 7, 8, 9, 10, or more) described herein comprises, consists essentially of, or consists of a polynucleotide sequence with about, exactly, or at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage derivable therein, identity to a contiguous stretch of at least 15, 16, 17, 18, 19, 20, 21, 22, 23, 24, 25, 26, 27, 28, 29, 30, 31, 32, 33, 34, 35, 36, 37, 38, 39, or 40 nucleotides of any one of SEQ ID NOs: 1-23.

SEQ ID NO: 1 - Wild type human IKZF1 isoform 1 amino acid sequence
(SEQ ID NO: 1)
MDADEGQDMSQVSGKESPPVSDTPDEGDEPMPIPEDLSTTSGGQQSSKSDRVVASNVKVETQ
SDEENGRACEMNGEECAEDLRMLDASGEKMNGSHRDQGSSALSGVGGIRLPNGKLKCDICGI
ICIGPNVLMVHKRSHTGERPFQCNQCGASFTQKGNLLRHIKLHSGEKPFKCHLCNYACRRRD
ALTGHLRTHSVGKPHKCGYCGRSYKQRSSLEEHKERCHNYLESMGLPGTLYPVIKEETNHSE
MAEDLCKIGSERSLVLDRLASNVAKRKSSMPQKFLGDKGLSDTPYDSSASYEKENEMMKSHV
MDQAINNAINYLGAESLRPLVQTPPGGSEVVPVISPMYQLHKPLAEGTPRSNHSAQDSAVEN
LLLLSKAKLVPSEREASPSNSCQDSTDTESNNEEQRSGLIYLTNHIAPHARNGLSLKEEHRA
YDLLRAASENSQDALRVVSTSGEQMKVYKCEHCRVLFLDHVMYTIHMGCHGFRDPFECNMCG
YHSQDRYEFSSHITRGEHRFHMS
SEQ ID NO: 2 - Wild type human IKZF1 isoform 1 mRNA sequence
(SEQ ID NO: 2)
actctaacaagtgactgcgcggcccgcgcccggggcggtgactgcggcaagccccctgggtc
cccgcgcggcgcatcccagcctgggcgggacgctcggccgcggcgaggcgggcaagcctggc
agggcagagggagccccggctccgaggttgctcttcgcacccgaggatcagtcttggcccca
aagcgcgacgcacaaatccacataacctgaggaccatggatgctgatgagggtcaagacatg
tcccaagtttcagggaaggaaagcccccctgtaagcgatactccagatgagggcgatgagcc
catgccgatccccgaggacctctccaccacctcgggaggacagcaaagctccaagagtgaca
gagtcgtggccagtaatgttaaagtagagactcagagtgatgaagagaatgggcgtgcctgt
gaaatgaatggggaagaatgtgcggaggatttacgaatgcttgatgcctcgggagagaaaat
gaatggctcccacagggaccaaggcagctcggctttgtcgggagttggaggcattcgacttc
ctaacggaaaactaaagtgtgatatctgtgggatcatttgcatcgggcccaatgtgctcatg
gttcacaaaagaagccacactggagaacggcccttccagtgcaatcagtgcggggcctcatt
cacccagaagggcaacctgctccggcacatcaagctgcattccggggagaagcccttcaaat
gccacctctgcaactacgcctgccgccggagggacgccctcactggccacctgaggacgcac
tccgttggtaaacctcacaaatgtggatattgtggccgaagctataaacagcgaagctcttt
agaggaacataaagagcgctgccacaactacttggaaagcatgggccttccgggcacactgt
acccagtcattaaagaagaaactaatcacagtgaaatggcagaagacctgtgcaagatagga
tcagagagatctctcgtgctggacagactagcaagtaacgtcgccaaacgtaagagctctat
gcctcagaaatttcttggggacaagggcctgtccgacacgccctacgacagcagcgccagct
acgagaaggagaacgaaatgatgaagtcccacgtgatggaccaagccatcaacaacgccatc
aactacctgggggccgagtccctgcgcccgctggtgcagacgcccccgggcggttccgaggt
ggtcccggtcatcagcccgatgtaccagctgcacaagccgctcgcggagggcaccccgcgct
ccaaccactcggcccaggacagcgccgtggagaacctgctgctgctctccaaggccaagttg
gtgccctcggagcgcgaggcgtccccgagcaacagctgccaagactccacggacaccgagag
caacaacgaggagcagcgcagcggtctcatctacctgaccaaccacatcgccccgcacgcgc
gcaacgggctgtcgctcaaggaggagcaccgcgcctacgacctgctgcgcgccgcctccgag
aactcgcaggacgcgctccgcgtggtcagcaccagcggggagcagatgaaggtgtacaagtg
cgaacactgccgggtgctcttcctggatcacgtcatgtacaccatccacatgggctgccacg
gcttccgtgatccttttgagtgcaacatgtgcggctaccacagccaggaccggtacgagttc
tcgtcgcacataacgcgaggggagcaccgcttccacatgagctaaagccctcccgcgccccc
accccagaccccgagccaccccaggaaaagcacaaggactgccgccttctcgctcccgccag
cagcatagactggactggaccagacaatgttgtgtttggatttgtaactgttttttgttttt
tgtttgagttggttgattggggtttgatttgcttttgaaaagatttttatttttagaggcag
ggctgcattgggagcatccagaactgctaccttcctagatgtttccccagaccgctggctga
gattccctcacctgtcgcttcctagaatccccttctccaaacgattagtctaaattttcaga
gagaaatagataaaacacgccacagcctgggaaggagcgtgctctaccctgtgctaagcacg
gggttcgcgcaccaggtgtctttttccagtccccagaagcagagagcacagcccctgctgtg
tgggtctgcaggtgagcagacaggacaggtgtgccgccacccaagtgccaagacacagcagg
gccaacaacctgtgcccaggccagcttcgagctacatgcatctagggcggagaggctgcact
tgtgagagaaaatactatttcaagtcatattctgcgtaggaaaatgaattggttggggaaag
tcgtgtctgtcagactgccctgggtggagggagacgccgggctagagcctttgggatcgtcc
tggattcactggctttgcggaggctgctcagatggcctgagcctcccgaggcttgctgcccc
gtaggaggagactgtcttcccgtgggcatatctggggagccctgttccccgctttttcactc
ccatacctttaatggcccccaaaatctgtcactacaatttaaacaccagtcccgaaatttgg
atcttctttctttttgaatctctcaaacggcaacattcctcagaaaccaaagctttatttca
aatctcttccttccctggctggttccatctagtaccagaggcctcttttcctgaagaaatcc
aatcctagccctcattttaattatgtacatctgtttgtagccacaagcctgaatttctcagt
gttggtaagtttctttacctaccctcactatatattattctcgttttaaaacccataaagga
gtgatttagaacagtcattaattttcaactcaatgaaatatgtgaagcccagcatctctgtt
gctaacacacagagctcacctgtttgaaaccaagctttcaaacatgttgaagctctttactg
taaaggcaagccagcatgtgtgtccacacatacataggatggctggctctgcacctgtagga
tattggaatgcacagggcaattgagggactgagccagaccttcggagagtaatgccaccaga
tcccctaggaaagaggaggcaaatggcactgcaggtgagaaccccgcccatccgtgctatga
catggaggcactgaagcccgaggaaggtgtgtggagattctaatcccaacaagcaagggtct
ccttcaagattaatgctatcaatcattaaggtcattactctcaaccacctaggcaatgaaga
atataccatttcaaatatttacagtacttgtcttcaccaacactgtcccaaggtgaaatgaa
gcaacagagaggaaattgtacataagtacctcagcatttaatccaaacaggggttcttagtc
tcagcactatgacattttgggctgactacttatttgttaggcgggagctctcctgtgcattg
taggataattagcagtatccctggtggctacccaatagacgccagtagcaccccgaattgac
aacccaaactctccagacatcaccaactgtcccctgcgaggagaaatcactcctgggggaga
accactgacccaaatgaattctaaaccaatcaaatgtctgggaagccctccaagaaaaaaaa
tagaaaagcacttgaagaatattcccaatattcccggtcagcagtatcaaggctgacttgtg
ttcatgtggagtcattataaattctataaatcaattattccccttcggtcttaaaaatatat
ttcctcataaacatttgagttttgttgaaaagatggagtttacaaagataccattcttgagt
catggatttctctgctcacagaagggtgtggcatttggaaacgggaataaacaaaattgctg
caccaatgcactgagtgaaggaagagagacagaggatcaagggctttagacagcactccttc
aatatgcaatcacagagaaagatgcgccttatccaagttaatatctctaaggtgagagcctt
cttagagtcagtttgttgcaaatttcacctactctgttcttttccatccatccccctgagtc
agttggttgaagggagttattttttcaagtggaattcaaacaaagctcaaaccagaactgta
aatagtgattgcaggaattcttttctaaactgctttgccctttcctctcactgccttttata
gccaatataaatgtctctttgcacaccttttgttgtggttttatattgtaacaccatttttc
tttgaaactattgtatttaaagtaaggtttcatattatgtcagcaagtaattaacttatgtt
taaaaggtggccatatcatgtaccaaaagttgctgaagtttctcttctagctggtaaagtag
gagtttgcatgacttcacactttttttgcgtagtttcttctgttgtatgatggcgtgagtgt
gtgtcttgggtaccgctgtgtactactgtgtgcctagattccatgcactctcgttgtgtttg
aagtaaatattggagaccggagggtaacaggttggcctgttgattacagctagtaatcgctg
tgtcttgttccgccccctccctgacaccccagcttcccaggatgtggaaagcctggatctca
gctccttgccccatatcccttctgtaatttgtacctaaagagtgtgattatcctaattcaag
agtcactaaaactcatcacattatcattgcatatcagcaaagggtaaagtcctagcaccaat
tgcttcacataccagcatgttccatttccaatttagaattagccacataataaaatcttaga
atcttccttgagaaagagctgcctgagatgtagttttgttatatggttccccaccgaccatt
tttgtgcttttttcttgttttgttttgttttgactgcactgtgagttttgtagtgtcctctt
cttgccaaaacaaacgcgagatgaactggacttatgtagacaaatcgtgatgccagtgtatc
cttcctttcttcagttccagcaataatgaatggtcaacttttttaaaatctagatctctctc
attcatttcaatgtatttttactttaagatgaaccaaaattattagacttatttaagatgta
caggcatcagaaaaaagaagcacataatgcttttggtgcgatggcactcactgtgaacatgt
gtaaccacatattaatatgcaatattgtttccaatactttctaatacagttttttataatgt
tgtgtgtggtgattgttcaggtcgaatctgttgtatccagtacagctttaggtcttcagctg
cccttctggcgagtacatgcacaggattgtaaatgagaaatgcagtcatatttccagtctgc
ctctatgatgatgttaaattattgctgtttagctgtgaacaagggatgtaccactggaggaa
tagagtatccttttgtacacattttgaaatgcttcttctgtagtgatagaacaaataaatgc
aacgaatactctgtctgccctatcccgtgaagtccacactggcgtaagagaaggcccagcag
agcaggaatctgcctagactttctcccaatgagatcccaatatgagagggagaagagatggg
cctcaggacagctgcaataccacttgggaacacatgtggtgtcttgatgtggccagcgcagc
agttcagcacaacgtacctcccatctacaacagtgctggacgtgggaattctaagtcccagt
cttgagggtgggtggagatggagggcaacaagagatacatttccagttctccactgcagcat
gcttcagtcattctgtgagtggccgggcccagggccctcacaatttcactaccttgtctttt
acatagtcataagaattatcctcaacatagccttttgacgctgtaaatcttgagtattcatt
tacccttttctgatctcctggaaacagctgcctgcctgcattgcacttctcttcccgaggag
tggggtaaatttaaaagtcaagttatagtttggatgttagtatagaattttgaaattgggaa
ttaaaaatcaggactggggactgggagaccaaaaatttctgatcccatttctgatggatgtg
tcacaccttttctgtcaaaataaaatgtcttggaggttatgactccttggtgaaa
SEQ ID NO: 3 - Wild type human IKZF1 isoform 1 polynucleotide coding
sequence
(SEQ ID NO: 3)
ATGGATGCTGATGAGGGTCAAGACATGTCCCAAGTTTCAGGGAAGGAAAGCCCCCCTGTAAG
CGATACTCCAGATGAGGGCGATGAGCCCATGCCGATCCCCGAGGACCTCTCCACCACCTCGG
GAGGACAGCAAAGCTCCAAGAGTGACAGAGTCGTGGCCAGTAATGTTAAAGTAGAGACTCAG
AGTGATGAAGAGAATGGGCGTGCCTGTGAAATGAATGGGGAAGAATGTGCGGAGGATTTACG
AATGCTTGATGCCTCGGGAGAGAAAATGAATGGCTCCCACAGGGACCAAGGCAGCTCGGCTT
TGTCGGGAGTTGGAGGCATTCGACTTCCTAACGGAAAACTAAAGTGTGATATCTGTGGGATC
ATTTGCATCGGGCCCAATGTGCTCATGGTTCACAAAAGAAGCCACACTGGAGAACGGCCCTT
CCAGTGCAATCAGTGCGGGGCCTCATTCACCCAGAAGGGCAACCTGCTCCGGCACATCAAGC
TGCATTCCGGGGAGAAGCCCTTCAAATGCCACCTCTGCAACTACGCCTGCCGCCGGAGGGAC
GCCCTCACTGGCCACCTGAGGACGCACTCCGTTGGTAAACCTCACAAATGTGGATATTGTGG
CCGAAGCTATAAACAGCGAAGCTCTTTAGAGGAACATAAAGAGCGCTGCCACAACTACTTGG
AAAGCATGGGCCTTCCGGGCACACTGTACCCAGTCATTAAAGAAGAAACTAATCACAGTGAA
ATGGCAGAAGACCTGTGCAAGATAGGATCAGAGAGATCTCTCGTGCTGGACAGACTAGCAAG
TAACGTCGCCAAACGTAAGAGCTCTATGCCTCAGAAATTTCTTGGGGACAAGGGCCTGTCCG
ACACGCCCTACGACAGCAGCGCCAGCTACGAGAAGGAGAACGAAATGATGAAGTCCCACGTG
ATGGACCAAGCCATCAACAACGCCATCAACTACCTGGGGGCCGAGTCCCTGCGCCCGCTGGT
GCAGACGCCCCCGGGCGGTTCCGAGGTGGTCCCGGTCATCAGCCCGATGTACCAGCTGCACA
AGCCGCTCGCGGAGGGCACCCCGCGCTCCAACCACTCGGCCCAGGACAGCGCCGTGGAGAAC
CTGCTGCTGCTCTCCAAGGCCAAGTTGGTGCCCTCGGAGCGCGAGGCGTCCCCGAGCAACAG
CTGCCAAGACTCCACGGACACCGAGAGCAACAACGAGGAGCAGCGCAGCGGTCTCATCTACC
TGACCAACCACATCGCCCCGCACGCGCGCAACGGGCTGTCGCTCAAGGAGGAGCACCGCGCC
TACGACCTGCTGCGCGCCGCCTCCGAGAACTCGCAGGACGCGCTCCGCGTGGTCAGCACCAG
CGGGGAGCAGATGAAGGTGTACAAGTGCGAACACTGCCGGGTGCTCTTCCTGGATCACGTCA
TGTACACCATCCACATGGGCTGCCACGGCTTCCGTGATCCTTTTGAGTGCAACATGTGCGGC
TACCACAGCCAGGACCGGTACGAGTTCTCGTCGCACATAACGCGAGGGGAGCACCGCTTCCA
CATGAGCTAA
SEQ ID NO: 4 - Wild type human IKZF1 isoform 2 (encoded by transcript
variants 2 and 14) amino acid sequence
(SEQ ID NO: 4)
MDADEGQDMSQVSGKESPPVSDTPDEGDEPMPIPEDLSTTSGGQQSSKSDRVVASNVKVETQ
SDEENGRACEMNGEECAEDLRMLDASGEKMNGSHRDQGSSALSGVGGIRLPNGKLKCDICGI
ICIGPNVLMVHKRSHTGERPFQCNQCGASFTQKGNLLRHIKLHSGEKPFKCHLCNYACRRRD
ALTGHLRTHSVIKEETNHSEMAEDLCKIGSERSLVLDRLASNVAKRKSSMPQKFLGDKGLSD
TPYDSSASYEKENEMMKSHVMDQAINNAINYLGAESLRPLVQTPPGGSEVVPVISPMYQLHK
PLAEGTPRSNHSAQDSAVENLLLLSKAKLVPSEREASPSNSCQDSTDTESNNEEQRSGLIYL
TNHIAPHARNGLSLKEEHRAYDLLRAASENSQDALRVVSTSGEQMKVYKCEHCRVLFLDHVM
YTIHMGCHGFRDPFECNMCGYHSQDRYEFSSHITRGEHRFHMS
SEQ ID NO: 5 - Wild type human IKZF1 isoform 2 (variant 2) mRNA
sequence
(SEQ ID NO: 5)
CCTTTCTCTGCACCCTGGTGGAGTGTGAAGGCAGCAGAGGAACCTTTTGGAGGAGGAAGAGG
ACACAGAGGCCCTGTAGCCAGGCACCAAGATCCCTCCCAGGTGGCTGGGTCTGAGGGGAACT
CCGAGCAGCCCTAGGTCCTCAAAGTCTGGATTTGTGTGGAAAAGGCAGCTCTCACTTGGCCT
TGGCGAGGCCTCGGTTGGTTGATAACCTGAGGACCATGGATGCTGATGAGGGTCAAGACATG
TCCCAAGTTTCAGGGAAGGAAAGCCCCCCTGTAAGCGATACTCCAGATGAGGGCGATGAGCC
CATGCCGATCCCCGAGGACCTCTCCACCACCTCGGGAGGACAGCAAAGCTCCAAGAGTGACA
GAGTCGTGGCCAGTAATGTTAAAGTAGAGACTCAGAGTGATGAAGAGAATGGGCGTGCCTGT
GAAATGAATGGGGAAGAATGTGCGGAGGATTTACGAATGCTTGATGCCTCGGGAGAGAAAAT
GAATGGCTCCCACAGGGACCAAGGCAGCTCGGCTTTGTCGGGAGTTGGAGGCATTCGACTTC
CTAACGGAAAACTAAAGTGTGATATCTGTGGGATCATTTGCATCGGGCCCAATGTGCTCATG
GTTCACAAAAGAAGCCACACTGGAGAACGGCCCTTCCAGTGCAATCAGTGCGGGGCCTCATT
CACCCAGAAGGGCAACCTGCTCCGGCACATCAAGCTGCATTCCGGGGAGAAGCCCTTCAAAT
GCCACCTCTGCAACTACGCCTGCCGCCGGAGGGACGCCCTCACTGGCCACCTGAGGACGCAC
TCCGTCATTAAAGAAGAAACTAATCACAGTGAAATGGCAGAAGACCTGTGCAAGATAGGATC
AGAGAGATCTCTCGTGCTGGACAGACTAGCAAGTAACGTCGCCAAACGTAAGAGCTCTATGC
CTCAGAAATTTCTTGGGGACAAGGGCCTGTCCGACACGCCCTACGACAGCAGCGCCAGCTAC
GAGAAGGAGAACGAAATGATGAAGTCCCACGTGATGGACCAAGCCATCAACAACGCCATCAA
CTACCTGGGGGCCGAGTCCCTGCGCCCGCTGGTGCAGACGCCCCCGGGCGGTTCCGAGGTGG
TCCCGGTCATCAGCCCGATGTACCAGCTGCACAAGCCGCTCGCGGAGGGCACCCCGCGCTCC
AACCACTCGGCCCAGGACAGCGCCGTGGAGAACCTGCTGCTGCTCTCCAAGGCCAAGTTGGT
GCCCTCGGAGCGCGAGGCGTCCCCGAGCAACAGCTGCCAAGACTCCACGGACACCGAGAGCA
ACAACGAGGAGCAGCGCAGCGGTCTCATCTACCTGACCAACCACATCGCCCCGCACGCGCGC
AACGGGCTGTCGCTCAAGGAGGAGCACCGCGCCTACGACCTGCTGCGCGCCGCCTCCGAGAA
CTCGCAGGACGCGCTCCGCGTGGTCAGCACCAGCGGGGAGCAGATGAAGGTGTACAAGTGCG
AACACTGCCGGGTGCTCTTCCTGGATCACGTCATGTACACCATCCACATGGGCTGCCACGGC
TTCCGTGATCCTTTTGAGTGCAACATGTGCGGCTACCACAGCCAGGACCGGTACGAGTTCTC
GTCGCACATAACGCGAGGGGAGCACCGCTTCCACATGAGCTAAAGCCCTCCCGCGCCCCCAC
CCCAGACCCCGAGCCACCCCAGGAAAAGCACAAGGACTGCCGCCTTCTCGCTCCCGCCAGCA
GCATAGACTGGACTGGACCAGACAATGTTGTGTTTGGATTTGTAACTGTTTTTTGTTTTTTG
TTTGAGTTGGTTGATTGGGGTTTGATTTGCTTTTGAAAAGATTTTTATTTTTAGAGGCAGGG
CTGCATTGGGAGCATCCAGAACTGCTACCTTCCTAGATGTTTCCCCAGACCGCTGGCTGAGA
TTCCCTCACCTGTCGCTTCCTAGAATCCCCTTCTCCAAACGATTAGTCTAAATTTTCAGAGA
GAAATAGATAAAACACGCCACAGCCTGGGAAGGAGCGTGCTCTACCCTGTGCTAAGCACGGG
GTTCGCGCACCAGGTGTCTTTTTCCAGTCCCCAGAAGCAGAGAGCACAGCCCCTGCTGTGTG
GGTCTGCAGGTGAGCAGACAGGACAGGTGTGCCGCCACCCAAGTGCCAAGACACAGCAGGGC
CAACAACCTGTGCCCAGGCCAGCTTCGAGCTACATGCATCTAGGGCGGAGAGGCTGCACTTG
TGAGAGAAAATACTATTTCAAGTCATATTCTGCGTAGGAAAATGAATTGGTTGGGGAAAGTC
GTGTCTGTCAGACTGCCCTGGGTGGAGGGAGACGCCGGGCTAGAGCCTTTGGGATCGTCCTG
GATTCACTGGCTTTGCGGAGGCTGCTCAGATGGCCTGAGCCTCCCGAGGCTTGCTGCCCCGT
AGGAGGAGACTGTCTTCCCGTGGGCATATCTGGGGAGCCCTGTTCCCCGCTTTTTCACTCCC
ATACCTTTAATGGCCCCCAAAATCTGTCACTACAATTTAAACACCAGTCCCGAAATTTGGAT
CTTCTTTCTTTTTGAATCTCTCAAACGGCAACATTCCTCAGAAACCAAAGCTTTATTTCAAA
TCTCTTCCTTCCCTGGCTGGTTCCATCTAGTACCAGAGGCCTCTTTTCCTGAAGAAATCCAA
TCCTAGCCCTCATTTTAATTATGTACATCTGTTTGTAGCCACAAGCCTGAATTTCTCAGTGT
TGGTAAGTTTCTTTACCTACCCTCACTATATATTATTCTCGTTTTAAAACCCATAAAGGAGT
GATTTAGAACAGTCATTAATTTTCAACTCAATGAAATATGTGAAGCCCAGCATCTCTGTTGC
TAACACACAGAGCTCACCTGTTTGAAACCAAGCTTTCAAACATGTTGAAGCTCTTTACTGTA
AAGGCAAGCCAGCATGTGTGTCCACACATACATAGGATGGCTGGCTCTGCACCTGTAGGATA
TTGGAATGCACAGGGCAATTGAGGGACTGAGCCAGACCTTCGGAGAGTAATGCCACCAGATC
CCCTAGGAAAGAGGAGGCAAATGGCACTGCAGGTGAGAACCCCGCCCATCCGTGCTATGACA
TGGAGGCACTGAAGCCCGAGGAAGGTGTGTGGAGATTCTAATCCCAACAAGCAAGGGTCTCC
TTCAAGATTAATGCTATCAATCATTAAGGTCATTACTCTCAACCACCTAGGCAATGAAGAAT
ATACCATTTCAAATATTTACAGTACTTGTCTTCACCAACACTGTCCCAAGGTGAAATGAAGC
AACAGAGAGGAAATTGTACATAAGTACCTCAGCATTTAATCCAAACAGGGGTTCTTAGTCTC
AGCACTATGACATTTTGGGCTGACTACTTATTTGTTAGGCGGGAGCTCTCCTGTGCATTGTA
GGATAATTAGCAGTATCCCTGGTGGCTACCCAATAGACGCCAGTAGCACCCCGAATTGACAA
CCCAAACTCTCCAGACATCACCAACTGTCCCCTGCGAGGAGAAATCACTCCTGGGGGAGAAC
CACTGACCCAAATGAATTCTAAACCAATCAAATGTCTGGGAAGCCCTCCAAGAAAAAAAATA
GAAAAGCACTTGAAGAATATTCCCAATATTCCCGGTCAGCAGTATCAAGGCTGACTTGTGTT
CATGTGGAGTCATTATAAATTCTATAAATCAATTATTCCCCTTCGGTCTTAAAAATATATTT
CCTCATAAACATTTGAGTTTTGTTGAAAAGATGGAGTTTACAAAGATACCATTCTTGAGTCA
TGGATTTCTCTGCTCACAGAAGGGTGTGGCATTTGGAAACGGGAATAAACAAAATTGCTGCA
CCAATGCACTGAGTGAAGGAAGAGAGACAGAGGATCAAGGGCTTTAGACAGCACTCCTTCAA
TATGCAATCACAGAGAAAGATGCGCCTTATCCAAGTTAATATCTCTAAGGTGAGAGCCTTCT
TAGAGTCAGTTTGTTGCAAATTTCACCTACTCTGTTCTTTTCCATCCATCCCCCTGAGTCAG
TTGGTTGAAGGGAGTTATTTTTTCAAGTGGAATTCAAACAAAGCTCAAACCAGAACTGTAAA
TAGTGATTGCAGGAATTCTTTTCTAAACTGCTTTGCCCTTTCCTCTCACTGCCTTTTATAGC
CAATATAAATGTCTCTTTGCACACCTTTTGTTGTGGTTTTATATTGTAACACCATTTTTCTT
TGAAACTATTGTATTTAAAGTAAGGTTTCATATTATGTCAGCAAGTAATTAACTTATGTTTA
AAAGGTGGCCATATCATGTACCAAAAGTTGCTGAAGTTTCTCTTCTAGCTGGTAAAGTAGGA
GTTTGCATGACTTCACACTTTTTTTGCGTAGTTTCTTCTGTTGTATGATGGCGTGAGTGTGT
GTCTTGGGTACCGCTGTGTACTACTGTGTGCCTAGATTCCATGCACTCTCGTTGTGTTTGAA
GTAAATATTGGAGACCGGAGGGTAACAGGTTGGCCTGTTGATTACAGCTAGTAATCGCTGTG
TCTTGTTCCGCCCCCTCCCTGACACCCCAGCTTCCCAGGATGTGGAAAGCCTGGATCTCAGC
TCCTTGCCCCATATCCCTTCTGTAATTTGTACCTAAAGAGTGTGATTATCCTAATTCAAGAG
TCACTAAAACTCATCACATTATCATTGCATATCAGCAAAGGGTAAAGTCCTAGCACCAATTG
CTTCACATACCAGCATGTTCCATTTCCAATTTAGAATTAGCCACATAATAAAATCTTAGAAT
CTTCCTTGAGAAAGAGCTGCCTGAGATGTAGTTTTGTTATATGGTTCCCCACCGACCATTTT
TGTGCTTTTTTCTTGTTTTGTTTTGTTTTGACTGCACTGTGAGTTTTGTAGTGTCCTCTTCT
TGCCAAAACAAACGCGAGATGAACTGGACTTATGTAGACAAATCGTGATGCCAGTGTATCCT
TCCTTTCTTCAGTTCCAGCAATAATGAATGGTCAACTTTTTTAAAATCTAGATCTCTCTCAT
TCATTTCAATGTATTTTTACTTTAAGATGAACCAAAATTATTAGACTTATTTAAGATGTACA
GGCATCAGAAAAAAGAAGCACATAATGCTTTTGGTGCGATGGCACTCACTGTGAACATGTGT
AACCACATATTAATATGCAATATTGTTTCCAATACTTTCTAATACAGTTTTTTATAATGTTG
TGTGTGGTGATTGTTCAGGTCGAATCTGTTGTATCCAGTACAGCTTTAGGTCTTCAGCTGCC
CTTCTGGCGAGTACATGCACAGGATTGTAAATGAGAAATGCAGTCATATTTCCAGTCTGCCT
CTATGATGATGTTAAATTATTGCTGTTTAGCTGTGAACAAGGGATGTACCACTGGAGGAATA
GAGTATCCTTTTGTACACATTTTGAAATGCTTCTTCTGTAGTGATAGAACAAATAAATGCAA
CGAATACTCTGTCTGCCCTATCCCGTGAAGTCCACACTGGCGTAAGAGAAGGCCCAGCAGAG
CAGGAATCTGCCTAGACTTTCTCCCAATGAGATCCCAATATGAGAGGGAGAAGAGATGGGCC
TCAGGACAGCTGCAATACCACTTGGGAACACATGTGGTGTCTTGATGTGGCCAGCGCAGCAG
TTCAGCACAACGTACCTCCCATCTACAACAGTGCTGGACGTGGGAATTCTAAGTCCCAGTCT
TGAGGGTGGGTGGAGATGGAGGGCAACAAGAGATACATTTCCAGTTCTCCACTGCAGCATGC
TTCAGTCATTCTGTGAGTGGCCGGGCCCAGGGCCCTCACAATTTCACTACCTTGTCTTTTAC
ATAGTCATAAGAATTATCCTCAACATAGCCTTTTGACGCTGTAAATCTTGAGTATTCATTTA
CCCTTTTCTGATCTCCTGGAAACAGCTGCCTGCCTGCATTGCACTTCTCTTCCCGAGGAGTG
GGGTAAATTTAAAAGTCAAGTTATAGTTTGGATGTTAGTATAGAATTTTGAAATTGGGAATT
AAAAATCAGGACTGGGGACTGGGAGACCAAAAATTTCTGATCCCATTTCTGATGGATGTGTC
ACACCTTTTCTGTCAAAATAAAATGTCTTGGAGGTTATGACTCCTTGGTGAAA
SEQ ID NO: 6 - Wild type human IKZF1 isoform 2 (variant 2)
polynucleotide coding sequence
(SEQ ID NO: 6)
ATGGATGCTGATGAGGGTCAAGACATGTCCCAAGTTTCAGGGAAGGAAAGCCCCCCTGTAAG
CGATACTCCAGATGAGGGCGATGAGCCCATGCCGATCCCCGAGGACCTCTCCACCACCTCGG
GAGGACAGCAAAGCTCCAAGAGTGACAGAGTCGTGGCCAGTAATGTTAAAGTAGAGACTCAG
AGTGATGAAGAGAATGGGCGTGCCTGTGAAATGAATGGGGAAGAATGTGCGGAGGATTTACG
AATGCTTGATGCCTCGGGAGAGAAAATGAATGGCTCCCACAGGGACCAAGGCAGCTCGGCTT
TGTCGGGAGTTGGAGGCATTCGACTTCCTAACGGAAAACTAAAGTGTGATATCTGTGGGATC
ATTTGCATCGGGCCCAATGTGCTCATGGTTCACAAAAGAAGCCACACTGGAGAACGGCCCTT
CCAGTGCAATCAGTGCGGGGCCTCATTCACCCAGAAGGGCAACCTGCTCCGGCACATCAAGC
TGCATTCCGGGGAGAAGCCCTTCAAATGCCACCTCTGCAACTACGCCTGCCGCCGGAGGGAC
GCCCTCACTGGCCACCTGAGGACGCACTCCGTCATTAAAGAAGAAACTAATCACAGTGAAAT
GGCAGAAGACCTGTGCAAGATAGGATCAGAGAGATCTCTCGTGCTGGACAGACTAGCAAGTA
ACGTCGCCAAACGTAAGAGCTCTATGCCTCAGAAATTTCTTGGGGACAAGGGCCTGTCCGAC
ACGCCCTACGACAGCAGCGCCAGCTACGAGAAGGAGAACGAAATGATGAAGTCCCACGTGAT
GGACCAAGCCATCAACAACGCCATCAACTACCTGGGGGCCGAGTCCCTGCGCCCGCTGGTGC
AGACGCCCCCGGGCGGTTCCGAGGTGGTCCCGGTCATCAGCCCGATGTACCAGCTGCACAAG
CCGCTCGCGGAGGGCACCCCGCGCTCCAACCACTCGGCCCAGGACAGCGCCGTGGAGAACCT
GCTGCTGCTCTCCAAGGCCAAGTTGGTGCCCTCGGAGCGCGAGGCGTCCCCGAGCAACAGCT
GCCAAGACTCCACGGACACCGAGAGCAACAACGAGGAGCAGCGCAGCGGTCTCATCTACCTG
ACCAACCACATCGCCCCGCACGCGCGCAACGGGCTGTCGCTCAAGGAGGAGCACCGCGCCTA
CGACCTGCTGCGCGCCGCCTCCGAGAACTCGCAGGACGCGCTCCGCGTGGTCAGCACCAGCG
GGGAGCAGATGAAGGTGTACAAGTGCGAACACTGCCGGGTGCTCTTCCTGGATCACGTCATG
TACACCATCCACATGGGCTGCCACGGCTTCCGTGATCCTTTTGAGTGCAACATGTGCGGCTA
CCACAGCCAGGACCGGTACGAGTTCTCGTCGCACATAACGCGAGGGGAGCACCGCTTCCACA
TGAGCTAA
SEQ ID NO: 7 - Wild type human NPM1 isoform 1 amino acid sequence
(SEQ ID NO: 7)
MEDSMDMDMSPLRPQNYLFGCELKADKDYHFKVDNDENEHQLSLRTVSLGAGAKDELHIVEA
EAMNYEGSPIKVTLATLKMSVQPTVSLGGFEITPPVVLRLKCGSGPVHISGQHLVAVEEDAE
SEDEEEEDVKLLSISGKRSAPGGGSKVPQKKVKLAADEDDDDDDEEDDDEDDDDDDFDDEEA
EEKAPVKKSIRDTPAKNAQKSNQNGKDSKPSSTPRSKGQESFKKQEKTPKTPKGPSSVEDIK
AKMQASIEKGGSLPKVEAKFINYVKNCFRMTDQEAIQDLWQWRKSL
SEQ ID NO: 8 - Wild type human NPM1 isoform 1 mRNA sequence
(SEQ ID NO: 8)
CTTTCCCTGGTGTGATTCCGTCCTGCGCGGTTGTTCTCTGGAGCAGCGTTCTTTTATCTCCG
TCCGCCTTCTCTCCTACCTAAGTGCGTGCCGCCACCCGATGGAAGATTCGATGGACATGGAC
ATGAGCCCCCTGAGGCCCCAGAACTATCTTTTCGGTTGTGAACTAAAGGCCGACAAAGATTA
TCACTTTAAGGTGGATAATGATGAAAATGAGCACCAGTTATCTTTAAGAACGGTCAGTTTAG
GGGCTGGTGCAAAGGATGAGTTGCACATTGTTGAAGCAGAGGCAATGAATTACGAAGGCAGT
CCAATTAAAGTAACACTGGCAACTTTGAAAATGTCTGTACAGCCAACGGTTTCCCTTGGGGG
CTTTGAAATAACACCACCAGTGGTCTTAAGGTTGAAGTGTGGTTCAGGGCCAGTGCATATTA
GTGGACAGCACTTAGTAGCTGTGGAGGAAGATGCAGAGTCAGAAGATGAAGAGGAGGAGGAT
GTGAAACTCTTAAGTATATCTGGAAAGCGGTCTGCCCCTGGAGGTGGTAGCAAGGTTCCACA
GAAAAAAGTAAAACTTGCTGCTGATGAAGATGATGACGATGATGATGAAGAGGATGATGATG
AAGATGATGATGATGATGATTTTGATGATGAGGAAGCTGAAGAAAAAGCGCCAGTGAAGAAA
TCTATACGAGATACTCCAGCCAAAAATGCACAAAAGTCAAATCAGAATGGAAAAGACTCAAA
ACCATCATCAACACCAAGATCAAAAGGACAAGAATCCTTCAAGAAACAGGAAAAAACTCCTA
AAACACCAAAAGGACCTAGTTCTGTAGAAGACATTAAAGCAAAAATGCAAGCAAGTATAGAA
AAAGGTGGTTCTCTTCCCAAAGTGGAAGCCAAATTCATCAATTATGTGAAGAATTGCTTCCG
GATGACTGACCAAGAGGCTATTCAAGATCTCTGGCAGTGGAGGAAGTCTCTTTAAGAAAATA
GTTTAAACAATTTGTTAAAAAATTTTCCGTCTTATTTCATTTCTGTAACAGTTGATATCTGG
CTGTCCTTTTTATAATGCAGAGTGAGAACTTTCCCTACCGTGTTTGATAAATGTTGTCCAGG
TTCTATTGCCAAGAATGTGTTGTCCAAAATGCCTGTTTAGTTTTTAAAGATGGAACTCCACC
CTTTGCTTGGTTTTAAGTATGTATGGAATGTTATGATAGGACATAGTAGTAGCGGTGGTCAG
ACATGGAAATGGTGGGGAGACAAAAATATACATGTGAAATAAAACTCAGTATTTTAATAAAG
TAGCACGGTTTCTATTGA
SEQ ID NO: 9 - Wild type human NPM1 isoform 1 polynucleotide coding
sequence
(SEQ ID NO: 9)
ATGGAAGATTCGATGGACATGGACATGAGCCCCCTGAGGCCCCAGAACTATCTTTTCGGTTG
TGAACTAAAGGCCGACAAAGATTATCACTTTAAGGTGGATAATGATGAAAATGAGCACCAGT
TATCTTTAAGAACGGTCAGTTTAGGGGCTGGTGCAAAGGATGAGTTGCACATTGTTGAAGCA
GAGGCAATGAATTACGAAGGCAGTCCAATTAAAGTAACACTGGCAACTTTGAAAATGTCTGT
ACAGCCAACGGTTTCCCTTGGGGGCTTTGAAATAACACCACCAGTGGTCTTAAGGTTGAAGT
GTGGTTCAGGGCCAGTGCATATTAGTGGACAGCACTTAGTAGCTGTGGAGGAAGATGCAGAG
TCAGAAGATGAAGAGGAGGAGGATGTGAAACTCTTAAGTATATCTGGAAAGCGGTCTGCCCC
TGGAGGTGGTAGCAAGGTTCCACAGAAAAAAGTAAAACTTGCTGCTGATGAAGATGATGACG
ATGATGATGAAGAGGATGATGATGAAGATGATGATGATGATGATTTTGATGATGAGGAAGCT
GAAGAAAAAGCGCCAGTGAAGAAATCTATACGAGATACTCCAGCCAAAAATGCACAAAAGTC
AAATCAGAATGGAAAAGACTCAAAACCATCATCAACACCAAGATCAAAAGGACAAGAATCCT
TCAAGAAACAGGAAAAAACTCCTAAAACACCAAAAGGACCTAGTTCTGTAGAAGACATTAAA
GCAAAAATGCAAGCAAGTATAGAAAAAGGTGGTTCTCTTCCCAAAGTGGAAGCCAAATTCAT
CAATTATGTGAAGAATTGCTTCCGGATGACTGACCAAGAGGCTATTCAAGATCTCTGGCAGT
GGAGGAAGTCTCTTTAA
SEQ ID NO: 10 - Wild type human NPM1 isoform 2 amino acid sequence
(SEQ ID NO: 10)
MEDSMDMDMSPLRPQNYLFGCELKADKDYHFKVDNDENEHQLSLRTVSLGAGAKDELHIVEA
EAMNYEGSPIKVTLATLKMSVQPTVSLGGFEITPPVVLRLKCGSGPVHISGQHLVAVEEDAE
SEDEEEEDVKLLSISGKRSAPGGGSKVPQKKVKLAADEDDDDDDEEDDDEDDDDDDFDDEEA
EEKAPVKKGQESFKKQEKTPKTPKGPSSVEDIKAKMQASIEKGGSLPKVEAKFINYVKNCFR
MTDQEAIQDLWQWRKSL
SEQ ID NO: 11 - Wild type human NPM1 isoform 2 mRNA sequence
(SEQ ID NO: 11)
CTTTCCCTGGTGTGATTCCGTCCTGCGCGGTTGTTCTCTGGAGCAGCGTTCTTTTATCTCCG
TCCGCCTTCTCTCCTACCTAAGTGCGTGCCGCCACCCGATGGAAGATTCGATGGACATGGAC
ATGAGCCCCCTGAGGCCCCAGAACTATCTTTTCGGTTGTGAACTAAAGGCCGACAAAGATTA
TCACTTTAAGGTGGATAATGATGAAAATGAGCACCAGTTATCTTTAAGAACGGTCAGTTTAG
GGGCTGGTGCAAAGGATGAGTTGCACATTGTTGAAGCAGAGGCAATGAATTACGAAGGCAGT
CCAATTAAAGTAACACTGGCAACTTTGAAAATGTCTGTACAGCCAACGGTTTCCCTTGGGGG
CTTTGAAATAACACCACCAGTGGTCTTAAGGTTGAAGTGTGGTTCAGGGCCAGTGCATATTA
GTGGACAGCACTTAGTAGCTGTGGAGGAAGATGCAGAGTCAGAAGATGAAGAGGAGGAGGAT
GTGAAACTCTTAAGTATATCTGGAAAGCGGTCTGCCCCTGGAGGTGGTAGCAAGGTTCCACA
GAAAAAAGTAAAACTTGCTGCTGATGAAGATGATGACGATGATGATGAAGAGGATGATGATG
AAGATGATGATGATGATGATTTTGATGATGAGGAAGCTGAAGAAAAAGCGCCAGTGAAGAAA
GGACAAGAATCCTTCAAGAAACAGGAAAAAACTCCTAAAACACCAAAAGGACCTAGTTCTGT
AGAAGACATTAAAGCAAAAATGCAAGCAAGTATAGAAAAAGGTGGTTCTCTTCCCAAAGTGG
AAGCCAAATTCATCAATTATGTGAAGAATTGCTTCCGGATGACTGACCAAGAGGCTATTCAA
GATCTCTGGCAGTGGAGGAAGTCTCTTTAAGAAAATAGTTTAAACAATTTGTTAAAAAATTT
TCCGTCTTATTTCATTTCTGTAACAGTTGATATCTGGCTGTCCTTTTTATAATGCAGAGTGA
GAACTTTCCCTACCGTGTTTGATAAATGTTGTCCAGGTTCTATTGCCAAGAATGTGTTGTCC
AAAATGCCTGTTTAGTTTTTAAAGATGGAACTCCACCCTTTGCTTGGTTTTAAGTATGTATG
GAATGTTATGATAGGACATAGTAGTAGCGGTGGTCAGACATGGAAATGGTGGGGAGACAAAA
ATATACATGTGAAATAAAACTCAGTATTTTAATAAAGTAGCACGGTTTCTATTGA
SEQ ID NO: 12 - Wild type human NPM1 isoform 2 polynucleotide coding
sequence
(SEQ ID NO: 12)
ATGGAAGATTCGATGGACATGGACATGAGCCCCCTGAGGCCCCAGAACTATCTTTTCGGTTG
TGAACTAAAGGCCGACAAAGATTATCACTTTAAGGTGGATAATGATGAAAATGAGCACCAGT
TATCTTTAAGAACGGTCAGTTTAGGGGCTGGTGCAAAGGATGAGTTGCACATTGTTGAAGCA
GAGGCAATGAATTACGAAGGCAGTCCAATTAAAGTAACACTGGCAACTTTGAAAATGTCTGT
ACAGCCAACGGTTTCCCTTGGGGGCTTTGAAATAACACCACCAGTGGTCTTAAGGTTGAAGT
GTGGTTCAGGGCCAGTGCATATTAGTGGACAGCACTTAGTAGCTGTGGAGGAAGATGCAGAG
TCAGAAGATGAAGAGGAGGAGGATGTGAAACTCTTAAGTATATCTGGAAAGCGGTCTGCCCC
TGGAGGTGGTAGCAAGGTTCCACAGAAAAAAGTAAAACTTGCTGCTGATGAAGATGATGACG
ATGATGATGAAGAGGATGATGATGAAGATGATGATGATGATGATTTTGATGATGAGGAAGCT
GAAGAAAAAGCGCCAGTGAAGAAAGGACAAGAATCCTTCAAGAAACAGGAAAAAACTCCTAA
AACACCAAAAGGACCTAGTTCTGTAGAAGACATTAAAGCAAAAATGCAAGCAAGTATAGAAA
AAGGTGGTTCTCTTCCCAAAGTGGAAGCCAAATTCATCAATTATGTGAAGAATTGCTTCCGG
ATGACTGACCAAGAGGCTATTCAAGATCTCTGGCAGTGGAGGAAGTCTCTTTAA
SEQ ID NO: 13 - Wild type human EZH2 isoform a (variant 1) amino
acid sequence
(SEQ ID NO: 13)
MGQTGKKSEKGPVCWRKRVKSEYMRLRQLKRFRRADEVKSMFSSNRQKILERTEILNQEWKQ
RRIQPVHILTSVSSLRGTRECSVTSDLDFPTQVIPLKTLNAVASVPIMYSWSPLQQNFMVED
ETVLHNIPYMGDEVLDQDGTFIEELIKNYDGKVHGDRECGFINDEIFVELVNALGQYNDDDD
DDDGDDPEEREEKQKDLEDHRDDKESRPPRKFPSDKIFEAISSMFPDKGTAEELKEKYKELT
EQQLPGALPPECTPNIDGPNAKSVQREQSLHSFHTLFCRRCFKYDCFLHRKCNYSFHATPNT
YKRKNTETALDNKPCGPQCYQHLEGAKEFAAALTAERIKTPPKRPGGRRRGRLPNNSSRPST
PTINVLESKDTDSDREAGTETGGENNDKEEEEKKDETSSSSEANSRCQTPIKMKPNIEPPEN
VEWSGAEASMFRVLIGTYYDNFCAIARLIGTKTCRQVYEFRVKESSIIAPAPAEDVDTPPRK
KKRKHRLWAAHCRKIQLKKDGSSNHVYNYQPCDHPRQPCDSSCPCVIAQNFCEKFCQCSSEC
QNRFPGCRCKAQCNTKQCPCYLAVRECDPDLCLTCGAADHWDSKNVSCKNCSIQRGSKKHLL
LAPSDVAGWGIFIKDPVQKNEFISEYCGEIISQDEADRRGKVYDKYMCSFLFNLNNDFVVDA
TRKGNKIRFANHSVNPNCYAKVMMVNGDHRIGIFAKRAIQTGEELFFDYRYSQADALKYVGI
EREMEIP
SEQ ID NO: 14 - Wild type human EZH2 isoform a (variant 1) mRNA
sequence
(SEQ ID NO: 14)
GTTTGGCGCTCGGTCCGGTCGCGTCCGACACCCGGTGGGACTCAGAAGGCAGTGGAGCCCCG
GCGGCGGCGGCGGCGGCGCGCGGGGGCGACGCGCGGGAACAACGCGAGTCGGCGCGCGGGAC
GAAGAATAATCATGGGCCAGACTGGGAAGAAATCTGAGAAGGGACCAGTTTGTTGGCGGAAG
CGTGTAAAATCAGAGTACATGCGACTGAGACAGCTCAAGAGGTTCAGACGAGCTGATGAAGT
AAAGAGTATGTTTAGTTCCAATCGTCAGAAAATTTTGGAAAGAACGGAAATCTTAAACCAAG
AATGGAAACAGCGAAGGATACAGCCTGTGCACATCCTGACTTCTGTGAGCTCATTGCGCGGG
ACTAGGGAGTGTTCGGTGACCAGTGACTTGGATTTTCCAACACAAGTCATCCCATTAAAGAC
TCTGAATGCAGTTGCTTCAGTACCCATAATGTATTCTTGGTCTCCCCTACAGCAGAATTTTA
TGGTGGAAGATGAAACTGTTTTACATAACATTCCTTATATGGGAGATGAAGTTTTAGATCAG
GATGGTACTTTCATTGAAGAACTAATAAAAAATTATGATGGGAAAGTACACGGGGATAGAGA
ATGTGGGTTTATAAATGATGAAATTTTTGTGGAGTTGGTGAATGCCCTTGGTCAATATAATG
ATGATGACGATGATGATGATGGAGACGATCCTGAAGAAAGAGAAGAAAAGCAGAAAGATCTG
GAGGATCACCGAGATGATAAAGAAAGCCGCCCACCTCGGAAATTTCCTTCTGATAAAATTTT
TGAAGCCATTTCCTCAATGTTTCCAGATAAGGGCACAGCAGAAGAACTAAAGGAAAAATATA
AAGAACTCACCGAACAGCAGCTCCCAGGCGCACTTCCTCCTGAATGTACCCCCAACATAGAT
GGACCAAATGCTAAATCTGTTCAGAGAGAGCAAAGCTTACACTCCTTTCATACGCTTTTCTG
TAGGCGATGTTTTAAATATGACTGCTTCCTACATCGTAAGTGCAATTATTCTTTTCATGCAA
CACCCAACACTTATAAGCGGAAGAACACAGAAACAGCTCTAGACAACAAACCTTGTGGACCA
CAGTGTTACCAGCATTTGGAGGGAGCAAAGGAGTTTGCTGCTGCTCTCACCGCTGAGCGGAT
AAAGACCCCACCAAAACGTCCAGGAGGCCGCAGAAGAGGACGGCTTCCCAATAACAGTAGCA
GGCCCAGCACCCCCACCATTAATGTGCTGGAATCAAAGGATACAGACAGTGATAGGGAAGCA
GGGACTGAAACGGGGGGAGAGAACAATGATAAAGAAGAAGAAGAGAAGAAAGATGAAACTTC
GAGCTCCTCTGAAGCAAATTCTCGGTGTCAAACACCAATAAAGATGAAGCCAAATATTGAAC
CTCCTGAGAATGTGGAGTGGAGTGGTGCTGAAGCCTCAATGTTTAGAGTCCTCATTGGCACT
TACTATGACAATTTCTGTGCCATTGCTAGGTTAATTGGGACCAAAACATGTAGACAGGTGTA
TGAGTTTAGAGTCAAAGAATCTAGCATCATAGCTCCAGCTCCCGCTGAGGATGTGGATACTC
CTCCAAGGAAAAAGAAGAGGAAACACCGGTTGTGGGCTGCACACTGCAGAAAGATACAGCTG
AAAAAGGACGGCTCCTCTAACCATGTTTACAACTATCAACCCTGTGATCATCCACGGCAGCC
TTGTGACAGTTCGTGCCCTTGTGTGATAGCACAAAATTTTTGTGAAAAGTTTTGTCAATGTA
GTTCAGAGTGTCAAAACCGCTTTCCGGGATGCCGCTGCAAAGCACAGTGCAACACCAAGCAG
TGCCCGTGCTACCTGGCTGTCCGAGAGTGTGACCCTGACCTCTGTCTTACTTGTGGAGCCGC
TGACCATTGGGACAGTAAAAATGTGTCCTGCAAGAACTGCAGTATTCAGCGGGGCTCCAAAA
AGCATCTATTGCTGGCACCATCTGACGTGGCAGGCTGGGGGATTTTTATCAAAGATCCTGTG
CAGAAAAATGAATTCATCTCAGAATACTGTGGAGAGATTATTTCTCAAGATGAAGCTGACAG
AAGAGGGAAAGTGTATGATAAATACATGTGCAGCTTTCTGTTCAACTTGAACAATGATTTTG
TGGTGGATGCAACCCGCAAGGGTAACAAAATTCGTTTTGCAAATCATTCGGTAAATCCAAAC
TGCTATGCAAAAGTTATGATGGTTAACGGTGATCACAGGATAGGTATTTTTGCCAAGAGAGC
CATCCAGACTGGCGAAGAGCTGTTTTTTGATTACAGATACAGCCAGGCTGATGCCCTGAAGT
ATGTCGGCATCGAAAGAGAAATGGAAATCCCTTGACATCTGCTACCTCCTCCCCCCTCCTCT
GAAACAGCTGCCTTAGCTTCAGGAACCTCGAGTACTGTGGGCAATTTAGAAAAAGAACATGC
AGTTTGAAATTCTGAATTTGCAAAGTACTGTAAGAATAATTTATAGTAATGAGTTTAAAAAT
CAACTTTTTATTGCCTTCTCACCAGCTGCAAAGTGTTTTGTACCAGTGAATTTTTGCAATAA
TGCAGTATGGTACATTTTTCAACTTTGAATAAAGAATACTTGAACTTGTC
SEQ ID NO: 15 - Wild type human EZH2 isoform a (variant 1)
polynucleotide coding sequence
(SEQ ID NO: 15)
ATGGGCCAGACTGGGAAGAAATCTGAGAAGGGACCAGTTTGTTGGCGGAAGCGTGTAAAATC
AGAGTACATGCGACTGAGACAGCTCAAGAGGTTCAGACGAGCTGATGAAGTAAAGAGTATGT
TTAGTTCCAATCGTCAGAAAATTTTGGAAAGAACGGAAATCTTAAACCAAGAATGGAAACAG
CGAAGGATACAGCCTGTGCACATCCTGACTTCTGTGAGCTCATTGCGCGGGACTAGGGAGTG
TTCGGTGACCAGTGACTTGGATTTTCCAACACAAGTCATCCCATTAAAGACTCTGAATGCAG
TTGCTTCAGTACCCATAATGTATTCTTGGTCTCCCCTACAGCAGAATTTTATGGTGGAAGAT
GAAACTGTTTTACATAACATTCCTTATATGGGAGATGAAGTTTTAGATCAGGATGGTACTTT
CATTGAAGAACTAATAAAAAATTATGATGGGAAAGTACACGGGGATAGAGAATGTGGGTTTA
TAAATGATGAAATTTTTGTGGAGTTGGTGAATGCCCTTGGTCAATATAATGATGATGACGAT
GATGATGATGGAGACGATCCTGAAGAAAGAGAAGAAAAGCAGAAAGATCTGGAGGATCACCG
AGATGATAAAGAAAGCCGCCCACCTCGGAAATTTCCTTCTGATAAAATTTTTGAAGCCATTT
CCTCAATGTTTCCAGATAAGGGCACAGCAGAAGAACTAAAGGAAAAATATAAAGAACTCACC
GAACAGCAGCTCCCAGGCGCACTTCCTCCTGAATGTACCCCCAACATAGATGGACCAAATGC
TAAATCTGTTCAGAGAGAGCAAAGCTTACACTCCTTTCATACGCTTTTCTGTAGGCGATGTT
TTAAATATGACTGCTTCCTACATCGTAAGTGCAATTATTCTTTTCATGCAACACCCAACACT
TATAAGCGGAAGAACACAGAAACAGCTCTAGACAACAAACCTTGTGGACCACAGTGTTACCA
GCATTTGGAGGGAGCAAAGGAGTTTGCTGCTGCTCTCACCGCTGAGCGGATAAAGACCCCAC
CAAAACGTCCAGGAGGCCGCAGAAGAGGACGGCTTCCCAATAACAGTAGCAGGCCCAGCACC
CCCACCATTAATGTGCTGGAATCAAAGGATACAGACAGTGATAGGGAAGCAGGGACTGAAAC
GGGGGGAGAGAACAATGATAAAGAAGAAGAAGAGAAGAAAGATGAAACTTCGAGCTCCTCTG
AAGCAAATTCTCGGTGTCAAACACCAATAAAGATGAAGCCAAATATTGAACCTCCTGAGAAT
GTGGAGTGGAGTGGTGCTGAAGCCTCAATGTTTAGAGTCCTCATTGGCACTTACTATGACAA
TTTCTGTGCCATTGCTAGGTTAATTGGGACCAAAACATGTAGACAGGTGTATGAGTTTAGAG
TCAAAGAATCTAGCATCATAGCTCCAGCTCCCGCTGAGGATGTGGATACTCCTCCAAGGAAA
AAGAAGAGGAAACACCGGTTGTGGGCTGCACACTGCAGAAAGATACAGCTGAAAAAGGACGG
CTCCTCTAACCATGTTTACAACTATCAACCCTGTGATCATCCACGGCAGCCTTGTGACAGTT
CGTGCCCTTGTGTGATAGCACAAAATTTTTGTGAAAAGTTTTGTCAATGTAGTTCAGAGTGT
CAAAACCGCTTTCCGGGATGCCGCTGCAAAGCACAGTGCAACACCAAGCAGTGCCCGTGCTA
CCTGGCTGTCCGAGAGTGTGACCCTGACCTCTGTCTTACTTGTGGAGCCGCTGACCATTGGG
ACAGTAAAAATGTGTCCTGCAAGAACTGCAGTATTCAGCGGGGCTCCAAAAAGCATCTATTG
CTGGCACCATCTGACGTGGCAGGCTGGGGGATTTTTATCAAAGATCCTGTGCAGAAAAATGA
ATTCATCTCAGAATACTGTGGAGAGATTATTTCTCAAGATGAAGCTGACAGAAGAGGGAAAG
TGTATGATAAATACATGTGCAGCTTTCTGTTCAACTTGAACAATGATTTTGTGGTGGATGCA
ACCCGCAAGGGTAACAAAATTCGTTTTGCAAATCATTCGGTAAATCCAAACTGCTATGCAAA
AGTTATGATGGTTAACGGTGATCACAGGATAGGTATTTTTGCCAAGAGAGCCATCCAGACTG
GCGAAGAGCTGTTTTTTGATTACAGATACAGCCAGGCTGATGCCCTGAAGTATGTCGGCATC
GAAAGAGAAATGGAAATCCCTTGA
SEQ ID NO: 16 - Wild type human EZH2 isoform b (variant 2) amino
acid sequence
(SEQ ID NO: 16)
MGQTGKKSEKGPVCWRKRVKSEYMRLRQLKRFRRADEVKSMFSSNRQKILERTEILNQEWKQ
RRIQPVHILTSVSSLRGTREVEDETVLHNIPYMGDEVLDQDGTFIEELIKNYDGKVHGDREC
GFINDEIFVELVNALGQYNDDDDDDDGDDPEEREEKQKDLEDHRDDKESRPPRKFPSDKIFE
AISSMFPDKGTAEELKEKYKELTEQQLPGALPPECTPNIDGPNAKSVQREQSLHSFHTLFCR
RCFKYDCFLHPFHATPNTYKRKNTETALDNKPCGPQCYQHLEGAKEFAAALTAERIKTPPKR
PGGRRRGRLPNNSSRPSTPTINVLESKDTDSDREAGTETGGENNDKEEEEKKDETSSSSEAN
SRCQTPIKMKPNIEPPENVEWSGAEASMFRVLIGTYYDNFCAIARLIGTKTCRQVYEFRVKE
SSIIAPAPAEDVDTPPRKKKRKHRLWAAHCRKIQLKKDGSSNHVYNYQPCDHPRQPCDSSCP
CVIAQNFCEKFCQCSSECQNRFPGCRCKAQCNTKQCPCYLAVRECDPDLCLTCGAADHWDSK
NVSCKNCSIQRGSKKHLLLAPSDVAGWGIFIKDPVQKNEFISEYCGEIISQDEADRRGKVYD
KYMCSFLFNLNNDFVVDATRKGNKIRFANHSVNPNCYAKVMMVNGDHRIGIFAKRAIQTGEE
LFFDYRYSQADALKYVGIEREMEIP
SEQ ID NO: 17 - Wild type human EZH2 isoform b (variant 2) mRNA
sequence
(SEQ ID NO: 17)
GTTTGGCGCTCGGTCCGGTCGCGTCCGACACCCGGTGGGACTCAGAAGGCAGTGGAGCCCCG
GCGGCGGCGGCGGCGGCGCGCGGGGGCGACGCGCGGGAACAACGCGAGTCGGCGCGCGGGAC
GAAGAATAATCATGGGCCAGACTGGGAAGAAATCTGAGAAGGGACCAGTTTGTTGGCGGAAG
CGTGTAAAATCAGAGTACATGCGACTGAGACAGCTCAAGAGGTTCAGACGAGCTGATGAAGT
AAAGAGTATGTTTAGTTCCAATCGTCAGAAAATTTTGGAAAGAACGGAAATCTTAAACCAAG
AATGGAAACAGCGAAGGATACAGCCTGTGCACATCCTGACTTCTGTGAGCTCATTGCGCGGG
ACTAGGGAGGTGGAAGATGAAACTGTTTTACATAACATTCCTTATATGGGAGATGAAGTTTT
AGATCAGGATGGTACTTTCATTGAAGAACTAATAAAAAATTATGATGGGAAAGTACACGGGG
ATAGAGAATGTGGGTTTATAAATGATGAAATTTTTGTGGAGTTGGTGAATGCCCTTGGTCAA
TATAATGATGATGACGATGATGATGATGGAGACGATCCTGAAGAAAGAGAAGAAAAGCAGAA
AGATCTGGAGGATCACCGAGATGATAAAGAAAGCCGCCCACCTCGGAAATTTCCTTCTGATA
AAATTTTTGAAGCCATTTCCTCAATGTTTCCAGATAAGGGCACAGCAGAAGAACTAAAGGAA
AAATATAAAGAACTCACCGAACAGCAGCTCCCAGGCGCACTTCCTCCTGAATGTACCCCCAA
CATAGATGGACCAAATGCTAAATCTGTTCAGAGAGAGCAAAGCTTACACTCCTTTCATACGC
TTTTCTGTAGGCGATGTTTTAAATATGACTGCTTCCTACATCCTTTTCATGCAACACCCAAC
ACTTATAAGCGGAAGAACACAGAAACAGCTCTAGACAACAAACCTTGTGGACCACAGTGTTA
CCAGCATTTGGAGGGAGCAAAGGAGTTTGCTGCTGCTCTCACCGCTGAGCGGATAAAGACCC
CACCAAAACGTCCAGGAGGCCGCAGAAGAGGACGGCTTCCCAATAACAGTAGCAGGCCCAGC
ACCCCCACCATTAATGTGCTGGAATCAAAGGATACAGACAGTGATAGGGAAGCAGGGACTGA
AACGGGGGGAGAGAACAATGATAAAGAAGAAGAAGAGAAGAAAGATGAAACTTCGAGCTCCT
CTGAAGCAAATTCTCGGTGTCAAACACCAATAAAGATGAAGCCAAATATTGAACCTCCTGAG
AATGTGGAGTGGAGTGGTGCTGAAGCCTCAATGTTTAGAGTCCTCATTGGCACTTACTATGA
CAATTTCTGTGCCATTGCTAGGTTAATTGGGACCAAAACATGTAGACAGGTGTATGAGTTTA
GAGTCAAAGAATCTAGCATCATAGCTCCAGCTCCCGCTGAGGATGTGGATACTCCTCCAAGG
AAAAAGAAGAGGAAACACCGGTTGTGGGCTGCACACTGCAGAAAGATACAGCTGAAAAAGGA
CGGCTCCTCTAACCATGTTTACAACTATCAACCCTGTGATCATCCACGGCAGCCTTGTGACA
GTTCGTGCCCTTGTGTGATAGCACAAAATTTTTGTGAAAAGTTTTGTCAATGTAGTTCAGAG
TGTCAAAACCGCTTTCCGGGATGCCGCTGCAAAGCACAGTGCAACACCAAGCAGTGCCCGTG
CTACCTGGCTGTCCGAGAGTGTGACCCTGACCTCTGTCTTACTTGTGGAGCCGCTGACCATT
GGGACAGTAAAAATGTGTCCTGCAAGAACTGCAGTATTCAGCGGGGCTCCAAAAAGCATCTA
TTGCTGGCACCATCTGACGTGGCAGGCTGGGGGATTTTTATCAAAGATCCTGTGCAGAAAAA
TGAATTCATCTCAGAATACTGTGGAGAGATTATTTCTCAAGATGAAGCTGACAGAAGAGGGA
AAGTGTATGATAAATACATGTGCAGCTTTCTGTTCAACTTGAACAATGATTTTGTGGTGGAT
GCAACCCGCAAGGGTAACAAAATTCGTTTTGCAAATCATTCGGTAAATCCAAACTGCTATGC
AAAAGTTATGATGGTTAACGGTGATCACAGGATAGGTATTTTTGCCAAGAGAGCCATCCAGA
CTGGCGAAGAGCTGTTTTTTGATTACAGATACAGCCAGGCTGATGCCCTGAAGTATGTCGGC
ATCGAAAGAGAAATGGAAATCCCTTGACATCTGCTACCTCCTCCCCCCTCCTCTGAAACAGC
TGCCTTAGCTTCAGGAACCTCGAGTACTGTGGGCAATTTAGAAAAAGAACATGCAGTTTGAA
ATTCTGAATTTGCAAAGTACTGTAAGAATAATTTATAGTAATGAGTTTAAAAATCAACTTTT
TATTGCCTTCTCACCAGCTGCAAAGTGTTTTGTACCAGTGAATTTTTGCAATAATGCAGTAT
GGTACATTTTTCAACTTTGAATAAAGAATACTTGAACTTGTC
SEQ ID NO: 18 - Wild type human EZH2 isoform b (variant 2)
polynucleotide coding sequence
(SEQ ID NO: 18)
ATGGGCCAGACTGGGAAGAAATCTGAGAAGGGACCAGTTTGTTGGCGGAAGCGTGTAAAATC
AGAGTACATGCGACTGAGACAGCTCAAGAGGTTCAGACGAGCTGATGAAGTAAAGAGTATGT
TTAGTTCCAATCGTCAGAAAATTTTGGAAAGAACGGAAATCTTAAACCAAGAATGGAAACAG
CGAAGGATACAGCCTGTGCACATCCTGACTTCTGTGAGCTCATTGCGCGGGACTAGGGAGGT
GGAAGATGAAACTGTTTTACATAACATTCCTTATATGGGAGATGAAGTTTTAGATCAGGATG
GTACTTTCATTGAAGAACTAATAAAAAATTATGATGGGAAAGTACACGGGGATAGAGAATGT
GGGTTTATAAATGATGAAATTTTTGTGGAGTTGGTGAATGCCCTTGGTCAATATAATGATGA
TGACGATGATGATGATGGAGACGATCCTGAAGAAAGAGAAGAAAAGCAGAAAGATCTGGAGG
ATCACCGAGATGATAAAGAAAGCCGCCCACCTCGGAAATTTCCTTCTGATAAAATTTTTGAA
GCCATTTCCTCAATGTTTCCAGATAAGGGCACAGCAGAAGAACTAAAGGAAAAATATAAAGA
ACTCACCGAACAGCAGCTCCCAGGCGCACTTCCTCCTGAATGTACCCCCAACATAGATGGAC
CAAATGCTAAATCTGTTCAGAGAGAGCAAAGCTTACACTCCTTTCATACGCTTTTCTGTAGG
CGATGTTTTAAATATGACTGCTTCCTACATCCTTTTCATGCAACACCCAACACTTATAAGCG
GAAGAACACAGAAACAGCTCTAGACAACAAACCTTGTGGACCACAGTGTTACCAGCATTTGG
AGGGAGCAAAGGAGTTTGCTGCTGCTCTCACCGCTGAGCGGATAAAGACCCCACCAAAACGT
CCAGGAGGCCGCAGAAGAGGACGGCTTCCCAATAACAGTAGCAGGCCCAGCACCCCCACCAT
TAATGTGCTGGAATCAAAGGATACAGACAGTGATAGGGAAGCAGGGACTGAAACGGGGGGAG
AGAACAATGATAAAGAAGAAGAAGAGAAGAAAGATGAAACTTCGAGCTCCTCTGAAGCAAAT
TCTCGGTGTCAAACACCAATAAAGATGAAGCCAAATATTGAACCTCCTGAGAATGTGGAGTG
GAGTGGTGCTGAAGCCTCAATGTTTAGAGTCCTCATTGGCACTTACTATGACAATTTCTGTG
CCATTGCTAGGTTAATTGGGACCAAAACATGTAGACAGGTGTATGAGTTTAGAGTCAAAGAA
TCTAGCATCATAGCTCCAGCTCCCGCTGAGGATGTGGATACTCCTCCAAGGAAAAAGAAGAG
GAAACACCGGTTGTGGGCTGCACACTGCAGAAAGATACAGCTGAAAAAGGACGGCTCCTCTA
ACCATGTTTACAACTATCAACCCTGTGATCATCCACGGCAGCCTTGTGACAGTTCGTGCCCT
TGTGTGATAGCACAAAATTTTTGTGAAAAGTTTTGTCAATGTAGTTCAGAGTGTCAAAACCG
CTTTCCGGGATGCCGCTGCAAAGCACAGTGCAACACCAAGCAGTGCCCGTGCTACCTGGCTG
TCCGAGAGTGTGACCCTGACCTCTGTCTTACTTGTGGAGCCGCTGACCATTGGGACAGTAAA
AATGTGTCCTGCAAGAACTGCAGTATTCAGCGGGGCTCCAAAAAGCATCTATTGCTGGCACC
ATCTGACGTGGCAGGCTGGGGGATTTTTATCAAAGATCCTGTGCAGAAAAATGAATTCATCT
CAGAATACTGTGGAGAGATTATTTCTCAAGATGAAGCTGACAGAAGAGGGAAAGTGTATGAT
AAATACATGTGCAGCTTTCTGTTCAACTTGAACAATGATTTTGTGGTGGATGCAACCCGCAA
GGGTAACAAAATTCGTTTTGCAAATCATTCGGTAAATCCAAACTGCTATGCAAAAGTTATGA
TGGTTAACGGTGATCACAGGATAGGTATTTTTGCCAAGAGAGCCATCCAGACTGGCGAAGAG
CTGTTTTTTGATTACAGATACAGCCAGGCTGATGCCCTGAAGTATGTCGGCATCGAAAGAGA
AATGGAAATCCCTTGA
SEQ ID NO: 19 - IKZF1 shRNA
(SEQ ID NO: 19)
gccgaagctataaacagcgaactcgagttcgctgtttatagcttcggc
SEQ ID NO: 20 - Wild type human NSUN2 isoform 2 amino acid sequence
(SEQ ID NO: 20)
MGRRSRGRRLQQQQRPEDAEDGAEGGGKRGEAGWEGGYPEIVKENKLFEHYYQELKIVPEGE
WGQFMDALREPLPATLRITGYKSHAKEILHCLKNKYFKELEDLEVDGQKVEVPQPLSWYPEE
LAWHTNLSRKILRKSPHLEKFHQFLVSETESGNISRQEAVSMIPPLLLNVRPHHKILDMCAA
PGSKTTQLIEMLHADMNVPFPEGFVIANDVDNKRCYLLVHQAKRLSSPCIMVVNHDASSIPR
LQIDVDGRKEILFYDRILCDVPCSGDGTMRKNIDVWKKWTTLNSLQLHGLQLRIATRGAEQL
AEGGRMVYSTCSLNPIEDEAVIASLLEKSEGALELADVSNELPGLKWMPGITQWKVMTKDGQ
WFTDWDAVPHSRHTQIRPTMFPPKDPEKLQAMHLERCLRILPHHQNTGGFFVAVLVKKSSMP
WNKRQPKLQGKSAETRESTQLSPADLTEGKPTDPSKLESPSFTGTGDTEIAHATEDLENNGS
KKDGVCGPPPSKKMKLFGFKEDPFVFIPEDDPLFPPIEKFYALDPSFPRMNLLTRTTEGKKR
QLYMVSKELRNVLLNNSEKMKVINTGIKVWCRNNSGEEFDCAFRLAQEGIYTLYPFINSRII
TVSMEDVKILLTQENPFFRKLSSETYSQAKDLAKGSIVLKYEPDSANPDALQCPIVLCGWRG
KASIRTFVPKNERLHYLRMMGLEVLGEKKKEGVILTNESAASTGQPDNDVTEGQRAGEPNSP
DAEEANSPDVTAGCDPAGVHPPR
SEQ ID NO: 21 - Wild type human NSUN2 isoform 1 polynucleotide coding
sequence
(SEQ ID NO: 21)
ATGGGGCGGCGGTCGCGGGGTCGGCGGCTCCAGCAACAGCAGCGGCCGGAGGACGCGGAGGA
TGGCGCCGAGGGTGGTGGAAAGCGCGGCGAGGCGGGCTGGGAAGGAGGCTACCCCGAGATCG
TCAAGGAGAACAAGCTGTTCGAGCACTACTACCAGGAGCTCAAGATCGTGCCCGAGGGCGAG
TGGGGCCAGTTCATGGACGCTCTCAGGGAGCCGCTCCCGGCCACTTTAAGAATTACTGGTTA
CAAAAGCCACGCAAAAGAGATTCTCCATTGCTTAAAGAACAAATATTTTAAGGAATTGGAGG
ACCTGGAGGTGGACGGTCAGAAAGTTGAAGTTCCACAGCCACTGAGTTGGTATCCTGAAGAA
CTTGCCTGGCACACAAATTTAAGTCGAAAAATCTTGAGAAAATCGCCACACTTGGAAAAGTT
TCATCAGTTTCTAGTTAGTGAAACAGAATCTGGAAATATTAGTCGTCAAGAAGCTGTTAGCA
TGATCCCACCACTGCTCCTCAACGTGCGGCCTCATCATAAGATCTTAGATATGTGTGCAGCA
CCTGGCTCAAAGACCACACAGTTAATTGAAATGCTACATGCCGACATGAATGTCCCCTTTCC
AGAGGGATTTGTTATTGCGAATGATGTGGACAACAAGCGCTGCTACCTGCTCGTCCATCAAG
CCAAGAGGCTGAGCAGCCCCTGCATCATGGTGGTCAACCATGATGCCTCCAGCATACCCAGG
CTCCAGATAGATGTGGACGGCAGGAAAGAGATCCTCTTCTATGATCGAATTTTATGTGATGT
CCCTTGCAGTGGAGACGGCACTATGAGAAAAAACATTGATGTTTGGAAAAAGTGGACCACCT
TAAATAGCTTGCAGCTACATGGCTTACAGCTGCGGATTGCAACACGCGGGGCTGAACAGCTG
GCTGAAGGTGGAAGGATGGTGTATTCCACGTGTTCACTAAACCCTATTGAGGATGAAGCAGT
CATAGCATCTTTACTGGAAAAAAGTGAAGGTGCTTTGGAGCTTGCTGATGTGTCTAATGAAC
TGCCAGGGCTGAAGTGGATGCCTGGAATCACACAGTGGAAGGTAATGACGAAAGATGGGCAG
TGGTTTACAGACTGGGACGCTGTTCCTCACAGCAGACACACCCAGATCCGACCTACCATGTT
CCCTCCGAAGGACCCAGAAAAGCTGCAGGCCATGCACCTGGAGCGATGCCTTAGGATATTAC
CCCATCATCAGAATACTGGAGGGTTTTTTGTGGCAGTATTGGTGAAAAAATCTTCAATGCCG
TGGAATAAACGTCAGCCAAAGCTTCAGGGTAAATCTGCAGAGACCAGAGAAAGCACACAGCT
GAGCCCTGCAGATCTCACAGAAGGGAAACCCACAGATCCCTCTAAGCTGGAAAGTCCGTCAT
TCACAGGAACTGGTGACACAGAAATAGCTCATGCAACTGAGGATTTAGAGAATAATGGCAGT
AAGAAAGATGGCGTGTGTGGTCCTCCTCCATCAAAGAAAATGAAGTTATTTGGATTTAAAGA
AGATCCATTTGTATTTATTCCTGAAGATGACCCATTATTTCCACCTATTGAGAAATTTTATG
CTTTGGATCCTTCATTCCCAAGGATGAATTTGTTAACTCGGACTACAGAAGGGAAGAAAAGG
CAGCTCTACATGGTTTCTAAGGAGTTGCGGAATGTGCTGCTGAATAACAGTGAGAAGATGAA
GGTTATTAACACGGGGATCAAAGTCTGGTGTAGAAATAACAGCGGTGAAGAGTTTGACTGTG
CTTTCCGGCTGGCACAGGAGGGAATATATACATTGTATCCATTTATTAACTCAAGAATTATT
ACTGTATCAATGGAAGATGTTAAGATACTGTTGACCCAGGAAAATCCCTTTTTTAGAAAACT
CAGCAGTGAGACCTACAGTCAAGCAAAGGACCTGGCAAAGGGAAGCATCGTGCTGAAGTATG
AACCAGATTCTGCGAATCCAGACGCTCTGCAGTGTCCCATCGTCTTATGCGGATGGCGGGGA
AAGGCCTCCATTCGAACTTTTGTGCCCAAGAATGAACGGCTTCATTATCTCAGGATGATGGG
GCTGGAGGTATTGGGAGAAAAGAAGAAGGAAGGGGTTATCCTCACAAATGAGAGTGCAGCCA
GCACCGGACAGCCAGACAATGACGTGACTGAGGGACAGAGAGCAGGAGAGCCCAACAGCCCA
GATGCAGAAGAGGCCAACAGTCCAGACGTGACAGCAGGCTGTGACCCGGCGGGGGTCCATCC
ACCCCGGTGA
SEQ ID NO: 22 - Wild type human NSUN2 isoform 2 amino acid sequence
(SEQ ID NO: 22)
MGRRSRGRRLQQQQRPEDAEDGAEGGGKRGEAGWEGGYPEIVKENKLFEHYYQELKIVPEGE
WGQFMDALREPLPATLRITGYKRYPEELAWHTNLSRKILRKSPHLEKFHQFLVSETESGNIS
RQEAVSMIPPLLLNVRPHHKILDMCAAPGSKTTQLIEMLHADMNVPFPEGFVIANDVDNKRC
YLLVHQAKRLSSPCIMVVNHDASSIPRLQIDVDGRKEILFYDRILCDVPCSGDGTMRKNIDV
WKKWTTLNSLQLHGLQLRIATRGAEQLAEGGRMVYSTCSLNPIEDEAVIASLLEKSEGALEL
ADVSNELPGLKWMPGITQWKVMTKDGQWFTDWDAVPHSRHTQIRPTMFPPKDPEKLQAMHLE
RCLRILPHHQNTGGFFVAVLVKKSSMPWNKRQPKLQGKSAETRESTQLSPADLTEGKPTDPS
KLESPSFTGTGDTEIAHATEDLENNGSKKDGVCGPPPSKKMKLFGFKEDPFVFIPEDDPLFP
PIEKFYALDPSFPRMNLLTRTTEGKKRQLYMVSKELRNVLLNNSEKMKVINTGIKVWCRNNS
GEEFDCAFRLAQEGIYTLYPFINSRIITVSMEDVKILLTQENPFFRKLSSETYSQAKDLAKG
SIVLKYEPDSANPDALQCPIVLCGWRGKASIRTFVPKNERLHYLRMMGLEVLGEKKKEGVIL
TNESAASTGQPDNDVTEGQRAGEPNSPDAEEANSPDVTAGCDPAGVHPPR
SEQ ID NO: 23 - Wild type human NSUN2 isoform 2 polynucleotide coding
sequence
(SEQ ID NO: 23)
ATGGGGCGGCGGTCGCGGGGTCGGCGGCTCCAGCAACAGCAGCGGCCGGAGGACGCGGAGGA
TGGCGCCGAGGGTGGTGGAAAGCGCGGCGAGGCGGGCTGGGAAGGAGGCTACCCCGAGATCG
TCAAGGAGAACAAGCTGTTCGAGCACTACTACCAGGAGCTCAAGATCGTGCCCGAGGGCGAG
TGGGGCCAGTTCATGGACGCTCTCAGGGAGCCGCTCCCGGCCACTTTAAGAATTACTGGTTA
CAAAAGGTATCCTGAAGAACTTGCCTGGCACACAAATTTAAGTCGAAAAATCTTGAGAAAAT
CGCCACACTTGGAAAAGTTTCATCAGTTTCTAGTTAGTGAAACAGAATCTGGAAATATTAGT
CGTCAAGAAGCTGTTAGCATGATCCCACCACTGCTCCTCAACGTGCGGCCTCATCATAAGAT
CTTAGATATGTGTGCAGCACCTGGCTCAAAGACCACACAGTTAATTGAAATGCTACATGCCG
ACATGAATGTCCCCTTTCCAGAGGGATTTGTTATTGCGAATGATGTGGACAACAAGCGCTGC
TACCTGCTCGTCCATCAAGCCAAGAGGCTGAGCAGCCCCTGCATCATGGTGGTCAACCATGA
TGCCTCCAGCATACCCAGGCTCCAGATAGATGTGGACGGCAGGAAAGAGATCCTCTTCTATG
ATCGAATTTTATGTGATGTCCCTTGCAGTGGAGACGGCACTATGAGAAAAAACATTGATGTT
TGGAAAAAGTGGACCACCTTAAATAGCTTGCAGCTACATGGCTTACAGCTGCGGATTGCAAC
ACGCGGGGCTGAACAGCTGGCTGAAGGTGGAAGGATGGTGTATTCCACGTGTTCACTAAACC
CTATTGAGGATGAAGCAGTCATAGCATCTTTACTGGAAAAAAGTGAAGGTGCTTTGGAGCTT
GCTGATGTGTCTAATGAACTGCCAGGGCTGAAGTGGATGCCTGGAATCACACAGTGGAAGGT
AATGACGAAAGATGGGCAGTGGTTTACAGACTGGGACGCTGTTCCTCACAGCAGACACACCC
AGATCCGACCTACCATGTTCCCTCCGAAGGACCCAGAAAAGCTGCAGGCCATGCACCTGGAG
CGATGCCTTAGGATATTACCCCATCATCAGAATACTGGAGGGTTTTTTGTGGCAGTATTGGT
GAAAAAATCTTCAATGCCGTGGAATAAACGTCAGCCAAAGCTTCAGGGTAAATCTGCAGAGA
CCAGAGAAAGCACACAGCTGAGCCCTGCAGATCTCACAGAAGGGAAACCCACAGATCCCTCT
AAGCTGGAAAGTCCGTCATTCACAGGAACTGGTGACACAGAAATAGCTCATGCAACTGAGGA
TTTAGAGAATAATGGCAGTAAGAAAGATGGCGTGTGTGGTCCTCCTCCATCAAAGAAAATGA
AGTTATTTGGATTTAAAGAAGATCCATTTGTATTTATTCCTGAAGATGACCCATTATTTCCA
CCTATTGAGAAATTTTATGCTTTGGATCCTTCATTCCCAAGGATGAATTTGTTAACTCGGAC
TACAGAAGGGAAGAAAAGGCAGCTCTACATGGTTTCTAAGGAGTTGCGGAATGTGCTGCTGA
ATAACAGTGAGAAGATGAAGGTTATTAACACGGGGATCAAAGTCTGGTGTAGAAATAACAGC
GGTGAAGAGTTTGACTGTGCTTTCCGGCTGGCACAGGAGGGAATATATACATTGTATCCATT
TATTAACTCAAGAATTATTACTGTATCAATGGAAGATGTTAAGATACTGTTGACCCAGGAAA
ATCCCTTTTTTAGAAAACTCAGCAGTGAGACCTACAGTCAAGCAAAGGACCTGGCAAAGGGA
AGCATCGTGCTGAAGTATGAACCAGATTCTGCGAATCCAGACGCTCTGCAGTGTCCCATCGT
CTTATGCGGATGGCGGGGAAAGGCCTCCATTCGAACTTTTGTGCCCAAGAATGAACGGCTTC
ATTATCTCAGGATGATGGGGCTGGAGGTATTGGGAGAAAAGAAGAAGGAAGGGGTTATCCTC
ACAAATGAGAGTGCAGCCAGCACCGGACAGCCAGACAATGACGTGACTGAGGGACAGAGAGC
AGGAGAGCCCAACAGCCCAGATGCAGAAGAGGCCAACAGTCCAGACGTGACAGCAGGCTGTG
ACCCGGCGGGGGTCCATCCACCCCGGTGA

### IV. Biomarkers and/or Targets for Therapeutic Intervention

In certain aspects, provided herein are methods of diagnosis and/or treatment comprising analysis and/or modification of RNA. In some aspects, methods of diagnosis and/or treatment comprising analysis and/or modification of RNA may be loci and/or sequence specific, or may be loci and/or sequence non-specific.

In certain aspects, provided herein are methods of identifying features, (e.g., such as biomarker features), and evaluating the presence, absence, or levels of said features in a sample. In some aspects, one or more (for example but not limited to, 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, or 1000, or more than 1000, or any range derivable therein) features are used to classify (e.g., diagnose) a disease state and/or identify one or more effective treatment options for a patient with a disease associated with aberrant transcription (e.g., aberrant transcription associated with increased euchromatin). In some aspects, one or more features are used to diagnose a disease state and/or identify one or more effective treatment options for a patient with cancer.

In some aspects, one or more features (for example but not limited to, 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, or 1000, or more than 1000, or any range derivable therein) are used to diagnose a disease state and/or identify one or more effective treatment options for a patient with a blood cancer. In some aspects, one or more features are used to diagnose a disease state and/or identify one or more effective treatment options for a patient with a leukemia. In some aspects, one or more features are used to diagnose a disease state and/or identify one or more effective treatment options for a patient with a myeloid malignancy. In some aspects, one or more features are used to diagnose a disease state and/or identify one or more effective treatment options for a patient with acute myeloid leukemia. In some aspects, one or more features are used to diagnose a disease state and/or identify one or more effective treatment options for a patient with chronic myelomonocytic leukemia. In some aspects, one or more features are used to diagnose a disease state and/or identify one or more effective treatment options for a patient with a glioma. In some aspects, one or more features are used to diagnose a disease state and/or identify one or more effective treatment options for a patient with glioblastoma.

In some aspects, one or more (for example but not limited to, 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, or 1000, or more than 1000, or any range derivable therein) features are used to diagnose a disease state and/or identify one or more effective treatment options for a patient diagnosed with a pre-cancerous condition, such as but not limited to, clonal hematopoiesis of indeterminate potential (CHIP).

In some aspects, a feature can be associated with 5-azaC sensitivity. In some aspects, a feature can be shared in, in greater than, or in less than, or 1, 2, 3, 4, 5, 6, 7, 8, 9, 10, or more leukemia cell lines that are susceptible to 5-azaC treatment. In some aspects, methods provided herein comprise determining the expression levels and/or mutational status of IKZF1, EZH2, NPM1, TET2, PDS5B, NF1, MED12, KMT2D, KRAS, FLG, FLT3, NOTCH1, ASXL1, NRAS, TTN, and/or TP53.

In some aspects, a biomarker comprises, consists essentially of, or consists of IKZF1, EZH2, NPM1, TET2, PDS5B, NF1, MED12, KMT2D, KRAS, FLG, FLT3, NOTCH1, ASXL1, NRAS, TTN, and/or TP53. In some aspects, biomarker features are determined using DNA sequencing of the genome of a cell or population of cells. In some aspects, biomarker features are determined using DNA sequencing of the genome of a cell or population of cells.

In certain aspects, provided herein are methods of treatment comprising analyzing and/or measuring IKZF1, EZH2, NPM1, TET2, PDS5B, NF1, MED12, KMT2D, KRAS, FLG, FLT3, NOTCH1, ASXL1, NRAS, TTN, and/or TP53 RNA expression and/or modification levels in suspected diseased cells, determining if RNA expression and/or modification levels are aberrant, and if RNA expression and/or modification levels are aberrant, contacting the cells with 5-azaC and/or one or more one or more IKZF1, EZH2, NPM1, TET2, NSUN2, and/or MBD6 inhibitors. In certain aspects, provided herein are methods of treatment comprising analyzing RNA expression and/or modification levels in suspected diseased cells, determining if RNA expression and/or modification levels are aberrant, and if RNA expression and/or modification levels are aberrant, not contacting the cells with 5-azaC as a monotherapy. In certain aspects, provided herein are methods of treatment comprising analyzing RNA expression and/or modification levels in suspected diseased cells, determining if RNA expression and/or modification levels are aberrant, and if RNA expression and/or modification levels are aberrant, contacting the cells with 5-azaC as a monotherapy. In certain aspects, provided herein are methods of treatment comprising analyzing RNA expression and/or modification levels in suspected diseased cells, determining if RNA expression and/or modification levels are aberrant, and if RNA expression and/or modification levels are aberrant, contacting the cells with 5-azaC as a co-therapy with one or more additional interventions.

### V. Vectors and Nanoparticles

### A. Vectors

Among other things, the present disclosure provides that in some aspects, polypeptides and/or oligonucleotides described herein are encoded by a polynucleotide, such as a vector comprising a polynucleotide (e.g., a polynucleotide construct). Vectors comprising polynucleotide constructs according to the present disclosure include all those known in the art, including cosmids, plasmids (e.g., naked or contained in liposomes) and viral constructs (e.g., lentiviral, retroviral, adenoviral, and adeno associated viral constructs) that incorporate a polynucleotide encoding functionality and/or an element described herein. Those of skill in the art will be capable of selecting suitable constructs, as well as cells, for making any of the polynucleotides described herein. In some aspects, a construct is a plasmid (i.e., a circular DNA molecule that can autonomously replicate inside a cell). In some aspects, a construct can be a cosmid (e.g., pWE or sCos series).

In some aspects, a construct is a viral construct. In some aspects, a viral construct is a lentivirus, retrovirus, adenovirus, or adeno-associated virus construct. In some aspects, a construct is an adeno-associated virus (AAV) construct (see, e.g., Asokan et al., Mol. Ther. 20: 699-7080, 2012, which is incorporated herein by reference for the purposes described herein). In some aspects, a viral construct is an adenovirus construct. In some aspects, a viral construct may also be based on or derived from an alphavirus. Alphaviruses include but are not limited to, Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference for the purposes described herein.

In some aspects, constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 4.5 kb to 5 kb, or about 4.7 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter.

### B. Nanoparticles

Certain aspects disclosed herein concern compositions comprising a nanoparticle, which may encapsulate a therapeutic agent, which can be any of the therapeutic agents disclosed herein (for example but not limited to, inhibitors of IKZF1, NPM1, EZH2, and/or TET2). The nanoparticle compositions may encapsulate therapeutic agents, which may be but are not limited to: engineered protein compositions, polynucleotides, and/or small molecules. In some aspects, engineered protein compositions, polynucleotides, and/or small molecules formulated in the nanoparticles can have improved pharmacokinetic and/or pharmacodynamic properties. In some aspects, engineered protein compositions, polynucleotides, and/or small molecules nanoparticles are better tolerated by a patient, including a cancer patient. In some aspects, engineered protein compositions, polynucleotides, and/or small molecules are formulated in the nanoparticles, and in some aspects, are more effectively delivered to cells to effect their function, such as effecting transcriptional changes, than naked engineered proteins, polynucleotides, and/or small molecules. In some aspects, a nanoparticle composition confers water solubility to hydrophobic agents, to combinations of hydrophobic agents, and/or to combinations of hydrophobic and hydrophilic agents. In some aspects, a nanoparticle composition comprises a liposomal and/or nano-emulsion composition of a therapeutic agent.

In some aspects, as disclosed elsewhere herein, a nanoparticle composition (e.g., a mixed micelle composition, a liposomal composition, solid lipid particles, oil-in-water emulsions, water-in-oil-in-water emulsions, water-in-oil emulsions, oil-in-water-in-oil emulsions, etc.) is provided to aid in the delivery of therapeutic agents. In some aspects, the nanoparticles comprise one or more (e.g., 1, 2, 3, 4, 5, 6, 7, 8, 9, 10, or more) therapeutic agents. In some aspects, a composition comprising the nanoparticles disclosed herein comprises a therapeutically effective amount of one or more (e.g., 1, 2, 3, 4, 5, 6, 7, 8, 9, 10, or more) therapeutic agents.

In some aspects, when formulated, the dry weight % of one or more therapeutic agents present in the nanoparticle compositions is equal to, is equal to at least, or is equal to at most: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or any range derivable therein. In several aspects, the therapeutic agents are provided in an aqueous composition. In several aspects, the wet weight % of one or more therapeutic agents present in the composition (with water included) is equal to or at least about: 0.5%, 1%, 2.5%, 5%, 7.5%, 10%, 12.5%, 15%, 20%, 22.5%, 25%, 27.5%, 30%, or any range derivable therein. In several aspects, the one or more therapeutic agents may be provided in the wet composition at a concentration of greater than or equal to about: 0.001 mg/mL, 0.005 mg/mL, 0.01 mg/mL, 0.05 mg/mL, 0.1 mg/mL, 0.5 mg/mL, 1 mg/mL, 5 mg/mL, 20 mg/mL, 30 mg/mL, 50 mg/mL, 100 mg/mL, or any range derivable therein.

In some aspects, the therapeutic agents, collectively or individually, are present in the aqueous nanoparticle composition at a concentration of less than or equal to about: 150 mg/mL, 100 mg/mL, 75 mg/mL, 50 mg/mL, 25 mg/mL, 20 mg/mL, 10 mg/mL, 5 mg/mL, 2.5 mg/mL, 2 mg/mL, 1.5 mg/mL, 1 mg/mL, 0.5 mg/mL, 0.1 mg/mL, 0.05 mg/mL, 0.01 mg/mL, or ranges including and/or spanning the aforementioned values. In several aspects, the one or more therapeutic agents, collectively or individually, are present in the aqueous composition at a concentration of greater than or equal to about: 150 mg/mL, 100 mg/mL, 75 mg/mL, 50 mg/mL, 25 mg/mL, 20 mg/mL, 10 mg/mL, 5 mg/mL, 2.5 mg/mL, 2 mg/mL, 1.5 mg/mL, 1 mg/mL, 0.5 mg/mL, 0.1 mg/mL, 0.05 mg/mL, 0.01 mg/mL, or ranges including and/or spanning the aforementioned values. In several aspects, the one or more therapeutic agents, collectively or individually, are present in the composition at a dry wt. % of equal to, at most, or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or ranges including and/or spanning the aforementioned values. In several aspects, the one or more therapeutic agents, collectively or individually, are present in the composition at a wet wt. % of equal to, or at most, or at least about: 0.1%, 0.5%, 1%, 2.5%, 5%, 7.5%, 10%, 12.5%, 15%, 20%, 22.5%, 25%, 27.5%, 30%, 32.5%, 35%, 37.5%, 40%, 42.5%, 45%, 47.5%, 50%, 52.5%, 55%, 57.5%, 60%, or ranges including and/or spanning the aforementioned values. In several aspects, as disclosed elsewhere herein, the composition is aqueous, while in others it has been dried into a powder (that is free of or substantially free of water). In several aspects, where the composition has been dried, it comprises a water content of less than or equal to 20%, 15%, 10%, 7.5%, 5%, 2.5%, 1%, or ranges including and/or spanning the aforementioned values.

In several aspects, as disclosed elsewhere herein, the composition is aqueous (e.g., contains water) while in other aspects, the composition is dry (lacks water or substantially lacks water). In several aspects, the composition has been dried (e.g., has been subjected to a process to remove most or substantially all water). In several aspects, the composition comprises nanoparticles in water (e.g., as a solution, suspension, or emulsion). In other aspects, the composition is provided as a powder (e.g., that may be constituted or reconstituted in water). In several aspects, as disclosed elsewhere herein, the water content (in wt. %) of the composition is less than or equal to about: 30%, 20%, 10%, 5%, 2.5%, 1%, 0.5%, 0.1%, 0%, or ranges including and/or spanning the aforementioned values. In several aspects, as disclosed elsewhere herein, the water content (in wt. %) of the composition is greater than or equal to about: 50%, 60%, 70%, 80%, 85%, 90%, 92.5%, 95%, 97.5%, or ranges including and/or spanning the aforementioned values. In several aspects, the water is nanopure, deionized, USP grade, WFI, and/or combinations of the foregoing. In some aspects, the composition is a dried composition comprising a nanoparticle having weight ratios of a first therapeutic agent: a nanoscale coordination polymer NCP: optionally a lipid source, and optionally a surfactant of 1 to 50:1 to 50:1 to 50:0 to 17.5.

In some aspects, a nanoparticle composition provides an oil-in-water emulsion (e.g., a nanoemulsion), water-in-oil emulsion, a water-in-oil-in-water emulsion, an oil-in-water-in-oil emulsion, a liposome (and variants including multi-lamellar, double liposome preparations, etc.), micelle, and/or solid lipid particles. Any one of these structures may be provided as a nanoparticle or microparticle.

In several aspects, as disclosed elsewhere herein, a nanoparticle composition is an oil-in-water emulsion, water-in-oil emulsion, water-in-oil-in-water emulsion, oil-in-water-in-oil emulsion, liposome, solid lipid particles formulation, etc. For brevity, these may just be referred to as the composition. In several aspects, a nanoparticle composition can be processed to comprises one or more of solid lipid nanoparticles, liposomes (and variants including multi-lamellar, double liposome preparations, etc.), niosomes, ethosomes, electrostatic particulates, microemulsions, nanoemulsions, microsuspensions, nanosuspensions, or combinations thereof. In several aspects, polymeric nanoparticles may be formed.

In several aspects, a solid lipid nanoparticle compositions comprises a lipid core matrix. In several aspects, the lipid core matrix is solid. In several aspects, the solid lipid comprises one or more ingredients as disclosed elsewhere herein. In several aspects, the core of the solid lipid comprises one or more lipids, surfactants, active ingredients, etc. In several aspects, the surfactant acts as an emulsifier. In several aspects, emulsifiers can be used to stabilize the lipid dispersion (with respect to charge and molecular weight). In several aspects, the core ingredients (e.g., the components of the core) are present in the composition (collectively or individually) at a dry wt. % of equal to or less than about: 0.5%, 1.0%, 2.5%, 5%, 10%, 15%, 20%, 30%, 40%, 50%, 60%, 80% or ranges including and/or spanning the aforementioned values. In several aspects, the core ingredients and/or the emulsifiers (collectively or individually) are present in the composition at a wet wt. % of equal to or less than about: 0.5%, 1.0% 2.5%, 5%, 7.5%, 10%, 12.5%, 15%, 20%, 30%, 40%, 60% or ranges including and/or spanning the aforementioned values.

In several aspects, a nanoparticle composition (e.g., when in water or dried) comprises multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In certain aspects, the nanoparticle compositions do not comprise multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In several aspects, the composition is characterized by having multiple types of particles (e.g., lamellar, emulsion, irregular, etc.). In other aspects, a majority of the particles present are emulsion particles. In several aspects, a majority of the particles present are lamellar (multilamellar and/or unilamellar). In other aspects, a majority of the particles present are irregular particles. In still other aspects, a minority of the particles present are emulsion particles. In several aspects, a minority of the particles present are lamellar (multilamellar and/or unilamellar). In other aspects, a minority of the particles present are irregular particles.

The nanoparticle compositions can comprise, but are not limited to, combinations of multilamellar nanoparticles, unilamellar nanoparticles, emulsion nanoparticles, micelle nanoparticles, irregular particles, and/or liposomes.

The percentages and/or concentrations of particles present in the composition may be purposefully modified. In some aspects, the percentage and/or concentration of the particles present in the composition are tailored to the active compound and/or the liquid comprising the particles. Such tailoring may lead to more homogenization and/or dispersion in the liquid. The tailoring may stabilize dispersion in the liquid.

In several aspects, the formulations and/or compositions disclosed herein are stable during sterilization. In several aspects, the sterilization may include one or more of ozonation, UV treatment, and/or heat treatment. In several aspects, the particle size and/or PDI after sterilization (e.g., exposure to techniques that allow sterilization of the composition) varies by less than or equal to about: 1%, 5%, 10%, 20%, 30%, or ranges including and/or spanning the aforementioned values. In several aspects, the therapeutic agent concentration after sterilization (e.g., exposure to techniques that allow sterilization of the composition) varies (e.g., drops) by less than or equal to about: 1%, 5%, 10%, 15%, or ranges including and/or spanning the aforementioned values.

In several aspects, the nanoparticle compositions (including after stabilization) disclosed herein have a shelf life of equal to or greater than 1 month, 3 months, 6 months, 12 months, 14 months, 16 months, 18 months, 19 months, or ranges including and/or spanning the aforementioned values. The shelf-life can be determined as the period of time in which there is 95% confidence that at least 50% of the response (active agent(s) concentration or particle size) is within the specification limit. This refers to a 95% confidence interval and when linear regression predicts that at least 50% of the response is within the set specification limit.

### VI. Histone Modifications

In certain aspects, provided herein are compositions and methods for modifying histone marks, such as but not limited to, histone ubiquitination and/or histone methylation. In some aspects, histone modifications can occur via inhibition of IKZF1, NPM1, EZH2, and/or TET2. In some aspects, inhibitors of IKZF1, NPM1, EZH2, and/or TET2 can be utilized to alter (decrease and/or increase) histone modifications including but not limited to: H3K4me2, H3K4me1, H2AK119ub, H3K27me3, H3K9me3, H3K27ac, H3K36me3, and/or H3K4me3.

In some aspects, technologies described herein may comprise increasing one or more histone marks at one or more loci, such as but not limited to, increasing histone marks by 0.1×, 0.2×, 0.3×, 0.4×, 0.5×, 0.6×, 0.7×, 0.8×, 0.9×, 1.0×, 2.0×, 3.0×, 4.0×, 5.0×, 6.0×, 7.0×, 8.0×, 9.0×, 10.0×, 15.0×, 20.0×, 25.0×, 50.0×, 100.0×, or greater than 100× (or any range derivable therein) relative to a control histone mark level, at 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, or 1000, or more than 1000, or any range derivable therein, histones.

In some aspects, technologies described herein may comprise increasing one or more H2AK119ub mark at one or more loci, such as but not limited to, increasing histone H2AK119ub by 0.1×, 0.2×, 0.3×, 0.4×, 0.5×, 0.6×, 0.7×, 0.8×, 0.9×, 1.0×, 2.0×, 3.0×, 4.0×, 5.0×, 6.0×, 7.0×, 8.0×, 9.0×, 10.0×, 15.0×, 20.0×, 25.0×, 50.0×, 100.0×, or greater than 100× (or any range derivable therein) relative to control H2AK119ub levels, at 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, or 1000, or more than 1000, or any range derivable therein, histones.

In some aspects, technologies described herein may comprise decreasing one or more H2AK119ub mark at one or more loci, such as but not limited to, decreasing histone H2AK119ub by 0.1×, 0.2×, 0.3×, 0.4×, 0.5×, 0.6×, 0.7×, 0.8×, 0.9×, 1.0×, 2.0×, 3.0×, 4.0×, 5.0×, 6.0×, 7.0×, 8.0×, 9.0×, 10.Ox, 15.0×, 20.0×, 25.0×, 50.0×, 100.0×, or greater than 100× (or any range derivable therein) relative to control H2AK119ub levels, at 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 550, 600, 650, 700, 750, 800, 850, 900, 950, or 1000, or more than 1000, or any range derivable therein, histones.

### VII. Compounds

Among other things, the present disclosure provides small molecule inhibitors of NSUN2. In various aspects, small molecule inhibitors of NSUN2 comprise a compound of Formula (I), or a pharmaceutically appropriate salt thereof.

Accordingly, in various aspects, a compound of Formula (I) is provided:

Ar-L-B (I) or a pharmaceutically appropriate salt thereof,

wherein Ar is a heteroaryl moiety or an aryl moiety substituted with a heteroaryl substituent, wherein Ar is optionally substituted with 1 to 3 Ry substituents, each Ry independently selected from halogen, cyano, nitro, alkyl, haloalkyl, alkoxy, haloalkoxy, alkoxycarbonyl, carboxy(C1-C6)alkoxy, aryl, heteroaryl, aryloxy, and aralkoxy, wherein the heteroaryl moiety or the heteroaryl substituent comprises at least one ring heteroatom selected from nitrogen, oxygen, and sulfur, and when the heteroaryl moiety or the heteroaryl substituent comprises a ring nitrogen atom, the ring nitrogen atom is unsubstituted or substituted with R_{N}, wherein R_{N} is selected from SO2Rx, C(═O)Rx, and C(═O)ORx, and wherein Rx is selected from substituted or unsubstituted C1-C6 alkyl, substituted or unsubstituted aryl, substituted or unsubstituted heteroaryl, and substituted or unsubstituted heterocyclyl; L is —CH═, wherein the carbon atom of L is singly bonded to Ar and doubly bonded to a carbon atom of B; and B is selected from a barbiturate moiety, a thiobarbiturate moiety, a Meldrum's acid moiety, a thiazolidinedione moiety, a pyrazolone moiety, an indane-1,3-dione moiety, a cyanoacrylic acid moiety, a cyanoacrylate moiety, and an aryl-substituted acrylonitrile moiety.

In various aspects, B may be a barbiturate moiety or a thiobarbiturate moiety.

In various aspects, B may be a pyrimidine-2,4,6(1H,3H,5H)-trione moiety or a 2-thioxodihydropyrimidine-4,6(1H,5H)-dione moiety. For example, in some aspects, B is

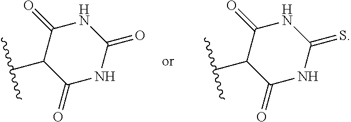 [SMILES: *C1C(=O)NC(=O)NC1=O ; *C1C(=O)NC(=S)NC1=O]

In various aspects, B may be a Meldrum's acid moiety, a thiazolidinedione moiety, a pyrazolone moiety, an indane-1,3-dione moiety, a cyanoacrylic acid moiety, a cyanoacrylate moiety, or an aryl-substituted acrylonitrile moiety. In various aspects, B may be O

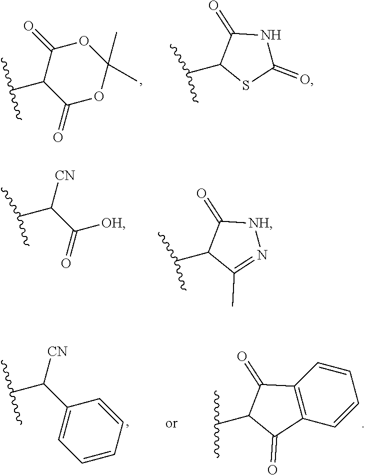

In various aspects, Ar may be a heteroaryl moiety. In various aspects, Ar may be a fused heteroaryl moiety comprising nitrogen. In various aspects, Ar may be an indolyl moiety or a pyrrolopyridinyl moiety. In various aspects, Ar may be an indol-3-yl moiety, an indol-2-yl moiety, an indol-4-yl moiety, an indol-5-yl moiety or an indol-6-yl moiety. In various aspects, Ar may be a fused heteroaryl moiety comprising sulfur. In various aspects, Ar may be a benzothiophenyl moiety. In various aspects, Ar may be a fused heteroaryl moiety comprising oxygen. In various aspects, Ar may be a dioxolo-fused indolyl moiety. In various aspects, Ar may be a pyrrolyl moiety or a thiazolyl moiety. In various aspects, Ar may be an aryl moiety substituted with a heteroaryl substituent. In various aspects, Ar may be a phenyl moiety substituted with a thiazolyl moiety.

In various aspects, the R_{N} substituent may be —SO₂R_{X}. In various aspects, the R_{N} substituent may be C(═O)R_{X}. In various aspects, the R_{N} substituent may be C(═O)OR_{X}.

In various aspects, R_{X} may be aryl, heteroaryl, or heterocyclyl. In various aspects, the aryl may be phenyl or naphthyl. In various aspects, R_{X} may be benzo[d][1,3]dioxol-5-yl. In various aspects, the heterocyclyl may be a nitrogen-containing heterocyclyl. In various aspects, the heterocyclyl may be substituted on a ring nitrogen atom with tert-butoxycarbonyl. In various aspects, the heterocyclyl may be piperidinyl. In various aspects, R_{X} may be substituted with 1 to 3 substituents independently selected from halogen, cyano, alkyl, alkoxy, haloalkyl, haloalkoxy, and aryloxy. In various aspects, R_{X} may be substituted or unsubstituted C1-C6 alkyl. In various aspects, R_{X} may be tert-butyl.

In various aspects, at least one R_{y} substituent may be selected from trifluoromethyl, trifluoromethoxy, methoxycarbonyl, benzyloxy, nitro, phenyl, furyl, methoxy, chloro, cyano, methyl, and phenoxy.

Exemplary compounds of Formula I are provided in the table herein below:

| Compound | | |
|---|---|---|
| No. | Structure | Name |
| Compound 1 | 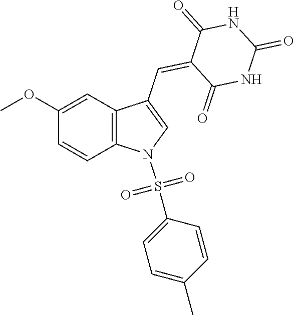 [SMILES: COc1ccc2c(c1)c(C=C1C(=O)NC(=O)NC1=O)cn2S(=O)(=O)c1ccc(C)cc1] | 5-((5-methoxy-1- tosyl-1H-indol-3- yl)methylene)pyrimidine- 2,4,6(1H,3H,5H)-trione |
| Compound 2 | 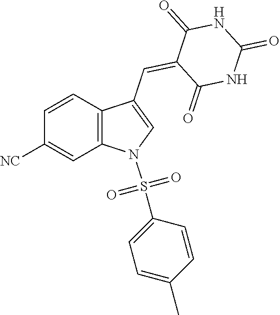 [SMILES: Cc1ccc(S(=O)(=O)n2cc(C=C3C(=O)NC(=O)NC3=O)c3ccc(C#N)cc32)cc1] | 1-tosyl-3-((2,4,6- trioxotetrahydropyrimidin-5(2H)- ylidene)methyl)-1H-indole-6- carbonitrile |
| Compound 3 | 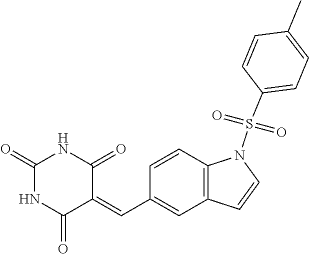 [SMILES: Cc1ccc(S(=O)(=O)n2ccc3cc(C=C4C(=O)NC(=O)NC4=O)ccc32)cc1] | 5-((1-tosyl-1H-indol-5- yl)methylene)pyrimidine- 2,4,6(1H,3H,5H)-trione |
| Compound 4 | 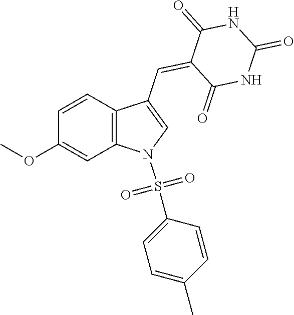 [SMILES: COc1ccc2c(C=C3C(=O)NC(=O)NC3=O)cn(S(=O)(=O)c3ccc(C)cc3)c2c1] | 5-((6-methoxy-1- tosyl-1H-indol-3- yl)methylene)pyrimidine- 2,4,6(1H,3H,5H)-trione |
| Compound 5 | 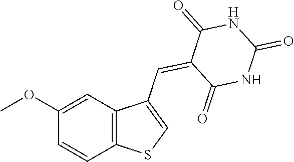 [SMILES: COc1ccc2scc(C=C3C(=O)NC(=O)NC3=O)c2c1] | 5-((5-methoxybenzo[b] thiophen-3- yl)methylene)pyrimidine- 2,4,6(1H,3H,5H)-trione |
| Compound 6 | 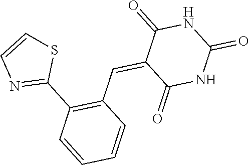 [SMILES: O=C1NC(=O)C(=Cc2ccccc2-c2nccs2)C(=O)N1] | 5-(2-(thiazol-2- yl)benzylidene)pyrimidine- 2,4,6(1H,3H,5H)-trione |
| Compound 7 | 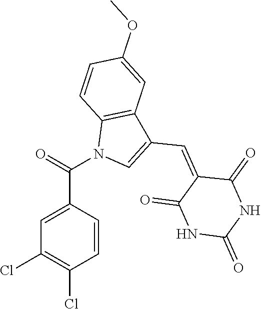 [SMILES: COc1ccc2c(c1)c(C=C1C(=O)NC(=O)NC1=O)cn2C(=O)c1ccc(Cl)c(Cl)c1] | 5-((1-(3,4- dichlorobenzoyl)-5-methoxy- 1H-indol-3-yl) methylene)pyrimidine- 2,4,6(1H,3H,5H)-trione |
| Compound 8 | 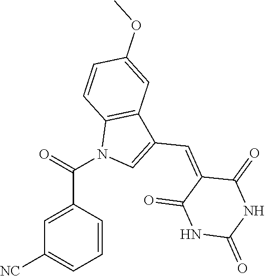 [SMILES: COc1ccc2c(c1)c(C=C1C(=O)NC(=O)NC1=O)cn2C(=O)c1cccc(C#N)c1] | 3-(5-methoxy-3-((2,4,6- trioxotetrahydropyrimidin- 5(2H)- ylidene)methyl)-1H-indole-1- carbonyl)benzonitrile |
| Compound 9 | 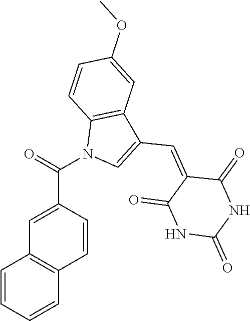 [SMILES: COc1ccc2c(c1)c(C=C1C(=O)NC(=O)NC1=O)cn2C(=O)c1ccc2ccccc2c1] | 5-((1-(2-naphthoyl)- 5-methoxy-1H- indol-3-yl)methylene) pyrimidine- 2,4,6(1H,3H,5H)-trione |
| Compound 10 | 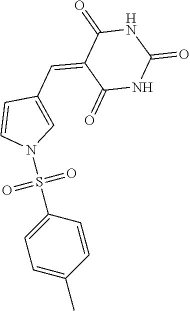 [SMILES: Cc1ccc(S(=O)(=O)n2ccc(C=C3C(=O)NC(=O)NC3=O)c2)cc1] | 5-((1-tosyl-1H-pyrrol-3- yl)methylene)pyrimidine- 2,4,6(1H,3H,5H)-trione |
| Compound 11 | 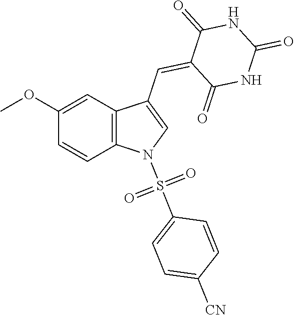 [SMILES: COc1ccc2c(c1)c(C=C1C(=O)NC(=O)NC1=O)cn2S(=O)(=O)c1ccc(C#N)cc1] | 4-((5-methoxy-3-((2,4,6- trioxotetrahydropyrimidin-5(2H)- ylidene)methyl)-1H-indol-1- yl)sulfonyl)benzonitrile |
| Compound 12 | 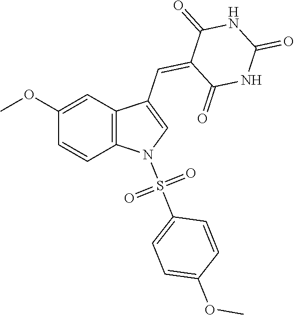 [SMILES: COc1ccc(S(=O)(=O)n2cc(C=C3C(=O)NC(=O)NC3=O)c3cc(OC)ccc32)cc1] | 5-((5-methoxy-1-((4- methoxyphenyl) sulfonyl)-1H-indol-3- yl)methylene)pyrimidine- 2,4,6(1H,3H,5H)-trione |
| Compound 13 | 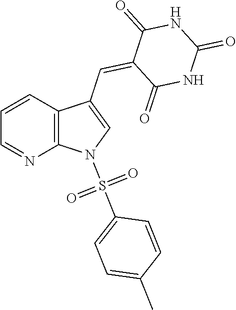 [SMILES: Cc1ccc(S(=O)(=O)n2cc(C=C3C(=O)NC(=O)NC3=O)c3cccnc32)cc1] | 5-((1-tosyl-1H- pyrrolo[2,3-b]pyridin-3- yl)methylene)pyrimidine- 2,4,6(1H,3H,5H)-trione |
| Compound 14 | 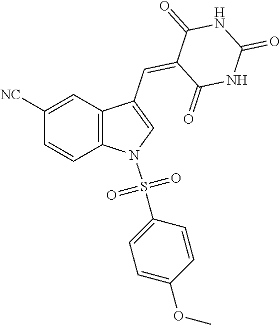 [SMILES: COc1ccc(S(=O)(=O)n2cc(C=C3C(=O)NC(=O)NC3=O)c3cc(C#N)ccc32)cc1] | 1-((4-methoxyphenyl) sulfonyl)-3- ((2,4,6- trioxotetrahydropyrimidin- 5(2H)-ylidene)methyl)- 1H-indole- 5-carbonitrile |
| Compound 15 | 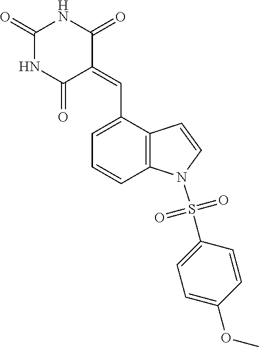 [SMILES: COc1ccc(S(=O)(=O)n2ccc3c(C=C4C(=O)NC(=O)NC4=O)cccc32)cc1] | 5-((1-((4-methoxyphenyl) sulfonyl)-1H- indol-4-yl)methylene) pyrimidine- 2,4,6(1H,3H,5H)-trione |
| Compound 16 | 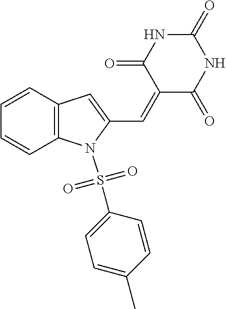 [SMILES: Cc1ccc(S(=O)(=O)n2c(C=C3C(=O)NC(=O)NC3=O)cc3ccccc32)cc1] | 5-((1-tosyl-1H-indol-2- yl)methylene)pyrimidine- 2,4,6(1H,3H,5H)-trione |
| Compound 17 | 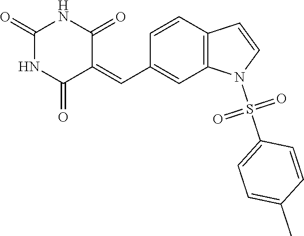 [SMILES: Cc1ccc(S(=O)(=O)n2ccc3ccc(C=C4C(=O)NC(=O)NC4=O)cc32)cc1] | 5-((1-tosyl-1H-indol-6- yl)methylene)pyrimidine- 2,4,6(1H,3H,5H)-trione |
| Compound 18 | 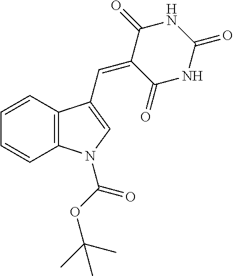 [SMILES: CC(C)(C)OC(=O)n1cc(C=C2C(=O)NC(=O)NC2=O)c2ccccc21] | tert-butyl 3-((2,4,6- trioxotetrahydropyrimidin- 5(2H)- ylidene)methyl)-1H-indole-1- carboxylate |
| Compound 19 | 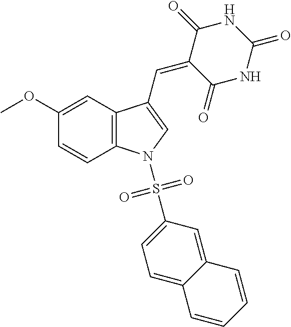 [SMILES: COc1ccc2c(c1)c(C=C1C(=O)NC(=O)NC1=O)cn2S(=O)(=O)c1ccc2ccccc2c1] | 5-((5-methoxy-1-(naphthalen-2- ylsulfonyl)-1H-indol-3- yl)methylene)pyrimidine- 2,4,6(1H,3H,5H)-trione |
| Compound 20 | 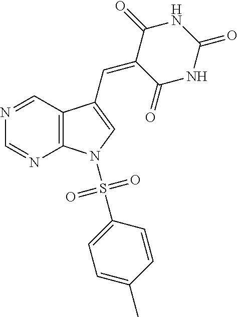 [SMILES: Cc1ccc(S(=O)(=O)n2cc(C=C3C(=O)NC(=O)NC3=O)c3cncnc32)cc1] | 5-((7-tosyl-7H- pyrrolo[2,3-d]pyrimidin- 5-yl)methylene)pyrimidine- 2,4,6(1H,3H,5H)-trione |
| Compound 21 | 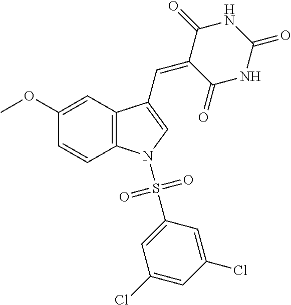 [SMILES: COc1ccc2c(c1)c(C=C1C(=O)NC(=O)NC1=O)cn2S(=O)(=O)c1cc(Cl)cc(Cl)c1] | 5-((1-((3,5- dichlorophenyl)sulfonyl)-5- methoxy-1H-indol-3- yl)methylene)pyrimidine- 2,4,6(1H,3H,5H)-trione |
| Compound 22 | 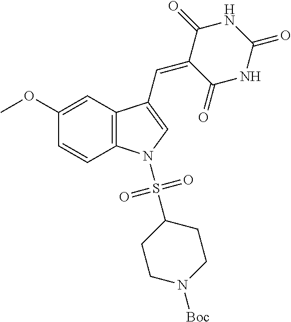 [SMILES: COc1ccc2c(c1)c(C=C1C(=O)NC(=O)NC1=O)cn2S(=O)(=O)C1CCN(C(=O)OC(C)(C)C)CC1] | tert-butyl 4-((5- methoxy-3-((2,4,6- trioxotetrahydropyrimidin- 5(2H)- ylidene)methyl)-1H-indol-1- yl)sulfonyl)piperidine- 1-carboxylate |
| Compound 23 | 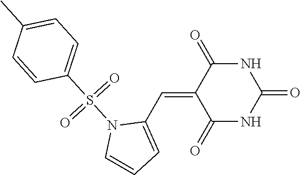 [SMILES: Cc1ccc(S(=O)(=O)n2cccc2C=C2C(=O)NC(=O)NC2=O)cc1] | 5-((1-tosyl-1H-pyrrol-2- yl)methylene)pyrimidine- 2,4,6(1H,3H,5H)-trione |
| Compound 24 | 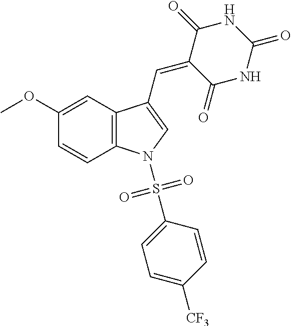 [SMILES: COc1ccc2c(c1)c(C=C1C(=O)NC(=O)NC1=O)cn2S(=O)(=O)c1ccc(C(F)(F)F)cc1] | 5-((5-methoxy-1-((4- (trifluoromethyl)phenyl) sulfonyl)-1H- indol-3-yl)methylene) pyrimidine- 2,4,6(1H,3H,5H)-trione |
| Compound 25 | 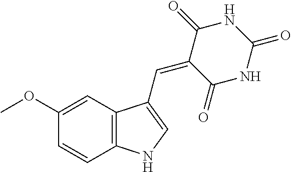 [SMILES: COc1ccc2[nH]cc(C=C3C(=O)NC(=O)NC3=O)c2c1] | 5-((5-methoxy-1H-indol-3- yl)methylene)pyrimidine- 2,4,6(1H,3H,5H)-trione |
| Compound 26 | 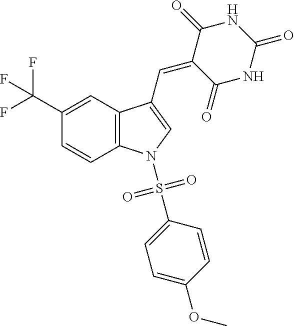 [SMILES: COc1ccc(S(=O)(=O)n2cc(C=C3C(=O)NC(=O)NC3=O)c3cc(C(F)(F)F)ccc32)cc1] | 5-((1-((4- methoxyphenyl)sulfonyl)-5- (trifluoromethyl)-1H-indol-3- yl)methylene)pyrimidine- 2,4,6(1H,3H,5H)-trione |
| Compound 27 | 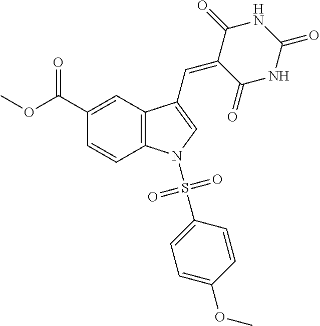 [SMILES: COC(=O)c1ccc2c(c1)c(C=C1C(=O)NC(=O)NC1=O)cn2S(=O)(=O)c1ccc(OC)cc1] | methyl 1-((4- methoxyphenyl)sulfonyl)- 3-((2,4,6- trioxotetrahydropyrimidin- 5(2H)-ylidene)methyl)- 1H-indole-5- carboxylate |
| Compound 28 | 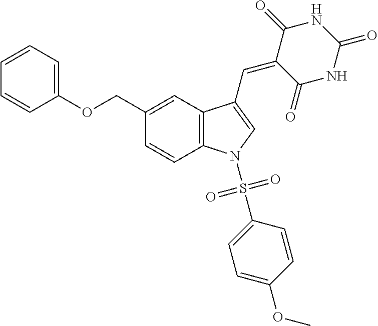 [SMILES: COc1ccc(S(=O)(=O)n2cc(C=C3C(=O)NC(=O)NC3=O)c3cc(COc4ccccc4)ccc32)cc1] | 5-((5-(benzyloxy)-1-((4- methoxyphenyl) sulfonyl)-1H-indol-3- yl)methylene)pyrimidine- 2,4,6(1H,3H,5H)-trione |
| Compound 29 | 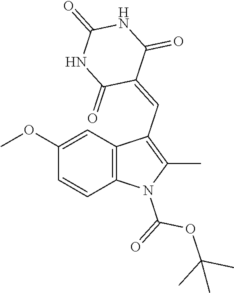 [SMILES: COc1ccc2c(c1)c(C=C1C(=O)NC(=O)NC1=O)c(C)n2C(=O)OC(C)(C)C] | tert-butyl 5-methoxy- 2-methyl-3-((2,4,6- trioxotetrahydropyrimidin- 5(2H)- ylidene)methyl)-1H-indole-1- carboxylate |
| Compound 30 | 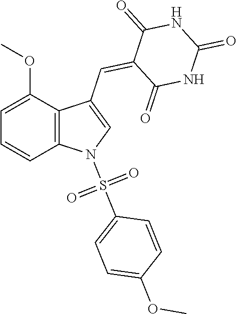 [SMILES: COc1ccc(S(=O)(=O)n2cc(C=C3C(=O)NC(=O)NC3=O)c3c(OC)cccc32)cc1] | 5-((4-methoxy-1-((4- methoxyphenyl) sulfonyl)-1H-indol-3- yl)methylene)pyrimidine- 2,4,6(1H,3H,5H)-trione |
| Compound 31 | 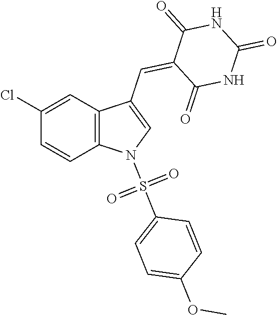 [SMILES: COc1ccc(S(=O)(=O)n2cc(C=C3C(=O)NC(=O)NC3=O)c3cc(Cl)ccc32)cc1] | 5-((5-chloro-1-((4- methoxyphenyl) sulfonyl)-1H-indol-3- yl)methylene)pyrimidine- 2,4,6(1H,3H,5H)-trione |
| Compound 32 | 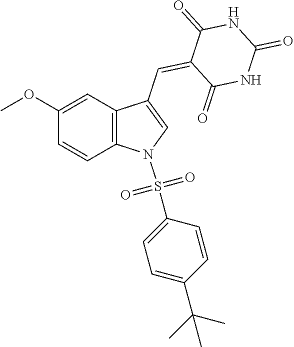 [SMILES: COc1ccc2c(c1)c(C=C1C(=O)NC(=O)NC1=O)cn2S(=O)(=O)c1ccc(C(C)(C)C)cc1] | 5-((1-((4-(tert-butyl) phenyl)sulfonyl)-5- methoxy-1H-indol-3- yl)methylene)pyrimidine- 2,4,6(1H,3H,5H)-trione |
| Compound 33 | 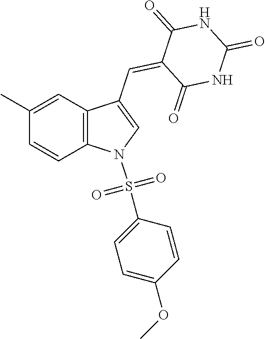 [SMILES: COc1ccc(S(=O)(=O)n2cc(C=C3C(=O)NC(=O)NC3=O)c3cc(C)ccc32)cc1] | 5-((1-((4- methoxyphenyl)sulfonyl)-5- methyl-1H-indol-3- yl)methylene)pyrimidine- 2,4,6(1H,3H,5H)-trione |
| Compound 34 | 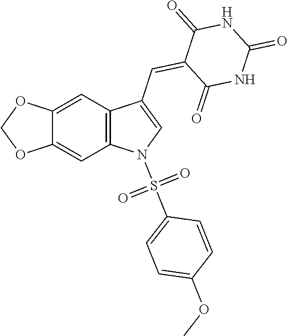 [SMILES: COc1ccc(S(=O)(=O)n2cc(C=C3C(=O)NC(=O)NC3=O)c3cc4c(cc32)OCO4)cc1] | 5-((5-((4-methoxyphenyl) sulfonyl)-5H- [1,3]dioxolo[4,5-f]indol-7- yl)methylene)pyrimidine- 2,4,6(1H,3H,5H)-trione |
| Compound 35 | 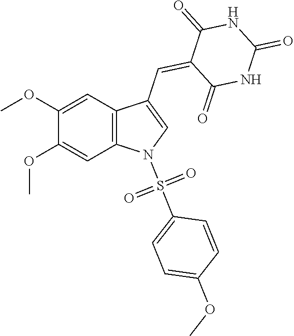 [SMILES: COc1ccc(S(=O)(=O)n2cc(C=C3C(=O)NC(=O)NC3=O)c3cc(OC)c(OC)cc32)cc1] | 5-((5,6-dimethoxy-1-((4- methoxyphenyl)sulfonyl)- 1H-indol-3- yl)methylene)pyrimidine- 2,4,6(1H,3H,5H)-trione |
| Compound 36 | 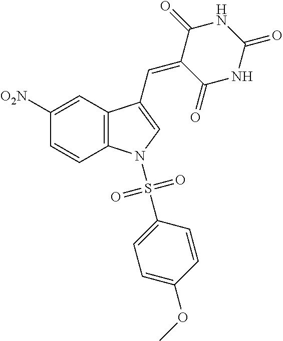 [SMILES: COc1ccc(S(=O)(=O)n2cc(C=C3C(=O)NC(=O)NC3=O)c3cc([N+](=O)[O-])ccc32)cc1] | 5-((1-((4- methoxyphenyl)sulfonyl)-5- nitro-1H-indol-3- yl)methylene)pyrimidine- 2,4,6(1H,3H,5H)-trione |
| Compound 37 | 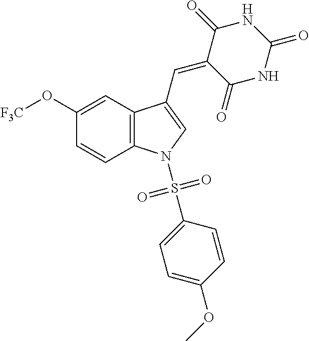 [SMILES: COc1ccc(S(=O)(=O)n2cc(C=C3C(=O)NC(=O)NC3=O)c3cc(OC(F)(F)F)ccc32)cc1] | 5-((1-((4- methoxyphenyl)sulfonyl)-5- (trifluoromethoxy)-1H-indol-3- yl)methylene)pyrimidine- 2,4,6(1H,3H,5H)-trione |
| Compound 38 | 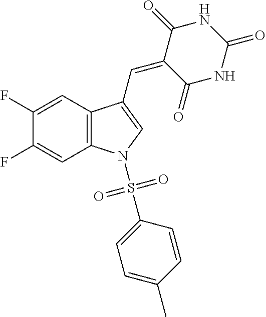 [SMILES: Cc1ccc(S(=O)(=O)n2cc(C=C3C(=O)NC(=O)NC3=O)c3cc(F)c(F)cc32)cc1] | 5-((5,6-difluoro- 1-tosyl-1H-indol-3- yl)methylene)pyrimidine- 2,4,6(1H,3H,5H)-trione |
| | NS-59 | |
| Compound 39 | 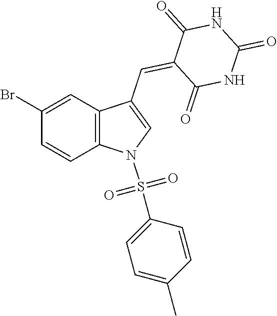 [SMILES: Cc1ccc(S(=O)(=O)n2cc(C=C3C(=O)NC(=O)NC3=O)c3cc(Br)ccc32)cc1] | 5-((5-bromo-1- tosyl-1H-indol-3- yl)methylene)pyrimidine- 2,4,6(1H,3H,5H)-trione |
| Compound 40 | 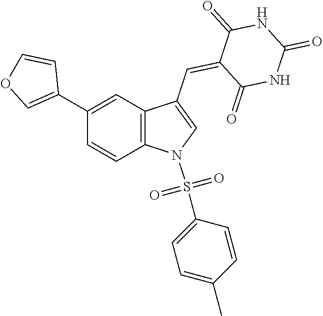 [SMILES: Cc1ccc(S(=O)(=O)n2cc(C=C3C(=O)NC(=O)NC3=O)c3cc(-c4ccoc4)ccc32)cc1] | 5-((5-(furan-3-yl)- 1-tosyl-1H-indol-3- yl)methylene)pyrimidine- 2,4,6(1H,3H,5H)-trione |
| Compound 41 | 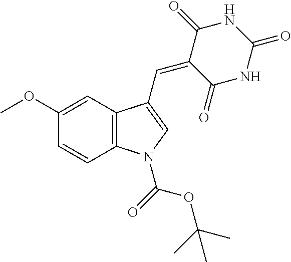 [SMILES: COc1ccc2c(c1)c(C=C1C(=O)NC(=O)NC1=O)cn2C(=O)OC(C)(C)C] | tert-butyl 5-methoxy-3-((2,4,6- trioxotetrahydropyrimidin- 5(2H)- ylidene)methyl)-1H-indole-1- carboxylate |
| Compound 42 | 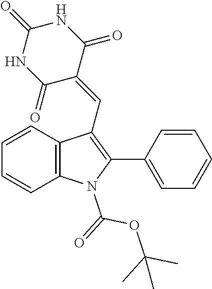 [SMILES: CC(C)(C)OC(=O)n1c(-c2ccccc2)c(C=C2C(=O)NC(=O)NC2=O)c2ccccc21] | tert-butyl 2-phenyl-3-((2,4,6- trioxotetrahydropyrimidin- 5(2H)- ylidene)methyl)-1H-indole-1- carboxylate |
| Compound 43 | 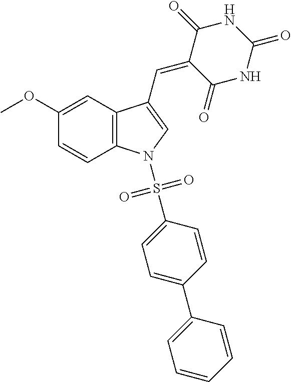 [SMILES: COc1ccc2c(c1)c(C=C1C(=O)NC(=O)NC1=O)cn2S(=O)(=O)c1ccc(-c2ccccc2)cc1] | 5-((1-([1,1′-biphenyl]- 4-ylsulfonyl)-5- methoxy-1H-indol-3- yl)methylene)pyrimidine- 2,4,6(1H,3H,5H)-trione |
| Compound 44 | 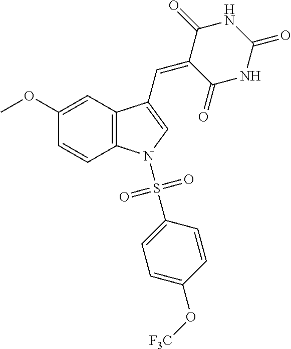 [SMILES: COc1ccc2c(c1)c(C=C1C(=O)NC(=O)NC1=O)cn2S(=O)(=O)c1ccc(OC(F)(F)F)cc1] | 5-((5-methoxy-1-((4- (trifluoromethoxy)phenyl) sulfonyl)-1H- indol-3-yl)methylene) pyrimidine- 2,4,6(1H,3H,5H)-trione |
| Compound 45 | 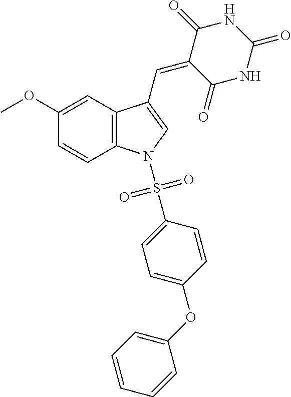 [SMILES: COc1ccc2c(c1)c(C=C1C(=O)NC(=O)NC1=O)cn2S(=O)(=O)c1ccc(Oc2ccccc2)cc1] | 5-((5-methoxy-1-((4- phenoxyphenyl)sulfonyl)- 1H-indol-3- yl)methylene)pyrimidine- 2,4,6(1H,3H,5H)-trione |
| Compound 46 | 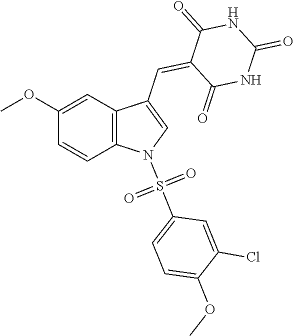 [SMILES: COc1ccc2c(c1)c(C=C1C(=O)NC(=O)NC1=O)cn2S(=O)(=O)c1ccc(OC)c(Cl)c1] | 5-((1-((3-chloro-4- methoxyphenyl)sulfonyl)- 5-methoxy- 1H-indol-3-yl)methylene) pyrimidine- 2,4,6(1H,3H,5H)-trione |
| Compound 47 | 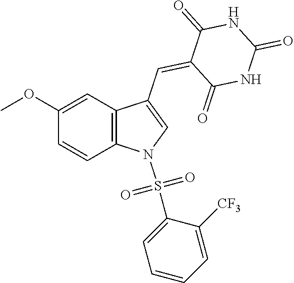 [SMILES: COc1ccc2c(c1)c(C=C1C(=O)NC(=O)NC1=O)cn2S(=O)(=O)c1ccccc1C(F)(F)F] | 5-((5-methoxy-1-((2- (trifluoromethyl) phenyl)sulfonyl)-1H- indol-3-yl)methylene) pyrimidine- 2,4,6(1H,3H,5H)-trione |
| Compound 48 | 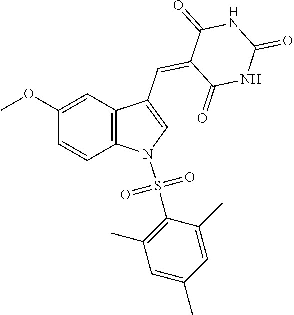 [SMILES: COc1ccc2c(c1)c(C=C1C(=O)NC(=O)NC1=O)cn2S(=O)(=O)c1c(C)cc(C)cc1C] | 5-((1-(mesitylsulfonyl)- 5-methoxy-1H- indol-3-yl)methylene) pyrimidine- 2,4,6(1H,3H,5H)-trione |
| Compound 49 | 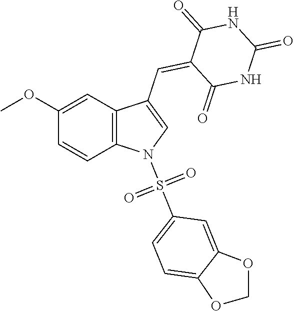 [SMILES: COc1ccc2c(c1)c(C=C1C(=O)NC(=O)NC1=O)cn2S(=O)(=O)c1ccc2c(c1)OCO2] | 5-((1-(benzo[d][1,3]dioxol-5- ylsulfonyl)-5-methoxy- 1H-indol-3- yl)methylene)pyrimidine- 2,4,6(1H,3H,5H)-trione |
| Compound 50 | 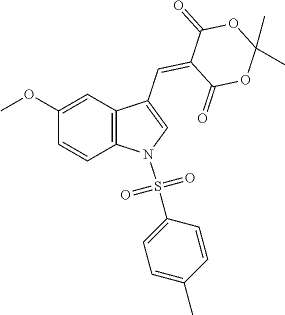 | 5-((5-methoxy-1- tosyl-1H-indol-3- yl)methylene)-2,2- dimethyl-1,3- dioxane-4,6-dione |
| Compound 51 | 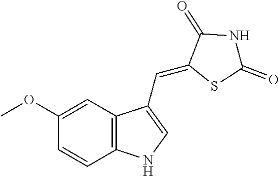 [SMILES: COc1ccc2[nH]cc(/C=C3\SC(=O)NC3=O)c2c1] | (Z)-5-((5-methoxy- 1H-indol-3- yl)methylene)thiazolidine- 2,4-dione |
| Compound 52 | 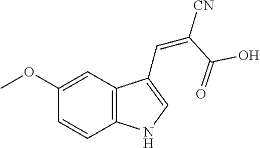 [SMILES: COc1ccc2[nH]cc(/C=C(/C#N)C(=O)O)c2c1] | (Z)-2-cyano-3-(5- methoxy-1H-indol-3- yl)acrylic acid |
| Compound 53 | 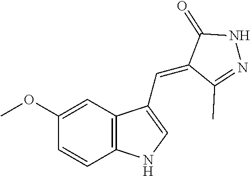 [SMILES: COc1ccc2[nH]cc(/C=C3/C(=O)NN=C3C)c2c1] | (E)-4-((5-methoxy- 1H-indol-3- yl)methylene)-5- methyl-2,4-dihydro- 3H-pyrazol-3-one |
| Compound 54 | 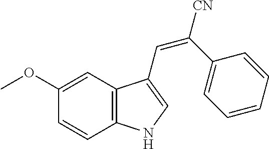 [SMILES: COc1ccc2[nH]cc(/C=C(/C#N)c3ccccc3)c2c1] | (E)-3-(5-methoxy- 1H-indol-3-yl)-2- phenylacrylonitrile |
| Compound 55 | 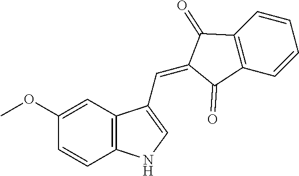 [SMILES: COc1ccc2[nH]cc(C=C3C(=O)c4ccccc4C3=O)c2c1] | 2-((5-methoxy-1H-indol-3- yl)methylene)-1H- indene-1,3(2H)-dione |
| Compound 56 | 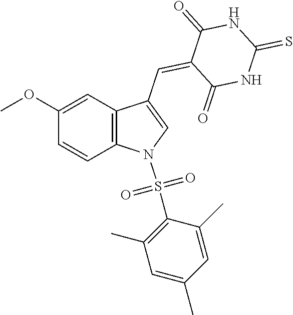 [SMILES: COc1ccc2c(c1)c(C=C1C(=O)NC(=S)NC1=O)cn2S(=O)(=O)c1c(C)cc(C)cc1C] | 5-((1-(mesitylsulfonyl)- 5-methoxy-1H- indol-3-yl)methylene)-2- thioxodihydropyrimidine- 4,6(1H,5H)-dione |
| Compound 57 | 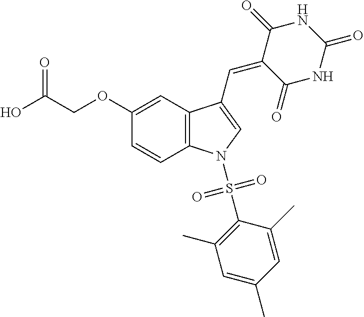 [SMILES: Cc1cc(C)c(S(=O)(=O)n2cc(C=C3C(=O)NC(=O)NC3=O)c3cc(OCC(=O)O)ccc32)c(C)c1] | 2-((1-(mesitylsulfonyl)- 3-((2,4,6- trioxotetrahydropyrimidin- 5(2H)- ylidene)methyl)-1H-indol-5- yl)oxy)acetic acid |

In various aspects, a compound of the resent disclosure is selected from

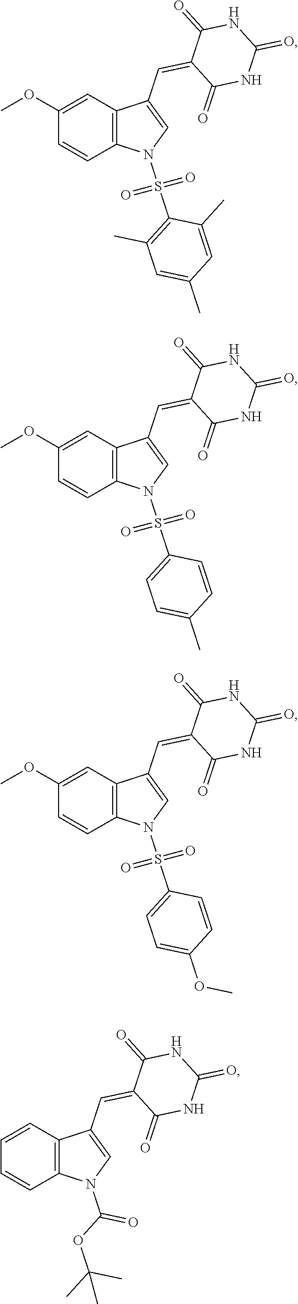

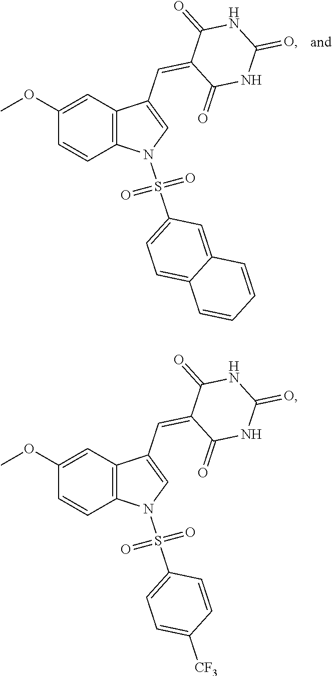 [SMILES: COc1ccc2c(c1)c(C=C1C(=O)NC(=O)NC1=O)cn2S(=O)(=O)c1ccc2ccccc2c1 ; COc1ccc2c(c1)c(C=C1C(=O)NC(=O)NC1=O)cn2S(=O)(=O)c1ccc(C(F)(F)F)cc1]

or is any pharmaceutically appropriate salt thereof.

In some aspects, a compound of the present disclosure comprises a structure of

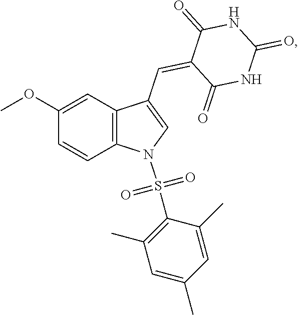 [SMILES: COc1ccc2c(c1)c(C=C1C(=O)NC(=O)NC1=O)cn2S(=O)(=O)c1c(C)cc(C)cc1C]

or any pharmaceutically appropriate salt thereof.

As noted, any of the compounds (e.g., NSUN2 inhibitors) may be provided as a pharmaceutically appropriate salt. “Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### VII. Methods of Use

In some aspects, provided herein are methods of using compositions and technologies disclosed herein, and/or new uses of additional compositions and/or methods known in the art.

In certain aspects, provided herein are methods of determining appropriate treatment regimens and/or treating diseases and/or disorders such as but not limited to: acute myeloid leukemias (AML), including at least AML with a translocation between chromosomes 8 and 21 [t(8;21)], AML with a translocation or inversion in chromosome 16 [t(16;16) or inv(16)], AML with a translocation or inversion in chromosome 16 [t(16;16) or inv(16)], AML with a translocation or inversion in chromosome 16 [t(16;16) or inv(16)], AML with a translocation between chromosomes 6 and 9 [t(6:9)], AML with a translocation or inversion in chromosome 3 [t(3;3) or inv(3)], AML (megakaryoblastic) with a translocation between chromosomes 1 and 22 [t(1:22)], AML with the BCR-ABL1 (BCR-ABL) fusion gene, AML with mutated NPM1 gene, AML with biallelic mutations of the CEBPA gene, AML with mutated RUNX1 gene, AML with myelodysplasia-related changes, AML related to previous chemotherapy or radiation, AML not otherwise specified, AML with minimal differentiation, AML without maturation, AML with maturation, Acute myelomonocytic leukemia, Acute monoblastic/monocytic leukemia, pure erythroid leukemia, acute megakaryoblastic leukemia, acute basophilic leukemia, acute panmyelosis with fibrosis, myeloid sarcoma, myeloid proliferations related to down syndrome, undifferentiated and biphenotypic acute leukemias, AML with mutations in IKZF1, NPM1, EZH2, TET2, TP53, RUNX1, and/or ASXL1.

In certain aspects, provided herein are methods of determining appropriate treatment regimens and/or treating diseases and/or disorders such as but not limited to chronic myeloid leukemia (CML) and/or chronic myelomonocytic leukemia.

In certain aspects, provided herein are methods of determining appropriate treatment regimens and/or treating diseases and/or disorders such as but not limited to gliomas such as but not limited to astrocytomas (including glioblastoma multiforme), brain stem gliomas, ependymomas, mixed gliomas, oligodendrogliomas, and/or optic pathway gliomas.

In certain aspects, provided herein are methods of determining appropriate treatment regimens and/or treating diseases and/or disorders such as but not limited to clonal hematopoiesis of indeterminate potential (CHIP) or disease states associated therewith (such as but not limited to atherosclerosis, myocardial fibrosis, and/or heart failure).

### A. Methods of Treatment

The present disclosure additionally provides methods of detecting, methods of measuring, methods of diagnosing, and/or methods of ameliorating and/or treating diseases. In some aspects, constructs, vectors, particles, polypeptides, polynucleotides, and/or compositions described herein may be comprised in a formulation with one or more additional therapeutic agents. In some aspects, constructs, vectors, particles, polypeptides, polynucleotides, and/or compositions described herein may be comprised in a formulation wherein the formulation comprises pharmaceutically acceptable excipients.

In some aspects, constructs, vectors, particles, polypeptides, polynucleotides, and/or compositions described herein may be administered to a cell in an in vitro environment. In some aspects, a cell may be derived from a subject. In some aspects, a cell is an immune cell, a stem cell, an induced pluripotent stem cell, a precursor cell, and/or a terminally differentiated cell. In some aspects, constructs, vectors, particles, polypeptides, polynucleotides, and/or compositions described herein may be administered to a cell in vivo via administration to a subject.

In some aspects, constructs, vectors, particles, polypeptides, polynucleotides, and/or compositions described herein are administered to a subject in need thereof. In some aspects, a subject may have, may be diagnosed with, or may be susceptible to a disease, such as an infectious disease, a genetic disorder, an autoimmune disease, and/or cancer.

In some aspects, a subject is a mammal. In some aspects, a subject is a domestic animal. In some aspects, a subject is a farm animal. In some aspects, a subject is a zoo animal. In some aspects, a subject is a dog or a cat. In some aspects, a subject is a cow, a horse, a sheep, or a goat. In some aspects, a subject can be but is not limited to, a dog, cat, ferret, rabbit, cow, duck, pig, goat, chicken, horse, llama, camel, ostrich, deer, turkey, dove, sheep, goose, oxen, and/or reindeer. In some aspects, a subject is a human. In some aspects, a subject is equal to, less than, or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age.

In some aspects, administration regimens comprising constructs, vectors, particles, polypeptides, polynucleotides, and/or compositions described herein comprise administering of more than one composition, such as 2 compositions, 3 compositions, 4 compositions, or more than 4 compositions. Various combinations of the agents may be employed. In some aspects, constructs, vectors, particles, polypeptides, polynucleotides, and/or compositions of the disclosure may be administered by the same route of administration or by different routes of administration. In some aspects, agents described herein and/or additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, constructs, vectors, particles, polypeptides, polynucleotides, and/or compositions described herein and/or additional therapeutic agents are administered intravenously, intramuscularly, subcutaneously, topically, orally, transdermally, intraperitoneally, intraorbitally, by implantation, by inhalation, intrathecally, intraventricularly, or intranasally. In some aspects, an appropriate dosage may be determined based on the type of disease to be treated and/or prevented, severity, and/or course of the disease, the clinical condition of the individual, the individual's clinical history and response to the treatment, and/or at the discretion of the attending physician.

In some aspects, administration to a subject may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, the quantity to be administered, both according to number of treatments and unit dose, depends on the treatment effect desired. An effective dose is understood to refer to an amount necessary to achieve a particular effect. In the practice in certain aspects, it is contemplated that doses in the range from 0.10 mg/kg to 200 mg/kg can affect the functionality of the described agents. In certain aspects, it is contemplated that doses may comprise a composition comprising an AAV particle in a concentration of about 10⁸ to about 10¹⁴ viral genomes per ml. Furthermore, such doses can be administered at multiple times during a day, and/or on multiple days, weeks, or months.

In certain aspects, precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, 12 week, or more than 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

In some aspects, wherein a composition is proteinaceous, the proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In some aspects, a pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some aspects, sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In some aspects, upon formulation, compositions described herein may be administered in a manner compatible with the dosage formulation and in such amount as is therapeutically or prophylactically effective. In some aspects, formulations are administered in a variety of dosage forms, such as the type of injectable solutions described above.

### 1. Diseases or Disorders

In some aspects, constructs, vectors, particles, polypeptides, polynucleotides, and/or compositions described herein, or otherwise known in the art, may be used in a method of preventing, treating, reducing the progression of, and/or reducing the risk of a disease or disorder. In some aspects, constructs, vectors, particles, polypeptides, polynucleotides, and/or compositions described herein, or those otherwise known in the art, may be used in treating a disease or disorder, wherein the disease or disorder is a neurodegenerative disease, an inflammatory disease, an autoimmune disease, a metabolic syndrome, a cancer, a vascular disease, a fibrotic disease, a viral infection, a bacterial infection, a fungal infection, a parasitic infection, a musculoskeletal disease (such as a myopathy), an ocular disease, or a genetic disorder.

In some aspects, the disease or disorder is a cancer. In some aspects, the cancer is pancreatic cancer, breast cancer, kidney cancer, bladder cancer, prostate cancer, testicular cancer, urothelial cancer, endometrial cancer, ovarian cancer, cervical cancer, renal cancer, esophageal cancer, gastrointestinal stromal tumor (GIST), multiple myeloma, cancer of secretory cells, thyroid cancer, gastrointestinal carcinoma, chronic myeloid leukemia, hepatocellular carcinoma, colon cancer, melanoma, malignant glioma, glioblastoma, glioblastoma multiforme, astrocytoma, dysplastic gangliocytoma of the cerebellum, Ewing's sarcoma, rhabdomyosarcoma, ependymoma, medulloblastoma, ductal adenocarcinoma, adenosquamous carcinoma, nephroblastoma, acinar cell carcinoma, neuroblastoma, or lung cancer. In some aspects, the cancer of secretory cells is non-Hodgkin's lymphoma, Burkitt's lymphoma, chronic lymphocytic leukemia, monoclonal gammopathy of undetermined significance (MGUS), plasmacytoma, lymphoplasmacytic lymphoma or acute lymphoblastic leukemia. In some aspects, the cancer comprises a blood cancer. In some aspects, the cancer comprises a leukemia. In some aspects, the blood cancer comprises a myeloid malignancy (e.g., acute myeloid leukemia, chronic myelomonocytic leukemia, or myelodysplastic syndrome).

In some aspects, a cancer comprises, consists essentially of, consists of, or expressly does not comprise, an acute myeloid leukemia (AML), including at least AML with a translocation between chromosomes 8 and 21 [t(8;21)], AML with a translocation or inversion in chromosome 16 [t(16;16) or inv(16)], AML with a translocation or inversion in chromosome 16 [t(16;16) or inv(16)], AML with a translocation or inversion in chromosome 16 [t(16;16) or inv(16)], AML with a translocation between chromosomes 6 and 9 [t(6:9)], AML with a translocation or inversion in chromosome 3 [t(3;3) or inv(3)], AML (megakaryoblastic) with a translocation between chromosomes 1 and 22 [t(1:22)], AML with the BCR-ABL1 (BCR-ABL) fusion gene, AML with mutated NPM1 gene, AML with biallelic mutations of the CEBPA gene, AML with mutated RUNX1 gene, AML with myelodysplasia-related changes, AML related to previous chemotherapy or radiation, AML not otherwise specified, AML with minimal differentiation, AML without maturation, AML with maturation, Acute myelomonocytic leukemia, Acute monoblastic/monocytic leukemia, pure erythroid leukemia, acute megakaryoblastic leukemia, acute basophilic leukemia, acute panmyelosis with fibrosis, myeloid sarcoma, myeloid proliferations related to down syndrome, undifferentiated and biphenotypic acute leukemias, AML with a rearrangement of KMT2A (MLL) at chromosome 11q23, including at least AML with t(9;11)(p22;q23) (KMT2A-MLLT3/AF9), AML with t(11;19)(q23;p13.3) (KMT2A-MLLT1/ENL), AML with t(11;19)(q23;p13.1) (KMT2A-ELL), AML with t(6;11)(q27;q23) (KMT2A-AFDN/AF6), AML with t(10;11)(p12;q23) (KMT2A-MLLT10/AF10), and AML with t(4;11)(q21;q23)(KMT2A-AFF1/AF4), AML with mutations in IKZF1, NPM1, EZH2, TET2, TP53, RUNX1, and/or ASXL1. In certain aspects, one or more of the aforementioned AML are expressly excluded from AMLs subject to treatment with methods and/or compositions described herein.

In some aspects, a cancer comprises, consists essentially of, consists of, or expressly does not comprise, a glioma, including at least astrocytomas (including glioblastoma multiforme), brain stem gliomas, ependymomas, mixed gliomas, oligodendrogliomas, and/or optic pathway gliomas.

In some aspects, the disease or disorder is a pre-cancerous disease or disorder. In some aspects, the disease or disorder comprises, consists essentially of, consists of, or expressly does not comprise, clonal hematopoiesis of indeterminate potential (CHIP) or disease states associated therewith (such as but not limited to atherosclerosis, myocardial fibrosis, and/or heart failure). In some aspects, methods described herein comprise treatment of CHIP and/or one or more disease states associated therewith, comprising inhibition of IKZF1, NPM1, EZH2, and/or TET2. In some aspects, methods described herein comprise treatment of CHIP and/or one or more disease states associated therewith, comprising inhibition of IKZF1. In some aspects, methods described herein comprise treatment of CHIP and/or one or more disease states associated therewith, comprising inhibition of NPM1. In some aspects, methods described herein comprise treatment of CHIP and/or one or more disease states associated therewith, comprising inhibition of EZH2.

In some aspects, a disease or disorder for treatment comprising technologies described herein is associated with diseased cells with one or more mutations in one or more genes encoding IKZF1, NPM1, EZH2, ten-eleven translocation (tet) methylcytosine dioxygenase 2 (TET2), ASXL transcriptional regulator 1 (ASXL1), isocitrate dehydrogenase 1 (IDH1), isocitrate dehydrogenase 2 (IDH2), tumor protein p53 (p53), DNA (cytosine-5-)-methyltransferase 3A (DNMT3A), Janus kinase 2 (JAK2), Protein Phosphatase Mn2+/Mg2+-Dependent 1D (PPM1D), Spliceosome Factor 3b1 (SF3B1), and/or Serine and Arginine Rich Splicing Factor 2 (SRSF2). In some aspects, a disease or disorder for treatment comprising technologies described herein is associated with diseased cells comprising a rearrangement of the lysine methyltransferase 2A (KMT2A; also known as mixed-lineage leukemia (MLL)) gene, including but not limited to fusions of KMT2A with one or more partner genes (e.g., MLLT3 (AF9), MLLT1 (ENL), ELL, AFDN (AF6), MLLT10 (AF10), and/or AFF1 (AF4)). In some aspects, a disease or disorder for treatment expressly does not comprise diseased cells with one or more mutations in IKZF1, NPM1, EZH2, TET2, IDH1, IDH2, DNMT3A, ASXL1, PPM1D, TP53, JAK2, SF3B1, and/or SRSF2. In some aspects, a disease or disorder for treatment expressly does not comprise diseased cells comprising a rearrangement of the lysine methyltransferase 2A (KMT2A; also known as mixed-lineage leukemia (MLL)) gene. In some aspects, a disease or disorder for treatment expressly does not comprise diseased cells with one or more mutations in TET2. In some aspects, a disease or disorder for treatment expressly does not comprise diseased cells with one or more mutations in IKZF1. In some aspects, a disease or disorder for treatment expressly does not comprise diseased cells with one or more mutations in NPM1. In some aspects, a disease or disorder for treatment expressly does not comprise diseased cells with one or more mutations in EZH2. In some aspects, a disease or disorder for treatment expressly does not comprise diseased cells with one or more mutations in IDH1. In some aspects, a disease or disorder for treatment expressly does not comprise diseased cells with one or more mutations in IDH2. In some aspects, a disease or disorder for treatment expressly does not comprise diseased cells with one or more mutations in DNMT3A. In some aspects, a disease or disorder for treatment expressly does not comprise diseased cells with one or more mutations in ASXL1. In some aspects, a disease or disorder for treatment expressly does not comprise diseased cells with one or more mutations in PPM1D. In some aspects, a disease or disorder for treatment expressly does not comprise diseased cells with one or more mutations in TP53. In some aspects, a disease or disorder for treatment expressly does not comprise diseased cells with one or more mutations in JAK2. In some aspects, a disease or disorder for treatment expressly does not comprise diseased cells with one or more mutations in SF3B1. In some aspects, a disease or disorder for treatment expressly does not comprise diseased cells with one or more mutations in SRSF2.

In some aspects, a disease or disorder for treatment comprising technologies described herein is associated with a diseased cell comprising one or more mutations in a TET2 encoding gene (e.g., one or more loss of function mutations). In some aspects, a disease or disorder for treatment comprising technologies described herein is associated with diseased cells with one or more mutations in IKZF1, NPM1, and/or EZH2. In some aspects, a disease or disorder for treatment comprising technologies described herein is associated with diseased cells with one or more mutations in TET2, IDH1, IDH2, DNMT3A, ASXL1, PPM1D, TP53, JAK2, SF3B1, and/or SRSF2. In some aspects, a disease or disorder for treatment comprising technologies described herein is associated with diseased cells with one or more mutations in one or more genes encoding ASXL1, p53, DNMT3A, JAK2, PPM1D, SF3B1, and/or SRSF2.

In some aspects, a disease or disorder for treatment comprising technologies described herein is associated with diseased cells with one or more mutations in TET2, IDH1, IDH2, DNMT3A, ASXL1, PPM1D, TP53, JAK2, SF3B1, and/or SRSF2, wherein the one or more mutations renders the diseased cells susceptible to synthetic lethality induced by inhibition of IKZF1, NPM1, and/or EZH2. In some aspects, a disease or disorder for treatment comprising technologies described herein is associated with diseased cells with one or more mutations in TET2, IDH1, and/or IDH2, wherein the one or more mutations renders the diseased cells susceptible to synthetic lethality induced by inhibition of IKZF1, NPM1, and/or EZH2.

In some aspects, a disease or disorder for treatment comprising technologies described herein is associated with diseased cells comprising one or more mutations in one or more genes encoding components of a canonical and/or non-canonical Polycomb Repressive Complex (PRC). In some aspects, the one or more mutations in one or more genes encoding components of PRC comprises one or more loss of function mutations. In some aspects, a diseased cell comprising one or more mutations in one or more genes encoding components of PRC comprises one or more mutations in E3 Ubiquitin Ligase RING1A/B, Polycomb Group Ring Finger 1 (PCGF1), Polycomb Group Ring Finger 2 (PCGF2), Polycomb Group Ring Finger 3 (PCGF3), Polycomb Group Ring Finger 4 (PCGF4), Polycomb Group Ring Finger 5 (PCGF5), and/or Polycomb Group Ring Finger 6 (PCGF6). In some aspects, the diseased cell comprises one or more mutations in one or more genes encoding Polycomb Repressive-Deubiquitinase (PR-DUB) complex associated components O-linked N-acetylglucosamine Transferase (OGT), Lysine Demethylase 1B (KDM1B), Forkhead Box K1 (FOXK1), Forkhead Box K2 (FOXK2), BRCA1 Associated Protein 1 (BAP1), ASXL Transcriptional Regulator 1 (ASXL1), ASXL Transcriptional Regulator 2 (ASXL2), ASXL Transcription Regulator 3 (ASXL3), and/or Host Cell Factor C1 (HCFC1). For instance, in some aspects, the diseased cell comprising one or more mutations in PR-DUB complex associated components OGT, KDM1B, FOXK1, FOXK2, BAP1, ASXL1, ASXL2, ASXL3, and/or HCFC1 comprises one or more gain of function mutations in those genes. In some aspects, a disease or disorder for treatment expressly does not comprise diseased cells with one or more mutations in OGT. In some aspects, a disease or disorder for treatment expressly does not comprise diseased cells with one or more mutations in KDM1B. In some aspects, a disease or disorder for treatment expressly does not comprise diseased cells with one or more mutations in FOXK1. In some aspects, a disease or disorder for treatment expressly does not comprise diseased cells with one or more mutations in FOXK2. In some aspects, a disease or disorder for treatment expressly does not comprise diseased cells with one or more mutations in BAP1. In some aspects, a disease or disorder for treatment expressly does not comprise diseased cells with one or more mutations in ASXL1. In some aspects, a disease or disorder for treatment expressly does not comprise diseased cells with one or more mutations in ASXL2. In some aspects, a disease or disorder for treatment expressly does not comprise diseased cells with one or more mutations in ASXL3. In some aspects, a disease or disorder for treatment expressly does not comprise diseased cells with one or more mutations in HCFC1. In some aspects, a disease or disorder for treatment expressly does not comprise diseased cells with one or more mutations in IKZF1. In some aspects, a disease or disorder for treatment expressly does not comprise diseased cells with one or more mutations in NPM1. In some aspects, a disease or disorder for treatment expressly does not comprise diseased cells with one or more mutations in EZH2.

In some aspects, the disease or disorder is an inflammatory disease. In some aspects, the inflammatory disease is, or expressly is not, arthritis, psoriatic arthritis, psoriasis, juvenile idiopathic arthritis, asthma, allergic asthma, bronchial asthma, tuberculosis, chronic airway disorder, cystic fibrosis, glomerulonephritis, membranous nephropathy, sarcoidosis, vasculitis, ichthyosis, transplant rejection, interstitial cystitis, atopic dermatitis, or inflammatory bowel disease. In some aspects, the inflammatory bowel disease is Crohn' disease, ulcerative colitis, inflammatory bowel disease, or celiac disease.

In some aspects, the disease or disorder is an autoimmune disease. In some aspects, the autoimmune disease is, or expressly is not, systemic lupus erythematosus, type 1 diabetes, multiple sclerosis, psoriasis/psoriatic arthritis, inflammatory bowel disease, Addison's disease, Graves' disease, Sjogren's syndrome, Hashimoto's thyroiditis, Myasthenia gravis, autoimmune vasculitis, pernicious anemia, celiac disease, or rheumatoid arthritis.

In some aspects, the disease or disorder is a metabolic syndrome. In some aspects, the metabolic syndrome is, or expressly is not, acute pancreatitis, chronic pancreatitis, alcoholic liver steatosis, obesity, glucose intolerance, insulin resistance, hyperglycemia, fatty liver, dyslipidemia, hyperlipidemia, hyperhomocysteinemia, or type 2 diabetes. In some aspects, the metabolic syndrome is alcoholic liver steatosis, obesity, glucose intolerance, insulin resistance, hyperglycemia, fatty liver, dyslipidemia, hyperlipidemia, hyperhomocysteinemia, or type 2 diabetes.

In some aspects, the disease or disorder is a musculoskeletal disease (such as a myopathy). In some aspects, the musculoskeletal disease is, or expressly is not, a myopathy, a muscular dystrophy, a muscular atrophy, a muscular wasting, or sarcopenia. In some aspects, the muscular dystrophy is, or expressly is not, Duchenne muscular dystrophy (DMD), Becker's disease, myotonic dystrophy, X-linked dilated cardiomyopathy, spinal muscular atrophy (SMA), or metaphyseal chondrodysplasia, Schmid type (MCDS). In some aspects, the myopathy is a skeletal muscle atrophy. In some aspects, the musculoskeletal disease (such as the skeletal muscle atrophy) is triggered by ageing, chronic diseases, stroke, malnutrition, bedrest, orthopedic injury, bone fracture, cachexia, starvation, heart failure, obstructive lung disease, renal failure, Acquired Immunodeficiency Syndrome (AIDS), sepsis, an immune disorder, a cancer, ALS, a burn injury, denervation, diabetes, muscle disuse, limb immobilization, mechanical unload, myositis, or a dystrophy.

In some aspects, the disease or disorder is, or expressly is not, a musculoskeletal disease. In some aspects, skeletal muscle mass, quality and/or strength are increased. In some aspects, synthesis of muscle proteins is increased. In some aspects, skeletal muscle fiber atrophy is inhibited.

In some aspects, the disease or disorder is, or expressly is not, a vascular disease. In some aspects, the vascular disease is atherosclerosis, abdominal aortic aneurism, carotid artery disease, deep vein thrombosis, Buerger's disease, chronic venous hypertension, vascular calcification, telangiectasia or lymphoedema.

In some aspects, the disease or disorder is genetic disorder. In some aspects, a genetic disorder is, or expressly is not, arrhythmogenic right ventricular dysplasia/cardiomyopathy, Brugada Syndrome, Charcot-Marie-Tooth Disease, Cleft Lip and Palate, Cleidocranial Dysplasia, Cystic Fibrosis, Familial Adenomatous Polyposis, Hirschsprungs Disease, Huntington's Disease, Klinefelter Syndrome, Kneist Syndrome, Marfan Syndrome, Mucopolysaccharidoses, Muscular Dystrophy, Sickle Cell Disease, Von Hippel-Lindau Syndrome, Congenital Deafness, Familial Hypercholesterolemia, Hemochromatosis, Neurofibromatosis type 1, Tay-Sachs Disease, Usher Syndrome, AA amyloidosis, Adrenoleukodystrophy, Ehlers-Danlos Syndrome, Lysosomal disorders, and/or Mitochondrial disorders.

In some aspects, the disease or disorder is, or expressly is not, an ocular disease. In some aspects, the ocular disease is, or expressly is not, glaucoma, age-related macular degeneration, inflammatory retinal disease, retinal vascular disease, diabetic retinopathy, uveitis, rosacea, Sjogren's syndrome, retinitis pigmentosa, retinoschisis, Stargardt disease, Leber congenital amaurosis, or neovascularization in proliferative retinopathy.

### 2. Pharmaceutical Compositions

In certain aspects, the constructs, vectors, particles, polypeptides, polynucleotides, and/or compositions (collectively described as “agents”) for use in the methods, such as methods of m⁵C RNA writing, reading, and/or erasing, and/or inhibition of NSUN1, NSUN2, MBD5, MBD6, and/or TET2, are suitably contained in a pharmaceutically acceptable carrier. In some aspects, the carrier is non-toxic, biocompatible and is selected so as not to detrimentally affect the biological activity of the agent. In some aspects, agents may be formulated into preparations for local delivery (i.e. to a specific location of the body, such as brain tissues, muscle tissue, fat tissue, etc.) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

In some aspects, the compounds of the disclosure, such as small molecule inhibitors described herein including compounds of Formula (I), may exist in various solid-state forms, including crystalline forms, polymorphic forms, amorphous forms, solvates, hydrates, and co-crystals. In some aspects, a compound of the disclosure is provided in a crystalline form. In some aspects, a compound of the disclosure is provided in an amorphous form. In some aspects, a compound of the disclosure may exist in more than one polymorphic form, and the disclosure expressly encompasses all such polymorphic forms. In some aspects, the solid-state form of a compound may affect one or more pharmaceutical properties, such as but not limited to solubility, dissolution rate, hygroscopicity, chemical stability, physical stability, oral bioavailability, and/or processability for manufacturing purposes. In some aspects, the disclosure encompasses all crystalline forms, polymorphic forms, amorphous forms, solvated forms, hydrated forms, and co-crystalline forms of the compounds described herein, as well as mixtures thereof.

In some aspects, the compounds of the disclosure, such as small molecule inhibitors including compounds of Formula (I), may be formulated as pharmaceutically acceptable salts. In some aspects, pharmaceutically acceptable acid addition salts of the compounds of the disclosure may be formed with inorganic acids, including but not limited to hydrochloric acid, hydrobromic acid, sulfuric acid, phosphoric acid, and nitric acid, or with organic acids including but not limited to acetic acid, propionic acid, oxalic acid, maleic acid, fumaric acid, succinic acid, tartaric acid, citric acid, benzoic acid, methanesulfonic acid, ethanesulfonic acid, benzenesulfonic acid, p-toluenesulfonic acid, and the like. In some aspects, where a compound of the disclosure contains an acidic moiety, pharmaceutically acceptable base addition salts may be formed with inorganic bases, including but not limited to sodium hydroxide, potassium hydroxide, calcium hydroxide, and magnesium hydroxide, or with organic bases such as lysine, arginine, glucamine, choline, and the like. In some aspects, selection of a particular salt form may be used to optimize one or more pharmaceutical properties of the compound, including but not limited to aqueous solubility, crystallinity, chemical stability, hygroscopicity, oral bioavailability, and/or ease of formulation and manufacturing.

In some aspects, the compounds of the disclosure may exist as stereoisomers, including enantiomers, diastereomers, geometric isomers (cis/trans), and/or atropisomers. In some aspects, the disclosure encompasses individual stereoisomers of the compounds described herein, as well as racemic and scalemic mixtures thereof. In some aspects, the compounds of the disclosure may be provided in substantially enantiomerically pure form. In some aspects, individual enantiomers or diastereomers of the compounds of the disclosure may be prepared or isolated by methods including asymmetric synthesis, use of chiral auxiliaries, chiral chromatographic separation, enzymatic resolution, and/or diastereomeric salt formation and recrystallization.

In some aspects, suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

In certain aspects, the carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In some aspects, compounds of the disclosure may be formulated or administered in the form of a prodrug. As used herein, a prodrug is a pharmacologically inactive or less active derivative or precursor of an active compound of the disclosure that is converted to the active compound in vivo, for example by enzymatic hydrolysis, chemical hydrolysis, reduction, or oxidation under physiological conditions. In some aspects, prodrug strategies may be employed to improve one or more pharmaceutical or pharmacokinetic properties of the compounds of the disclosure, including but not limited to aqueous solubility, membrane permeability, oral bioavailability, metabolic stability, duration of action, and/or tissue-selective or cell-type-selective delivery. In some aspects, prodrug forms include, but are not limited to, ester prodrugs, carbamate prodrugs, carbonate prodrugs, amide prodrugs, phosphate prodrugs, and N-oxide prodrugs. The disclosure encompasses all prodrug forms of the compounds described herein.

In some aspects, the pharmacokinetic properties of the compounds of the disclosure, including absorption, distribution, metabolism, and excretion (ADME), are relevant to the design, formulation, and dosing of the pharmaceutical compositions described herein. In some aspects, when a compound of the disclosure exhibits limited aqueous solubility or low oral bioavailability, one or more solubility-enhancing or bioavailability-enhancing formulation strategies may be employed, including but not limited to: use of cyclodextrins or cyclodextrin derivatives (e.g., hydroxypropyl-β-cyclodextrin) as solubilizers or complexing agents; preparation of amorphous solid dispersions using hot-melt extrusion, spray-drying, or co-precipitation techniques; use of lipid-based drug delivery systems including self-emulsifying drug delivery systems (SEDDS) and self-microemulsifying drug delivery systems (SMEDDS); particle size reduction or nanosizing techniques such as milling, microfluidization, or high-pressure homogenization; and/or use of co-solvents, surfactants, and/or solubilizing excipients. In some aspects, the metabolic stability of a compound of the disclosure, including susceptibility to first-pass metabolism and the nature of predominant metabolic pathways (e.g., CYP450-mediated oxidation, glucuronidation, or sulfation), may be taken into account in determining the appropriate route of administration and dosing regimen.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In some aspects, solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

In some aspects, non-limiting examples of non-aqueous solvents are propylene glycol, polyethylene glycol, vegetable oil and injectable organic esters such as ethyloleate. In some aspects, non-limiting examples of aqueous carriers include water, alcoholic/aqueous solutions, saline solutions, parenteral vehicles such as sodium chloride, Ringer's dextrose, etc. In some aspects, intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

In certain aspects, formulations comprising constructs described herein and/or co-administered formulations may be suitable for oral administration. In some aspects, oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In some aspects, oral formulations of the compounds of the disclosure may further comprise one or more pharmaceutical excipients selected from binders (such as but not limited to hydroxypropyl methylcellulose, hydroxypropyl cellulose, polyvinylpyrrolidone, microcrystalline cellulose, and/or starch), disintegrants (such as but not limited to croscarmellose sodium, crospovidone, and/or sodium starch glycolate), glidants (such as but not limited to colloidal silicon dioxide), lubricants (such as but not limited to magnesium stearate, stearic acid, and/or sodium stearyl fumarate), and/or diluents (such as but not limited to lactose, microcrystalline cellulose, dicalcium phosphate, and/or mannitol). In some aspects, oral formulations of the compounds of the disclosure may be provided as immediate release (IR) formulations designed to release the active compound without intentional delay or modification. In some aspects, oral formulations may be provided as modified release formulations, including extended release (ER) formulations, controlled release (CR) formulations, delayed release formulations, and/or pulsatile release formulations, designed to achieve a desired release rate or absorption profile. In some aspects, tablets may be provided as film-coated tablets, for example to improve palatability, protect the active compound from moisture or light, facilitate swallowing, and/or mask taste; or as enteric-coated tablets designed to resist dissolution in the acidic environment of the stomach and to release the active compound in the small intestine. In some aspects, capsule formulations may comprise hard gelatin or hydroxypropyl methylcellulose (HPMC) capsule shells filled with the compound in powder, pellet, granule, or minitablet form.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### IX. Kits

Certain aspects of the present disclosure also concern kits containing compositions of the disclosure or compositions to implement methods disclosed herein. In some aspects, disclosed are kits that can be used detect and/or modify one or more RNAs, such as mRNAs. In some aspects, disclosed are kits that can be used to quantify, detect, and/or modify target RNA. In some aspects, a kit may also include additional components that are useful for purifying, amplifying, or sequencing RNA or DNA, or for other applications of the present disclosure as described herein.

The kit may optionally provide additional components that are useful in a procedure. These optional components include buffers, capture reagents, developing reagents, labels, reacting surfaces, means for detection, control samples, instructions, and interpretive information. In some aspects, a kit can be used to detect, for example, the absence, presence, and/or level of one or more features described herein. In certain aspects, a kit contains, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 350, 400, 450, 500, 550, 600, 650, 700, 750, 800, 850, 900, 950, 1,000, or more than 1,000, probes, primers or primer sets, synthetic molecules or inhibitors, or any value or range and combination derivable therein.

In some aspects, a kit may comprise a number of agents for assessing differential RNA levels and/or modifying RNA levels in any feature described herein. In some aspects, a kit may comprise reagents for detection of 1, 2, 3, 4, 5, 6, 7, 8, 9, 10, 11, 12, 13, 14, 15, 16, 17, 18, 19, 20, 21, 22, 23, 24 and/or 25 features.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

In certain aspects, negative and/or positive control nucleic acids, probes, and inhibitors are included in some kit aspects. In addition, a kit may include a sample that is a negative or positive control, for example a nucleic acid that does not comprise a m⁵C mark may be included as a negative control and a nucleic acid that does comprise a m⁵C mark may be included as a positive control.

It is specifically contemplated that a kit of the present disclosure may exclude any one or more of the described components in certain aspects.

### EXAMPLES

The following examples are included to demonstrate certain aspects of inventions disclosed herein. It should be appreciated by those of skill in the art that the techniques disclosed in the examples which follow represent techniques discovered by the inventor to function well in the practice of inventions disclosed herein, and thus can be considered to constitute certain modes for its practice. However, those of skill in the art should, in light of the present disclosure, appreciate that many changes can be made in the specific aspects which are disclosed and still obtain a like or similar result without departing from the spirit and scope of inventions described herein.

### Example 1—Materials and Methods

Unless otherwise stated, where applicable, assays and experiments described in the following examples were performed as described here and/or as outlined in PCT/US2024/049478 filed Oct. 1, 2024, which is incorporated herein by reference in its entirety.

### Cell Culture and Cell Line Construction

The following cell lines were used: MA9.3, MA9.3ITD, MA9.3RAS, MA9.6, MA9.6ITD, MA9.6RAS, KOPN1, SKNO1, KOCL69, JURKAT, KOCL50, TF1, HEL, and NB4. K562, MOLM13, OCI-AML3, SKM1, NOMO1, CEM/C1, CCRF/CEM, HL60, THP1, CESS, ARH77, KOPN1, KOCL69, JURKAT, KOCL50, HEL, and NB4 were maintained in RPMI1640 medium supplemented with 10% FBS and 50 U/mL Penicillin-Streptomycin. KASUMI1 cells were cultured in RPMI1640 with 10% FBS and 50 U/mL P/S. TF1 cells were kept in RPMI1640 with 10% FBS, 50 U/mL P/S and 2 ng/mL human GM-CSF. SKNO1 cells were grown in RPMI1640 with 10% FBS, 50 U/mL P/S and 10 ng/mL human GM-CSF. MA9.3 and MA9.6 cells were cultured in IMDM supplemented with 20% FBS, 50 U/mL P/S, 10 ng/mL each of human cytokines SCF, TPO, FLT3L, IL-3, and IL-6. MA9.3ITD, MA9.3RAS, MA9.6ITD and MA9.6RAS were kept in IMDM with 20% FBS and 50 U/mL P/S. Mycoplasma contamination was routinely tested using qPCR every half a year.

Lentiviral transduction was performed using viral particles packaged with HEK293T cells with pCMV-VSV-G and pCMV-dR8.2 plasmids (Addgene 8454 and 8455). Viral particles were precipitated using TransDux MAX Lentivirus Transduction Reagent and used immediately following overnight precipitation.

Transfection of K562 cells was conducted using Lipofectamine™ LTX Reagent with PLUS™ Reagent, following the manufacturer's recommended protocol for K562. Cells were cultured to reach a concentration of 0.8 million/mL, and suspended in 25 mL Opti-MEM™ I Reduced Serum Medium. A DNA mixture containing 30 pg plasmid and 30 μL PLUS™ Reagent in 3.75 mL Opti-MEM™ medium was incubated at room temperature (RT) for 10 mins. Subsequently, 120 μL LTX Reagent was added and incubated for 25 mins at RT before being introduced to the cell culture. Transfection was conducted for 8 hours before replacing the media with standard growth medium.

Nucleofection of HEL cells was performed using Lonza 4D-Nucleofector System with the SF cell line 4D-nucleofector kit according to the manufacturer's protocol. Each reaction utilized 1 million cells freshly passaged at 0.2 million/mL the day before. Cells were electroporated using program DC102.

Cell Proliferation Assay and 5-azaC Sensitivity Screen

Cell lines expressing Dox-induced shRNA were treated with the indicated compounds following 5 days of Dox induction with replenishment every 24 hours. As an exception, proliferation of MOLM13 with NSUN2 depletion was measured from the first treatment of Dox. In continuous measurement of proliferation, cells were passaged every 2 days, and the cumulative proliferation was adjusted by accounting for passaging ratios during proliferation measurement. HEL cells subjected to gene overexpression were treated with drugs 24 hours post-nucleofection. All proliferation assays were initiated at a seeding density of 0.2 million/mL and maintained with drug treatments for the specified time course. Cell proliferation was quantified using CellTiter-Glo® 2.0 Cell Viability Assay, according to the manufacturer's protocol with modifications for enhanced lysis efficiency. Specifically, CellTiter-Glo® 2.0 reagent was freshly diluted in RIPA buffer (25 mM Tris-HCl pH 7.6, 150 mM NaCl, 1% NP-40, 1% Na-deoxycholate, 0.1% SDS) at a 3:4 volume ratio. For each measurement, 100 μL of diluted reagent was added to 100 μL of cell culture, mixed vigorously for 2 mins using orbital shaking in “fast” mode, and incubated at RT for 10 mins. Luminescence was recorded using a BioTek Synergy HTX plate reader.

Proliferation responses to 5-azaC and decitabine across 26 leukemia cells lines were evaluated after 24-hour treatment across five concentrations. Proliferation profiles were clustered based on normalized luminescence signals across doses. The top 50 recurrent leukemia-associated mutations were selected for visualization, based on the number of mutated cases in leukemia reported by the curated set of non-redundant studies reported in cBioPortal⁶⁶⁻⁶⁸ Mutation annotations were retrieved from the DepMap database⁶⁹ and supplemented using data from COSMIC⁷⁰ and Cellosaurus⁷¹. Silent mutations were excluded from the final integrated mutation dataset.

### Human Primary AML Patient Samples

Human primary AML patient samples were collected at the time of diagnosis, relapse or remission after written informed consent at City of Hope Hospital or MD Anderson Cancer Center incongruence with the protocol approved by the institutional review board (IRB). Characteristics of the AML patients are provided in Table S3. The AML patient samples were cryopreserved in liquid nitrogen for further study. AML #2017-129, #2016-35 and #6303 cells were cultured in StemSpan™ SFEMII supplemented with 10 ng/mL of human SCF, mouse TPO, mouse FLT3L, human IL-3, human IL-6, 35 nM of UM171, 500 nM of Stem regenin1 (SR1) and 50 U/mL of P/S.

### Animals

The global Nsun2 KO mouse model was used. These mice used in this study were backcrossed for more than 6 generations with C57BL/6 mice and applied throughout this study, including both male and female. All animal studies were performed with the approval from the Institutional Animal Care and Use Committee (IACUC) at The University of Texas Health Science Center at San Antonio (UTHSCSA) and conducted in accordance with the institutional and national guidelines and regulations.

### In Vitro Colony-Forming Assay

Bone marrow (BM) cells were collected from wild-type or Nsun2 KO mice and plated in triplicate in methylcellulose medium supplemented with 100 ng/mL mouse SCF, 10 ng/mL mouse IL-3, 50 ng/mL mouse TPO, 10 ng/mL mouse GM-CSF, 4 U/mL human EPO, and 50 ng/mL human interleukin-6. The colonies were imaged by STEMvision™ (STEMCELL Technologies) and scored on day 7. Colony cells were harvested and analyzed for expression of stem and progenitor markers and erythroid linage markers by flow cytometry.

### Competitive Repopulation Assay

Competitive repopulation assay was performed to assess the effect of NSUN2 on the repopulating potential of HSPCs in vivo. 5×10⁵ total BM cells isolated from WT or Nsun2 KO mice (CD45.2) were mixed with 5×10⁵ BoyJ (CD45.1) BM cells and transplanted into lethally irradiated (800 cGy) BoyJ recipients through the tail vein injection. The CD45.2/CD45.1 chimera in the PB were monitored monthly for 32 weeks. Recipients were sacrificed 32 weeks after transplantation to analyze the CD45.2/CD45.1 chimera in BM, as well as HSPCs and lineage cell distributions within CD45.1+ or CD45.2+ cell populations.

### In Vitro Erythroid Differentiation

The hematopoietic stem and progenitor Lin⁻c-Kit⁺ (LK) cell derived from BM were in suspension cultured for in vitro erythroid differentiation following protocols described previously^{73,74} with slight modifications. Briefly, LK cells were isolated from total BM cells of 6-9 months old mice using the Direct Lineage Cell Depletion Kit (Miltenyi Biotec, 130-110-470), and then the lineage-negative cells (Lin⁻) were sorted with c-Kit (CD117) MicroBeads (Miltenyi Biotec, 130-091-224) by magnetic-activated cell sorting with autoMACS® Pro Separator (Miltenyi Biotec). LK cells were cultured in Iscove modified Dulbecco's medium (IMDM) supplemented with 10% FBS, 100 ng/mL mSCF, 40 ng/mL mIGF-1 and 2 U/mL hEPO for expansion. After 4 days, cells were cultured in IMDM supplemented with 10% FBS, 100 ng/mL mSCF, 40 ng/mL mIGF-1 and 6 U/mL hEPO for erythroid differentiation. Erythroid cells were analyzed over time by FACS and Wright-Giemsa staining (Fisher Scientific, 23-123869).

### Flow Cytometry Analysis

Flow cytometric analysis of mouse BM cells was performed as previously described⁷⁵ Briefly, for HSPC cells, BM cells were stained with APC-anti-Lineage cocktail, PE-Cy™ 7-anti-Sca1, PE-anti-c-kit, Alexa Flour 700-anti-CD34, APC-Cy7-anti-CD16/32, BV421-anti-CD135. For analysis of myeloid lineage cells, BM cells were stained with PerCP-Cy™ 5.5-anti-Gr-1 and PE-anti-CD11b (Mac-1). APC-anti-Ter119 and PE-anti-CD71 were used for analyzing erythroid populations. APC-anti-B220 and APC-Cy7-anti-IgM were used for analyzing B lymphocytes. PE-anti-CD8a and PE-Cy™ 7-anti-CD4 were used for staining T lymphocytes.

### Immunoblotting

Samples were collected with equal cell numbers and washed with PBS. For histone analysis, cells were lysed directly in 50 μL of 2× NuPAGE™ LDS sample buffer supplemented with β-mercaptoethanol (O-ME) per 1 million cells. To measure γH2A.X levels after UV damage, cells were exposed too 50 J/m² UVC in PBS, then immediately incubated in complete medium for 5 mins prior to collection for immunoblotting. For analysis of non-chromatin-associated proteins, cells were lysed in 50 μL of RIPA buffer supplemented with 100× proteinase inhibitor cocktail per 1 million cells on ice for 15 mins. Lysates were then centrifuged at 12,000×g for 15 mins at 4° C. The supernatant was mixed with NuPAGE™ LDS sample buffer containing β-ME to a final 1× concentration. Samples were denatured by heating at 95° C. for 20 mins and sonicated until no longer viscous. The denatured protein samples were separated on 4-12% NuPAGE Bis-Tris gels (Invitrogen™, NP0335BOX) and transferred to 0.45 μm nitrocellulose membrane (Bio-Rad, 1620115) using a semi-dry transfer system at 25 V for 45 mins. Membranes were blocked in 5% BSA in PBSTat RT for 30 mins, followed by overnight incubation at 4° C. with primary antibody diluted in PBST containing 3% BSA. After three 10-min washes in PBST, membranes were incubated with secondary antibody diluted in PBST containing 1% BSA for 40 mins at RT, washed three more times for 10 mins each, and developed using SuperSignal™ West Dura Extended Duration Substrate kit. Signals were visualized with the Invitrogen™ iBright imaging system. The SRSF2 antibody used for immunoblotting is Cat #ab204916 (Abcam).

Cellular Fractionation and Purification of caRNA

Cellular fractionation was performed based on previously described protocol⁷⁶ with slight modifications. A total of 20 million cells were washed with PBS and pelleted by centrifugation at 500×g. Cells were resuspended in 300 μL of ice-cold NP-40 lysis buffer (10 mM Tris-HCl pH 7.5, 150 mM NaCl, 0.05% NP40) by gently flicking the tube, avoiding pipetting. After 5 mins of incubation on ice, the suspension was layered onto ice-cold NP-40 lysis buffer containing 24% sucrose and centrifuged at 15,000×g for 10 mins at 4° C. The supernatant was collected as the cytoplasmic fraction. The pellet was washed with ice-cold PBS and resuspended in 150 μL of ice-cold glycerol buffer (20 mM Tris-HCl pH 8.0, 75 mM NaCl, 0.5 mM EDTA, 50% glycerol) by flicking the tube. Next, 300 μL of ice-cold nuclei lysis buffer (10 mM HEPES pH 7.6, 7.5 mM MgCl2, 0.2 mM EDTA, 300 mM NaCl, 1M Urea, 1% NP40) was added to the suspension, which was then vortexed at maximum speed for 20 seconds, incubated on ice for 2 mins, and centrifuged at 15,000×g for 2 mins at 4° C. The supernatant was collected as the nuclear fraction. The remaining pellet was washed with PBS and harvested as the chromatin fraction.

To isolate caRNA, the chromatin pellet was treated with 2 U TURBO DNase™ at 37° C. for 1 hour, followed by digestion with proteinase K at 55° C. for 1 hour after supplementing 1% SDS to the buffer. TRIzol™ reagent was added to the mixture, which was then incubated at 50° C. for 20 mins for resolution of pellets. Following TRIzol™ extraction with chloroform, the aqueous phase was collected, and RNA was purified using the RNA Clean & Concentrator-25 kit (RCC-25) including in-column DNase I digestion according to the manufacturer's instructions.

### UHPLC-MS/MS

70 ng of RNA was digested with nuclease P1 at 37° C. overnight, followed by treatment with 1 U shrimp alkaline phosphatase (rSAP) at 37° C. for 2 hours. Genomic DNA, purified from PureLink™ Genomic DNA Mini Kit, was digested with nuclease P1 at 37° C. overnight. This was followed by sequential digestion with 0.001 U phosphodiesterase I in 100 mM ammonium bicarbonate at 37° C. for 2 hours, and 1 U rSAP at 37° C. for 2 additional hours. Prior to analysis, all samples were diluted with LC-grade water and filtered through a 0.22 μm PVDF filter (Millipore, SLGVR04NL) prior to injection into a C18 reversed-phase column connected to a SCIEX 6500+ Triple Quadrupole Mass Spectrometer operating in positive electrospray ionization mode. Nucleosides were quantified based on retention time and specific nucleoside-to-base ion transitions (RNA: 268→136 for A; 258→126 for m⁵C; DNA: 252→136 for dA, 242→126 for 5mdC). Relative nucleoside levels were determined by peak area comparison within the same batch of reactions.

### TUNEL Assay

TUNEL assay was performed using the DeadEnd Fluorometric TUNEL System according to the manufacturer's instructions, with the modification that the PBS wash buffer was supplemented with 0.5% BSA. Following rTdT labeling, the reaction was terminated using 20 mM EDTA containing 0.5% BSA in PBS to prevent cell clumping.

### EU-Based Nascent RNA Labeling Analysis

Nascent RNA labeling for fluorescence detection was performed using the Click-iT™ RNA Alexa Fluor™ 488 Imaging Kit. Cells were incubated with 1 mM EU for 30 mins prior to harvesting. The procedure followed the manufacturer's instructions, with modifications for flow cytometry analysis. At each wash step, cells were washed using 0.5% BSA in PBS to minimize cell clumping, and centrifuged at 3,000×g for 3 mins at RT-except during the rinsing step, where the Click-iT® reaction rinse buffer was used as instructed.

### EdU-Based Cell Cycle Analysis and Unscheduled DNA Repair Analysis

For cell cycle analysis, cells were incubated with 20 μM EdU for 1 hour, followed by a 30-min chase in complete media supplemented with 10 μM thymidine to dilute unincorporated EdU. Unscheduled DNA repair analysis was performed based on a published protocol⁷⁷ with modifications. After treatment under designated conditions, cells were irradiated with 50 J/m² 254 nm UVC in PBS, followed by immediate addition of complete media containing 20 μM EdU and 1 μM 5-Fluoro-2′-deoxyuridine. Cells were incubated for 1 hour under standard culture conditions, then washed twice with PBS and chased in complete media supplemented with 10 μM thymidine for 30 mins. At harvest, EdU detection was performed using Click-iT™ RNA Alexa Fluor™ 488 Imaging Kit, following a flow cytometry-adapted protocol as described for EU-based nascent RNA labeling. Post click chemistry, cells were stained with 2 μg/mL Hoechst 33342 for 15 mins at RT, washed, and analyzed by flow cytometry. Hoechst signal was quantified in linear scale. Cells with increased EdU incorporation and intermediate Hoechst intensity were classified as S phase, while 1N and 2N populations with basal level EdU intensity were designated as G0/G1 and G2/M phases, respectively. For unscheduled DNA repair analysis, only G0/G1 or G2/M populations were included in the quantification.

### Immunofluorescence Staining

Chamber slides were pre-coated with 0.1 mg/mL poly-D-lysine overnight to promote cell adherence. After harvest, cells were fixed with 4% paraformaldehyde (PFA) in PBS at RT for 10 mins, washed, and resuspended in PBS at a concentration of 1 million cells/mL. Cells were added to the pre-treated chambers and allowed to settle by gravity for 30 mins at RT without disturbance. Following sedimentation, excess PBS was gently removed, and cells were permeabilized by 0.5% Triton X-100 in PBS for 10 min, followed by PBS washes at RT. Cells were then blocked with 0.5% BSA in PBS or 20 min, and subsequently incubated with primary antibodies diluted in blocking buffer:anti-SRSF2 (ab11826) at ratio of 1:200 and anti-p300 at ratio of 1:400 for 1 hour at RT. After PBS washing, cells were incubated with secondary antibodies diluted 1:1000 in blocking buffer for 1 hour: goat anti-mouse IgG-AF 546 and goat anti-rabbit IgG-AF 647. Cells were then stained with 2 μg/mL Hoechst 33342 in PBS for 15 mins at RT. Following final PBS washes, ProLong™ Glass Antifade Mountant was applied to minimize photobleaching. Fluorescence images were acquired using a Leica SP8 laser scanning confocal microscope. The fluorescence intensity was measured by ImageJ.

### Proximity Ligation Assay

Proximity ligation assay was conducted using the Duolink® Proximity Ligation Assay kit. Cells were fixed with 4% PFA in PBS at RT for 10 mins, followed by gravity sedimentation at a concentration of 1 million cells/mL onto chamber slides pre-coated with 0.1 mg/mL poly-D-lysine overnight. After permeabilization with 0.5% Triton X-100 in PBS for 10 min, the assay was carried out according to the manufacturer's protocol. Primary antibodies used were anti-SRSF2(ab11826, 1:200) and anti-p300 (1:400). Following amplification, nuclei were stained with 2 μg/mL Hoechst 33342 for 15 mins, and slides were mounted using ProLong™ Glass Antifade Mountant. Fluorescence imaging was conducted using a Leica SP8 laser scanning confocal microscope.

### Immunoprecipitation

Immunoprecipitation was performed using 50 μL of Dynabeads™ Protein G or Dynabeads™ Protein A, washed with lysis buffer (50 mM Tris-HCl pH 7.5, 100 mM NaCl, 1% NP-40 substitute, 0.5% sodium deoxycholate, 100× proteinase inhibitor cocktail). Beads were conjugated with SRSF2 antibodies-20 μL of the one from Abcam (ab204916), 5 μg of the one from Proteintech or 5 μL of the one from Sigma-Aldrich-and Rabbit IgG or Mouse (G3A1) mAb IgG1 Isotype Control as controls. Protein G beads were used for mouse antibodies and protein A beads were used for rabbit antibodies. 20 million SKM1 cells per sample were lysed in 800 μL lysis buffer at 4° C. with end-to-end rotation for 30 mins. Lysates were centrifuged at 15,000×g for 15 min at 4° C., and the supernatant was collected. 40 μL of lysate was reserved as input, and the remainder was incubated with the antibody-conjugated beads at 4° C. for 4 hours. Beads were washed three times with wash buffer (50 mM Tris-HCl pH 7.5, 300 mM KCl, 0.05% NP-40 substitute, 1000× proteinase inhibitor cocktail). washed beads were suspended in 50 μL of 1× Laemmli sample buffer supplemented with β-ME and heated at 95° C. for 10 minutes. The input sample was mixed with 4× Laemmli sample buffer supplemented with β-ME to a final 1× concentration and heated the same. 5 μL of input and 20 μL of eluted sample were loaded for immunoblotting.

### RNA-Seq

RNA extraction was performed using TRIzol™ reagent following the designated treatment. The RNA was extracted with chloroform, and the aqueous phase was retained for RNA purification using RCC-25. In-column DNase I digestion was conducted to remove DNA. For studies involving gene depletion, KD efficiency was assessed by RT-qPCR. For RNA-seq after 5-azaC treatment, 2 μL of a 1/20,000 dilution of ERCC RNA spike-in mix was added to 10 ng of RNA. The RNA was then proceeded for library construction using the SMARTer® Stranded Total RNA-Seq Kit v2—Pico Input Mammalian. Next-generation sequencing was performed on Illumina NovaSeqX with pair-end 150 bp reads.

### Nascent RNA-Seq

MOLM13 and SKM1 cells were treated with 3 μM 5-azaC for 0, 4, 8 and 16 hours. Following treatment, 1 mM EU was added to the media for nascent RNA labeling. In parallel, drosophila cells were labeled similarly to serve as a spike-in control. Cells were harvested and purified using TRIzol™ reagent following the manufacturer's protocol with isopropanol precipitation. Nascent RNA was enriched using the Click-iT™ Nascent RNA Capture Kit for gene expression analysis, following the manufacturer's protocol with modifications. 4.5 pg of EU-labeled RNA was reacted with 2.2 μL biotin azide in a 25 μL click reaction and purified using RCC-25. 600 ng of RNA was mixed with drosophila spike-in at a 200:1 ratio and processed for nascent RNA enrichment. Streptavidin beads provided by the kit were washed with B&W buffer (5 mM Tris-HCl pH 7.5, 0.5 mM EDTA, 1M NaCl) three times, followed by solution A (100 mM NaOH, 50 mM NaCl) twice for 2 mins each. The beads were then washed with 100 mM NaCl at the same volume of solution A, followed by another round of washing with B&W buffer three times. Beads were suspended in the wash buffer 2 provided by the kit, and RNA was mixed with RNaseOUT in RNA binding buffer and denatured at 70° C. for 5 mins before being added to the beads. After 30 mins of incubation at RT with end-to-end rotation, the beads were washed with low salt buffer (10 mM Tris-HCl pH 7.5, 50 mM NaCl, 0.1% NP-40, 0.2 U/μL SUPERase·In™ RNase Inhibitor), high salt buffer (10 mM Tris-HCl pH 7.5, 500 mM NaCl, 0.1% NP-40, 0.2 U/μL SUPERase·In™ RNase Inhibitor), and B&W buffer supplemented with 0.2 U/μL SUPERase·In™ RNase Inhibitor, washing five times each. The beads were then washed with the wash buffer 1 and wash buffer 2 supplied by the kit and eluted in 50 μL of 95% formamide supplemented with 2.5 mM EDTA by heating at 65° C. for 5 mins, followed by 95° C. for 5 mins. The supernatant was immediately harvested while still hot, and RNA was extracted using 1 mL TRIzol™ reagent. RNA was purified following the manufacturer's protocol with isopropanol precipitation facilitated by glycogen. The library was constructed using the SMARTer® Stranded Total RNA-Seq Kit v2—Pico Input Mammalian, and sequencing was performed using the Illumina NovaSeqX with pair-end 50 bp reads.

### m⁵C MeRIP-Seq

m⁵C MeRIP-seq was performed as described in previous publication⁷⁸. For MOLM13 cells, 0.02 million drosophila S2 cells were added to 20 million MOLM13 cells before proceeding with cellular fractionation to enrich caRNA, as outlined earlier. rRNA removal was carried out using RiboMinus Eukaryote System v2. 2 pg ribo-depleted caRNA was then used for m⁵C immunoprecipitation. RNA was fragmented using 1×NEBNext® Magnesium RNA Fragmentation buffer at 94° C. for 2 mins, and 5% of the sample was saved as input. The RNA was heated to 72° C. for 2 mins and then placed immediately on ice for denaturation. Next, the RNA was added to protein G dynabeads conjugated with 2 pg anti-m⁵C antibody in 300 μL IP buffer (10 mM Tris-HCl pH 7.5, 150 mM NaCl, 0.05% Triton X-100, 1 mM spermidine, 0.2 U/μL SUPERase·In™ RNase Inhibitor), and incubated with end-to-end rotation at 4° C. for 2 hours. The beads were washed three times with IP buffer and eluted with buffer RLT. Both input and MeRIP libraries were constructed using the SMARTer® Stranded Total RNA-Seq Kit v2—Pico Input Mammalian, and sequencing was performed with the Illumina NovaSeqX with pair-end 150 bp reads.

### CUT&Tag

CUT&Tag was performed using the CUT&Tag-IT Assay Kit following the manufacturer's instructions. 0.2 million cells were used per sample. For 5-azaC-treated CUT&Tag profiling of H2AK119ub and H3K27ac, 4,000 drosophila nuclei from CUT&Tag-IT® Spike-In Control were added as a spike-in control. Primary antibodies used included 1 μL H2AK119ub antibody, 1 μL H3K27ac antibody (Cat #39034) or 0.25 μL SRSF2 antibody (Cat #ab11826). Libraries were constructed according to the kit protocol and sequenced on the Illumina NovaSeqX platform with pair-end 150 bp reads.

CLIP-Seq and CLIP with End Ligation of pCP-AF498 Fluorophore

For CLIP-seq, 100 million cells were used for each replicate. Following the designated treatment, cells were crosslinked in PBS using 4500 J/m² UV at 254 nm. Cells were lysed in 200 μL iCLIP lysis buffer (50 mM Tris-HCl pH 7.5, 100 mM NaCl, 1% NP-40 substitute, 0.1% SDS, 0.5% sodium deoxycholate, 100× proteinase inhibitor cocktail and 0.2 U/μL SUPERase·In™ RNase Inhibitor) at 4° C. for 15 mins with end-to-end rotation. SDS were then added to reach a final concentration of 1% for protein release from chromatin. Samples were sonicated (35% amplitude, 2s on/4s off cycles, 1 min on ice) and diluted 10-fold with SDS-free iCLIP lysis buffer to reduce SDS back to 0.1%. After centrifugation at 15,000×g for 30 mins at 4° C., the supernatant was harvested and treated with 0.2 U/μL RNase T1 at 22° C. for 15 mins, then quenched on ice for 5 mins. 2% of the lysate was saved as input and digested with 10 U/μL RNase T1 at 22° C. for 10 mins, followed by proteinase K digestion, RNA purification and end repair. The remaining lysate was incubated overnight at 4° C. with protein A beads conjugated to 30 μL MBD6 antibodies per sample. Beads were washed three times with CLIP wash buffer (50 mM Tris-HCl pH 7.5, 300 mM KCl, 0.05% NP-40, 1000× protein inhibitor cocktail and 0.2 U/μL SUPERase·In™ RNase Inhibitor), followed by digestion with 10 U/μL RNase T1 at 22° C. for 10 mins. Beads were further washed for three times each with CLIP high salt buffer (50 mM Tris-HCl pH 7.5, 500 mM KCl, 0.05% NP-40, 100× protein inhibitor cocktail and 0.2 U/μL SUPERase·In™ RNase Inhibitor) and PNK buffer without DTT, and subjected to end-repair using T4 PNK. After proteinase K digestion, RNA was purified for library preparation. The input and CLIP libraries were constructed using the NEBNext® Small RNA Library Prep Set and sequenced with Illumina NovaSeqX, pair-end 150 bp.

For CLIP with end ligation of pCp-AF488 fluorophore, K562 cells with SRSF2-myc overexpression after DMSO or 5-azaC treatment were counted to ensure the same number of cells used for experiment in each sample. Approximately 15 million cells were crosslinked by 4500 J/m² UV at 254 nm in PBS. Samples were lysed with iCLIP lysis buffer, and lysates were cleared by centrifugation. Supernatants were treated with 0.2 U/μL RNase T1 at 22° C. for 15 mins, quenched on ice, and incubated at 4° C. for 4 hours with protein G beads conjugated to 4 μL Myc-tag antibodies. Following immunoprecipitation, beads were washed for three times with CLIP wash buffer, digested with 10 U/μL RNase T1 at 22° C. for 8 mins, and further washed for three times each with CLIP high salt buffer and PNK buffer without DTT. Beads were subjected to RNA dephosphorylation using T4 PNK, then ligated overnight at 16° C. in a 26 μL ligation reaction containing 2.4 μL T4 RNA ligase 1, 0.5 μL pCp-AF488, 0.3 μL 100 mM ATP, 0.4 μL SUPERase·In™ RNase Inhibitor, 0.6 μL 1% Tween-20, 0.9 μL DMSO and 9 μL 50% PEG8000. After ligation, samples were washed, denatured in 2×LDS sample buffer with β-ME at 70° C. for 10 mins, and analyzed on a 4-12% NuPAGE Bis-Tris gel for fluorescence detection.

### Data Analysis of RNA-Seq

Adapter trimming was performed using Cutadapt⁷⁹, and reads were mapped to the hg38 human genome or mm10 mouse genome using HISAT2. Gene-level read counts were quantified using featureCounts⁸¹ based on the hg38 refGene annotation. For differential gene expression analysis, DESeq2⁸² was applied to datasets without spike-in, while RUVSeq⁸³ was used for normalization and analysis of datasets that included ERCC RNA spike-in mix. GSEA was conducted using the GSEA software suite^{84,85}

### Data Analysis of Nascent RNA-Seq

Adapter trimming was performed using Cutadapt⁷⁹, and reads were mapped to hg38 human genome and dm6 drosophila genome by HISAT2⁸⁰. Gene-level read counts were quantified using featureCounts⁸¹ based on the hg38 refGene annotation. For differential gene expression analysis, DESeq2⁸² was applied using the read counts mapped to drosophila chromosomes as spike-in normalization. GSEA was conducted using the GSEA software suite^{84,85}.

### Data Analysis of m⁵C MeRIP-Seq

Adapter trimming was performed using Cutadapt⁷⁹, and reads were deduplicated and aligned to the hg38 human genome and dm6 drosophila genome using HISAT2⁸⁰. Gene-level read counts were quantified using featureCounts⁸¹ based on the hg38 refGene annotation. In parallel, reads mapping to repetitive elements were analyzed using Bowtie2⁸⁶ followed by RepEnrich2⁸⁷. input samples were analyzed as caRNA-seq for differential expression analysis of caRNA, covering both coding genes and repetitive elements. DESeq2⁸² was used for differential expression analysis. For samples from MOLM13 cells with drosophila spike-in, total read numbers mapped to each drosophila chromosome were used for normalization in DESeq2 analysis.

MeRIP peaks were identified using MACS3⁸⁸, and read counts from both input and MeRIP libraries were quantified at peak regions to assess enrichment. Peaks were normalized to total mapped reads, or additionally normalized by drosophila spike-in for MOLM13 samples. Regions with MeRIP/input >0 in either control or 5-azaC-treated samples were considered putative m⁵C sites. Sites with an enrichment fold change of 5-azaC control <0.5 were defined as decreased m⁵C sites. Metaplots were generated using deepTools⁸⁹. The IKZF1 ChIP data was downloaded from ENCODE experiment ENCSR948VFL.

### Data Analysis of CUT&Tag

Adapter trimming was performed using Cutadapt⁷⁹, and reads were aligned to the hg38 human genome and dm6 drosophila genome using bowtie2⁸⁶. Duplicate reads were removed using Picard MarkDuplicates⁹¹. For 5-azaC-treated histone modification profiling, signal normalization was performed based on the total number of reads mapped to drosophila genome. Peak calling was performed using MACS3⁸⁸. For histone modifications, broad peaks were identified using the parameters “—broad —broad-cutoff 0.01” was used. Differential peak analysis was conducted using DiffBind. For quantile-based analysis, peaks were annotated to the nearest RefSeq genes using Homer annotatePeaks.pl⁹⁰, and assigned as their putative regulatory elements. Metaplots were generated with deepTools⁹¹.

### Data Analysis of CLIP-Seq

Adapter trimming was performed using Cutadapt⁷⁹, and reads were aligned to the hg38 human genome using HISAT2⁸⁰. Duplicate reads were removed using Picard MarkDuplicates⁹¹. Metaplots were generated with deepTools⁹¹.

### Inference of Gene Network

A gene regulatory network was inferred using SJARACNe⁹², based on gene expression profiles of all myeloid lineage leukemia cell lines available in the DepMap⁶⁹ database. Candidate hub genes were selected from commonly mutated genes identified in MDS, AML and Myeloproliferative neoplasm, curated from non-redundant studies in cBioPortal⁶⁶⁻⁶⁸. Selection criteria included mutation frequency >1%, mutation cases >5 and total detected cases >100. The network was further integrated with gene expression data from leukemia cell lines categorized as sensitive and resistant to 5-azaC, analyzed using NetBID2⁹³

### Antibodies

The antibodies used in this study are summarized below: APC-anti-Lineage cocktail (BD Biosciences, Cat #5508074); PE-Cy™ 7-anti-Sca1 (BD Biosciences, Cat #558162); PE-anti-c-kit (BD Biosciences, Cat #553869); Alexa Flour 700-anti-CD34 (BD Biosciences, Cat #560518); APC-Cy7-anti-CD16/32 (BioLegend, Cat #101327); BV421-anti-CD135 (BD Biosciences, Cat #562898); PerCP-Cy™ 5.5-anti-Gr-1 (BD Biosciences, Cat #552093); PE-anti-CD11b (Mac-1)(BD Biosciences, Cat #553311); APC-anti-Ter119 (BD Biosciences, Cat #557909); PE-anti-CD71 (BD Biosciences, Cat #553267); anti-CHK1 (Cell Signaling Technology, Cat #2360S); anti-RAD51 (Cell Signaling Technology, Cat #8875S); anti-H3 (Cell Signaling Technology, Cat #12648S); anti-H2A.X (Cell Signaling Technology, Cat #2595S); anti-γH2A.X (Cell Signaling Technology, Cat #9718S); anti-H2AK119ub (Cell Signaling Technology, Cat #8240S); anti-H3K27ac (Abcam, Cat #ab4729); anti-β-TUB (Proteintech, Cat #HRP-66240); anti-SRSF2 (Abcam, Cat #ab204916); anti-Myc-tag (Cell Signaling Technology, Cat #2276S); anti-rabbit IgG (Cell Signaling Technology, Cat #7074S); anti-mouse IgG (Cell Signaling Technology, Cat #7076S); anti-NSUN2 (Proteintech, Cat #20854-1-AP); anti-SRSF2 (Abcam, Cat #ab11826); anti-p300 (Cell Signaling Technology, Cat #86377S); goat anti-mouse IgG-AF 546 (Thermo Scientific, Cat #A-11030); goat anti-rabbit IgG-AF 647 (Thermo Scientific, Cat #A-21244); anti-SRSF2 (Proteintech, Cat #20371-1-AP); anti-SRSF2 (Sigma-Aldrich, Cat #S4045); Rabbit IgG (Cell Signaling Technology, Cat #2729S); Mouse (G3A1) mAb IgG1 Isotype Control (Cell Signaling Technology, Cat #5415S); anti-m5C (Diagenode, Cat #MAb-081-100); anti-H3K27ac (Active motif, Cat #39034); PerCP-Cy™ 5.5-anti-CD45.2 (BD Biosciences, Cat #552950); FITC-anti-CD45.1 (BD Biosciences, Cat #553775); PE-Cy™ 7-anti-Gr-1 (BD Biosciences, Cat #565033); APC-anti-B220 (BD Biosciences, Cat #553092); PE-anti-CD8a (BD Biosciences, Cat #553033); PE-Cy™ 7-anti-CD4 (BD Biosciences, Cat #552775); APC-Cy7-anti-IgM (BioLegend, Cat #406516); anti-MBD6. All antibodies were applied at a dilution fold according to the manufacturer's suggestions for specific use unless specified in the methods section.

### Example 2-5-azaC Induced RNA m⁵C Depletion and Leukemia Cell Death at Early Time Points

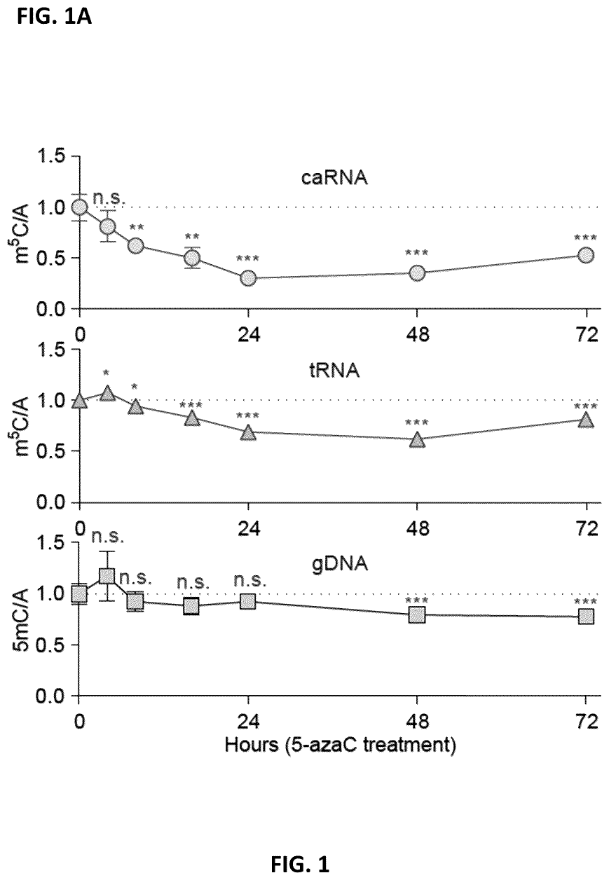

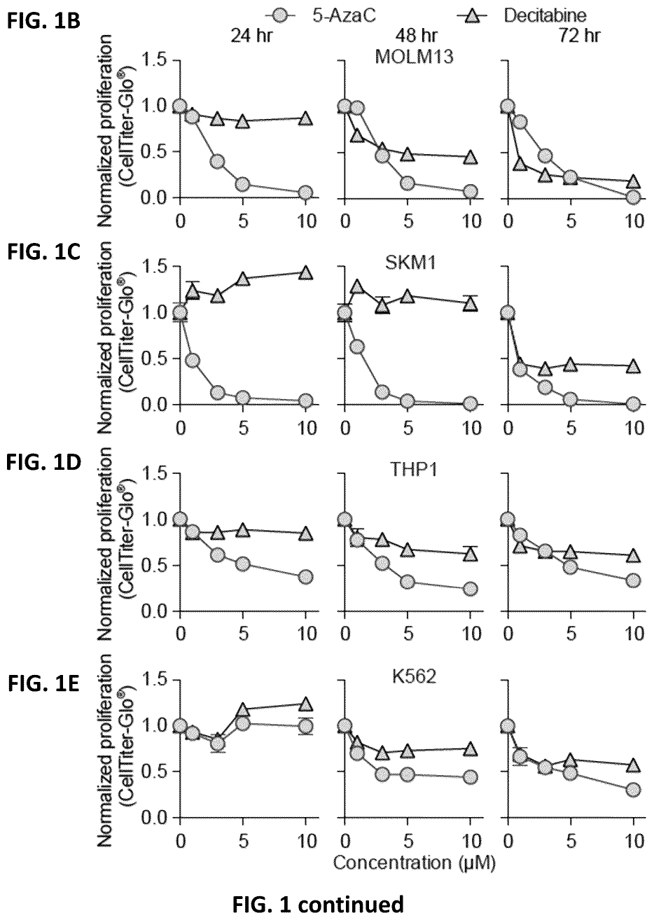

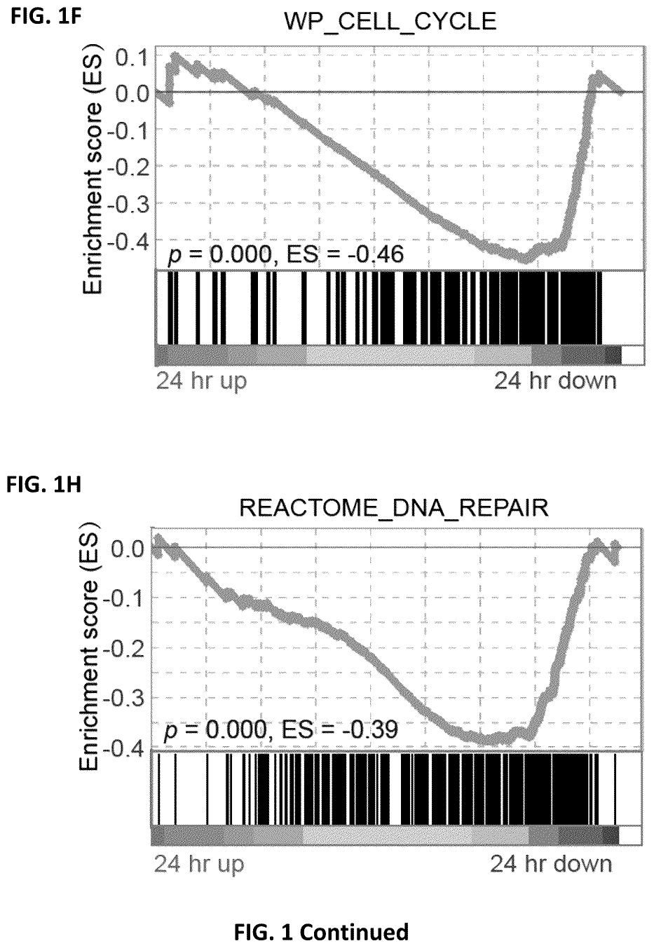

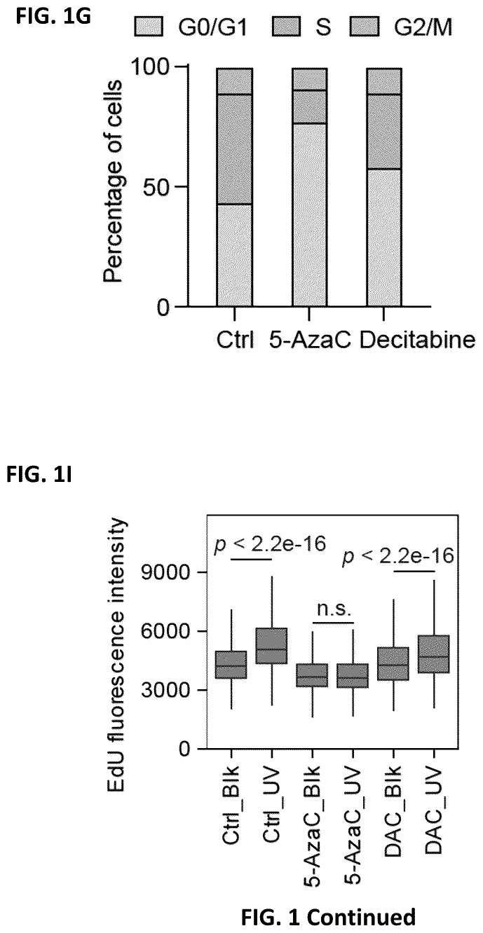

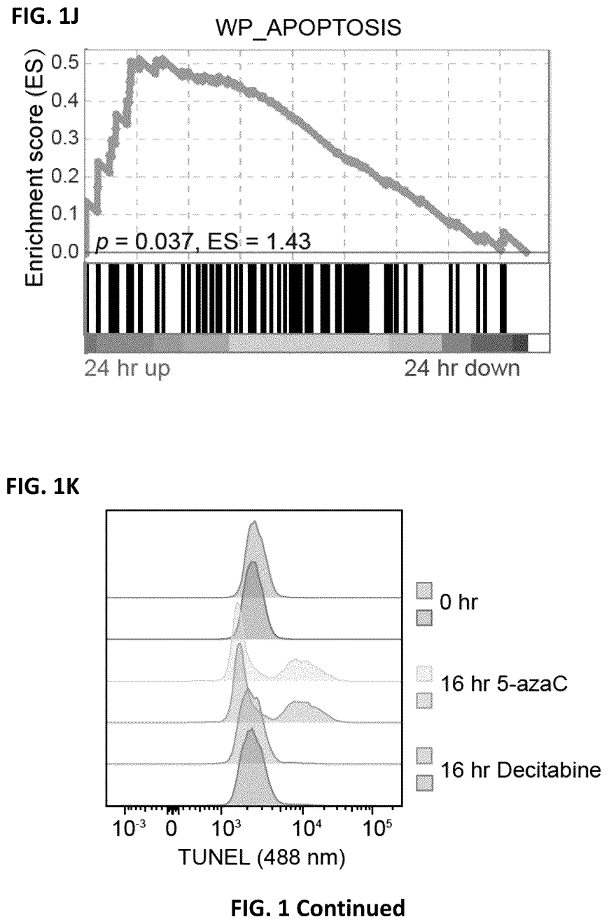

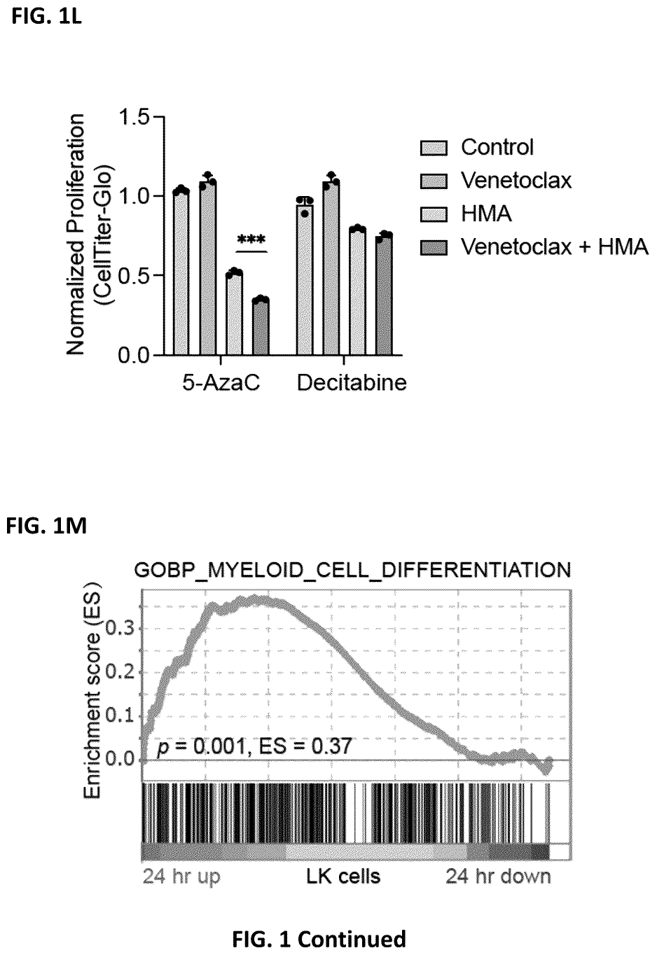

The inventors reasoned that 5-azaC could be readily incorporated into nascent RNA, whereas its incorporation into DNA requires prior conversion to its deoxy form. The RNA-mediated effects of 5-azaC might be best observed at early time points of treatment when little 5-azaC has been integrated into DNA. The inventors first measured RNA m⁵C and DNA 5mC levels using ultra-high-performance liquid chromatography-tandem mass spectrometry (UHPLC-MS/MS) given that 5-azaC could covalently inhibit both RNA and DNA cytosine methyltransferases. 5-azaC was administered at 3 μM, a concentration relevant to its plasma levels (~11 μM) in standard treatment³⁰. Both tRNA and caRNA exhibited significantly decreased m⁵C levels starting at 8 hours, with caRNA showing a greater decrease (FIG. 1A). In contrast, 5mC levels in genomic DNA were not significantly changed within 24 hours (FIG. 1A), supporting the theory that the early effects of 5-azaC treatment should arise from RNA m⁵C changes.

Next, the inventors performed proliferation inhibition assays using different leukemia cell lines. The data showed that 5-azaC induced significant cell death within 24 hours in different leukemia cell lines; whereas its deoxy form decitabine, which could only be incorporated into DNA, had little impact within the same treatment window (FIGS. 1B-1E). These findings support that the RNA-dependent pathways alone can cause leukemia cell death. Sensitivity to 5-azaC varied across leukemia cell lines: MOLM13 and SKM-1, both derived from AML patients with prior MDS progression, were more susceptible to 5-azaC treatment at early time points (FIGS. 1B-1C). In contrast, the acute monocytic leukemia cell line THP1 displayed lower sensitivity (FIG. 1D), and K562 showed no response within 24 hours (FIG. 1E).

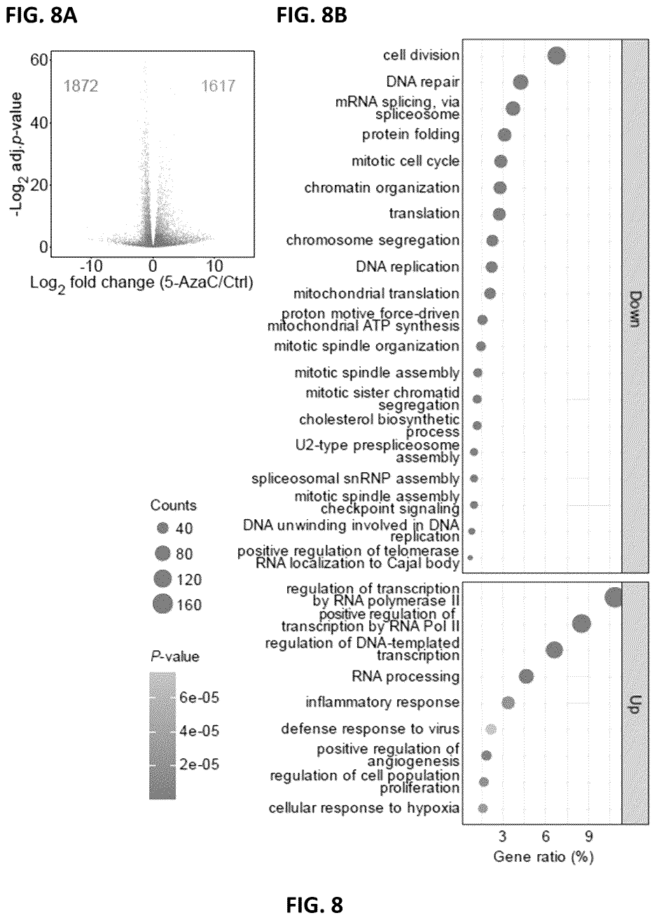

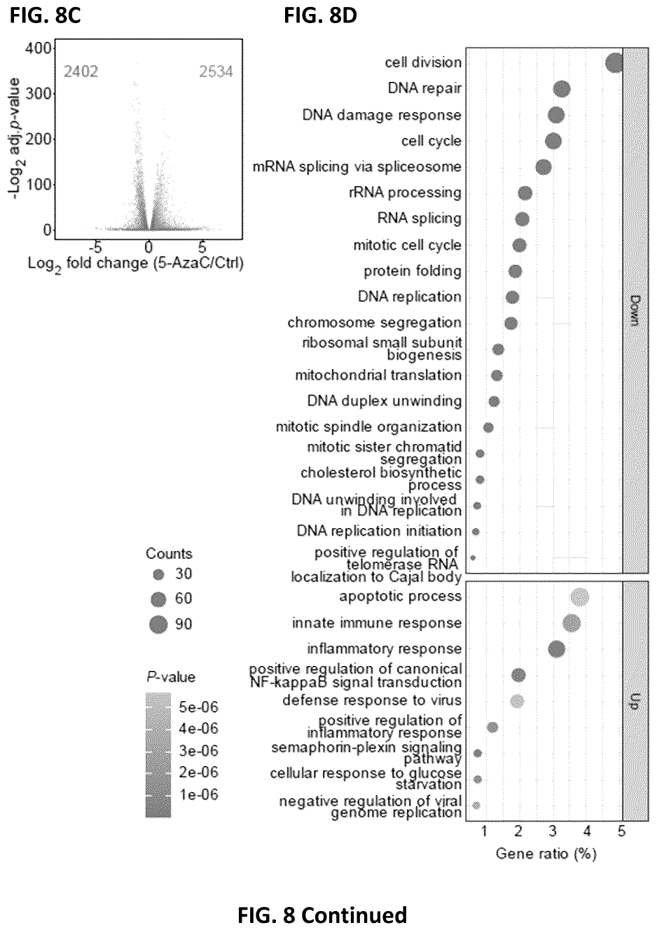

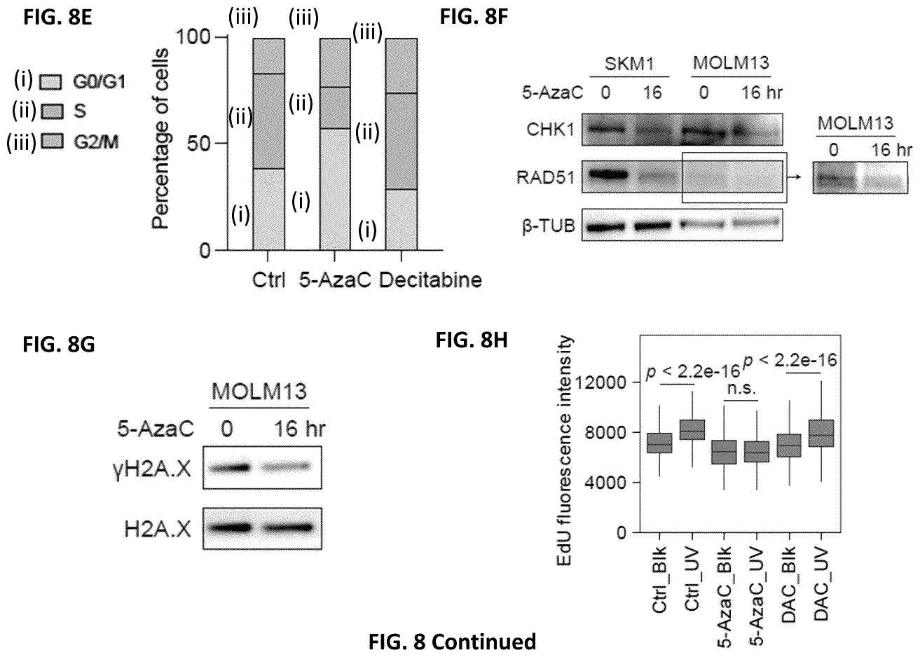

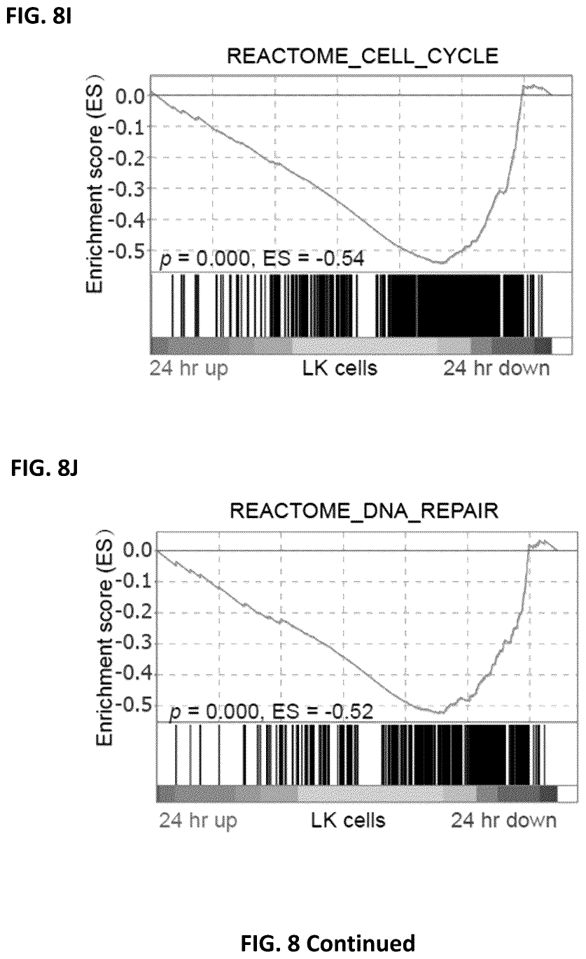

These results prompted the inventors to further explore and seek to understand the RNA-dependent pathways underlying the cytotoxicity of 5-azaC. The inventors performed RNA-seq following 24-hour 5-azaC treatment in MOLM13 and SKM1, and the data was analyzed, differentially expressed genes (DEGs) were identified, with both sets enriching pathways related to cancer cell survival. Gene ontology (GO) analysis of biological processes showed that downregulated DEGs were involved in cell division, DNA repair, and RNA processing, while upregulated DEGs were linked to the inflammatory response (FIGS. 8A-8D).

To explain the reduced proliferation with 5-azaC treatment, the inventors firstly focused on cell division. Multiple pathways related to cell division were downregulated, including mitotic cell cycle, DNA replication and chromosome segregation (FIGS. 8B and 8D). Gene set enrichment analysis (GSEA) revealed downregulation of cell cycle-related genes (FIG. 1F). A 16-hour treatment with 5-azaC significantly decreased the S-phase cell population in MOLM13 and SKM1, whereas decitabine treatment only caused a minor reduction in MOLM13 proliferation (FIGS. 1G and 8E). These observations suggest that 5-azaC treatment at early time points prolonged the leukemia cell cycle, thereby inhibiting proliferation.

Additionally, GO analysis revealed downregulation of DNA repair genes 24 hours after 5-azaC treatment (FIGS. 8B and 8D). GSEA showed a collective downregulation of DNA repair pathway genes (FIG. 1H). The downregulation was further confirmed by immunoblotting of several prominently changed genes, including the central checkpoint kinase CHK1 and DNA repair protein RAD51 (FIG. 8F). Notably, UVC irradiation elicited diminished γH2A.X signaling following 5-azaC treatment in MOLM13 cells (FIG. 8G). UVC-induced unscheduled DNA synthesis (UDS), measured by EdU incorporation followed by click chemistry, revealed impaired DNA repair activity following 16-hour exposure to 5-azaC but not decitabine (FIGS. 1I and 8H). These findings suggested that the early effects of 5-azaC disrupted DNA damage response in leukemia cells. The accumulation of DNA lesions may further impede cell cycle progression, enhancing the prolonged cell cycle observed with 5-azaC treatment (FIGS. 1G and 8E).

As previously established, defective cell cycle progression induces apoptosis. In parallel, apoptosis also arises from unsuccessful DNA repair, and DNA damage response inhibitors have been used in cancer therapy to promote cell death (31). The defective cell cycle regulation and DNA repair may explain the cytotoxic effects of 5-azaC by inducing apoptosis. Consistently, GSEA showed upregulation of apoptotic genes (FIG. 1J). The terminal deoxynucleotidyl transferase dUTP nick end labeling (TUNEL) assay detected apoptosis in MOLM13 cells after 16-hour treatment with 5-azaC but not decitabine (FIG. 1K). Intriguingly, 5-azaC has been combined with the BCL2 inhibitor venetoclax in clinical usage, resulting in improved recovery in AML patients (32,33). Indeed, when the inventors combined 5-azaC and venetoclax the data showed enhanced efficacy of proliferation inhibition for MOLM13 cells after 24 hours, whereas no synergistic effect was observed for decitabine and venetoclax under the same conditions (FIG. 1L). These results revealed RNA-dependent cytotoxicity of 5-azaC in leukemia cells at early time points, and how this effect can synergize with pro-apoptotic agents.

To further explore the RNA-dependent effect of 5-azaC under physiological conditions, the inventors performed 24-hour 5-azaC treatment of the Lin-KIT+ (LK) cells purified from mouse bone marrow (BM). GSEA analysis revealed downregulation of cell cycle and DNA repair genes similar to those observed in leukemia cells (FIGS. 8I-8J). Additionally, the inventors detected an upregulation of genes related to myeloid differentiation (FIG. 1M). This was consistent with the reports that 5-azaC helps restore the differentiation of leukemia (34). These findings uncovered the previously overlooked cytotoxicity of 5-azaC mediated through RNA m5C depletion.

### Example 3—5-azaC Induced Transcriptional Repression Through caRNA m⁵C Depletion

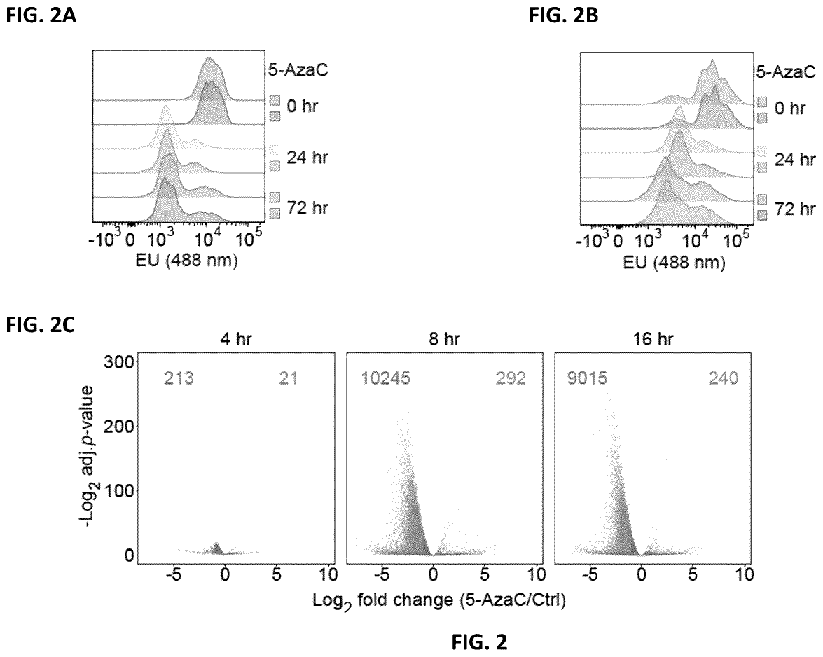

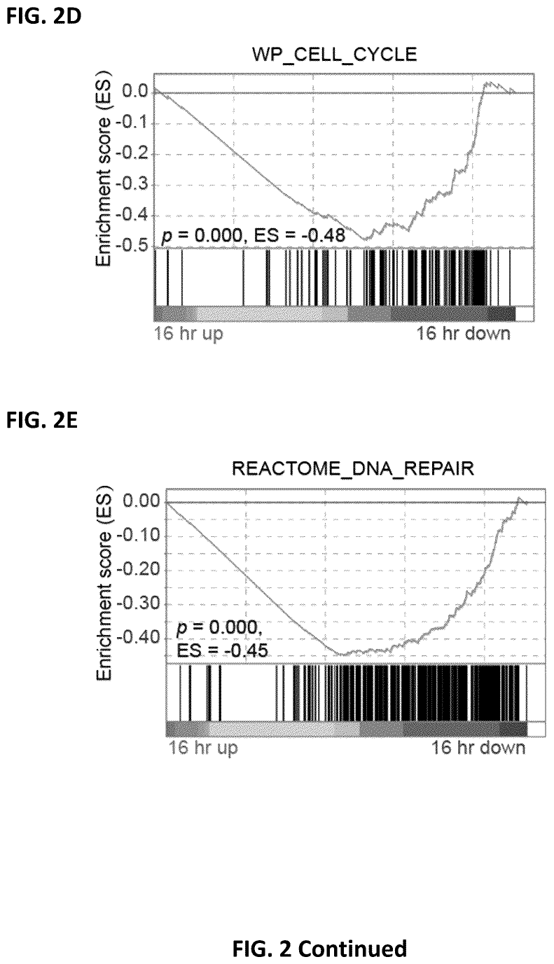

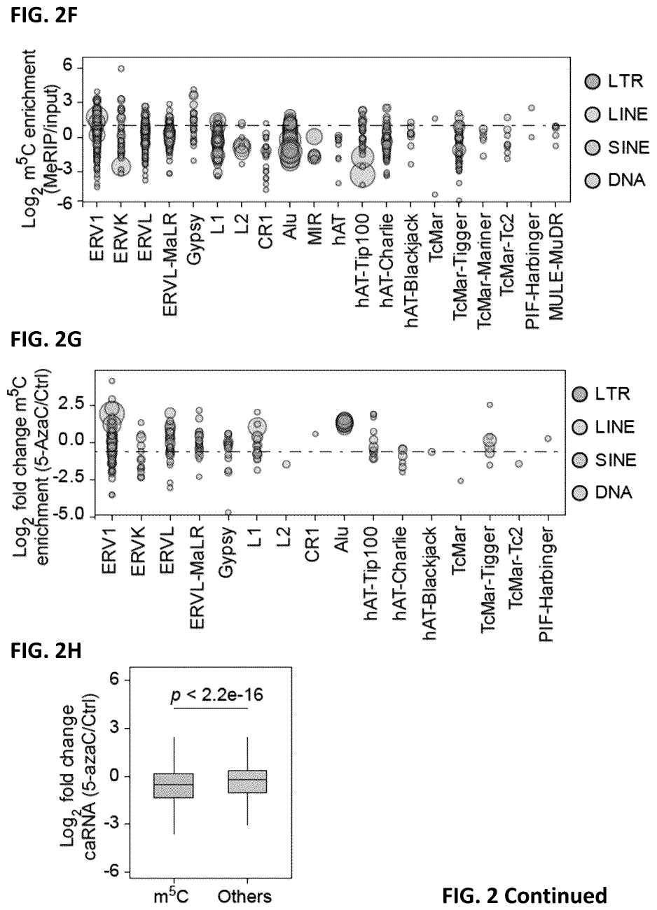

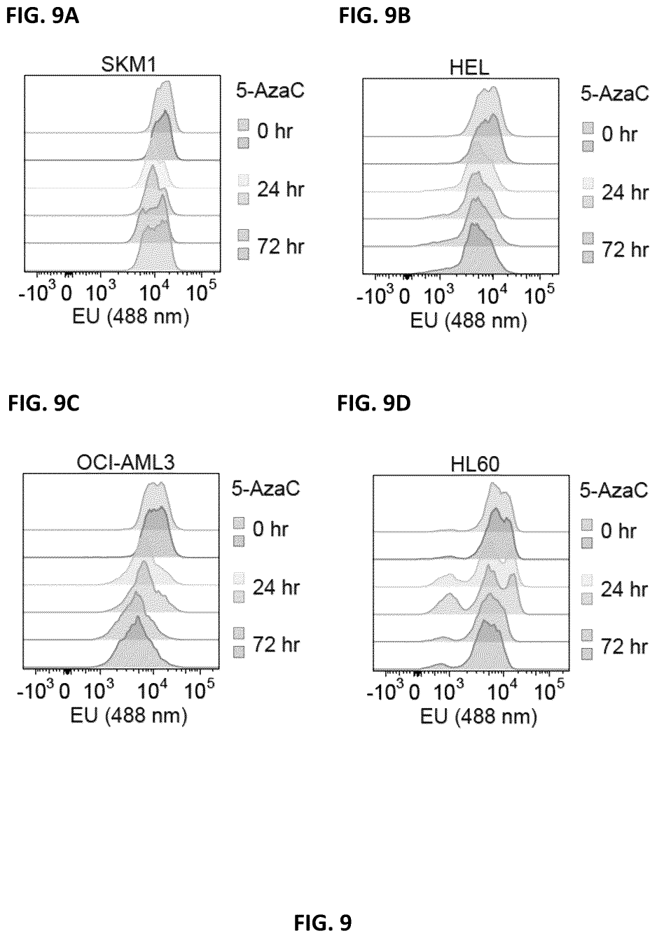

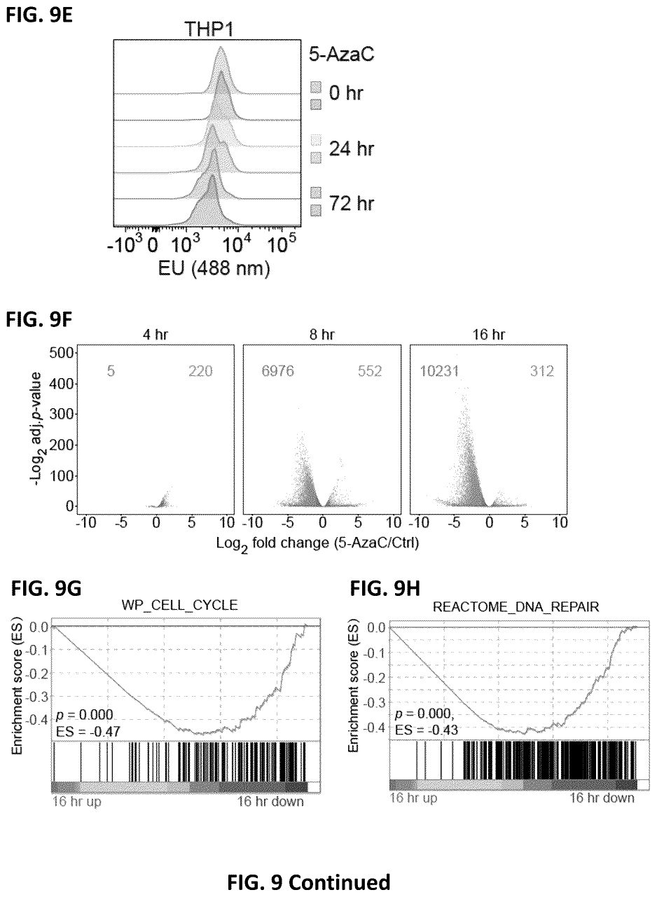

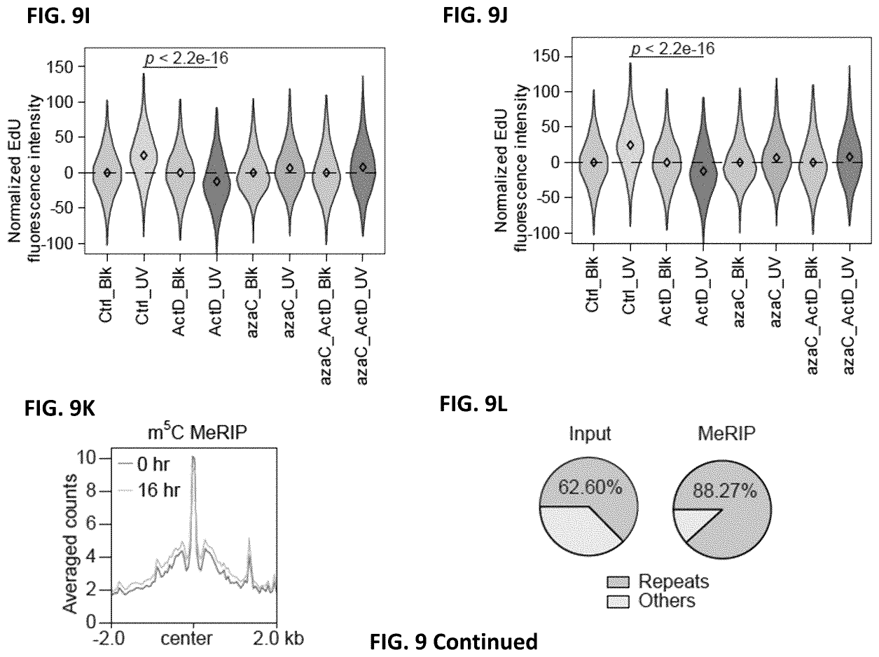

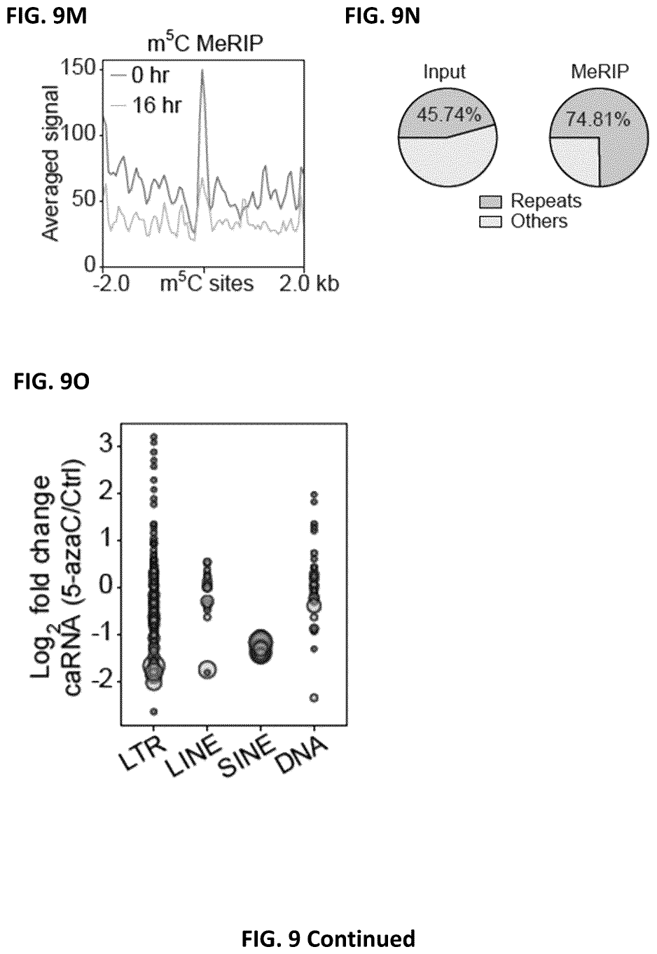

5-azaC has long been recognized as a hypomethylating agent, with its clinical function largely attributed to transcriptional regulation (35). Considering that its RNA-dependent effects significantly altered the transcriptome, and that caRNA m⁵C depletion was more pronounced than tRNA m⁵C (FIG. 1E), the inventors asked how the RNA-dependent process regulates transcription. Contrary to its long-term DNA hypomethylation effect, 5-azaC led to transcriptional downregulation across leukemia cell lines, as measured by EU labeling of nascent RNA, after 24 hours of treatment (FIGS. 2A and 9A-9E). Interestingly, despite the anticipated transcriptional upregulation due to DNA demethylation at 72 hours, RNA-dependent effects appeared to dominate, and transcriptional levels remained decreased after 72 hours in all six tested cell lines. This aligns with previous reports of inconsistencies observed between differential gene expression and DNA methylation changes following 5-azaC treatment (1-4), as well as clinical cases where some MDS patients benefited from 5-azaC treatment without significant DNA demethylation (5). Leukemia patient samples exhibited similar transcriptional downregulation after 24- and 72-hour 5-azaC treatment (FIG. 2B), highlighting the clinical relevance of these RNA-dependent effects. These results underscore the importance of the RNA-dependent mechanisms in 5-azaC-induced gene expression dysregulation.

To assess gene-specific transcriptional changes, the inventors performed nascent RNA-seq (FIGS. 2C and 9F), which captures newly synthesized RNA through biotin labeling of incorporated EU. The nascent RNA levels were normalization to viable cell numbers by adding EU-labeled drosophila spike-in. The nascent RNA-seq revealed significant transcriptional downregulation emerging from 8 hours of 5-azaC treatment, consistent with the starting time of the RNA m⁵C decrease (FIG. 1E). GSEA showed transcriptional downregulation of cell cycle genes and DNA repair genes (FIGS. 2D-2E, and 9G-9H), suggesting that the differential gene expression of corresponding pathways induced by 5-azaC is indeed derived from transcriptional regulation.

One of the DNA repair pathways, nucleotide excision repair (NER), is mediated by either global genome repair or transcription-coupled repair (TCR), with the latter one dependent on transcriptional activity (36). The inventors reasoned that the overall transcriptional downregulation might contribute to the impaired DNA damage response caused by 5-azaC. Compared with controls, UDS after UVC irradiation was less susceptible to transcription inhibition in 5-azaC-treated cells (FIGS. 9I-9J), implying the role of transcriptional suppression on the early DNA repair defects induced by 5-azaC.

To explore the mechanisms underlying transcriptional repression, the inventors identified m⁵C sites on caRNA using methylated RNA immunoprecipitation sequencing (MeRIP-seq) as previously reported (28). RNA m⁵C levels were reduced after 16-hour 5-azaC treatment in MOLM13 (FIG. 9K), with 7,095 downregulated and 4,567 upregulated sites out of 21,158 peaks identified from control and treated samples. Notably, repetitive elements accounted for 62.6% input reads and 88.27% MeRIP-enriched reads (FIG. 9L), highlighting repeat RNA as a crucial group of caRNA that may exert transcriptional regulation. The increased proportion of repeat RNA in MeRIP samples further suggested preferential m⁵C installation. Similar to MOLM13, SKM1 cells also exhibited significant caRNA m⁵C downregulation after 16 hours of 5-azaC treatment (FIG. 9M), with repeat RNA preferentially enriched in m⁵C MeRIP-seq (FIG. 9N). Further analysis identified long terminal repeat (LTR) elements as the most modified repeat RNA (FIG. 2F), consistent with the inventors previous findings (28), while some subfamilies of long interspersed nuclear element (LINEs), short interspersed nuclear elements (SINEs), and DNA transposons were also enriched. 5-AzaC treatment caused reduced m⁵C levels among repeat RNAs (FIG. 2G), consistent with the reduced overall caRNA m⁵C level measured with LC-MS/MS (FIG. 1E). RNA modifications on repeat RNA have emerged as key elements in the regulation of local chromatin state and transcription (28,37-39), and caRNA m⁵C have been associated with maintaining an open chromatin state (28). Therefore, the 5-azaC-induced m⁵C downregulation may drive the observed transcriptional repression. Indeed, 5-azaC treatment caused the downregulation of caRNA repeat subfamilies bearing m⁵C, particularly in the LTR family (FIG. 9O). Furthermore, genes adjacent to m⁵C sites exhibited greater downregulation than those farther away (FIG. 2H), reinforcing the link between m5C loss and transcriptional repression of local chromatin regions.

### Example 4—5-azaC Induced Transcriptional Repression Through m⁵C-Dependent H2AK119Ub Regulation

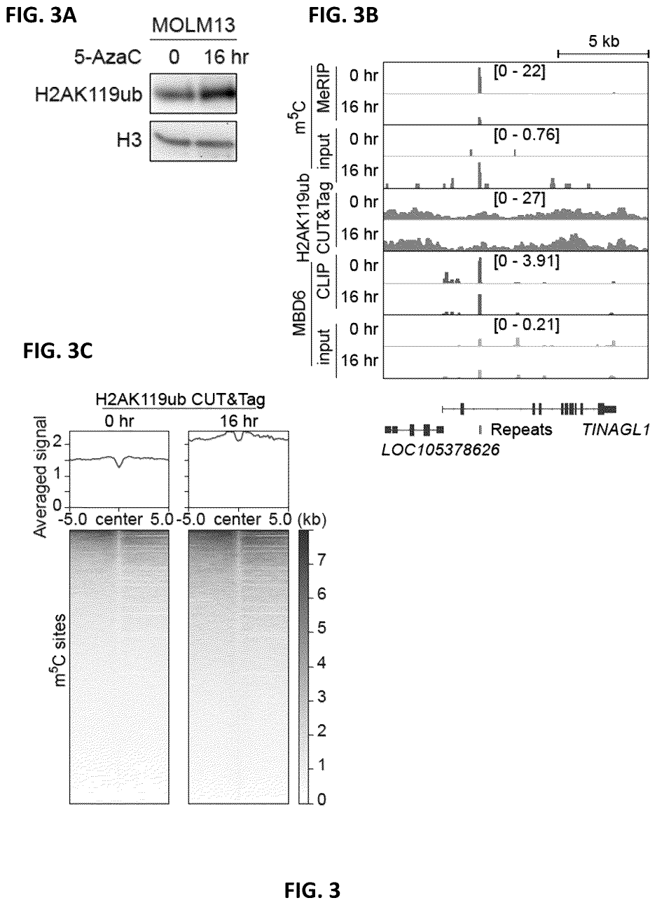

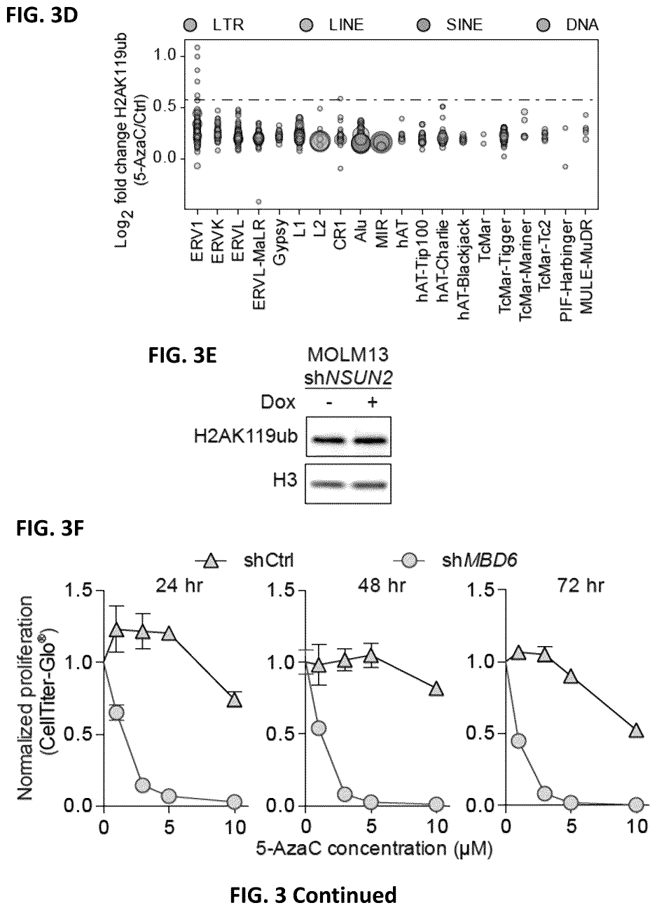

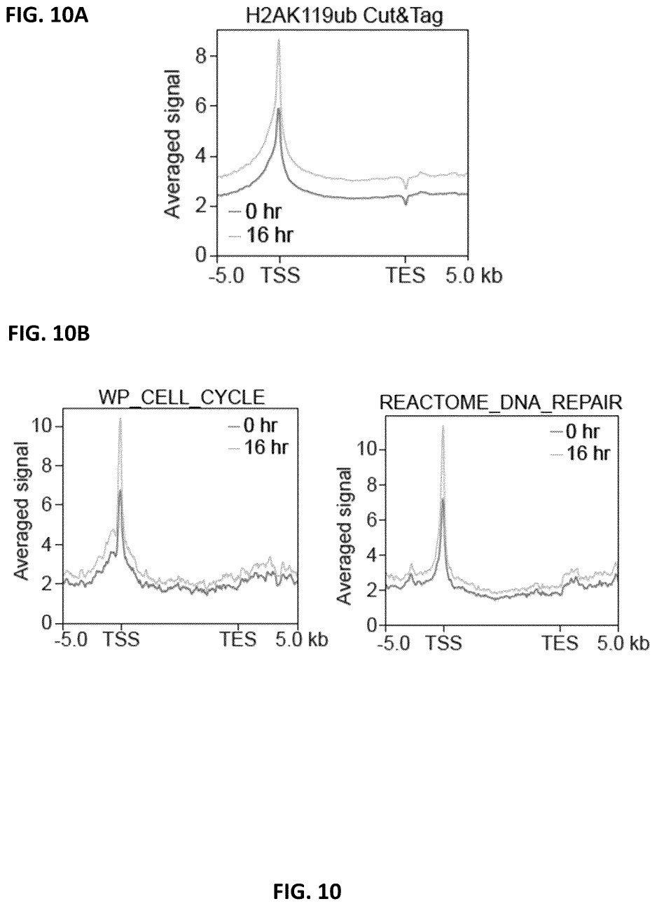

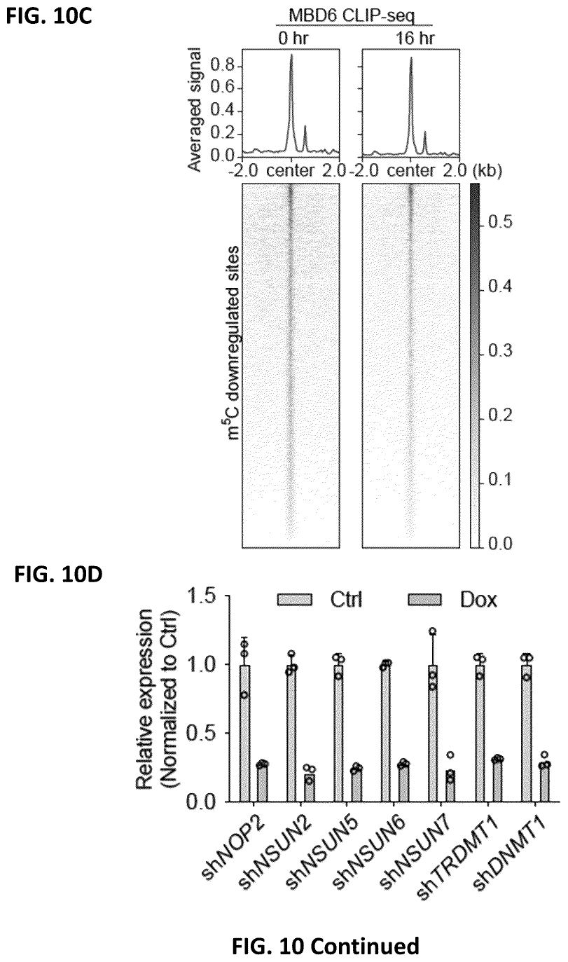

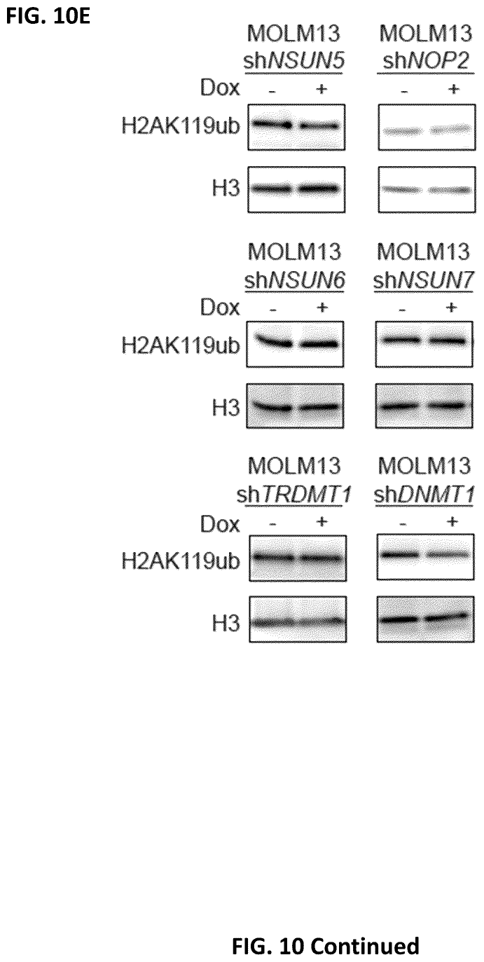

The inventors have recently shown that caRNA m⁵C regulates transcription by recruiting the m⁵C-binding protein MBD6 and its interacting partner, the PR-DUB deubiquitylase complex, which mediates removal of the histone modification H2AK119ub to promote transcriptional activation (28). Inhibition of RNA methyltransferase that installs caRNA m⁵C by 5-azaC would reduce PR-DUB recruitment and suppress transcription. Indeed, the inventors observed an increase of the global H2AK119ub level following 16-hour 5-azaC treatment by immunoblotting (FIG. 3A). H2AK119ub CUT&Tag analysis demonstrated elevated H2AK119ub levels near genomic regions of m⁵C-modified caRNA (FIGS. 3B-3C). Consistent with the previously suggested role of LTRs in H2AK119ub regulation (28), the data showed the most significant upregulation of H2AK119ub at specific LTR subfamilies (FIG. 3D). This upregulation was detected across gene loci and was particularly evident at transcription start sites (TSS) (FIG. 10A), supporting its role in transcriptional regulation. Particularly, the inventors observed enrichment of gene sets related to cell cycles and DNA repair pathways (FIG. 10B), supporting that H2AK119ub changes contribute to the transcriptional dysregulation of these pathways. Cross-linking immunoprecipitation followed by high-throughput sequencing (CLIP-seq) revealed that 5-azaC induced decreased MBD6 binding at m⁵C sites (FIGS. 3B and 10C), consistent with its role as an m⁵C-binding protein involved in H2AK119ub regulation.

To further validate the role of m⁵C in this process, the inventors depleted NSUN2, the primary methyltransferase responsible for installing m⁵C on caRNA (28). NSUN2 knockdown (KD) resulted in H2AK119ub upregulation (FIG. 3E), supporting a causal role for m⁵C in this regulation. Notably, depletion of other m⁵C methyltransferase, including NSUN5, NOP2, NSUN6, NSUN7 and TRDMT1, did not induce similar H2AK119ub upregulation (FIGS. 10D-10E), indicating that NSUN2 plays the primary role in the m⁵C-mediated regulation of H2AK119ub. Furthermore, depletion of the DNA 5mC methyltransferase DNMT1 led to a reduction in H2AK119ub levels, reinforcing that the early time point effect of 5-azaC on H2AK119ub goes through the RNA-dependent pathway rather than DNA hypomethylating activity.

MBD6 depletion has been shown to rescue the differentiation delay defect of hematopoietic stem and progenitor cells (HSPCs) and inhibit leukemogenesis in leukemia cells with Tet2 deletion or mutation, implying a role for MBD6 inhibition in leukemia treatment. Consistently, MBD6 KD sensitized MOLM13 cells to 5-azaC treatment (FIG. 3F), indicating that combining 5-azaC with an MBD6 inhibitor could be a promising therapeutic approach for treating leukemia.

Overall, these findings support that 5-azaC represses transcription through caRNA m⁵C depletion that affects the m⁵C/MBD6/PR-DUB/H2AK119ub axis.

### Example 5—5-azaC Induced Transcriptional Repression Through Downregulation of H3K27ac

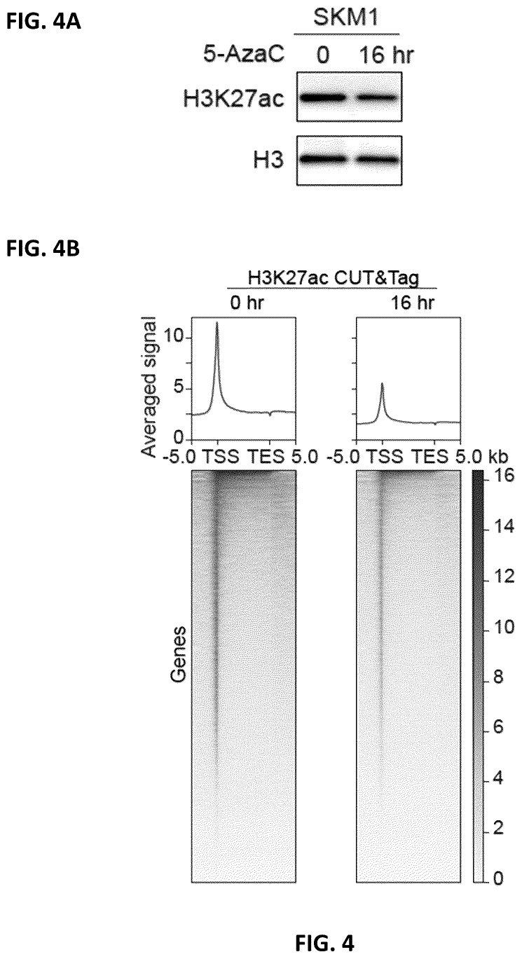

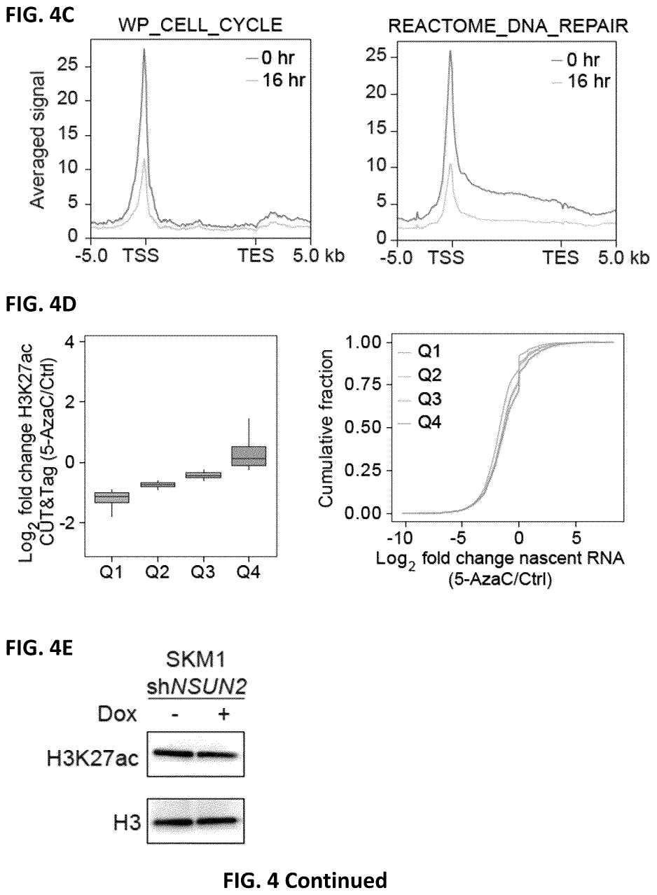

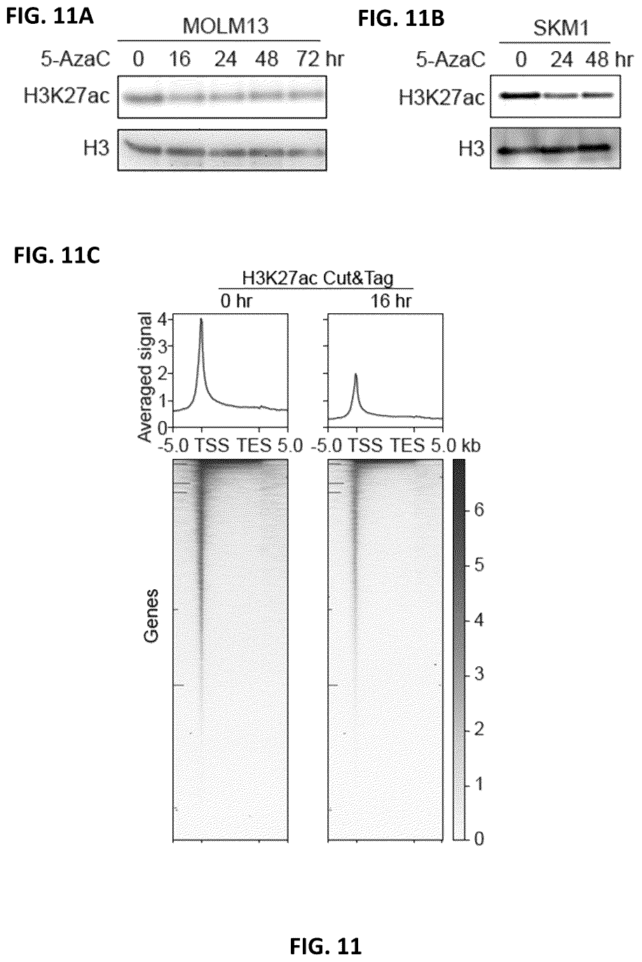

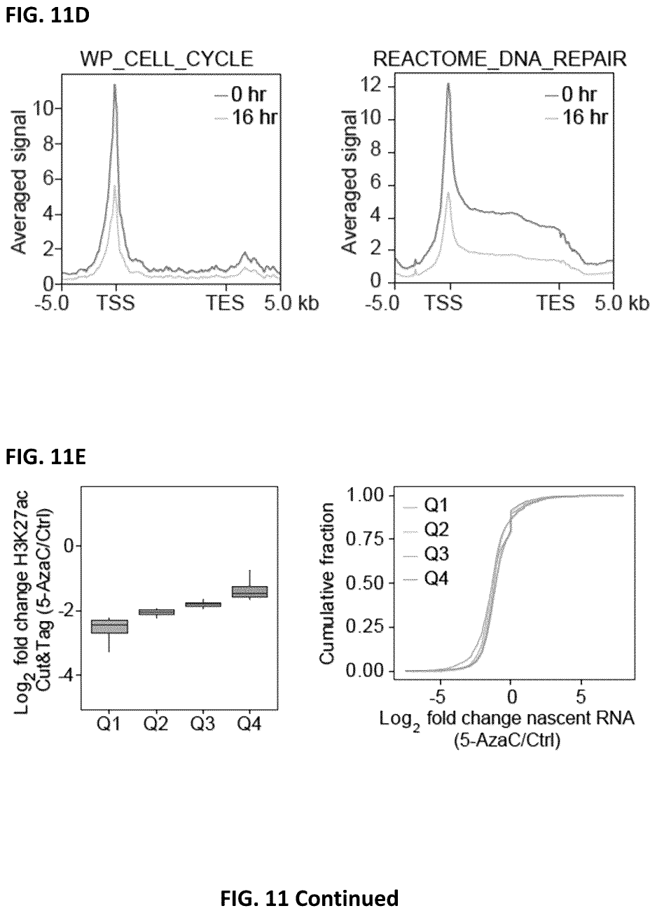

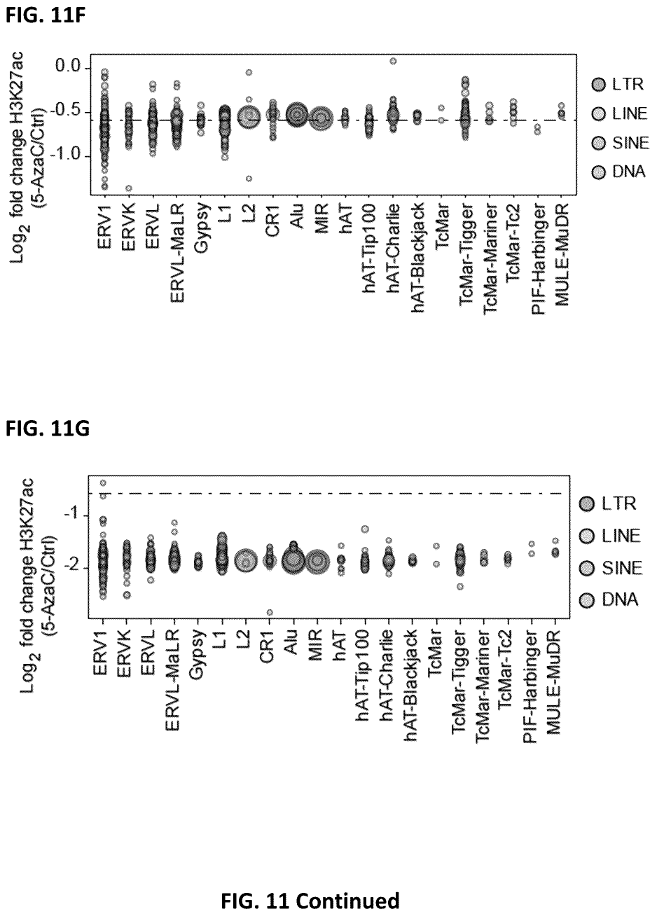

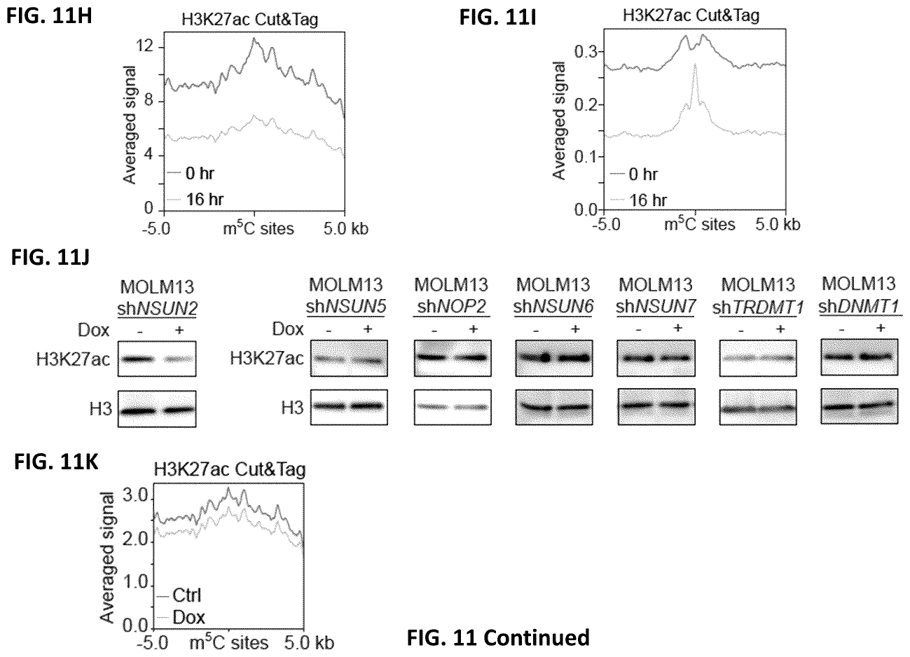

It has been reported that inhibitors of CBP/p300 exhibited a synergistic effect with 5-azaC but not with decitabine (40), suggesting a link between the RNA-dependent function of 5-azaC and H3K27ac regulation. Indeed, the inventors observed overall decreased H3K27ac levels in MOLM13 and SKM1 cells after 16-hour 5-azaC treatment (FIGS. 4A and 11A). This finding indicated that H3K27ac downregulation contributes to the m⁵C-dependent transcriptional repression by 5-azaC. The reduction of H3K27ac persisted at later time points (FIGS. 11A-11B), aligning with the predominant transcriptional repression effect of 5-azaC over DNA hypomethylation. H3K27ac CUT&Tag analysis confirmed a global downregulation of H3K27ac (FIGS. 4B and 11C), particularly affecting gene sets related to cell cycles and DNA repair (FIGS. 4C and 11D). To elucidate the relationship between H3K27ac downregulation and transcriptional repression, the inventors grouped genes into four quantiles based on the degree of the adjacent H3K27ac peak changes, with Q1 representing the most downregulated genes and Q4 the most upregulated. A comparison with nascent RNA-seq data revealed a correlation between H3K27ac loss and transcriptional repression in downregulated genes (FIGS. 4D and 11E). Most downregulation of H3K27ac occurred in regions enriched with LTRs and LINEs (FIGS. 11F-11G). Decreased H3K27ac was observed at genomic loci corresponding to m⁵C-modified sites in both MOLM13 and SKM1 (FIGS. 11H-11I), supporting RNA m⁵C-dependent H3K27ac downregulation by 5-azaC treatment.

To further elucidate the causal relationship between m⁵C and H3K27ac, the inventors examined NSUN2, the primary caRNA m⁵C methyltransferase (28). NSUN2 depletion caused a reduction of the H3K27ac level (FIGS. 4E and 11J). Among other m5C methyltransferases, the inventors observed a subtle decrease of H3K27ac following NSUN7 KD (FIG. 11K), consistent with its reported role in eRNA m⁵C modification (41). Depleting NSUN5, NOP2, NSUN6 or TRDMT1 did not induce H3K27ac reduction, suggesting their RNA targets are not involved in this regulation. Notably, depletion of DNMT1 resulted in H3K27ac upregulation, further confirming that the early time point H3K27ac disruption observed with 5-azaC treatment is RNA-dependent rather than DNA-dependent. As NSUN2 depletion showed the strongest effect on H3K27ac, the inventors performed H3K27ac CUT&Tag to explore genomic effects. NSUN2 depletion led to H3K27ac downregulation at m⁵C sites (FIG. 11L), reinforcing the causal relationship between m⁵C and H3K27ac.

### Example 6—5-azaC Downregulated H3K27Ac Through SRSF2-Mediated p300 Recruitment

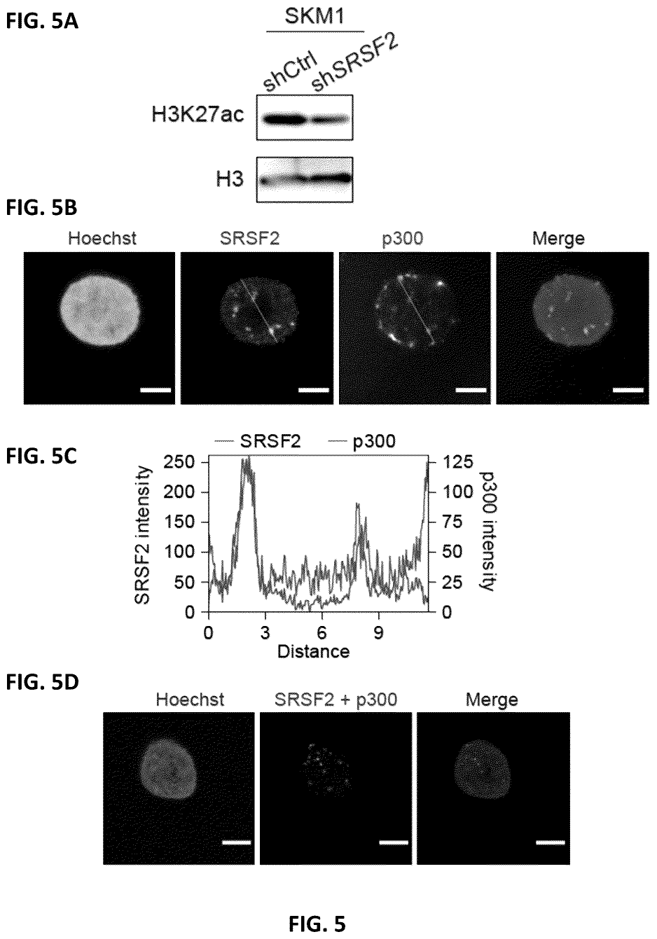

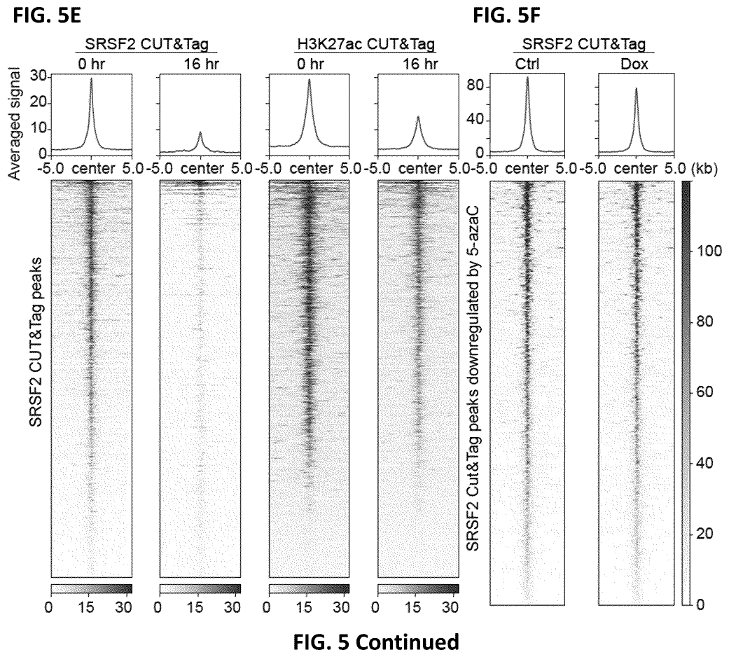

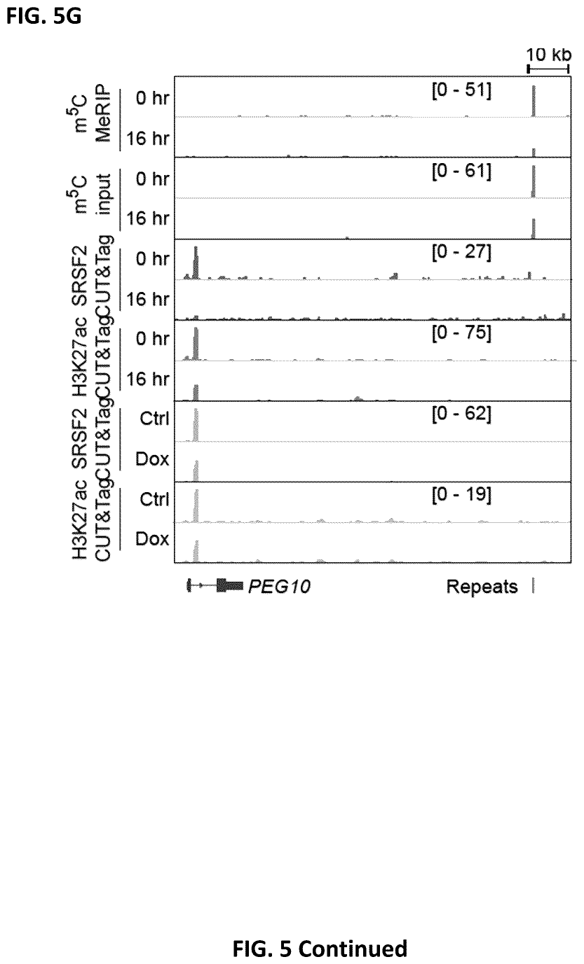

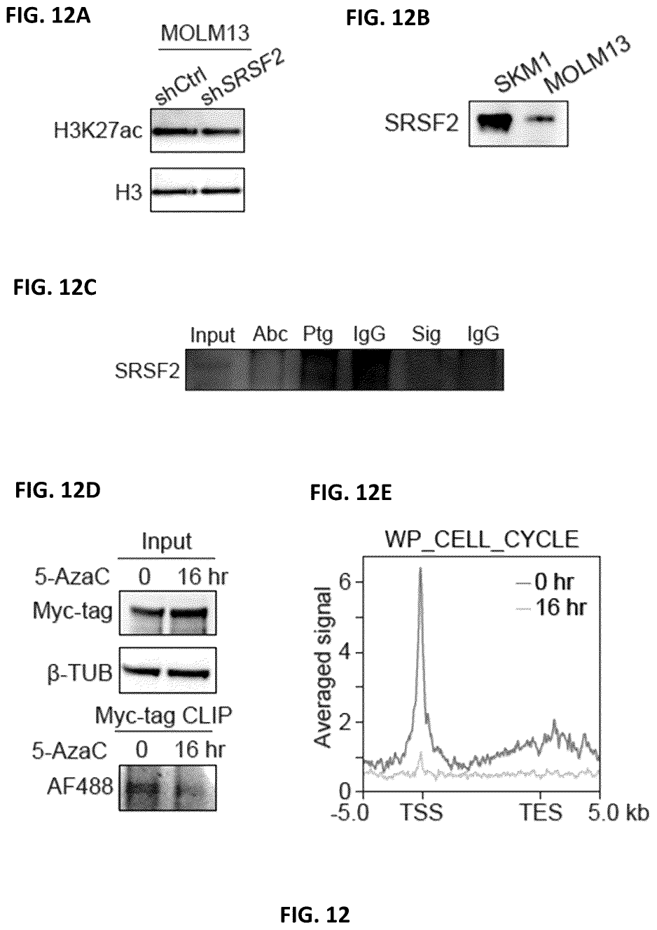

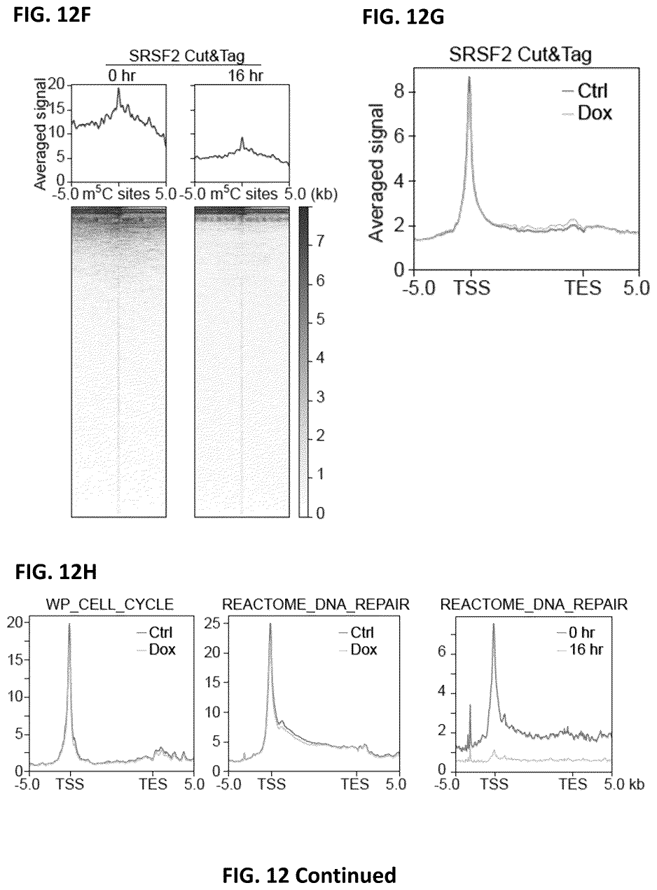

To elucidate the underlying mechanism connecting caRNA m⁵C to H3K27ac, the inventors examined a nuclear localized m⁵C-binding protein SRSF2 (24). SRSF2 depletion resulted in a significant reduction of H3K27ac (FIGS. 5A and 12A), suggesting its role in H3K27ac regulation. Given that SKM1 cells exhibited significantly higher SRSF2 expression than MOLM13 cells (FIG. 12B), the inventors selected SKM1 to further explore SRSF2-dependent H3K27ac regulation. Previous studies have shown that SRSF2 associates with p300, thereby influencing H3K27ac deposition (42,43). Consistently, immunofluorescence analysis revealed colocalization of SRSF2 and p300 (FIGS. 5B-5C). Proximity ligation assay (PLA) confirmed their interaction (FIG. 5D), supporting that SRSF2 recruits p300 to regulate H3K27ac deposition.

Based on these observations, the inventors hypothesized that 5-azaC disrupts m⁵C-guided genomic binding of SRSF2, thereby impairing 300 localization and H3K27ac installation. Due to the inability of commercial SRSF2 antibodies to efficiently immunoprecipitate SRSF2(FIG. 12C), the inventors overexpressed SRSF2-Myc in K562 cells to assess its RNA binding following m⁵C-depletion by 5-azaC. After 16 hours of treatment, SRSF2 exhibited reduced RNA binding, as detected by cross-linking immunoprecipitation (CLIP) with end ligation of pCp-AF488 fluorophore (FIG. 12D). Given that SRSF2 binding to chromatin is known to be RNA-dependent (44), the inventors hypothesized that impaired RNA binding of SRSF2 leads to reduced chromatin binding and subsequent loss of H3K27ac deposition. To test this, the inventors performed SRSF2 CUT&Tag after 16 hours of 5-azaC treatment. Indeed, the data showed a significant reduction in SRSF2 chromatin binding, which correlated with decreased H3K27ac (FIGS. 5E and 5G). SRSF2 was highly enriched at promoters of genes involved in cell cycle and DNA repair pathways, and its binding at these sites was diminished following 5-azaC treatment (FIG. 12E). This is consistent with the recent findings that genes involved in DNA damage repair are particularly sensitive to transcriptional regulation by SRSF2 (45). Furthermore, SRSF2 binding was downregulated at chromatin regions adjacent to m⁵C sites (FIG. 12F), supporting the model in which caRNA m⁵C facilitates SRSF2 recruitment and H3K27ac deposition. Depletion of caRNA m⁵C would therefore reduce H3K27ac.

To establish a direct link between m⁵C and SRSF2 chromatin binding, the inventors performed SRSF2 CUT&Tag after NSUN2 depletion in SKM1 cells. SRSF2 chromatin binding was reduced at regions where its occupancy was also downregulated by 5-azaC (FIGS. 5F-5G), reinforcing the role of m⁵C in guiding SRSF2 localization to chromatin. The reduction was most pronounced at TSS (FIGS. 12G-12H), particularly for genes involved in cell cycle and DNA repair pathways, aligning with early transcriptional changes induced by 5-azaC.

In summary, in addition to the m⁵C/MBD6/PR-DUB/H2AK119ub pathway, as disclosed herein, the inventors have uncovered a parallel pathway of 5-azaC-induced transcriptional repression through the m⁵C/SRSF2/p300/H3K27ac axis, highlighting the role of RNA m⁵C in epigenetic regulation and transcriptional repression effect of 5-azaC.

### Example 7—NSUN2 Regulated Leukemia Proliferation and Hematopoietic Lineage Commitment

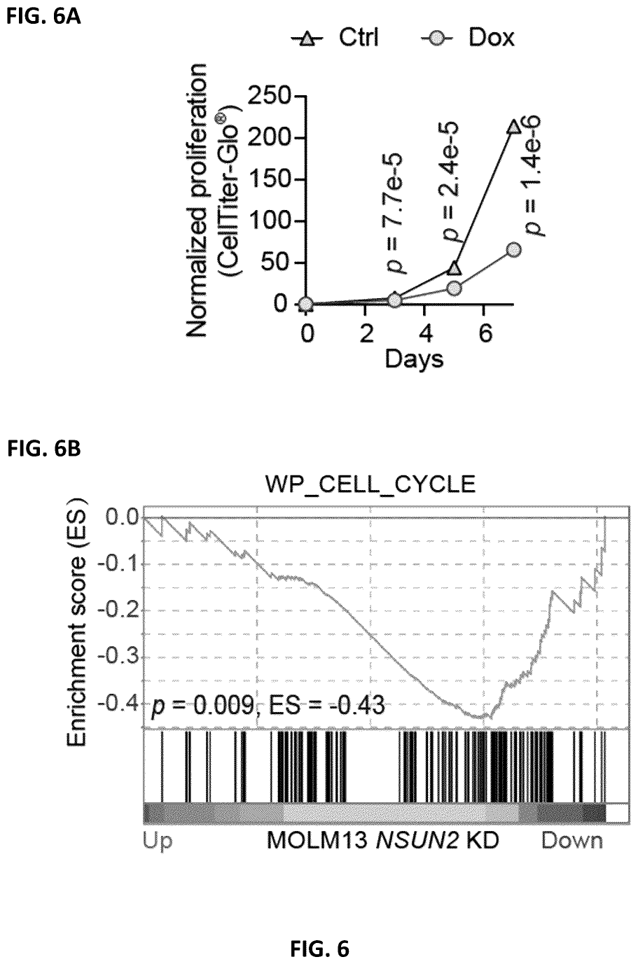

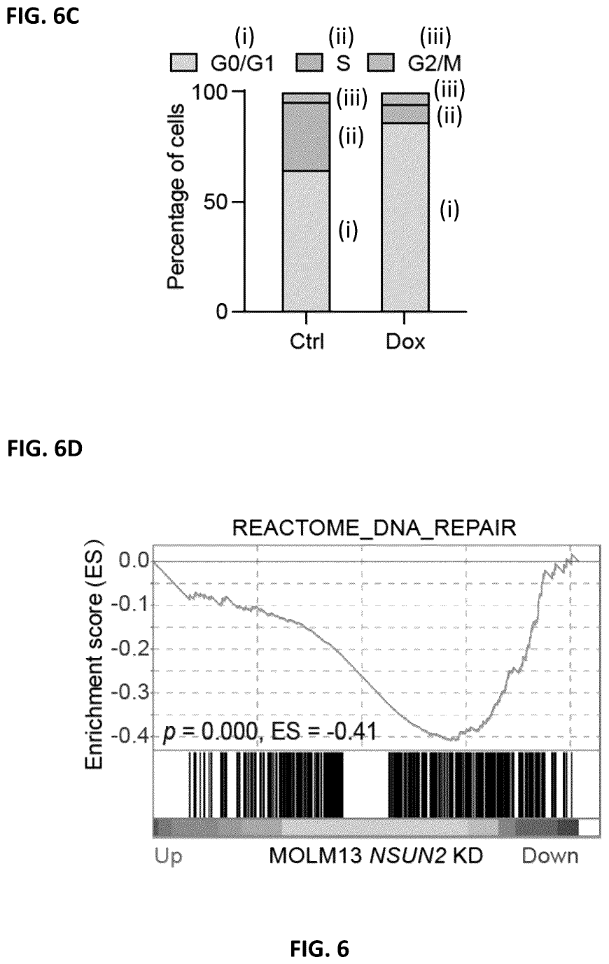

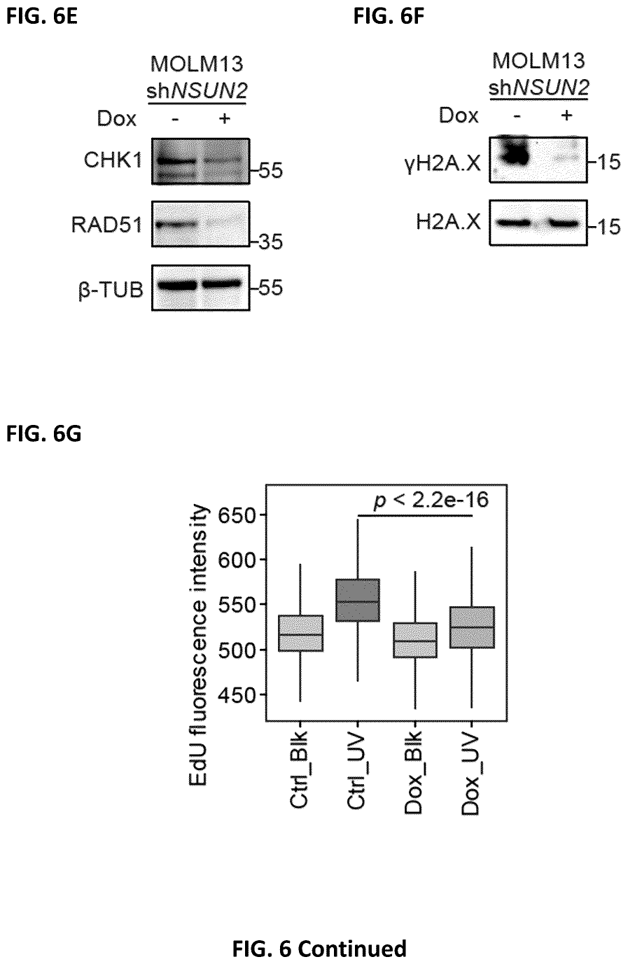

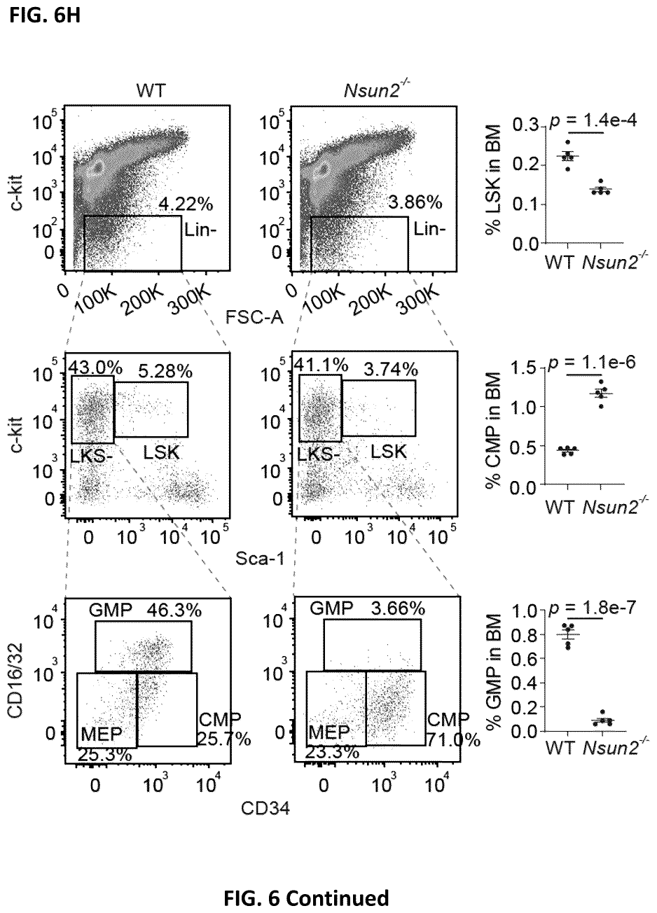

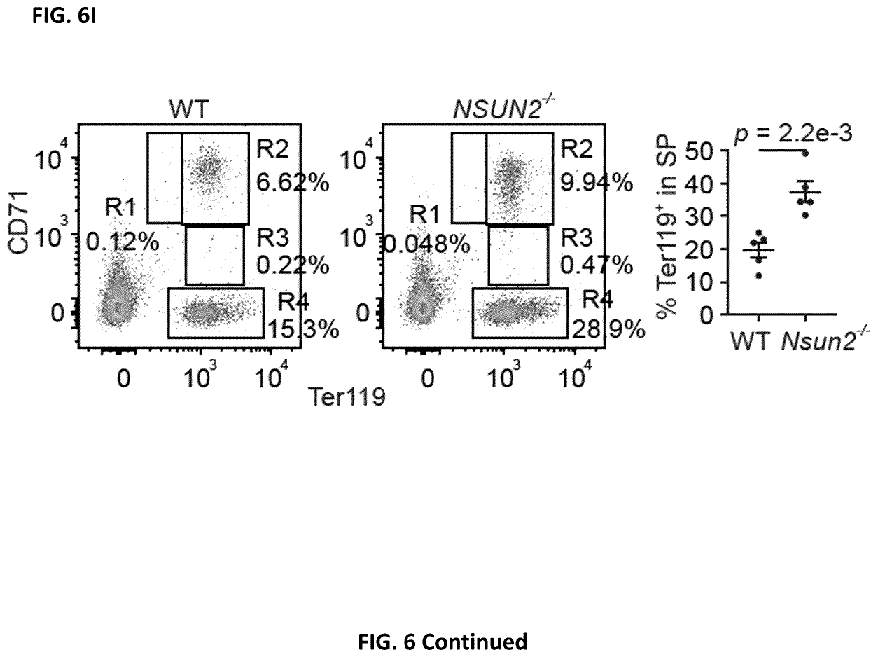

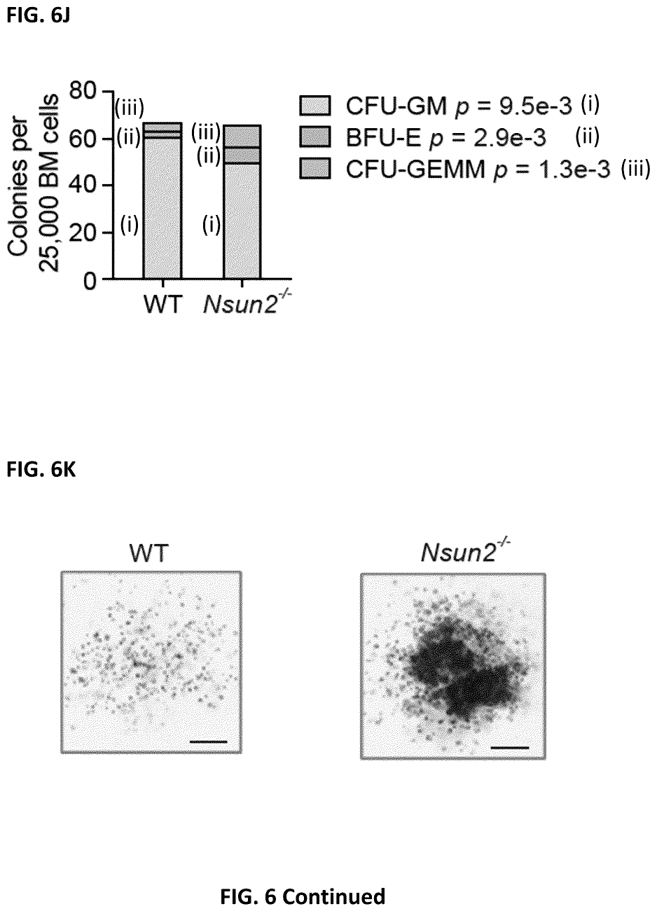

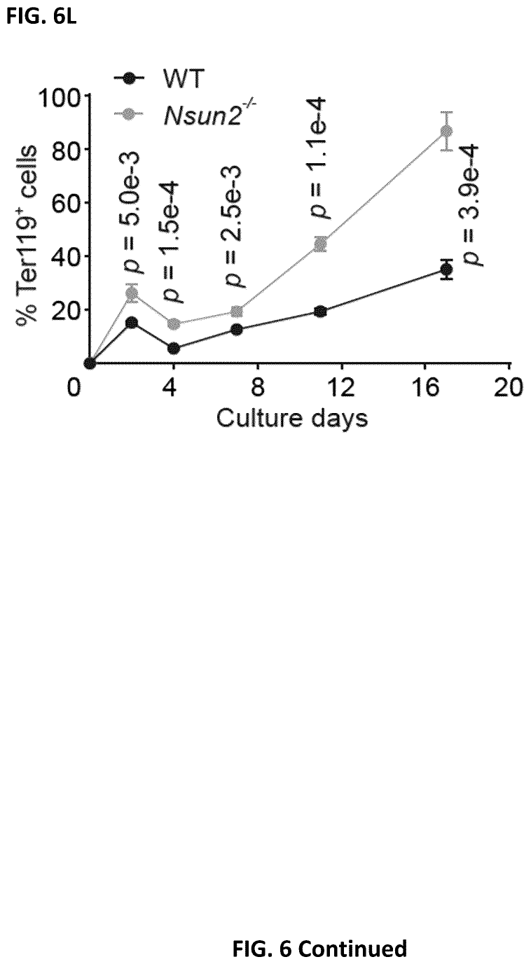

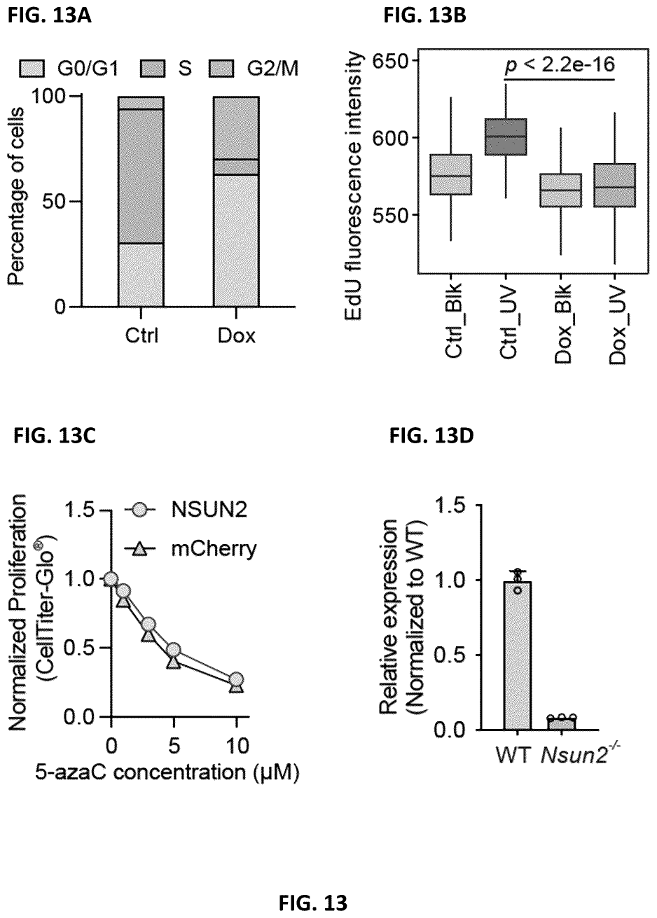

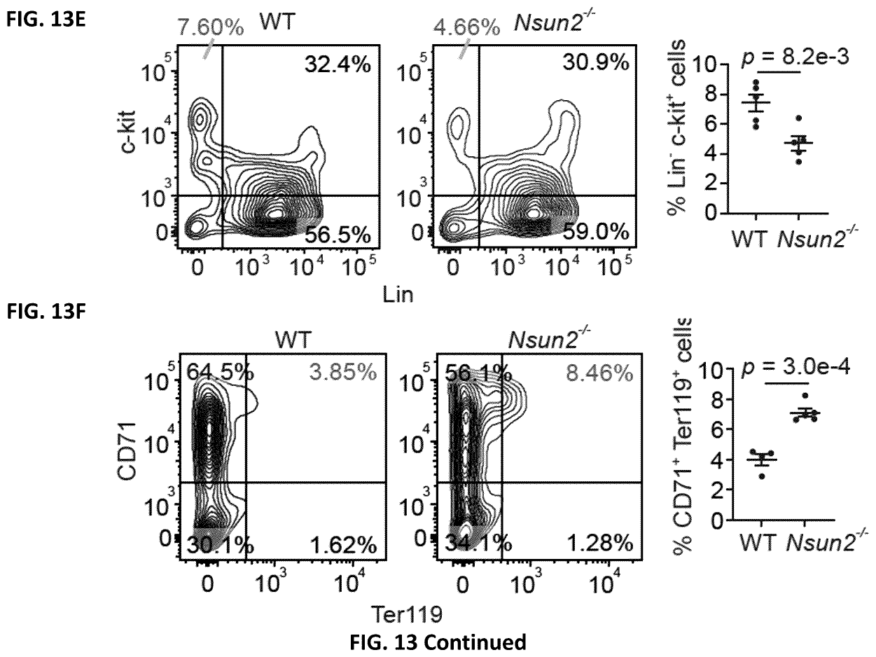

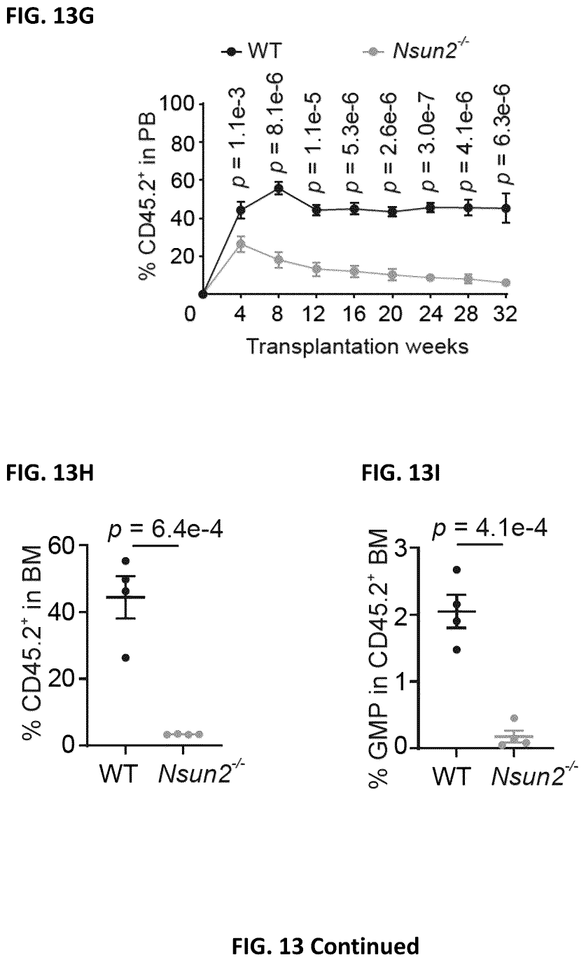

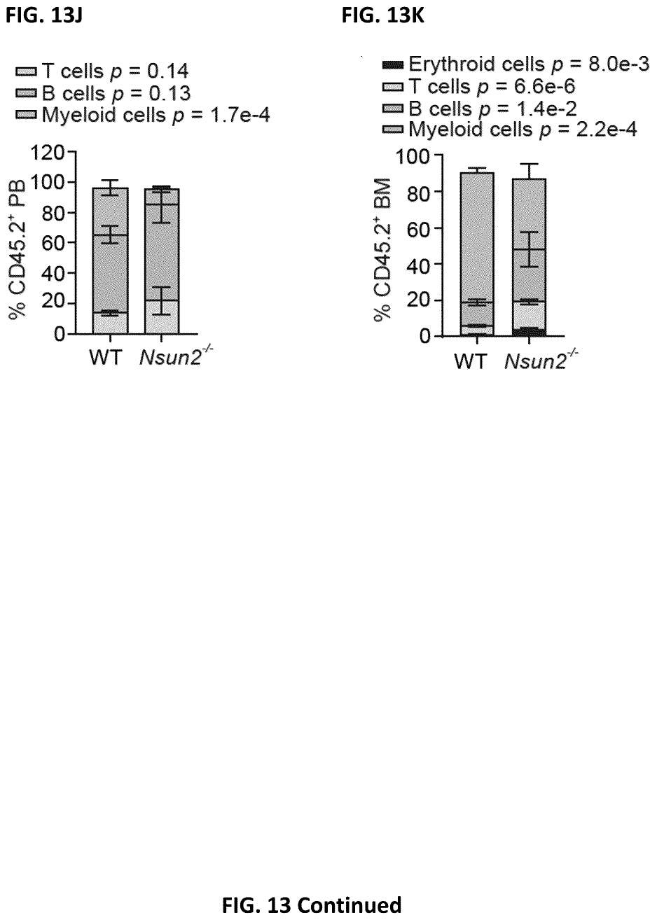

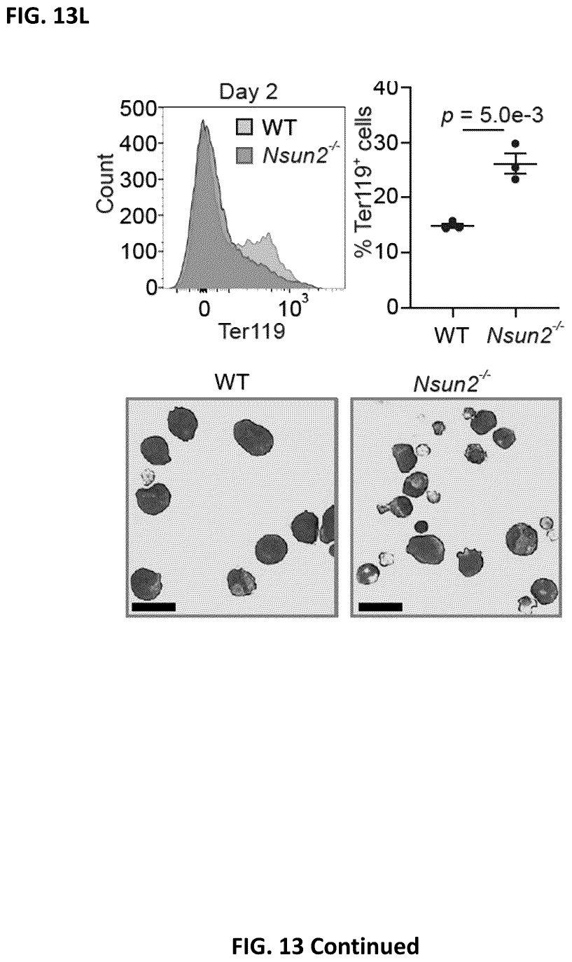

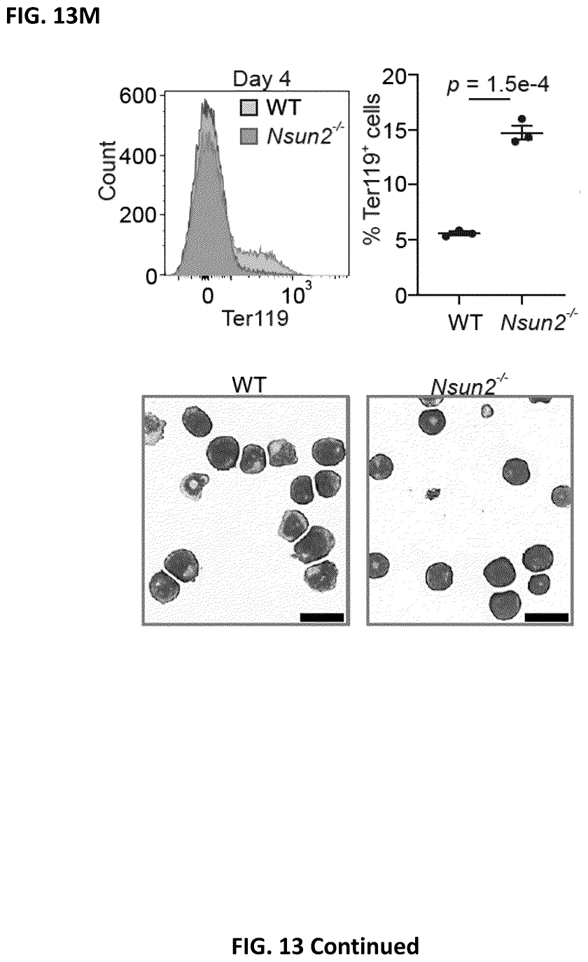

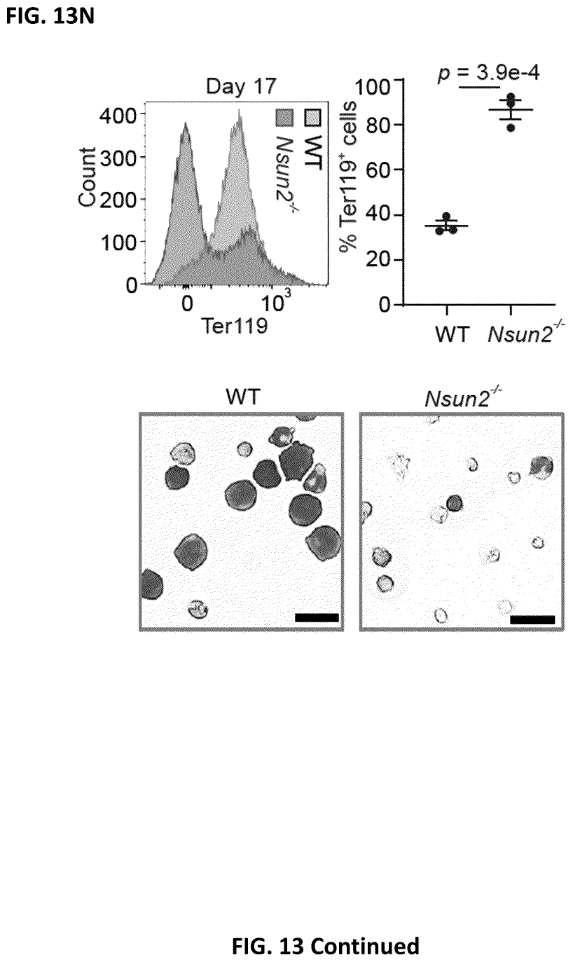

The inventors reasoned that, given that NSUN2 is the primary m⁵C methyltransferase responsible for the 5-azaC-mediated transcriptional repression, NSUN2 depletion should recapitulate the reduced proliferation of leukemia cells observed with 5-azaC treatment. Indeed, proliferation inhibition was observed after NSUN2 depletion in MOLM13 (FIG. 6A). This aligns with previous reports in other cancer cell types, where NSUN2 depletion inhibits cell proliferation (46,47). RNA-seq of NSUN2 KD MOLM13 cells followed by GSEA revealed a downregulation of cell cycle genes (FIG. 6B). NSUN2 depletion led to a decreased S-phase population in MOLM13 and SKM1 cells (FIGS. 6C and 13A), suggesting prolonged cell cycles progression. Additionally, GSEA indicated a downregulation of DNA repair pathways (FIG. 6D). The reduced expression of key DNA repair regulators CHK1 and RAD51 was validated through immunoblotting (FIG. 6E). Upon UVC irradiation, NSUN2 depletion caused diminished γH2A.X signaling (FIG. 6F) and defective DNA repair activity, as demonstrated by EdU incorporation followed by click chemistry (FIGS. 6G and 13B). The cytotoxicity of 5-azaC could be partially rescued by NSUN2 overexpression (FIG. 13C), reinforcing the role of NSUN2 in the RNA-dependent effects of 5-azaC.

Further, the inventors reasoned that if NSUN2 is one of the key functional targets of 5-azaC, its depletion may mimic effects of 5-azaC during hematopoiesis. MDS are typically characterized by ineffective erythropoiesis (48), and monocytosis has been associated with earlier transformation to AML (49). The use of 5-azaC helps restore erythroid differentiation (34,50). The hematopoietic lineage commitment in Nsun2 knockout (KO) mice was investigated. BM cells purified from 6-month-old Nsun2 KO mice (FIG. 13D) exhibited a reduced frequency of Lin−Sca1+c-Kit+ (LSK) cells (FIG. 6H), indicative of impaired HSPC maintenance. A decrease in granulocyte-monocyte progenitor (GMP) cells and an increase in common myeloid progenitor (CMP) cells was also observed within the Lin−Sca1−c-Kit+ (LKS-) population. Additionally, Nsun2 KO mice exhibited a higher population of Ter119+ erythroid cells in the spleen (FIG. 6I), suggesting a shift toward erythropoiesis as reported when using 5-azaC in patients. In vitro colony-forming unit cell (CFU-C) assays using Nsun2 KO BM cells revealed a lower frequency and number of CFU-granulocyte-macrophage (CFU-GM) colonies, accompanied by an increase in burst-forming unit-erythroid (BFU-E) and CFU-granulocyte-erythrocyte-monocyte-megakaryocyte (CFU-GEMM) colonies (FIG. 6J). This is consistent with the observed reduction in GMPs and expansion of CMPs in Nsun2KO mice. Furthermore, CFU-GEMM and BFU-E colonies derived from Nsun2 KO BM cells were larger in size (FIG. 6K), suggesting altered differentiation dynamics. The progeny of Nsun2 KO CFU-C cultures exhibited a reduced Lin−c-Kit+ (LK) population (FIG. 13E) and an increased proportion of Ter119+ erythroid cells (FIG. 13F), further supporting a shift toward erythropoiesis with impeded myelopoiesis after Nsun2 deletion.

To further evaluate the impact of Nsun2loss on the repopulating capacity of HSPCs, the inventors performed in vivo competitive repopulation assays. Total donor BM cells (CD45.2⁺) from either wildtype (WT) or Nsun2 KO mice were mixed with equal numbers of competitor BM cells (CD45.1⁺) and transplanted into lethally irradiated recipient mice (CD45.1⁺). While WT donor cell (CD45.2⁺) chimerism in peripheral blood (PB) of recipients remained at ~50% starting 4 weeks after transplantation, CD45.2⁺ cells from Nsun2 KO BM progressively declined, reaching less than 10% at 32 weeks after transplantation (FIG. 13G). Similar trends in donor cell (CD45.2⁺) chimerism were observed in BM when recipients were sacrificed 32 weeks after transplantation (FIG. 13H). Furthermore, compared to WT BM cells, the Nsun2KO BM cells produced significantly lower percentage of GMP cells in recipient BM (FIG. 13I), leading to significantly fewer myeloid (Gr-1⁺/CD11b⁺) cells in PB and BM, as well as increased Ter119⁺ erythroid cells in BM (FIGS. 13J and 13K). These results suggested an important role for NSUN2 in hematopoiesis maintenance and HSPC function in vivo, particularly in myeloid and erythroid lineage commitment and differentiation.

Given the established role of erythropoiesis in MDS, the inventors were particularly interested in the impact of NSUN2 on erythroid differentiation. By conducting in vitro erythroid differentiation using HSPCs derived from the BM of Nsun2 KO and WT mice, the inventors observed dramatically enhanced erythroid differentiation upon Nsun2 deficiency (FIGS. 6L and 13L-13N), consistent with the increased erythropoiesis observed in CFU-C assay using BM from Nsun2 KO mice (FIGS. 6J, 6K and 13F). Overall, the results demonstrated that NSUN2 depletion significantly promoted erythroid lineage commitment, with effects consistent with those observed with 5-azaC treatment.

### Example 8—Correlation of Leukemia Cell Line Mutations with 5-azaC Sensitivity

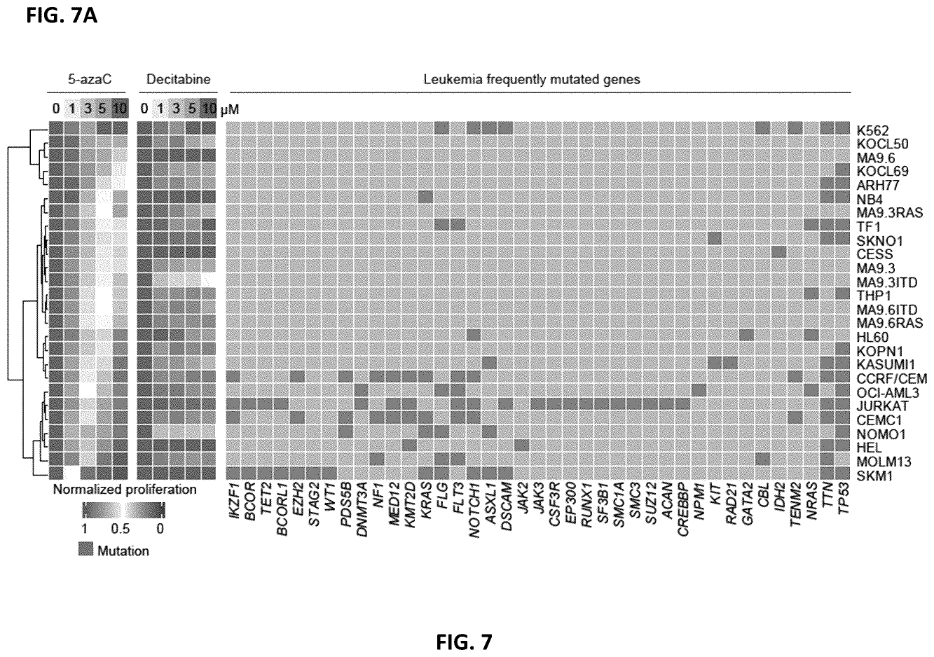

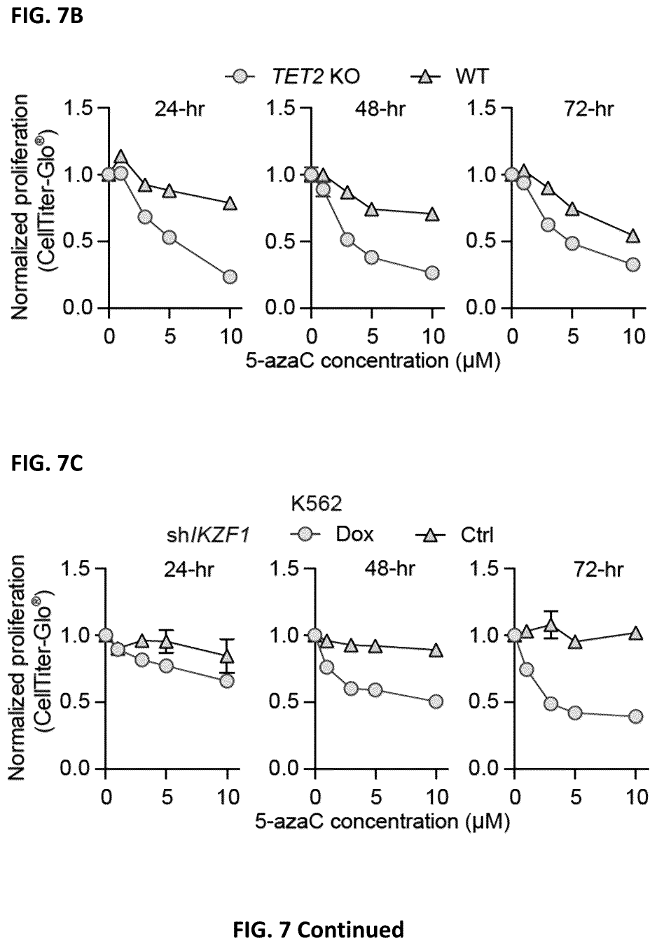

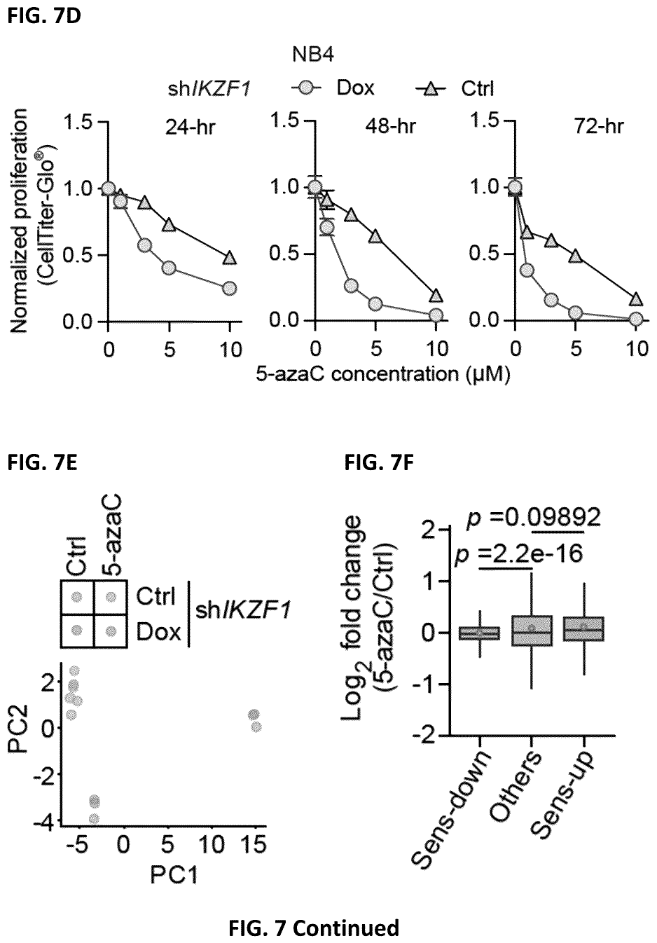

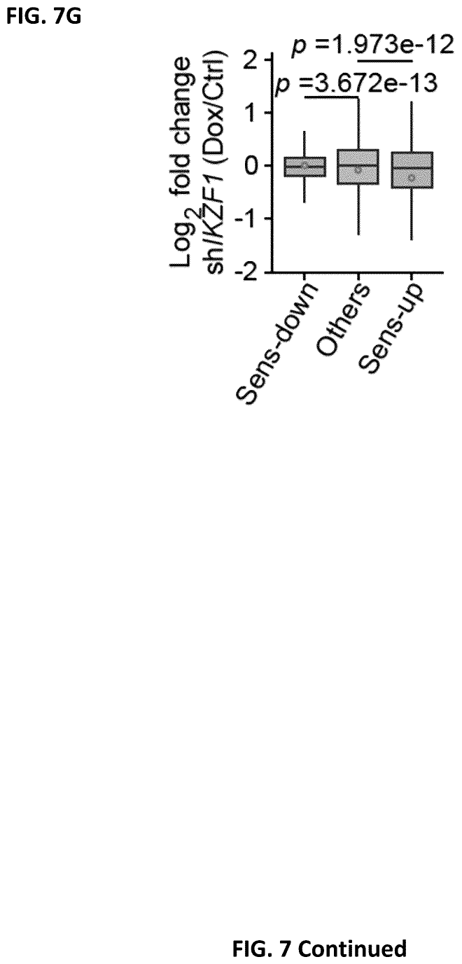

Leukemia cell lines exhibit varying sensitivity to 5-azaC (FIGS. 1A-1D). While the DNA-dependent cytotoxicity of 5-azaC has been extensively studied (51-55), the factors influencing sensitivity to its RNA-dependent effects remain poorly understood. To address this and draw direct connection of the new pathways that were uncovered with 5-azaC sensitivity, the inventors screened 26 different leukemia cell lines for their response to 5-azaC at early time points (FIG. 7A). Consistent with the above reported findings, 24-hour decitabine treatment had little impact on proliferation, whereas 5-azaC led to varying levels of growth inhibition across different cell lines. The inventors integrated publicly available mutation data and identified mutations enriched in 5-azaC sensitive cells that were frequently observed in clinical MDS and AML samples. This included TET2, which catalyzes oxidation of RNA m⁵C. This aligns with previous reports that MDS and AML patients harboring TET2 mutations tend to respond more effectively to 5-azaC treatment (34,51,56), and agrees with the m⁵C/MBD6/PR-DUB/H2AK119ub regulatory axis the inventors reported recently (28) and in this study. Consistently, TET2 KO THP1 cells exhibited increased sensitivity to 5-azaC, particularly at early time points (FIG. 7B). These findings indicate that TET2 mutation is likely a reliable biomarker for the 5-azaC sensitivity. Beyond TET2, mutations in ASXL1, a component of the PR-DUB complex, were also enriched in 5-azaC-sensitive cells. The presence of mutations affecting RNA m⁵C/MBD6/PR-DUB/H2AK119ub pathway components further supports the role of caRNA m⁵C disruption in 5-azaC-induced effects.

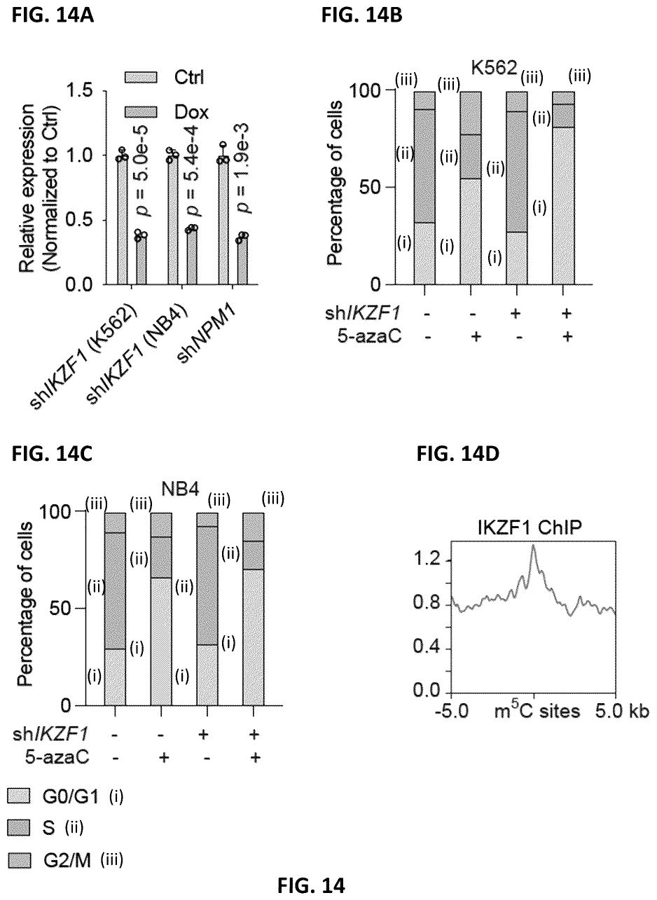

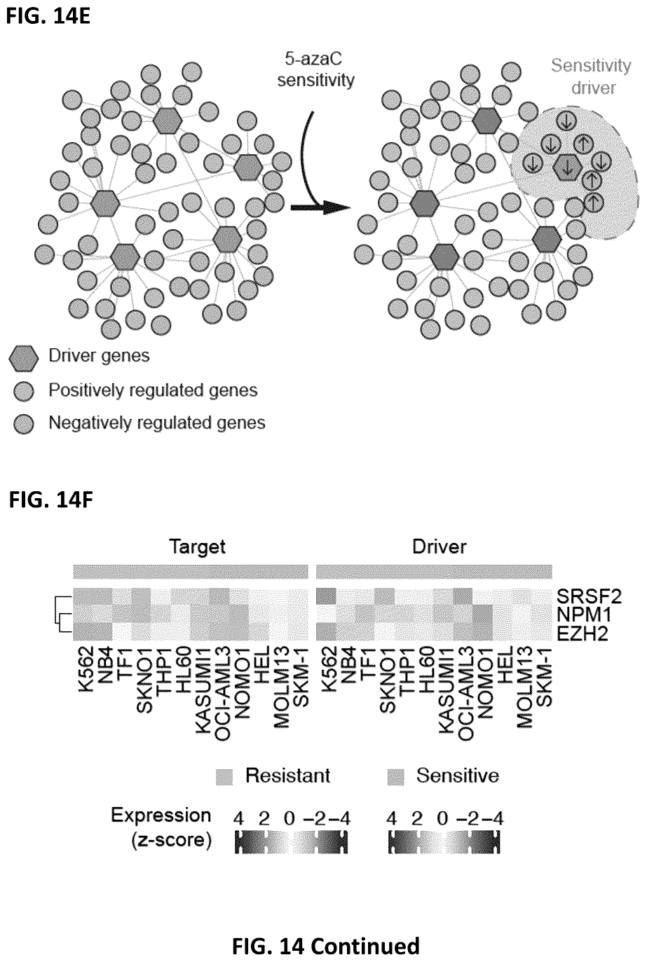

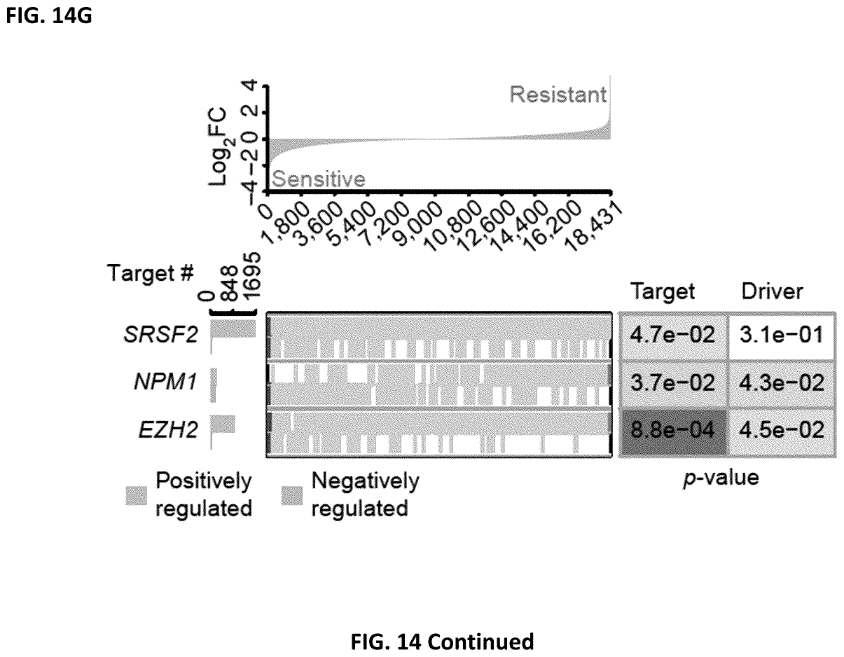

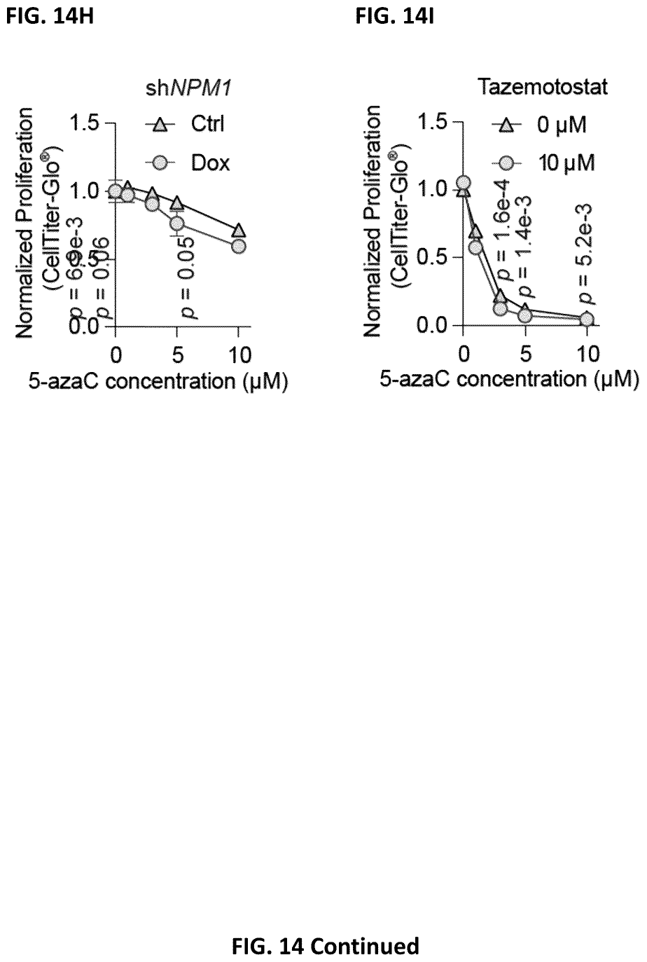

In addition to RNA m⁵C related pathways, the inventors identified IKZF1 as a relevant mutation. While IKZF1 mutations are more commonly observed in acute lymphoblastic leukemia (ALL) (57), they have also been identified in AML as an independent marker of adverse risk with unique clinical phenotypes (58). Moreover, IKZF1 degrader have shown efficacy in eliminating MLL1-rearranged (MLL-r) AML cells (61). The proliferation assays described herein revealed that IKZF1 depletion sensitized K562 cells (FIGS. 7C and 14A) and NB4 cells (FIG. 7D) to 5-azaC treatment. Cell cycle analysis described herein demonstrated stronger decrease of S-phase population in IKZF1 depleted cells (FIG. 14B and C). RNA-seq revealed that treatment with 3 μM 5-azaC induced only subtle transcriptomic changes in K562 cells at 24 hours, consistent with its resistance to 5-azaC. In contrast, IKZF1 depletion markedly amplified the transcriptional response (FIG. 7E). Genes significantly up-or down-regulated by 5-azaC in IKZF1-depleted K562 cells, denoted as Sens-up and Sens-down, respectively, showed only mild up- and down-regulated in control cells (FIG. 7F). However, the Sens-up and Sens-down genes were notably down- and up-regulated after IKZF1 depletion (FIG. 7G), which, without being bound to any theory, suggested that IKZF1 depletion may reprogram the transcriptomic landscape, rendering cells more reliant on Sens-down genes and more vulnerable to Sens-up gene activation, thereby increasing sensitivity to 5-azaC. IKZF1 ChIP-seq from the ENCODE database revealed enrichment of IKZF1 binding at genomic loci containing m⁵C sites previously identified in K562 cells⁷⁸ (FIG. 14D), which suggested a functional interplay between IKZF1 and RNA m⁵C-mediated regulation. These findings highlight IKZF1 inhibition as a viable candidate for combination therapy with 5-azaC.

To further investigate potential drivers of 5-azaC sensitivity, the inventors applied the network inference algorithm NetBID2 (60) to model regulatory activity of frequently mutated genes in MDS and AML. A leukemia-specific gene regulatory network was constructed with SJARACNe algorithm, using RNA-seq dataset of 72 myeloid-origin leukemia cell lines from DepMap portal (61). After overlaying this network with the five most 5-azaC sensitive (SKM-1, MOLM13, HEL, NOMO1, OCI-AML3) and six most 5-azaC resistant (K562, NB4, TF1, SKNO1, THP1, HL60) leukemia cell lines, the inventors assessed hub gene activity based on two criteria: 1) differential expression between sensitive and resistant cell lines; and 2) differential expression of target genes between the two groups (FIG. 14E). This analysis identified three candidate drivers of 5-azaC sensitivity: SRSF2, NPM1 and EZH2 (FIG. 14F-14G). The expression of these genes and their targets correlated 5-azaC sensitivity (FIG. 14F). Positively regulated target genes of these candidate drivers were expressed at lower levels in sensitive cell lines, while negatively regulated targets were more highly expressed (FIG. 14G). Additionally, EZH2 mutations were enriched in 5-azaC-sensitive leukemia cells (FIG. 7A). Further validation revealed that NPM1 KD increased THP1 sensitivity to 5-azaC (FIG. 14H), and pretreatment of HEL cells with EZH2 inhibitor tazemetostat let to a modest increase in 5-azaC sensitivity (FIG. 14I).

These analyses and results further support the RNA-dependent SRSF2/p300/H3K27ac pathway uncovered in this study. Additionally, NSUN2 inhibition would increase H2AK119ub, which is known to recruit PRC2 for H3K27me3 deposition. The EZH2 mutation may be associated with this RNA-dependent pathway as well, which requires future studies. Overall, the leukemia cell line screen and network analysis disclosed herein identified new candidate mutations that can serve as biomarkers for predicting 5-azaC sensitivity and/or as potential therapeutic targets for combination treatments.

### Example 9—Discussion

As a ribonucleoside analogue, 5-azacytidine (5-azaC) can be incorporated into both nascent RNA and nascent DNA to covalently inhibit RNA and DNA methyltransferases. While traditionally regarded as a DNA hypomethylating agent with consolidated DNA hypomethylation effects after a period of time, 5-azaC leads to differential gene expression that could not be explained by its DNA hypomethylation changes (1-4). Notably, 5-azaC must be converted into decitabine to be incorporated into DNA, whereas more than 80% of 5-azaC is directly incorporated into RNA, inducing more significant inhibition of RNA m⁵C installation. Here, amongst other things, the inventors have demonstrated that: i) 5-azaC treatment causes RNA m⁵C hypomethylation within the first 24 hours, and this effect is sufficient to drive cytotoxicity in leukemia cells, independent of DNA hypomethylation; ii) the RNA m⁵C hypomethylation explains the gene repression effects of 5-azaC; iii) two identified pathways were responsible for the observed gene repression by 5-azaC, depletion of m⁵C in caRNA disrupts the MBD6-mediated H2AK119ub deubiquitination, leading to globally elevated H2AK119ub levels, while in parallel it impairs SRSF2 binding to chromatin, reducing p300 recruitment for H3K27ac deposition; iv) NSUN2 is the primary target responsible for 5-azaC-induced gene repression, and animal model studies demonstrate that Nsun2 depletion in HSPCs causes hematopoietic lineage commitment towards erythroid; and v) that TET2 and/or IKZF1 mutation can sensitize 5-azaC treatment, consistent with the RNA-dependent cytotoxicity of 5-azaC in leukemia cells. In summary, the findings reported herein highlight the transcription repression effects of 5-azaC through caRNA m⁵C depletion, providing insights into its mechanism of action, predictors of treatment efficacy, and future directions for therapy developments based on 5-azaC.

Some patients that benefited from 5-azaC treatment did not exhibit DNA demethylation (5,62), and hypomethylated samples displayed differential gene expression patterns that could not be explained by DNA methylation changes (1-4). By examining transcriptional dysregulation at early time points, the inventors have demonstrated that the NSUN2-mediated RNA methylation pathways are responsible for transcriptional repression observed with 5-azaC treatment. The RNA-dependent effects may be more transient due to the dynamic nature of RNA synthesis and degradation, in contrast to the heritable stability of DNA methylation. This potentially explains the necessity for continuous administration of 5-azaC in treatment (63). The opposing effects of RNA- and DNA-dependent transcriptional regulation can be complicated and may amplify or attenuate each other. It might be beneficial to develop small molecule inhibitors that selectively target the RNA m⁵C-dependent pathways, as decitabine showed less efficacy than 5-azaC in clinical treatment in general (16-18).

Mechanistically, the inventors have uncovered that RNA-dependent transcriptional repression leads to the downregulation of cell cycle and DNA repair genes, impairing leukemia cell survival, increasing cellular stress, and promoting apoptosis. This also supports the contribution of RNA-dependent transcription suppression pathway to the enhanced efficacy of 5-azaC when combined with venetoclax. The transcriptional repression by 5-azaC was mediated by two pathways: the m⁵C/MBD6/PR-DUB/H2AK119ub axis and the m5C/SRSF2/p300/H3K27ac axis. The elevated H2AK119ub and reduced DNA 5mC are two opposing forces that may neutralize the effects. Notably, the inventors observed persistent H3K27ac reduction during prolonged 5-azaC treatment. H3K27ac is associated with actively transcribed genes, and its local chromatin environment may be less impacted by the DNA hypomethylating effects of 5-azaC, leading to a sustained decrease in transcriptional activity. The varying contributions of H2AK119ub and H3K27ac regulation across heterogeneous leukemia cases may serve as predictive markers for the relative dominance of DNA- and RNA-dependent effects during long-term treatment.

Furthermore, NSUN2-dependent regulation of hematopoietic lineage commitment aligns with the ability of 5-azaC to restore erythropoiesis, reinforcing the therapeutic relevance of RNA m⁵C regulation in hematological disorders.

This study primarily focused on the caRNA-dependent transcriptional repression by 5-azaC, but its effects on other RNA species (mRNA, rRNA and tRNA) should be explored in the future. Herein and in their other works the inventors have shown that NSUN2 is the primary enzyme responsible for the caRNA demethylation and transcription repression caused by 5-azaC treatment. The inventors have carefully examined Nsun2 knockout mice and observed shifted hematopoietic lineage commitment towards erythropoiesis, consistent with the role of 5-azaC in restoring erythroid differentiation (34,50).

The heterogeneity of myeloid malignancies caused varying response to 5-azaC treatment. A previous study comparing 5-azaC-sensitive and -resistant cell lines found differences in RNA m⁵C methyltransferase levels and their related transcriptional regulation (19). The immediate results fill in a critical missing piece in understanding the RNA-dependent transcription regulation by 5-azaC, which leads to cell cycle arrest and attenuated DNA damage repair. Consistent with the importance of the RNA-dependent function, the leukemia cell line screen and gene network inference reported herein revealed mutations related to RNA m⁵C regulation, including TET2, ASXL1 and SRSF2. These findings illustrated the essential role of the RNA-dependent mechanisms in determining 5-azaC sensitivity. Additionally, the inventors have identified EZH2, the methyltransferase for H3K27me3, which is closely linked to the regulation of H2AK119ub (64) described above. The results have further confirmed TET2 as a predictive biomarker for 5-azaC sensitivity. In parallel, the inventors have also revealed that IKZF1 depletion sensitizes leukemia cells to 5-azaC, identifying IKZF1 as a new target to synergize with 5-azaC.

In summary, this study unveils that 5-azaC mediates transcriptional repression through RNA m⁵C-dependent regulation of H2AK119ub and H3K27ac. The RNA-dependent pathways disrupt cell cycle and DNA repair pathways, contribute to 5-azaC-induced cytotoxicity, and play a crucial role in modulating sensitivity of myeloid malignant cells to 5-azaC.

### Example 10—Design and Testing of IKZF1, EZH2, and/or NPM1 Targeting Proteolysis Targeting Chimera

As a method to inhibit of ikaros family zinc finger protein 1 (IKZF1), enhancer of zeste homolog 2 (EZH2), and/or nucleophosmin 1 (NPM1), proteolysis targeting chimera (PROTAC) are designed and tested. A chemical library is screened for affinity to IKZF1, EZH2, and/or NPM1 to identify a ligand binding domain. IKZF1, EZH2, and/or NPM1 ligands are covalently linked to a molecule that binds E3 ubiquitin ligase using methods described in the art. The PROTAC will be tested in vitro for degradation of IKZF1, EZH2, and/or NPM1 in cultured cells before administering in vivo (e.g., in a cancer mouse model). Following administration of the IKZF1, EZH2, and/or NPM1 targeting PROTAC, diseased cells are attenuated and/or sensitized to the effects of 5-azaC.

### Example 11—Identification and Testing of NSUN2 Inhibitors

In the following examples, a novel class of small-molecule modulators targeting RNA m³C methyltransferases, NSUN2 and NSUN6, are identified. Through high-throughput screening and optimization, compounds such as NS71 have been identified with favorable biochemical, cellular, and pharmacokinetic properties. These compounds exhibited selective target engagement, modulate RNA m³C levels, and demonstrated anti-proliferative activity in leukemia models. Collectively, the disclosed findings indicated RNA m³C methyltransferases as actionable therapeutic targets and support the development of NSUN2/6 inhibitors as potential treatments for cancer and other diseases associated with dysregulated RNA methylation.

Through fluorescence polarization (FP)-based high-throughput screening for NSUN2 inhibitors, followed by structure-guided optimization, a novel small-molecule compound, designated NS71, was identified. NS71 exhibited a strong binding affinity against NSUN2, with an IC₅₀ of approximately 0.2 μM (Table 1). In addition, NS71 demonstrated potent anti-proliferative activity in acute myeloid leukemia (AML) cell models (Table 2).

TABLE 1
IC₅₀ values of the compounds with NSUN2

| | IC₅₀ |
|---|---|
| Compd. | (μM) |
| NS-13 | 3.8 |
| NS-14 | 2 |
| NS-15 | 2.2 |
| NS-17 | 18 |
| NS-18 | >20 |
| NS-19 | >20 |
| NS-20 | >20 |
| NS-21 | 15.5 |
| NS-22 | >20 |
| NS-31 | 1.1 |
| NS-32 | 1.6 |
| NS-33 | 0.5 |
| NS-34 | 1.7 |
| NS-35 | 1.1 |
| NS-36 | 3.3 |
| NS-37 | 1.8 |
| NS-38 | 2.5 |
| NS-39 | 0.6 |
| NS-40 | 0.65 |
| NS-41 | 5.2 |
| NS-42 | 0.56 |
| NS-43 | >20 |
| NS-44 | 2.5 |
| NS-45 | 0.33 |
| NS-46 | 6.3 |
| NS-47 | 1.3 |
| NS-48 | 0.74 |
| NS-49 | 0.63 |
| NS-50 | 0.94 |
| NS-51 | 1.2 |
| NS-52 | 0.64 |
| NS-53 | 0.72 |
| NS-54 | 0.63 |
| NS-55 | 0.65 |
| NS-56 | 1.1 |
| NS-57 | 0.72 |
| NS-58 | 1 |
| NS-59 | 0.68 |
| NS-60 | 0.62 |
| NS-61 | 0.59 |
| NS-62 | 10 |
| NS-64 | 1.1 |
| NS-65 | 2.1 |
| NS-66 | 0.65 |
| NS-67 | 0.75 |
| NS-68 | 0.6 |
| NS-69 | 0.52 |
| NS-70 | 0.74 |
| NS-71 | 0.26 |
| NS-72 | 0.78 |
| NS-73 | >20 |
| NS-74 | >20 |
| NS-75 | >20 |
| NS-76 | >20 |
| NS-77 | >20 |
| NS-84 | 0.45 |
| NS-91 | 2.2 |

TABLE 2
IC₅₀ values of the compounds in Molm13 cells.

| Compd. | IC₅₀ (μM) |
|---|---|
| NS-13 | >50 |
| NS-15 | >50 |
| NS-22 | 3.5 |
| NS-31 | 23 |
| NS-32 | 15.5 |
| NS-33 | 4.3 |
| NS-34 | 6.5 |
| NS-35 | 6 |
| NS-36 | 8.5 |
| NS-37 | 5.1 |
| NS-38 | 6.5 |
| NS-39 | 5.5 |
| NS-44 | 6 |
| NS-47 | 5.5 |
| NS-49 | 7.4 |
| NS-50 | 4 |
| NS-51 | 2.8 |
| NS-52 | 6 |
| NS-53 | 5.7 |
| NS-54 | 2.8 |
| NS-55 | 40 |
| NS-56 | 15.3 |
| NS-60 | >50 |
| NS-64 | >50 |
| NS-65 | 0.9 |
| NS-66 | 9.2 |
| NS-67 | 4.9 |
| NS-68 | 4.3 |
| NS-69 | 4.8 |
| NS-70 | 14.3 |
| NS-71 | 2.4 |
| NS-72 | 5.4 |
| NS-84 | 7.4 |

NS71 exhibited inhibitory activity against NSUN2 and NSUN6, but not NSUN5 (Tables 3-4), indicating selectivity within the NSUN family. Notably, leukemia models harboring TET2 mutations, engineered TET2 knockout cell lines, or rearrangements involving MLL showed markedly increased sensitivity to NSUN2 inhibition compared to wild-type counterparts. These findings support the potential clinical utility of NSUN2 inhibitors, including NS71, in targeting molecularly defined patient populations. In particular, leukemias characterized by TET2 alterations or MLL rearrangements may represent responsive subgroups, enabling biomarker-driven patient stratification and precision therapeutic intervention.

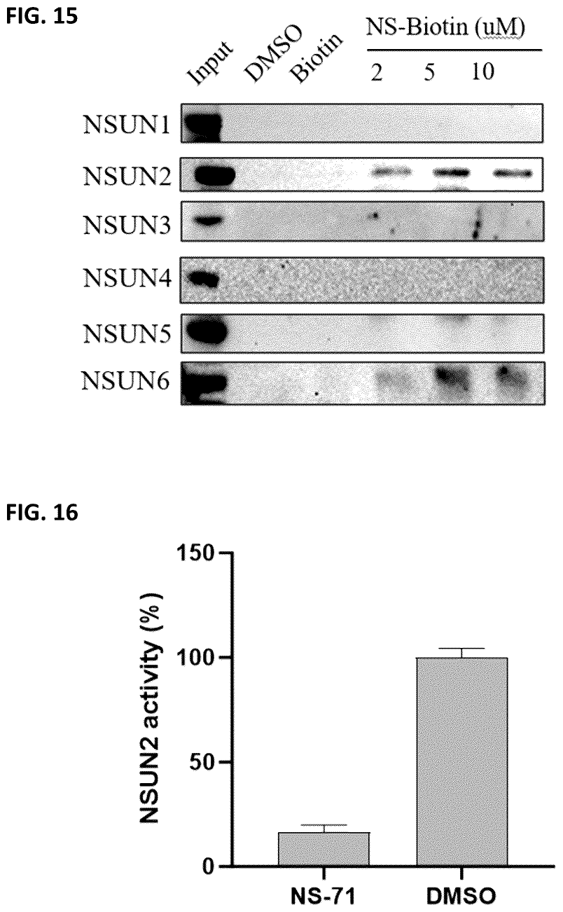

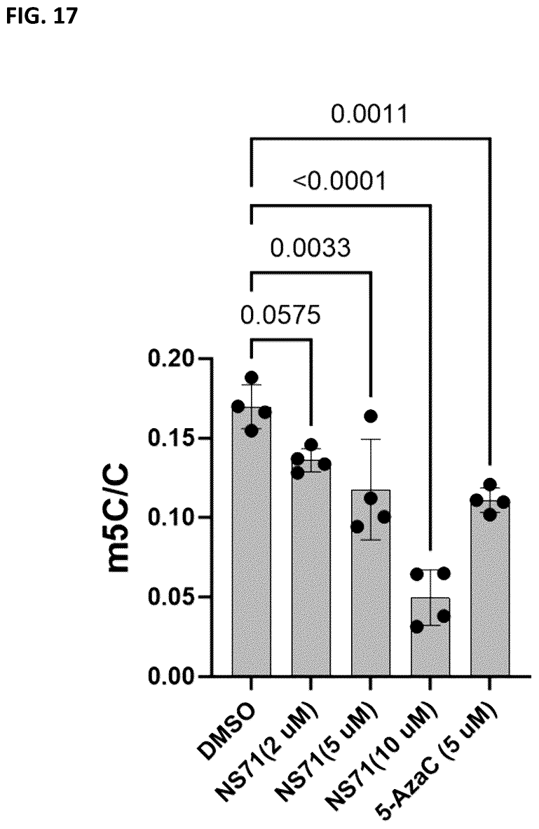

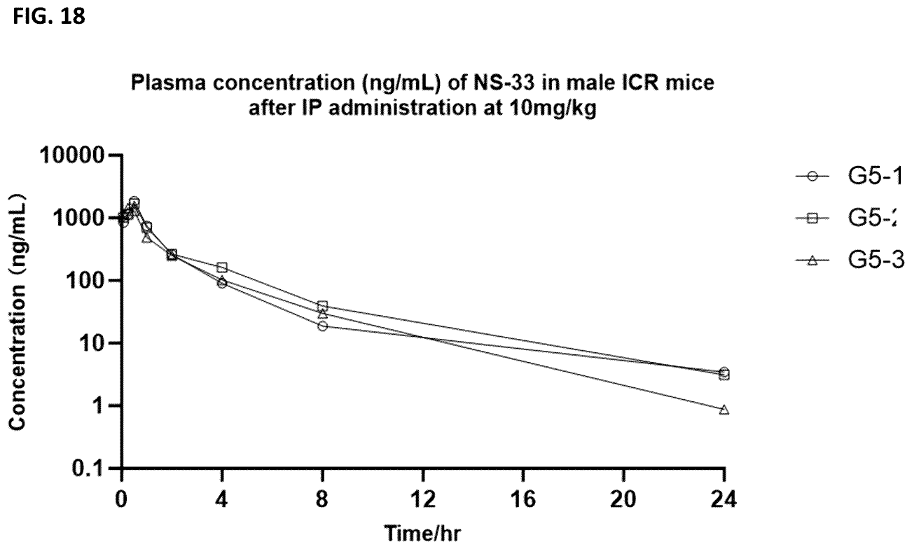

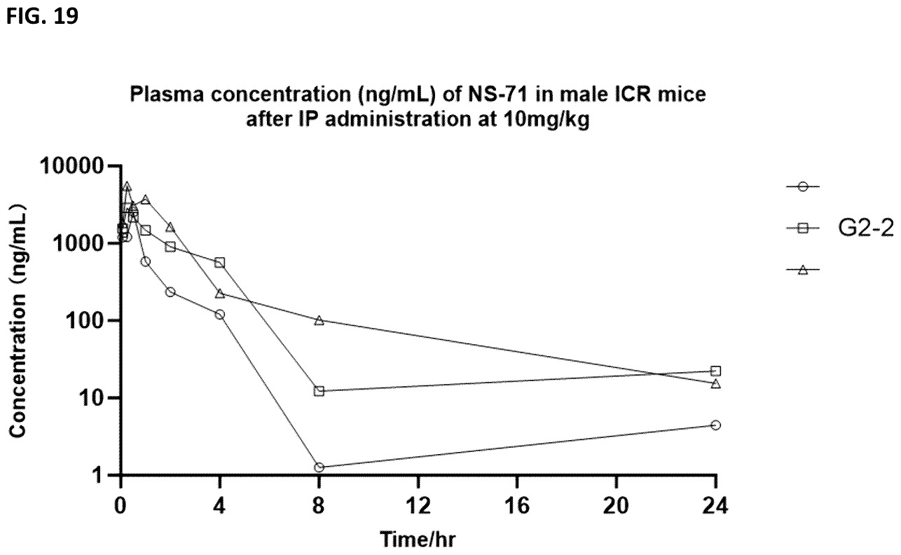

To further characterize target engagement, a biotinylated derivative of NS71 (NS-Biotin) was synthesized and evaluated in pull-down assays. The results indicated that NS-Biotin selectively enriches NSUN2 and NSUN6 proteins, which supports the selective interaction of NS71 with RNA m³C methyltransferases (FIG. 15). Washout experiments further suggested that NS71 forms a covalent interaction with NSUN2 protein, as evidenced by decreased methyltransferase activity of NSUN2 following complete compound removal (FIG. 16). In cellular systems, NS71 induced a dose-dependent decreased in RNA mC levels (FIG. 17), consistent with inhibition of NSUN2/6-mediated methylation activity. Pharmacokinetic studies indicated that NS71 exhibited favorable in vivo exposure and pharmacokinetic properties (FIGS. 18-19). These results support its potential for further therapeutic development.

TABLE 3
IC₅₀ values of NS71 in a panel of leukemia cells.

| Key genetic | Cell | IC₅₀ | Key genetic | Cell | IC₅₀ |
|---|---|---|---|---|---|
| features | line | (μM) | features | line | (μM) |
| TET2 Mutant | SKM1 | 3.2 | PML-RARA | NB4 | 4.9 |
| MDS | | | | | |
| Engineered | THP1- | 3 | NPM1 | OCI-AML3 | 5 |
| TET2 KO | TET2_KO | | | | |
| | K562- | 3.1 | RUNX1-RUNX1T1 | KASUMI1 | 9 |
| | TET2_KO | | fusion | | |
| MLL-AF9 | MOLM13 | 2.4 | JAK2 V617F | HEL | 9.3 |
| MLL-AF9 | MA9.3 | 2.3 | NOTCH1 activation TLL | CCRF/CEM | 10 |
| (engineered | MA9.3RAS | 2.1 | GM-CSF-dependent | TF1 | 11.6 |
| CD34+) | MA9.3ITD | 5.3 | BCR-ABL fusion | K562 | 16 |
| | MA9.6 | 5.8 | MYC amplification | HL60 | 16 |
| | MA9.6RAS | 4.6 | monocytic leukemia | U937 | 17 |
| MLL-AF9 | THP1 | 8.1 | RUNX1-RUNX1T1 | SKNO1 | 8.4 |
| MLL-AF9 | NOMO1 | 5.5 | Normal cells | hPBMC | 15 |
| MLL-AF6 | ML2 | 5 | PDX CELL | PDX2016 | 23 |
| MLL-AF6 | OCI-AML2 | 3.8 | PDX CELL | PDX2017 | 1.3 |
| MLL-ENL | KOCL50 | 7.3 | | | |

TABLE 4
IC₅₀ values of the compounds with NSUN2.

| | IC₅₀ |
|---|---|
| Compd. | (μM) |
| NS-12 | 4.34 |
| NS-33 | 4.43 |
| NS-39 | 3.65 |
| NS-40 | 5.2 |
| NS-45 | 4.5 |
| NS-71 | 3.8 |

TABLE 5
IC₅₀ values of NS71 with NSUN5 and NSUN6.

| | IC₅₀ |
|---|---|
| Compd. | (μM) |
| NSUN5 | >50 |
| NSUN6 | 14 |

### Example 12—Synthesis of NSUN2 Inhibitors

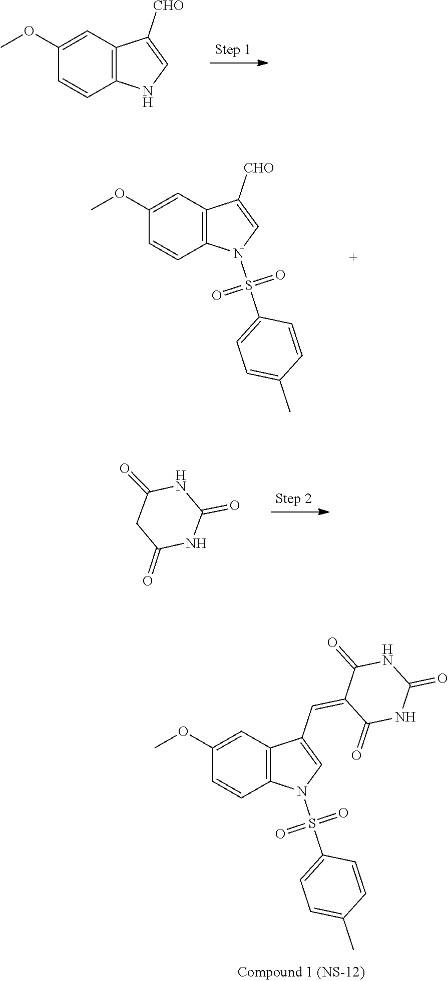 [SMILES: COc1ccc2[nH]cc(C=O)c2c1 ; COc1ccc2c(c1)c(C=O)cn2S(=O)(=O)c1ccc(C)cc1 ; O=C1CC(=O)NC(=O)N1 ; COc1ccc2c(c1)c(C=C1C(=O)NC(=O)NC1=O)cn2S(=O)(=O)c1ccc(C)cc1]

### Step 1. 5-methoxy-1-tosyl-1H-indole-3-carbaldehyde

To a solution of 5-methoxy-1H-indole-3-carbaldehyde (0.2 mg, 1.1 mmol) in DMF (10 mL) was added NaH (68 mg, 60%, 1.7 mmol) in portions at 0° C., the mixture was stirred at this temperature for 15 min. This was followed by the slowly addition of 4-methylbenzenesulfonyl chloride (0.33 g, 1.7 mmol). The resulting solution was stirred for 0.5 h at ambient temperature. Then the reaction was quenched by saturated NH4Cl solution, which was followed by the extraction with ethyl acetate (50 mL*3), and the organic layers was combined and washed by water (50 mL*3) and brine (50 mL). The mixture was dried over anhydrous sodium sulfate, filtered, and concentrated. The obtained crude was purified by silica column chromatography (230-400 mesh), eluting with EtOAc (0-10%) in heptane, to afford INT-1 as a white solid. (ESI): m/z=330.1 [M+H]⁺.

### Step 2. 5-((5-methoxy-1-tosyl-1H-indol-3-yl)methylene)pyrimidine-2,4,6(1H,3H,5H)-trione (Compound 1 (NS-12))

The mix solution of INT-1 (50 mg, 0.15 mmol) and pyrimidine-2,4,6(1H,3H,5H)-trione (19 mg, 0.15 mmol) in EtOH (2 mL) was stirred at 100° C. for 0.5 h, then the precipitated solid was collected by filtration to give NS-12 as a yellow solid. ¹H NMR (400 MHz, DMSO) δ 11.40 (d, J=1.9 Hz, 1H), 11.35 (d, J=1.7 Hz, 1H), 9.62 (s, 1H), 8.46 (s, 1H), 7.97-7.90 (m, 2H), 7.89 (d, J=9.0 Hz, 1H), 7.45 (d, J=8.1 Hz, 2H), 7.34 (d, J=2.5 Hz, 1H), 7.06 (dd, J=9.0, 2.4 Hz, 1H), 3.85 (s, 3H), 2.34 (s, 4H).

### Example 13—Exemplary NSUN2 Inhibitors

The molecules in the following examples were prepared according to the procedures described in Example 12.

TABLE 6
Exemplary compounds

| Compound | | | (ESI): |
|---|---|---|---|
| number | Structure | ¹H NMR | m/z |
| Compound 2 (NS-13) | 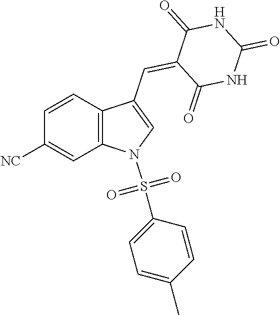 [SMILES: Cc1ccc(S(=O)(=O)n2cc(C=C3C(=O)NC(=O)NC3=O)c3ccc(C#N)cc32)cc1] | ¹H NMR (400 MHz, DMSO) δ 11.46 (d, J = 1.8 Hz, 1H), 11.42 (d, J = 1.9 Hz, 1H), 9.74 (d, J = 0.8 Hz, 1H), 8.49 (dd, J = 1.5, 0.7 Hz, 1H), 8.44 (s, 1H), 8.12 (dd, J = 8.3, 5.6 Hz, 3H), 7.82 (dd, J = 8.3, 1.4 Hz, 1H), 7.52-7.40 (m, 2H), 2.36 (s, 3H). | 434.1 |
| Compound 3 (NS-14) | 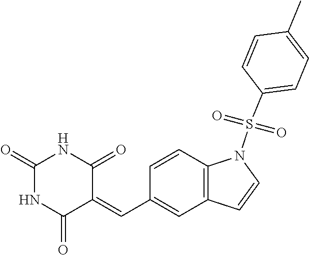 [SMILES: Cc1ccc(S(=O)(=O)n2ccc3cc(C=C4C(=O)NC(=O)NC4=O)ccc32)cc1] | ¹H NMR (400 MHz, DMSO) δ 11.37 (d, J = 1.9 Hz, 1H), 11.22 (d, J = 1.9 Hz, 1H), 8.54 (d, J = 1.7 Hz, 1H), 8.36 (s, 1H), 8.10 (dd, J = 8.9, 1.8 Hz, 1H), 7.98 (d, J = 8.8 Hz, 1H), 7.96-7.92 (m, 2H), 7.91 (d, J = 3.7 Hz, 1H), 7.45-7.37 (m, 2H), 6.95 (dd, J = 3.7, 0.8 Hz, 1H), 2.33 (s, 3H). | 410.1 |
| Compound 4 (NS-15) | 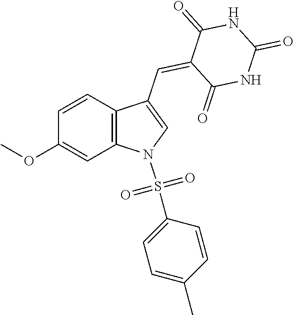 [SMILES: COc1ccc2c(C=C3C(=O)NC(=O)NC3=O)cn(S(=O)(=O)c3ccc(C)cc3)c2c1] | ¹H NMR (400 MHz, DMSO) δ 11.54- 11.16 (m, 2H), 9.54 (d, J = 0.7 Hz, 1H), 8.42 (s, 1H), 7.97 (d, J = 8.4 Hz, 2H), 7.80 (d, J = 8.8 Hz, 1H), 7.47 (d, J = 8.2 Hz, 2H), 7.43 (d, J = 2.3 Hz, 1H), 7.08 (dd, J = 8.8, 2.3 Hz, 1H), 3.87 (s, 3H), 2.35 (s, 3H). | 440.1 |
| Compound 5 (NS-17) | 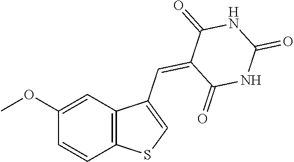 [SMILES: COc1ccc2scc(C=C3C(=O)NC(=O)NC3=O)c2c1] | ¹H NMR (400 MHz, DMSO) δ 11.42- 11.24 (m, 2H), 8.60 (s, 1H), 8.42 (s, 1H), 7.98 (d, J = 8.9 Hz, 1H), 7.48 (d, J = 2.5 Hz, 1H), 7.22 (dd, J = 8.9, 2.5 Hz, 1H), 3.86 (s, 3H). | 303.0 |
| Compound 6 (NS-18) | 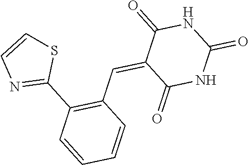 [SMILES: O=C1NC(=O)C(=Cc2ccccc2-c2nccs2)C(=O)N1] | ¹H NMR (400 MHz, DMSO) δ 9.88 (s, 1H), 9.47 (s, 1H), 8.32-8.19 (m, 2H), 8.06 (d, J = 3.6 Hz, 1H), 7.69-7.54 (m, 2H), 7.55-7.44 (m, 1H), 7.12 (s, 1H). | 281.1 |
| Compound 7 (NS-20) | 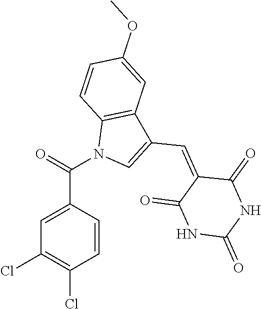 [SMILES: COc1ccc2c(c1)c(C=C1C(=O)NC(=O)NC1=O)cn2C(=O)c1ccc(Cl)c(Cl)c1] | 1H NMR (400 MHz, DMSO) δ 11.38 (d, J = 1.9 Hz, 1H), 11.24 (s, 1H), 9.42 (s, 1H), 8.55 (s, 1H), 8.27 (d, J = 9.0 Hz, 1H), 8.18 (d, J = 2.0 Hz, 1H), 7.94 (d, J = 8.3 Hz, 1H), 7.87 (dd, J = 8.3, 2.0 Hz, 1H), 7.43 (d, J = 2.5 Hz, 1H), 7.14 (dd, J = 9.0, 2.5 Hz, 1H), 3.93 (s, 3H). | 458.0 |
| Compound 8 (NS-21) | 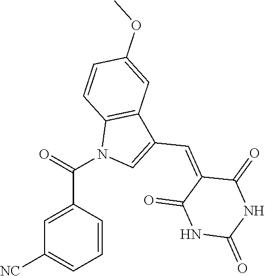 [SMILES: COc1ccc2c(c1)c(C=C1C(=O)NC(=O)NC1=O)cn2C(=O)c1cccc(C#N)c1] | ¹H NMR (400 MHz, DMSO) δ 11.37 (d, J = 1.8 Hz, 1H), 11.24 (d, J = 1.8 Hz, 1H), 9.36 (s, 1H), 8.53 (s, 1H), 8.37 (q, J = 1.1 Hz, 1H), 8.28 (d, J = 9.0 Hz, 1H), 8.25- 8.17 (m, 2H), 7.86 (d, J = 0.6 Hz, 1H), 7.42 (d, J = 2.5 Hz, 1H), 7.16 (s, 1H), 3.93 (s, 3H). | 415.1 |
| Compound 9 (NS-22) | 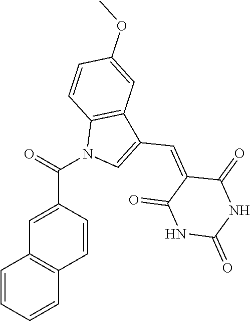 [SMILES: COc1ccc2c(c1)c(C=C1C(=O)NC(=O)NC1=O)cn2C(=O)c1ccc2ccccc2c1] | ¹H NMR (400 MHz, DMSO) δ 11.35 (d, J = 1.9 Hz, 1H), 11.20-11.05 (m, 1H), 9.57 (s, 1H), 8.58 (s, 1H), 8.53 (d, J = 1.7 Hz, 1H), 8.29 (d, J = 9.0 Hz, 1H), 8.23-8.08 (m, 3H), 7.93 (dd, J = 8.5, 1.8 Hz, 1H), 7.73 (ddd, J = 18.0, 8.0, 1.3 Hz, 2H), 7.44 (d, J = 2.5 Hz, 1H), 7.15 (dd, J = 9.0, 2.5 Hz, 1H), 3.94 (s, 3H). | 440.1 |
| Compound 10 (NS-31) | 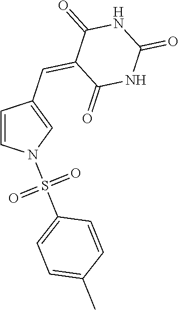 [SMILES: Cc1ccc(S(=O)(=O)n2ccc(C=C3C(=O)NC(=O)NC3=O)c2)cc1] | ¹H NMR (400 MHz, DMSO) δ 11.27 (d, J = 1.9 Hz, 1H), 11.23-11.13 (m, 1H), 8.73 (t, J = 1.9 Hz, 1H), 8.16 (s, 1H), 7.97- 7.87 (m, 2H), 7.57-7.43 (m, 3H), 7.28 (dd, J = 3.4, 1.5 Hz, 1H), 2.40 (s, 3H). | 360.0 |
| Compound 11 (NS-32) | 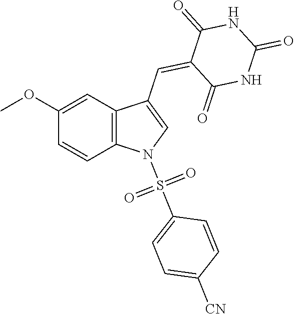 [SMILES: COc1ccc2c(c1)c(C=C1C(=O)NC(=O)NC1=O)cn2S(=O)(=O)c1ccc(C#N)cc1] | ¹H NMR (400 MHz, DMSO) δ 11.48- 11.30 (m, 2H), 9.58 (s, 1H), 8.45 (s, 1H), 8.29-8.19 (m, 2H), 8.19-8.01 (m, 3H), 7.92 (d, J = 9.1 Hz, 1H), 7.37 (d, J = 2.5 Hz, 1H), 7.08 (dd, J = 9.1, 2.5 Hz, 1H), 3.86 (s, 4H). | 451.0 |
| Compound 12 (NS-33) | 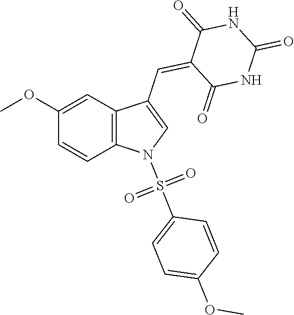 [SMILES: COc1ccc(S(=O)(=O)n2cc(C=C3C(=O)NC(=O)NC3=O)c3cc(OC)ccc32)cc1] | 1H NMR (400 MHz, DMSO) δ 11.40 (s, 1H), 11.34 (s, 1H), 9.62 (s, 1H), 8.47 (s, 1H), 8.05-7.94 (m, 2H), 7.90 (d, J = 9.0 Hz, 1H), 7.35 (d, J = 2.4 Hz, 1H), 7.19- 7.10 (m, 2H), 7.07 (dd, J = 9.1, 2.4 Hz, 1H), 3.86 (s, 3H), 3.81 (s, 3H). | 456.1 |
| Compound 13 (NS-34) | 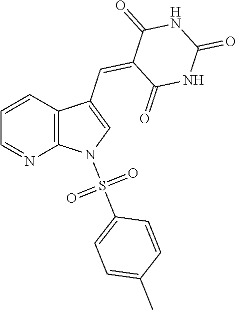 [SMILES: Cc1ccc(S(=O)(=O)n2cc(C=C3C(=O)NC(=O)NC3=O)c3cccnc32)cc1] | 1H NMR (400 MHz, DMSO) δ 11.46- 11.35 (m, 2H), 9.79 (s, 1H), 8.49 (d, J = 4.8 Hz, 1H), 8.46 (s, 1H), 8.40 (dd, J = 8.1, 1.5 Hz, 1H), 8.11 (d, J = 8.4 Hz, 2H), 7.52- 7.41 (m, 3H), 2.37 (s, 3H). | 411.1 |
| Compound 14 (NS-35) | 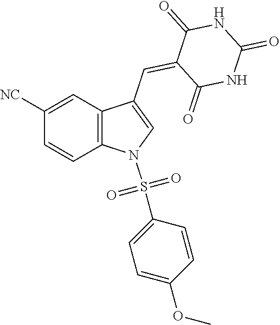 [SMILES: COc1ccc(S(=O)(=O)n2cc(C=C3C(=O)NC(=O)NC3=O)c3cc(C#N)ccc32)cc1] | 1H NMR (400 MHz, DMSO) δ 11.45 (d, J = 1.8 Hz, 1H), 11.40 (s, 1H), 9.69 (s, 1H), 8.56 (d, J = 1.5 Hz, 1H), 8.48 (s, 1H), 8.19 (d, J = 8.7 Hz, 1H), 8.08 (d, J = 9.1 Hz, 2H), 7.91-7.83 (m, 1H), 7.17 (d, J = 9.1 Hz, 2H), 3.83 (s, 3H). | 440.1 |
| Compound 15 (NS-36) | 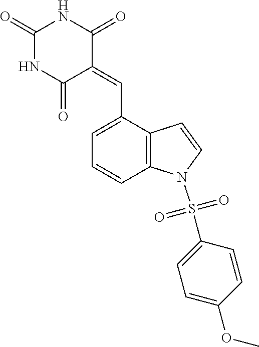 [SMILES: COc1ccc(S(=O)(=O)n2ccc3c(C=C4C(=O)NC(=O)NC4=O)cccc32)cc1] | ¹H NMR (400 MHz, DMSO) δ 11.41 (s, 1H), 11.18 (s, 1H), 8.49 (s, 1H), 8.07 (d, J = 8.3 Hz, 1H), 8.02-7.83 (m, 4H), 7.41 (t, J = 8.0 Hz, 1H), 7.10 (d, J = 8.5 Hz, 2H), 6.88 (d, J = 3.7 Hz, 1H), 3.80 (s, 3H). | 426.1 |
| Compound 16 (NS-37) | 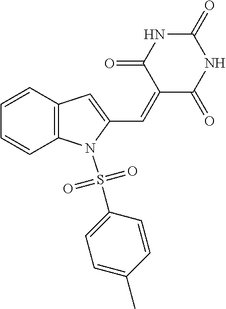 [SMILES: Cc1ccc(S(=O)(=O)n2c(C=C3C(=O)NC(=O)NC3=O)cc3ccccc32)cc1] | ¹H NMR (400 MHz, DMSO) δ 11.47 (s, 1H), 11.29 (s, 1H), 8.85 (s, 1H), 8.25 (s, 1H), 8.15 (d, J = 8.6 Hz, 1H), 7.76 (d, J = 7.9 Hz, 1H), 7.64 (d, J = 8.4 Hz, 2H), 7.55 (s, 1H), 7.36 (s, 1H), 7.31 (d, J = 8.1 Hz, 2H), 2.29 (s, 4H). | 410.1 |
| Compound 17 (NS-38) | 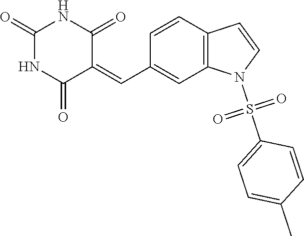 [SMILES: Cc1ccc(S(=O)(=O)n2ccc3ccc(C=C4C(=O)NC(=O)NC4=O)cc32)cc1] | ¹H NMR (400 MHz, DMSO) δ 11.40 (d, J = 1.9 Hz, 1H), 11.33 (d, J = 1.9 Hz, 1H), 9.29 (s, 1H), 8.43 (s, 1H), 8.08-7.95 (m, 3H), 7.89-7.79 (m, 1H), 7.68 (d, J = 8.2 Hz, 1H), 7.39 (d, J = 8.3 Hz, 2H), 6.91 (dd, J = 3.6, 0.8 Hz, 1H), 2.32 (s, 3H). | 410.1 |
| Compound 18 (NS-39) | 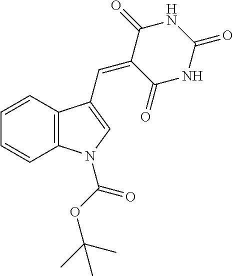 [SMILES: CC(C)(C)OC(=O)n1cc(C=C2C(=O)NC(=O)NC2=O)c2ccccc21] | ¹H NMR (400 MHz, DMSO) δ 11.38 (d, J = 1.9 Hz, 1H), 11.34-11.17 (m, 1H), 9.70 (s, 1H), 8.57 (s, 1H), 8.23-8.10 (m, 1H), 7.97-7.83 (m, 1H), 7.47 (ddd, J = 8.7, 7.5, 1.4 Hz, 2H), 1.68 (s, 9H). | 356.1 |
| Compound 19 (NS-40) | 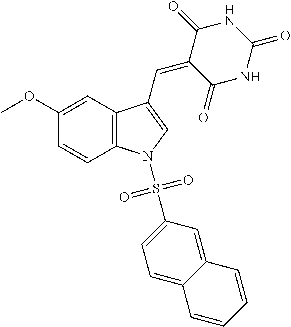 [SMILES: COc1ccc2c(c1)c(C=C1C(=O)NC(=O)NC1=O)cn2S(=O)(=O)c1ccc2ccccc2c1] | ¹H NMR (400 MHz, DMSO) δ 11.49- 11.29 (m, 2H), 9.70 (s, 1H), 8.99 (d, J = 2.0 Hz, 1H), 8.46 (s, 1H), 8.29 (d, J = 8.0 Hz, 1H), 8.15 (d, J = 8.8 Hz, 1H), 8.04 (d, J = 7.9 Hz, 2H), 7.98 (d, J = 9.1 Hz, 1H), 7.87 (dd, J = 8.8, 2.1 Hz, 1H), 7.81-7.69 (m, 3H), 7.33 (d, J = 2.5 Hz, 1H), 7.06 (dd, J = 9.1, 2.5 Hz, 1H), 3.83 (s, 3H). | 476.1 |
| Compound 20 (NS-41) | 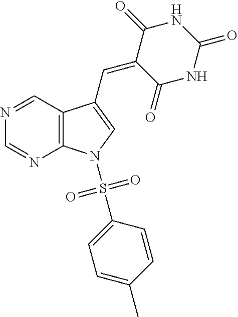 [SMILES: Cc1ccc(S(=O)(=O)n2cc(C=C3C(=O)NC(=O)NC3=O)c3cncnc32)cc1] | ¹H NMR (400 MHz, DMSO) δ 11.47 (s, 1H), 11.43 (s, 1H), 9.63 (s, 1H), 9.44 (s, 1H), 9.08 (s, 1H), 8.49 (s, 1H), 8.12 (d, J = 8.3 Hz, 2H), 7.51 (d, J = 8.2 Hz, 2H), 2.38 (s, 3H). | 412.1 |
| Compound 21 (NS-42) | 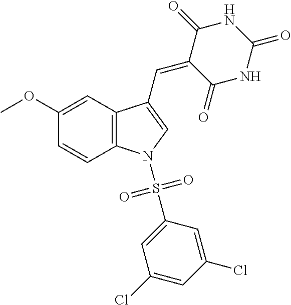 [SMILES: COc1ccc2c(c1)c(C=C1C(=O)NC(=O)NC1=O)cn2S(=O)(=O)c1cc(Cl)cc(Cl)c1] | ¹H NMR (400 MHz, DMSO) δ 11.43 (d, J = 1.8 Hz, 1H), 11.38 (d, J = 1.8 Hz, 1H), 9.52 (s, 1H), 8.45 (s, 1H), 8.15 (d, J = 1.8 Hz, 2H), 8.09 (t, J = 1.9 Hz, 1H), 8.01- 7.97 (m, 1H), 7.36 (d, J = 2.5 Hz, 1H), 7.09 (dd, J = 9.1, 2.4 Hz, 1H), 3.86 (s, 3H). | 494.0 |
| Compound 22 (NS-43) | 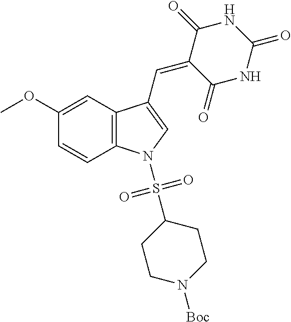 [SMILES: COc1ccc2c(c1)c(C=C1C(=O)NC(=O)NC1=O)cn2S(=O)(=O)C1CCN(C(=O)OC(C)(C)C)CC1] | ¹H NMR (400 MHz, DMSO) δ 11.41 (s, 1H), 11.34 (s, 1H), 9.44 (s, 1H), 8.53 (s, 1H), 7.84 (d, J = 9.1 Hz, 1H), 7.43 (d, J = 2.4 Hz, 1H), 7.12 (dd, J = 9.1, 2.5 Hz, 1H), 4.11 (d, J = 12.7 Hz, 1H), 3.97 (d, J = 13.4 Hz, 2H), 3.91 (s, 3H), 3.32 (s, 3H), 2.74 (s, 2H), 1.87 (d, J = 12.2 Hz, 2H), 1.57-1.40 (m, 2H), 1.35 (s, 10H). | 533.2 |
| Compound 23 (NS-44) | 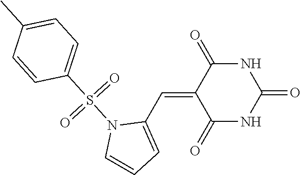 [SMILES: Cc1ccc(S(=O)(=O)n2cccc2C=C2C(=O)NC(=O)NC2=O)cc1] | 1H NMR (400 MHz, DMSO) δ 11.33 (s, 1H), 11.17 (s, 1H), 8.84 (s, 1H), 8.45 (dd, J = 4.0, 1.5 Hz, 1H), 8.12 (dd, J = 3.2, 1.6 Hz, 1H), 8.03-7.92 (m, 2H), 7.86-7.75 (m, 1H), 7.69 (t, J = 7.9 Hz, 2H), 6.72 (t, J = 3.6 Hz, 1H). | 360.1 |
| Compound 24 (NS-45) | 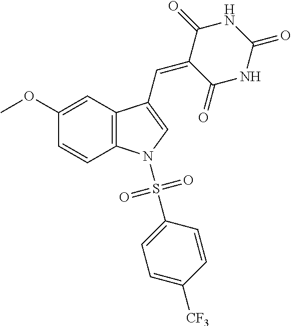 [SMILES: COc1ccc2c(c1)c(C=C1C(=O)NC(=O)NC1=O)cn2S(=O)(=O)c1ccc(C(F)(F)F)cc1] | 1H NMR (400 MHz, DMSO) δ 11.44 (s, 1H), 11.38 (s, 1H), 9.61 (s, 1H), 8.45 (s, 1H), 8.28 (d, J = 8.3 Hz, 2H), 8.03 (d, J = 8.2 Hz, 2H), 7.91 (d, J = 9.0 Hz, 1H), 7.35 (d, J = 2.4 Hz, 1H), 7.08 (dd, J = 9.1, 2.4 Hz, 1H), 3.85 (s, 3H). | 440.1 |
| Compound 25 (NS-46) | 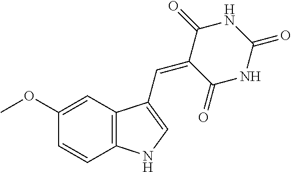 [SMILES: COc1ccc2[nH]cc(C=C3C(=O)NC(=O)NC3=O)c2c1] | 1H NMR (400 MHz, DMSO) δ 12.64 (s, 1H), 11.08 (d, J = 2.0 Hz, 1H), 10.99 (s, 1H), 9.46 (d, J = 3.5 Hz, 1H), 8.68 (s, 1H), 7.49 (d, J = 8.8 Hz, 1H), 7.33 (d, J = 2.4 Hz, 1H), 6.94 (dd, J = 8.8, 2.3 Hz, 1H), 3.88 (s, 4H). | 286.1 |
| Compound 26 (NS-47) | 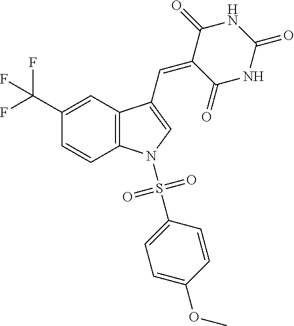 [SMILES: COc1ccc(S(=O)(=O)n2cc(C=C3C(=O)NC(=O)NC3=O)c3cc(C(F)(F)F)ccc32)cc1] | 1H NMR (400 MHz, DMSO) δ 11.45 (d, J = 1.8 Hz, 1H), 11.40 (s, 1H), 9.71 (s, 1H), 8.52 (s, 1H), 8.31 (s, 1H), 8.24 (d, J = 8.7 Hz, 1H), 8.12-7.99 (m, 2H), 7.81 (dd, J = 8.8, 1.7 Hz, 1H), 7.22-7.10 (m, 2H), 3.82 (s, 3H). | 494.1 |
| Compound 27 (NS-48) | 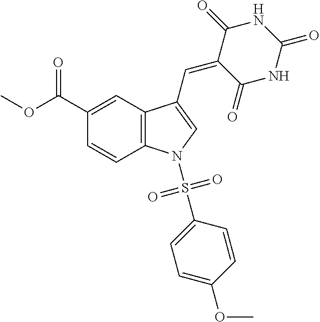 [SMILES: COC(=O)c1ccc2c(c1)c(C=C1C(=O)NC(=O)NC1=O)cn2S(=O)(=O)c1ccc(OC)cc1] | ¹H NMR (400 MHz, DMSO) δ 11.47 (d, J = 1.8 Hz, 1H), 11.42 (d, J = 1.9 Hz, 1H), 9.68 (s, 1H), 8.47 (s, 1H), 8.42 (d, J = 1.6 Hz, 1H), 8.16 (d, J = 8.8 Hz, 1H), 8.07 (dd, J = 8.7, 1.6 Hz, 1H), 8.04-7.96 (m, 2H), 7.47 (d, J = 8.3 Hz, 2H), 3.91 (s, 3H), 2.35 (s, 3H). | 484.1 |
| Compound 28 (NS-49) | 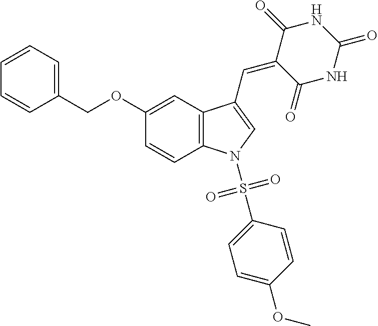 [SMILES: COc1ccc(S(=O)(=O)n2cc(C=C3C(=O)NC(=O)NC3=O)c3cc(OCc4ccccc4)ccc32)cc1] | ¹H NMR (400 MHz, DMSO) δ 11.39 (s, 1H), 11.34 (s, 1H), 9.63 (s, 1H), 8.47 (s, 1H), 8.04-7.97 (m, 2H), 7.91 (d, J = 9.1 Hz, 1H), 7.53-7.44 (m, 3H), 7.40 (t, J = 7.4 Hz, 2H), 7.35 (d, J = 7.3 Hz, 1H), 7.15 (dd, J = 9.2, 2.6 Hz, 3H), 5.22 (s, 2H), 3.82 (s, 3H). | 532.1 |
| Compound 29 (NS-50) | 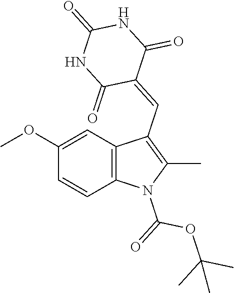 [SMILES: COc1ccc2c(c1)c(C=C1C(=O)NC(=O)NC1=O)c(C)n2C(=O)OC(C)(C)C] | ¹H NMR (400 MHz, DMSO) δ 11.40- 11.24 (m, 1H), 11.13 (d, J = 1.8 Hz, 1H), 8.39 (s, 1H), 7.94 (d, J = 9.0 Hz, 1H), 6.97- 6.88 (m, 1H), 6.87 (d, J = 2.5 Hz, 1H), 3.78 (s, 3H), 1.66 (s, 9H). | 400.1 |
| Compound 30 (NS-51) | 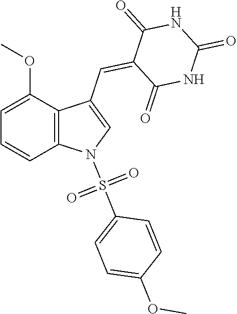 [SMILES: COc1ccc(S(=O)(=O)n2cc(C=C3C(=O)NC(=O)NC3=O)c3c(OC)cccc32)cc1] | ¹H NMR (400 MHz, DMSO) δ 11.30 (d, J = 15.2 Hz, 2H), 9.68 (s, 1H), 9.26 (s, 1H), 8.07-7.90 (m, 2H), 7.59 (d, J = 8.3 Hz, 1H), 7.40 (t, J = 8.2 Hz, 1H), 7.23-7.06 (m, 2H), 7.00 (d, J = 8.2 Hz, 1H), 3.94 (s, 3H), 3.82 (s, 3H). | 456.1 |
| Compound 31 (NS-52) | 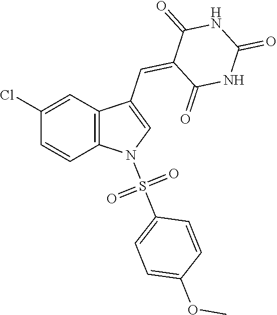 [SMILES: COc1ccc(S(=O)(=O)n2cc(C=C3C(=O)NC(=O)NC3=O)c3cc(Cl)ccc32)cc1] | ¹H NMR (400 MHz, DMSO) δ 11.42 (d, J = 1.9 Hz, 1H), 11.38 (d, J = 1.9 Hz, 1H), 9.64 (s, 1H), 8.42 (s, 1H), 8.09-8.01 (m, 3H), 7.98 (d, J = 2.1 Hz, 1H), 7.51 (dd, J = 8.8, 2.1 Hz, 1H), 7.19-7.11 (m, 2H), 3.82 (s, 3H). | 460.0 |
| Compound 32 (NS-53) | 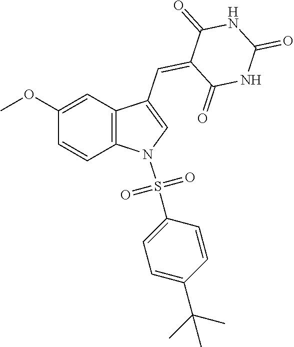 [SMILES: COc1ccc2c(c1)c(C=C1C(=O)NC(=O)NC1=O)cn2S(=O)(=O)c1ccc(C(C)(C)C)cc1] | ¹H NMR (400 MHz, DMSO) δ 11.41 (d, J = 1.9 Hz, 1H), 11.36 (d, J = 1.9 Hz, 1H), 9.63 (s, 1H), 8.47 (s, 1H), 8.02-7.95 (m, 2H), 7.92 (d, J = 9.1 Hz, 1H), 7.73-7.62 (m, 2H), 7.35 (d, J = 2.5 Hz, 1H), 7.08 (dd, J = 9.0, 2.4 Hz, 1H), 3.86 (s, 3H), 1.23 (s, 9H). | 482.1 |
| Compound 33 (NS-54) | 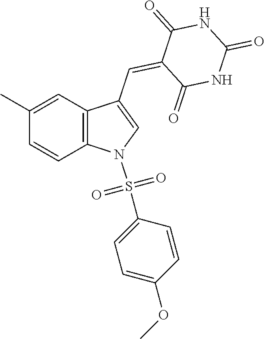 [SMILES: COc1ccc(S(=O)(=O)n2cc(C=C3C(=O)NC(=O)NC3=O)c3cc(C)ccc32)cc1] | ¹H NMR (400 MHz, DMSO) δ 11.40 (d, J = 1.9 Hz, 1H), 11.35 (d, J = 1.9 Hz, 1H), 9.63 (s, 1H), 8.48 (s, 1H), 8.09-7.94 (m, 2H), 7.89 (d, J = 8.5 Hz, 1H), 7.69 (s, 1H), 7.30 (dd, J = 8.7, 1.6 Hz, 1H), 7.19-7.11 (m, 2H), 3.81 (s, 3H), 2.44 (s, 3H). | 440.1 |
| Compound 34 (NS-55) | 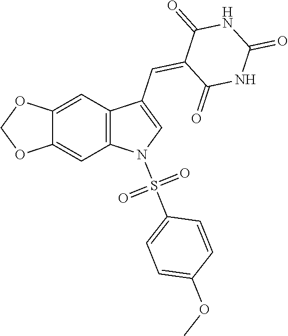 [SMILES: COc1ccc(S(=O)(=O)n2cc(C=C3C(=O)NC(=O)NC3=O)c3cc4c(cc32)OCO4)cc1] | ¹H NMR (400 MHz, DMSO) δ 11.38 (d, J = 1.8 Hz, 1H), 11.32 (s, 1H), 9.51 (s, 1H), 8.37 (s, 1H), 8.07-7.99 (m, 2H), 7.51 (s, 1H), 7.38 (s, 1H), 7.20-7.13 (m, 2H), 6.12 (s, 2H), 3.83 (s, 4H). | 470.1 |
| Compound 35 (NS-56) | 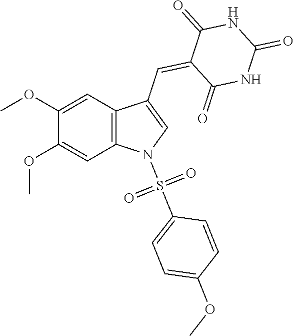 [SMILES: COc1ccc(S(=O)(=O)n2cc(C=C3C(=O)NC(=O)NC3=O)c3cc(OC)c(OC)cc32)cc1] | 1H NMR (400 MHz, DMSO) δ 11.38 (s, 1H), 11.32 (s, 1H), 9.50 (s, 1H), 8.45 (s, 1H), 8.02 (d, J = 9.0 Hz, 2H), 7.46 (s, 1H), 7.34 (s, 1H), 7.20-7.10 (m, 2H), 3.90 (s, 3H), 3.87 (s, 3H), 3.81 (s, 3H). | 486.1 |
| Compound 36 (NS-57) | 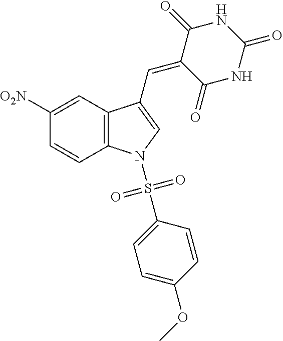 [SMILES: COc1ccc(S(=O)(=O)n2cc(C=C3C(=O)NC(=O)NC3=O)c3cc([N+](=O)[O-])ccc32)cc1] | 1H NMR (400 MHz, DMSO) δ 11.48 (s, 1H), 11.43 (s, 1H), 9.71 (s, 1H), 8.76 (d, J = 2.2 Hz, 1H), 8.50 (s, 1H), 8.35-8.23 (m, 2H), 8.10 (d, J = 9.0 Hz, 2H), 7.18 (d, J = 9.0 Hz, 2H), 3.83 (s, 4H). | 471.0 |
| Compound 37 (NS-58) | 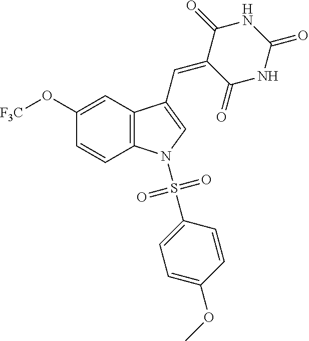 [SMILES: COc1ccc(S(=O)(=O)n2cc(C=C3C(=O)NC(=O)NC3=O)c3cc(OC(F)(F)F)ccc32)cc1] | 1H NMR (400 MHz, DMSO) δ 11.43 (s, 1H), 11.39 (s, 1H), 9.70 (s, 1H), 8.44 (s, 1H), 8.13 (d, J = 9.0 Hz, 1H), 8.10-8.01 (m, 2H), 7.97 (d, J = 2.3 Hz, 1H), 7.48 (dd, J = 8.7, 2.0 Hz, 1H), 7.23-7.09 (m, 2H), 3.82 (s, 3H). | 456.1 |
| Compound 38 (NS-59) | 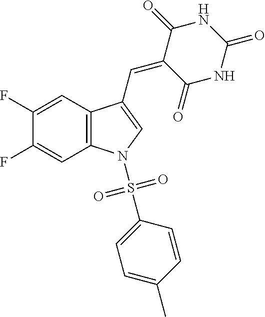 [SMILES: Cc1ccc(S(=O)(=O)n2cc(C=C3C(=O)NC(=O)NC3=O)c3cc(F)c(F)cc32)cc1] | 1H NMR (400 MHz, DMSO) δ 11.43 (d, J = 1.9 Hz, 1H), 11.38 (d, J = 1.9 Hz, 1H), 9.61 (s, 1H), 8.37 (s, 1H), 8.12-7.97 (m, 4H), 7.48 (d, J = 8.2 Hz, 2H), 2.36 (s, 3H). | 446.0 |
| | Exact Mass: 445.0544 | | |
| | NS-59 | | |
| Compound 39 (NS-60) | 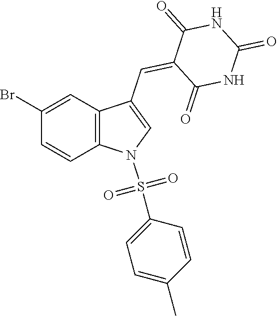 [SMILES: Cc1ccc(S(=O)(=O)n2cc(C=C3C(=O)NC(=O)NC3=O)c3cc(Br)ccc32)cc1] | 1H NMR (400 MHz, DMSO) δ 11.43 (d, J = 1.9 Hz, 1H), 11.38 (d, J = 1.9 Hz, 1H), 9.61 (s, 1H), 8.41 (s, 1H), 8.12 (d, J = 1.9 Hz, 1H), 7.97 (dd, J = 8.7, 2.5 Hz, 3H), 7.63 (dd, J = 8.8, 1.9 Hz, 1H), 7.47 (d, J = 8.2 Hz, 2H), 2.35 (s, 3H). | 428.1 |
| Compound 40 (NS-61) | 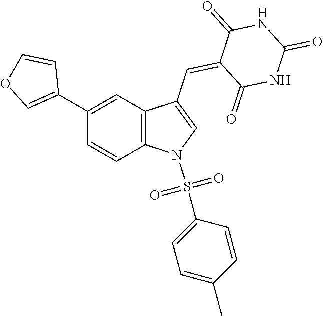 [SMILES: Cc1ccc(S(=O)(=O)n2cc(C=C3C(=O)NC(=O)NC3=O)c3cc(-c4ccoc4)ccc32)cc1] | ¹H NMR (400 MHz, DMSO) δ 11.42 (d, J = 1.9 Hz, 1H), 11.40-11.31 (m, 1H), 9.66 (s, 1H), 8.56 (s, 1H), 8.31 (t, J = 1.2 Hz, 1H), 8.11 (d, J = 1.7 Hz, 1H), 7.99 (dd, J = 8.6, 4.4 Hz, 3H), 7.76 (t, J = 1.7 Hz, 1H), 7.72 (dd, J = 8.6, 1.6 Hz, 1H), 7.46 (d, J = 8.3 Hz, 2H), 7.10 (dd, J = 1.9, 0.9 Hz, 1H), 2.35 (s, 3H). | 476.1 |
| Compound 41 (NS-62) | 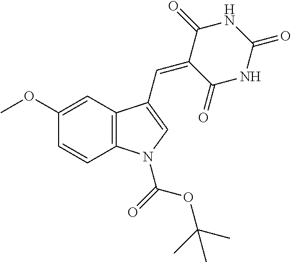 [SMILES: COc1ccc2c(c1)c(C=C1C(=O)NC(=O)NC1=O)cn2C(=O)OC(C)(C)C] | ¹H NMR (400 MHz, DMSO) δ 11.36 (s, 1H), 11.29 (s, 1H), 9.67 (s, 1H), 8.53 (s, 1H), 8.04 (d, J = 9.0 Hz, 1H), 7.36 (d, J = 2.5 Hz, 1H), 7.08 (dd, J = 9.1, 2.5 Hz, 1H), 3.89 (s, 3H), 1.67 (s, 9H). | 386.1 |
| Compound 42 (NS-65) | 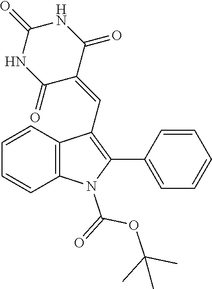 [SMILES: CC(C)(C)OC(=O)n1c(-c2ccccc2)c(C=C2C(=O)NC(=O)NC2=O)c2ccccc21] | ¹H NMR (400 MHz, DMSO) δ 11.30 (d, J = 1.7 Hz, 1H), 11.19 (s, 1H), 8.17 (d, J = 8.3 Hz, 1H), 7.75 (s, 1H), 7.56 (dd, J = 5.0, 1.8 Hz, 3H), 7.42 (ddt, J = 6.4, 4.8, 2.6 Hz, 3H), 7.36-7.25 (m, 2H), 1.25 (s, 9H). | 432.1 |
| Compound 42 (NS-66) | 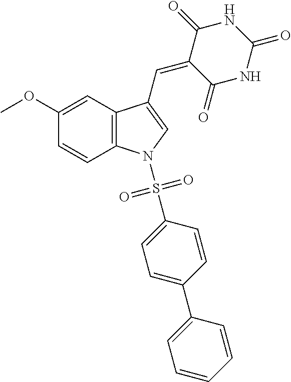 [SMILES: COc1ccc2c(c1)c(C=C1C(=O)NC(=O)NC1=O)cn2S(=O)(=O)c1ccc(-c2ccccc2)cc1] | ¹H NMR (400 MHz, DMSO) δ 11.41 (d, J = 1.9 Hz, 1H), 11.37 (d, J = 1.8 Hz, 1H), 9.67 (s, 1H), 8.47 (s, 1H), 8.18-8.05 (m, 2H), 7.94 (dd, J = 10.3, 8.8 Hz, 3H), 7.75- 7.63 (m, 2H), 7.55-7.41 (m, 3H), 7.36 (d, J = 2.4 Hz, 1H), 7.10 (dd, J = 9.1, 2.4 Hz, 1H), 3.86 (s, 3H). | 502.1 |
| Compound 43 (NS-67) | 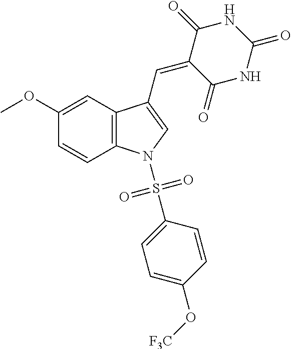 [SMILES: COc1ccc2c(c1)c(C=C1C(=O)NC(=O)NC1=O)cn2S(=O)(=O)c1ccc(OC(F)(F)F)cc1] | ¹H NMR (400 MHz, DMSO) δ 11.42 (d, J = 1.9 Hz, 1H), 11.37 (d, J = 1.9 Hz, 1H), 9.61 (s, 1H), 8.46 (s, 1H), 8.31-8.12 (m, 2H), 7.91 (d, J = 9.1 Hz, 1H), 7.70-7.59 (m, 2H), 7.37 (d, J = 2.5 Hz, 1H), 7.09 (dd, J = 9.0, 2.4 Hz, 1H), 3.86 (s, 3H). | 510.1 |
| Compound 44 (NS-68) | 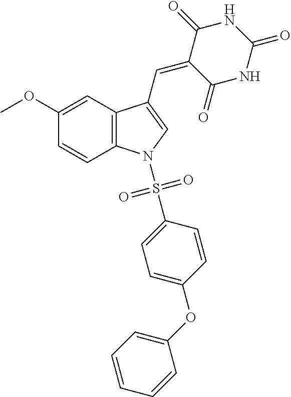 [SMILES: COc1ccc2c(c1)c(C=C1C(=O)NC(=O)NC1=O)cn2S(=O)(=O)c1ccc(Oc2ccccc2)cc1] | ¹H NMR (400 MHz, DMSO) δ 11.54- 11.21 (m, 2H), 9.62 (s, 1H), 8.48 (s, 1H), 8.13-8.01 (m, 2H), 7.90 (d, J = 9.1 Hz, 1H), 7.50-7.40 (m, 2H), 7.37 (d, J = 2.4 Hz, 1H), 7.29 (d, J = 7.6 Hz, 1H), 7.18- 7.03 (m, 5H), 3.87 (s, 3H). | 518.1 |
| Compound 45 (NS-69) | 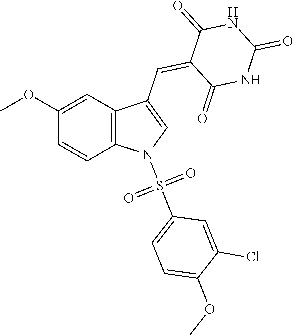 [SMILES: COc1ccc2c(c1)c(C=C1C(=O)NC(=O)NC1=O)cn2S(=O)(=O)c1ccc(OC)c(Cl)c1] | ¹H NMR (400 MHz, Dioxane) δ 12.45 (d, J = 1.9 Hz, 1H), 12.40 (d, J = 1.9 Hz, 1H), 10.62 (s, 1H), 9.50 (s, 1H), 9.16 (d, J = 2.5 Hz, 1H), 9.09 (dd, J = 8.9, 2.5 Hz, 1H), 8.98 (d, J = 9.1 Hz, 1H), 8.44-8.36 (m, 2H), 8.12 (dd, J = 9.1, 2.5 Hz, 1H), 4.96 (s, 3H), 4.90 (s, 3H). | 490.0 |
| Compound 46 (NS-70) | 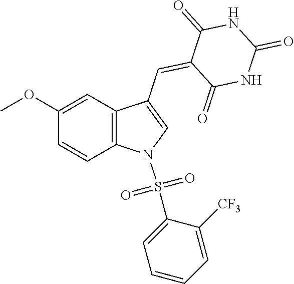 [SMILES: COc1ccc2c(c1)c(C=C1C(=O)NC(=O)NC1=O)cn2S(=O)(=O)c1ccccc1C(F)(F)F] | ¹H NMR (400 MHz, DMSO) δ 11.42 (d, J = 1.9 Hz, 1H), 11.36 (d, J = 1.8 Hz, 1H), 9.66 (s, 1H), 8.51 (s, 1H), 8.14 (dd, J = 7.9, 1.5 Hz, 1H), 8.11-8.05 (m, 1H), 8.05- 7.90 (m, 2H), 7.70 (d, J = 9.0 Hz, 1H), 7.43 (d, J = 2.5 Hz, 1H), 7.06 (dd, J = 9.1, 2.4 Hz, 1H), 3.88 (s, 3H). | 494.0 |
| Compound 47 (NS-71) | 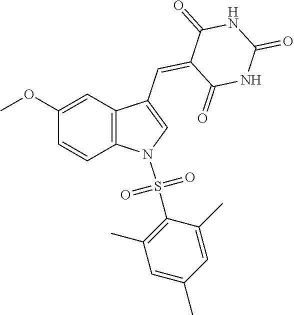 [SMILES: COc1ccc2c(c1)c(C=C1C(=O)NC(=O)NC1=O)cn2S(=O)(=O)c1c(C)cc(C)cc1C] | ¹H NMR (400 MHz, DMSO) δ 11.38 (d, J = 1.9 Hz, 1H), 11.31 (d, J = 1.9 Hz, 1H), 9.68 (s, 1H), 8.53 (s, 1H), 7.40 (d, J = 2.4 Hz, 1H), 7.30 (d, J = 9.1 Hz, 1H), 7.20 (s, 2H), 7.02 (dd, J = 9.0, 2.4 Hz, 1H), 3.86 (s, 3H), 2.51-2.50 (m, 6H), 2.29 (s, 3H). | 468.1 |
| Compound 48 (NS-72) | 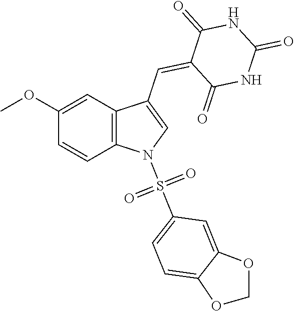 [SMILES: COc1ccc2c(c1)c(C=C1C(=O)NC(=O)NC1=O)cn2S(=O)(=O)c1ccc2c(c1)OCO2] | ¹H NMR (400 MHz, DMSO) δ 11.40 (d, J = 1.9 Hz, 1H), 11.34 (d, J = 1.9 Hz, 1H), 9.58 (s, 1H), 8.47 (s, 1H), 7.92 (d, J = 9.1 Hz, 1H), 7.67 (dd, J = 8.3, 2.1 Hz, 1H), 7.53 (d, J = 2.0 Hz, 1H), 7.35 (d, J = 2.5 Hz, 1H), 7.13 (d, J = 8.3 Hz, 1H), 7.07 (dd, J = 9.1, 2.4 Hz, 1H), 6.16 (s, 2H), 3.86 (s, 3H). | 470.0 |
| Compound 49 (NS-73) | 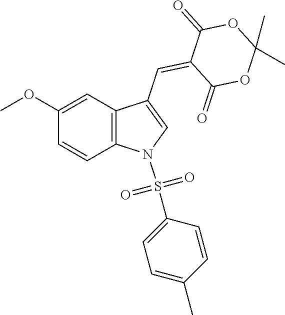 | ¹H NMR (400 MHz, DMSO) δ 9.39 (s, 1H), 8.58 (s, 1H), 7.98 (d, J = 8.4 Hz, 2H), 7.88 (d, J = 9.0 Hz, 1H), 7.46 (d, J = 8.0 Hz, 3H), 7.07 (dd, J = 9.0, 2.4 Hz, 1H), 3.85 (s, 3H), 2.35 (s, 3H), 1.76 (s, 6H). | 466.1 |
| Compound 50 (NS-74) | 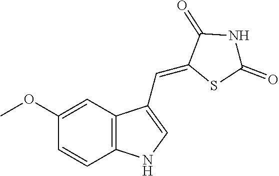 [SMILES: COc1ccc2[nH]cc(/C=C3\SC(=O)NC3=O)c2c1] | ¹H NMR (400 MHz, DMSO) δ 12.25 (s, 1H), 12.06-11.91 (m, 1H), 8.10 (s, 1H), 7.67 (d, J = 3.1 Hz, 1H), 7.47-7.33 (m, 2H), 6.87 (dd, J = 8.8, 2.4 Hz, 1H), 3.83 (s, 3H). | 275.0 |
| Compound 51 (NS-75) | 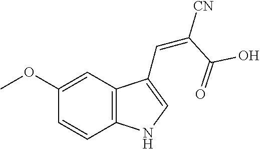 [SMILES: COc1ccc2[nH]cc(/C=C(/C#N)C(=O)O)c2c1] | ¹H NMR (400 MHz, DMSO) δ 8.34 (s, 2H), 7.43 (d, J = 8.8 Hz, 1H), 7.35 (d, J = 2.4 Hz, 1H), 6.86 (dd, J = 8.8, 2.4 Hz, 1H), 3.83 (s, 4H). | 243.0 |
| Compound 52 (NS-76) | 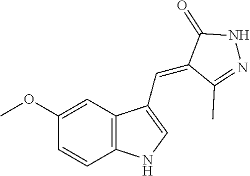 [SMILES: COc1ccc2[nH]cc(/C=C3/C(=O)NN=C3C)c2c1] | ¹H NMR (400 MHz, DMSO) δ 12.23 (s, 1H), 10.98 (s, 1H), 9.75 (s, 1H), 7.94 (s, 1H), 7.66 (d, J = 2.4 Hz, 1H), 7.45 (d, J = 8.7 Hz, 1H), 6.90 (dd, J = 8.8, 2.4 Hz, 1H), 3.87 (s, 3H), 2.27 (s, 3H). | 255.1 |
| Compound 53 (NS-77) | 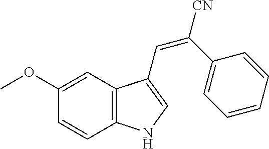 [SMILES: COc1ccc2[nH]cc(/C=C(/C#N)c3ccccc3)c2c1] | ¹H NMR (400 MHz, DMSO) δ 12.57 (s, 1H), 8.63 (s, 1H), 8.55 (s, 1H), 7.88-7.77 (m, 2H), 7.73-7.63 (m, 1H), 7.59 (dd, J = 8.3, 6.9 Hz, 2H), 7.49 (d, J = 8.8 Hz, 1H), 7.43 (d, J = 2.4 Hz, 1H), 6.93 (dd, J = 8.8, 2.4 Hz, 1H), 3.81 (s, 3H). | 275.1 |
| Compound 54 (NS-79) | 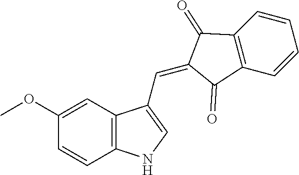 [SMILES: COc1ccc2[nH]cc(C=C3C(=O)c4ccccc4C3=O)c2c1] | ¹H NMR (400 MHz, DMSO) δ 12.64 (s, 1H), 9.59 (d, J = 3.5 Hz, 1H), 8.19 (s, 1H), 7.94-7.76 (m, 4H), 7.55 (d, J = 2.4 Hz, 1H), 7.48 (d, J = 8.7 Hz, 1H), 6.92 (dd, J = 8.7, 2.4 Hz, 1H), 3.89 (s, 3H). | 304.1 |
| Compound 54 (NS-84) | 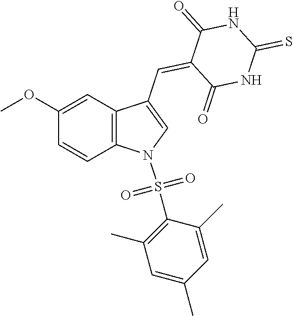 [SMILES: COc1ccc2c(c1)c(C=C1C(=O)NC(=S)NC1=O)cn2S(=O)(=O)c1c(C)cc(C)cc1C] | ¹H NMR (400 MHz, DMSO) δ 12.47 (d, J = 1.7 Hz, 1H), 12.42 (d, J = 1.7 Hz, 1H), 9.75 (s, 1H), 8.56 (s, 1H), 7.44 (d, J = 2.5 Hz, 1H), 7.27 (d, J = 9.0 Hz, 1H), 7.21 (s, 2H), 7.02 (dd, J = 9.1, 2.4 Hz, 1H), 3.86 (s, 3H), 2.51 (s, 6H), 2.30 (s, 3H). | 484.1 |
| Compound 54 (NS-91) | 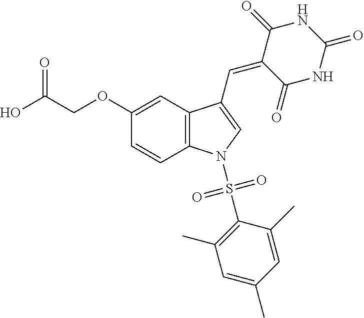 [SMILES: Cc1cc(C)c(S(=O)(=O)n2cc(C=C3C(=O)NC(=O)NC3=O)c3cc(OCC(=O)O)ccc32)c(C)c1] | ¹H NMR (400 MHz, DMSO) δ 13.34 (s, 1H), 11.20 (d, J = 1.9 Hz, 1H), 11.11 (d, J = 1.9 Hz, 1H), 9.49 (s, 1H), 8.36 (s, 1H), 7.62 (d, J = 8.9 Hz, 1H), 7.35 (d, J = 2.2 Hz, 1H), 7.17 (s, 2H), 6.96 (dd, J = 8.9, 2.3 Hz, 1H), 5.33 (s, 2H), 2.49 (s, 6H), 2.32 (s, 3H). | 512.1 |

### Example 14—Methods for Examples 10 to 13

### Fluorescence Polarization Assay

A fluorescence polarization (FP)-based assay was used to screen NSUN2 inhibitors. Briefly, purified His-tagged NSUN2 protein was diluted to 30 nM in FP binding buffer (10 mM Tris-HCl, pH 7.5, 100 mM KCl, 0.1 mM DTT, 1 mM MgCl₂, and 5% glycerol) and 34.2 μL was dispensed into each well of a 384-well black plate. Test compounds (0.8 μL, 5 mM in DMSO) were added and incubated at 37° C. for 15 min. Subsequently, 5 μL of FAM-labeled RNA probe (final concentration 16 nM) was added and incubated at 37° C. for an additional 15 min. Fluorescence polarization signals were then measured, and inhibition was determined based on the reduction in polarization relative to control samples. The cures were fitted and IC₅₀ values were calculated by Graphpad Prism 9. The determined IC₅₀ values of the compounds are included in Table 1.

### Cell Proliferation Assay

Cells was seeded in 96-well microplates at a density of 5000 cells/well. Compounds with serial 1/2 dilution in DMSO (1000×) was diluted for 50-fold with the cell culture medium. The prepared compound solution was added to cell solution in triplicate. The plates were incubated for 3 days at 37° C. and 5% CO₂, and the cell viability was measured by MTS Assay Kit (Cell Proliferation). The average cell viability value for each sample was calculated by setting the DMSO wells at 100%. The cures were fitted and IC₅₀ values were calculated by Graphpad Prism 9. The determined IC₅₀ values of the compounds are included in Table 2

### MTase-Glo Methyltransferase Assay

An MTase-Glo methyltransferase assay was used to evaluate NSUN enzymatic inhibition. Briefly, purified NSUN protein was incubated with RNA substrate and S-adenosylmethionine (SAM) in an appropriate reaction buffer at 37° C. for a defined period to allow methylation. Test compounds were added at the desired concentrations and pre-incubated with the enzyme prior to initiating the reaction. Following the methylation reaction, MTase-Glo reagent was added according to the manufacturer's instructions to convert the reaction byproduct S-adenosylhomocysteine (SAH) into a luminescent signal. Luminescence was measured using a plate reader, and inhibition of NSUN activity was determined based on the decrease in signal relative to control reactions without inhibitor.

### Biotin Pull Down Assay

Biotin pull-down assays were performed to identify target proteins of the biotinylated small molecule. Briefly, cells were lysed in ice-cold lysis buffer (e.g., 50 mM Tris-HCl pH 7.5, 150 mM NaCl, 0.5% NP-40, 1 mM DTT, and protease inhibitor cocktail) and clarified by centrifugation. The supernatant was incubated with the biotinylated compound NS-Biotin (2, 5, 10 μM) at 4° C. for 2 h with rotation. To remove excess free small molecules, the mixture was passed through a desalting spin column according to the manufacturer's instructions Streptavidin magnetic beads were pre-washed and added to the mixture, followed by incubation at 4° C. for an additional 1 h. The beads were then washed multiple times with lysis buffer or high-salt buffer to reduce non-specific binding. Bound proteins were eluted by boiling in SDS loading buffer and analyzed by Western blot.

### Washout Assay for Assessing Covalent Target Engagement

NSUN2 protein (20 μM) was incubated with DMSO or NS71 (100 μM) a in reaction buffer (e.g., 50 mM Tris-HCl pH 7.5, 150 mM NaCl, 0.1 mM DTT) at room temperature for 0.5 h. Following incubation, the reaction mixture was subjected to ultrafiltration using a centrifugal filter unit (10 kDa MWCO) to remove unbound small molecules. The protein retentate was washed multiple times with fresh buffer for 5 cycles to ensure complete removal of free compound. The retained protein was then collected and tested in MTase-Glo methyltransferase assay. Relative NSUN2 activity was normalized to the DMSO-treated control.

Quantification of m⁵C in tRNA by UHPLC—MS/MS

Molm13 cells were treated with NS71 at different concentrations for 48 h. Total RNA was extracted from cells using TRIzol reagent, and tRNA was purified by size selection. Purified tRNA was enzymatically digested into single nucleosides by incubation with nuclease P1 (2 U) at 37° C. for 2 h, followed by addition of alkaline phosphatase (0.5 U) and incubation at 37° C. for an additional 1 h. Prior to analysis, all samples were diluted with LC-grade water and filtered through a 0.22 μm PVDF filter (Millipore, SLGVR04NL) prior to injection into a C18 reversed-phase column connected to a SCIEX 6500+ Triple Quadrupole Mass Spectrometer operating in positive electrospray ionization mode. Nucleosides were quantified based on retention time and specific nucleoside-to-base ion transitions (RNA: 268→136 for A; 126 for m⁵C). Relative nucleoside levels were determined by peak area comparison within the same batch of reactions.

### Pharmacokinetic Study

Pharmacokinetic (PK) studies were conducted to evaluate the in vivo exposure of the test compound. Briefly, the compounds (10 mg/kg) were administered to mice via intraperitoneal injection. Blood samples were collected at predetermined time points post-dose and processed to obtain plasma by centrifugation. Plasma samples were precipitated with organic solvent containing an internal standard, followed by centrifugation, and the supernatant was analyzed by LC-MS/MS to quantify compound concentrations.

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

All references cited herein, the extent that they provide exemplary procedural or other details supplementary to those set forth herein, are specifically incorporated herein by reference.
1. Komashko, V. M., and Farnham, P. J. (2010). 5-azacytidine treatment reorganizes genomic histone modification patterns. Epigenetics 5, 229-240. 10.4161/epi.5.3.11409.
2. Grövdal, M., Karimi, M., Tobiasson, M., Reinius, L., Jansson, M., Ekwall, K., Ungerstedt, J., Kere, J., Greco, D., and Hellström-Lindberg, E. (2014). Azacitidine induces profound genome-wide hypomethylation in primary myelodysplastic bone marrow cultures but may also reduce histone acetylation. Leukemia 28, 411-413. 10.1038/leu.2013.265.
3. Tobiasson, M., Abdulkadir, H., Lennartsson, A., Katayama, S., Marabita, F., De Paepe, A., Karimi, M., Krjutskov, K., Einarsdottir, E., Grövdal, M., et al. (2017). Comprehensive mapping of the effects of azacitidine on DNA methylation, repressive/permissive histone marks and gene expression in primary cells from patients with MDS and MDS-related disease. Oncotarget 8, 28812-28825. 10.18632/oncotarget.15807.
4. Leung, K. K., Nguyen, A., Shi, T., Tang, L., Ni, X., Escoubet, L., MacBeth, K.J., DiMartino, J., and Wells, J. A. (2019). Multiomics of azacitidine-treated AML cells reveals variable and convergent targets that remodel the cell-surface proteome. Proceedings of the National Academy of Sciences 116, 695-700. 10.1073/pnas.1813666116.
5. Stresemann, C., Bokelmann, I., Mahlknecht, U., and Lyko, F. (2008). Azacytidine causes complex DNA methylation responses in myeloid leukemia. Molecular Cancer Therapeutics 7, 2998-3005. 10.1158/1535-7163.MCT-08-0411.
6. Fenaux, P., Haase, D., Santini, V., Sanz, G. F., Platzbecker, U., and Mey, U. (2021). Myelodysplastic syndromes: ESMO Clinical Practice Guidelines for diagnosis, treatment and follow-up. Annals of Oncology 32, 142-156. 10.1016/j.annonc.2020.11.002.
7. Sekeres, M. A., Guyatt, G., Abel, G., Alibhai, S., Altman, J. K., Buckstein, R., Choe, H., Desai, P., Erba, H., Hourigan, C. S., et al. (2020). American Society of Hematology 2020 guidelines for treating newly diagnosed acute myeloid leukemia in older adults. Blood Advances 4, 3528-3549. 10.1182/bloodadvances.2020001920.
8. Fenaux, P., Mufti, G.J., Hellström-Lindberg, E., Santini, V., Finelli, C., Giagounidis, A., Schoch, R., Gattermann, N., Sanz, G., List, A., et al. (2009). Efficacy of azacitidine compared with that of conventional care regimens in the treatment of higher-risk myelodysplastic syndromes: a randomised, open-label, phase III study. The Lancet Oncology 10, 223-232. https://doi.org/10.1016/S1470-2045(09)70003-8.
9. Ivanoff, S., Gruson, B., Chantepie, S.P., Lemasle, E., Merlusca, L., Harrivel, V., Charbonnier, A., Votte, P., Royer, B., and Marolleau, J. P. (2013). 5-Azacytidine treatment for relapsed or refractory acute myeloid leukemia after intensive chemotherapy. Am J Hematol 88, 601-605. 10.1002/ajh.23464.
10. Li, L. H., Olin, E. J., Buskirk, H. H., and Reineke, L. M. (1970). Cytotoxicity and Mode of Action of 5-Azacytidine on L1210 Leukemia. Cancer Research 30, 2760-2769.
11. Taylor, S. M., and Jones, P. A. (1982). Mechanism of action of eukaryotic DNA methyltransferase. Use of 5-azacytosine-containing DNA. J Mol Biol 162, 679-692. 10.1016/0022-2836(82)90395-3.
12. Santi, D. V., Norment, A., and Garrett, C.E. (1984). Covalent bond formation between a DNA-cytosine methyltransferase and DNA containing 5-azacytosine. Proceedings of the National Academy of Sciences 81, 6993-6997. 10.1073/pnas.81.22.6993.
13. Jackson-Grusby, L., Laird, P. W., Magge, S. N., Moeller, B. J., and Jaenisch, R. (1997). Mutagenicity of 5-aza-2′-deoxycytidine is mediated by the mammalian DNA methyltransferase. Proceedings of the National Academy of Sciences 94, 4681-4685. 10.1073/pnas.94.9.4681.
14. Lu, L.-J. W., and Randerath, K. (1979). Effects of 5-Azacytidine on Transfer RNA Methyltransferases. Cancer Research 39, 940-949.
15. Lu, L. J., and Randerath, K. (1980). Mechanism of 5-azacytidine-induced transfer RNA cytosine-5-methyltransferase deficiency. Cancer Res 40, 2701-2705.
16. Saba, H. I. (2007). Decitabine in the treatment of myelodysplastic syndromes. Ther Clin Risk Manag 3, 807-817.
17. Ronit, G., Liat, V., Anat, G.-G., Yulia, B., Moshe, Y., Pia, R., and Ofer, S. (2010). 5-azacitidine prolongs overall survival in patients with myelodysplastic syndrome—a systematic review and meta-analysis. Haematologica 95, 303-310. 10.3324/haematol.2009.010611.
18. Kantarjian, H. M., Thomas, X. G., Dmoszynska, A., Wierzbowska, A., Mazur, G., Mayer, J., Gau, J. P., Chou, W. C., Buckstein, R., Cermak, J., et al. (2012). Multicenter, randomized, open-label, phase III trial of decitabine versus patient choice, with physician advice, of either supportive care or low-dose cytarabine for the treatment of older patients with newly diagnosed acute myeloid leukemia. Journal of clinical oncology: official journal of the American Society of Clinical Oncology 30, 2670-2677. 10.1200/jco.2011.38.9429.
19. Cheng, J. X., Chen, L., Li, Y., Cloe, A., Yue, M., Wei, J., Watanabe, K. A., Shammo, J. M., Anastasi, J., Shen, Q. J., et al. (2018). RNA cytosine methylation and methyltransferases mediate chromatin organization and 5-azacytidine response and resistance in leukaemia. Nature Communications 9, 1163. 10.1038/s41467-018-03513-4.
20. Chen, Y.-S., Yang, W.-L., Zhao, Y.-L., and Yang, Y.-G. (2021). Dynamic transcriptomic m5C and its regulatory role in RNA processing. WIREs RNA 12, e1639. https://doi.org/10.1002/wrna.1639.
21. Gao, Y., and Fang, J. (2021). RNA 5-methylcytosine modification and its emerging role as an epitranscriptomic mark. RNA Biol 18, 117-127. 10.1080/15476286.2021.1950993.
22. Sharma, S., Yang, J., Watzinger, P., Kotter, P., and Entian, K.-D. (2013). Yeast Nop2 and Rcm1 methylate C2870 and C2278 of the 25S rRNA, respectively. Nucleic Acids Research 41, 9062-9076. 10.1093/nar/gkt679.
23. Tuorto, F., Liebers, R., Musch, T., Schaefer, M., Hofmann, S., Kellner, S., Frye, M., Helm, M., Stoecklin, G., and Lyko, F. (2012). RNA cytosine methylation by Dnmt2 and NSun2 promotes tRNA stability and protein synthesis. Nature Structural & Molecular Biology 19, 900-905. 10.1038/nsmb.2357.
24. Ma, H.-L., Bizet, M., Soares Da Costa, C., Murisier, F., de Bony, E. J., Wang, M.-K., Yoshimi, A., Lin, K.-T., Riching, K. M., Wang, X., et al. (2023). SRSF2 plays an unexpected role as reader of m5C on mRNA, linking epitranscriptomics to cancer. Molecular Cell 83, 4239-4254.e4210. 10.1016/j.molcel.2023.11.003.
25. Yang, X., Yang, Y., Sun, B.-F., Chen, Y.-S., Xu, J.-W., Lai, W.-Y., Li, A., Wang, X., Bhattarai, D. P., Xiao, W., et al. (2017). 5-methylcytosine promotes mRNA export —NSUN2 as the methyltransferase and ALYREF as an m5C reader. Cell Research 27, 606-625. 10.1038/cr.2017.55.
26. Selmi, T., Hussain, S., Dietmann, S., Heiß, M., Borland, K., Flad, S., Carter, J. M., Dennison, R., Huang, Y. L., Kellner, S., et al. (2021). Sequence- and structure-specific cytosine-5 mRNA methylation by NSUN6. Nucleic Acids Res 49, 1006-1022. 10.1093/nar/gkaa1193.
27. Chen, X., Li, A., Sun, B.-F., Yang, Y., Han, Y.-N., Yuan, X., Chen, R.-X., Wei, W.-S., Liu, Y., Gao, C.-C., et al. (2019). 5-methylcytosine promotes pathogenesis of bladder cancer through stabilizing mRNAs. Nature Cell Biology 21, 978-990. 10.1038/s41556-019-0361-y.
28. Zou, Z., Dou, X., Li, Y., Zhang, Z., Wang, J., Gao, B., Xiao, Y., Wang, Y., Zhao, L., Sun, C., et al. (2024). RNA m5C oxidation by TET2 regulates chromatin state and leukemogenesis. Nature 634, 986-994. 10.1038/s41586-024-07969-x.
29. Guo, J., Zou, Z., Dou, X., Zhao, X., Wang, Y., Wei, L., Pi, Y., Wang, Y., He, C., and Guo, S. (2024). Zebrafish Mbd5 binds to RNA m5C and regulates histone deubiquitylation and gene expression in development metabolism and behavior. Nucleic Acids Research 52, 4257-4275. 10.1093/nar/gkae093.
30. Marcucci, G., Silverman, L., Eller, M., Lintz, L., and Beach, C.L. (2005). Bioavailability of azacitidine subcutaneous versus intravenous in patients with the myelodysplastic syndromes. J Clin Pharmacol 45, 597-602. 10.1177/0091270004271947.
31. Drew, Y., Zenke, F. T., and Curtin, N.J. (2025). DNA damage response inhibitors in cancer therapy: lessons from the past, current status and future implications. Nature Reviews Drug Discovery 24, 19-39. 10.1038/s41573-024-01060-w.
32. DiNardo, C.D., Jonas, B. A., Pullarkat, V., Thirman, M.J., Garcia, J.S., Wei, A.H., Konopleva, M., Dohner, H., Letai, A., Fenaux, P., et al. (2020). Azacitidine and Venetoclax in Previously Untreated Acute Myeloid Leukemia. N Engl J Med 383, 617-629. 10.1056/NEJMoa2012971.
33. Samra, B., Konopleva, M., Isidori, A., Daver, N., and DiNardo, C. (2020). Venetoclax-Based Combinations in Acute Myeloid Leukemia: Current Evidence and Future Directions. Front Oncol 10, 562558. 10.3389/fonc.2020.562558.
34. Reilly, B.M., Luger, T., Park, S., Lio, C. J., González-Avalos, E., Wheeler, E.C., Lee, M., Williamson, L., Tanaka, T., Diep, D., et al. (2021). 5-Azacytidine Transiently Restores Dysregulated Erythroid Differentiation Gene Expression in TET2-Deficient Erythroleukemia Cells. Mol Cancer Res 19, 451-464. 10.1158/1541-7786.Mcr-20-0453.
35. Stomper, J., Rotondo, J. C., Greve, G., and Lübbert, M. (2021). Hypomethylating agents (HMA) for the treatment of acute myeloid leukemia and myelodysplastic syndromes: mechanisms of resistance and novel HMA-based therapies. Leukemia 35, 1873-1889. 10.1038/s41375-021-01218-0.
36. Fousteri, M., and Mullenders, L. H. F. (2008). Transcription-coupled nucleotide excision repair in mammalian cells: molecular mechanisms and biological effects. Cell Research 18, 73-84. 10.1038/cr.2008.6.
37. Liu, J., Dou, X., Chen, C., Chen, C., Liu, C., Xu, M. M., Zhao, S., Shen, B., Gao, Y., Han, D., and He, C. (2020). N6-methyladenosine of chromosome-associated regulatory RNA regulates chromatin state and transcription. Science 367, 580-586. 10.1126/science.aay6018.
38. Xu, W., Li, J., He, C., Wen, J., Ma, H., Rong, B., Diao, J., Wang, L., Wang, J., Wu, F., et al. (2021). METTL3 regulates heterochromatin in mouse embryonic stem cells. Nature 591, 317-321. 10.1038/s41586-021-03210-1.
39. Wei, J., Yu, X., Yang, L., Liu, X., Gao, B., Huang, B., Dou, X., Liu, J., Zou, Z., Cui, X.-L., et al. (2022). FTO mediates LINE1 m6A demethylation and chromatin regulation in mESCs and mouse development. Science 376, 968-973. 10.1126/science.abe9582.
40. Diesch, J., Le Pannérer, M. M., Winkler, R., Casquero, R., Muhar, M., van der Garde, M., Maher, M., Herráez, C. M., Bech-Serra, J. J., Fellner, M., et al. (2021). Inhibition of CBP synergizes with the RNA-dependent mechanisms of Azacitidine by limiting protein synthesis. Nat Commun 12, 6060. 10.1038/s41467-021-26258-z.
41. Aguilo, F., Li, S., Balasubramaniyan, N., Sancho, A., Benko, S., Zhang, F., Vashisht, A., Rengasamy, M., Andino, B., Chen, C.-h., et al. (2016). Deposition of 5-Methylcytosine on Enhancer RNAs Enables the Coactivator Function of PGC-la. Cell Reports 14, 479-492. 10.1016/j.celrep.2015.12.043.
42. Wang, Z., Li, K., Chen, W., Wang, X., Huang, Y., Wang, W., Wu, W., Cai, Z., and Huang, W. (2020). Modulation of SRSF2 expression reverses the exhaustion of TILs via the epigenetic regulation of immune checkpoint molecules. Cell Mol Life Sci 77, 3441-3452. 10.1007/s00018-019-03362-4.
43. Li, K., and Wang, Z. (2021). Speckles and paraspeckles coordinate to regulate HSV-1 genes transcription. Communications Biology 4, 1207. 10.1038/s42003-021-02742-6.
44. Ji, X., Zhou, Y., Pandit, S., Huang, J., Li, H., Lin, C. Y., Xiao, R., Burge, C. B., and Fu, X.-D. (2013). SR Proteins Collaborate with 7SK and Promoter-Associated Nascent RNA to Release Paused Polymerase. Cell 153, 855-868. 10.1016/j.cell.2013.04.028.
45. Wagner, R. E., Arnetzl, L., Britto-Borges, T., Heit-Mondrzyk, A., Bakr, A., Sollier, E., Gkatza, N. A., Panten, J., Delaunay, S., Sohn, D., et al. (2024). SRSF2 safeguards efficient transcription of DNA damage and repair genes. Cell Reports 43, 114869. https://doi.org/10.1016/j.celrep.2024.114869.
46. Mei, L., Shen, C., Miao, R., Wang, J.-Z., Cao, M.-D., Zhang, Y.-S., Shi, L.-H., Zhao, G.-H., Wang, M.-H., Wu, L.-S., and Wei, J.-F. (2020). RNA methyltransferase NSUN2 promotes gastric cancer cell proliferation by repressing p57Kip2 by an m5C-dependent manner. Cell Death & Disease 11, 270. 10.1038/s41419-020-2487-z.
47. Yang, M., Wei, R., Zhang, S., Hu, S., Liang, X., Yang, Z., Zhang, C., Zhang, Y., Cai, L., and Xie, Y. (2023). NSUN2 promotes osteosarcoma progression by enhancing the stability of FABP5 mRNA via m5C methylation. Cell Death & Disease 14, 125. 10.1038/s41419-023-05646-x.
48. Cappellini, M. D., Taher, A. T., Verma, A., Shah, F., and Hermine, O. (2023). Erythropoiesis in lower-risk myelodysplastic syndromes and beta-thalassemia. Blood Reviews 59, 101039. https://doi.org/10.1016fj.blre.2022.101039.
49. Kasprzak, A., Assadi, C., Nachtkamp, K., Rudelius, M., Haas, R., Giagounidis, A., Götze, K., Gattermann, N., and Germing, U. (2023). Monocytosis at the time of diagnosis has a negative prognostic impact in myelodysplastic syndromes with less than 5% bone marrow blasts. Annals of Hematology 102, 99-106. 10.1007/s00277-022-05043-y.
50. Baiocchi, M., Di Rico, C., Di Pietro, R., Di Baldassarre, A., and Migliaccio, A. R. (2003). 5-azacytidine reactivates the erythroid differentiation potential of the myeloid-restricted murine cell line 32D Ro. Exp Cell Res 285, 258-267. 10.1016/s0014-4827(03)00032-6.
51. Itzykson, R., Kosmider, O., Cluzeau, T., Mansat-De Mas, V., Dreyfus, F., Beyne-Rauzy, O., Quesnel, B., Vey, N., Gelsi-Boyer, V., Raynaud, S., et al. (2011). Impact of TET2 mutations on response rate to azacitidine in myelodysplastic syndromes and low blast count acute myeloid leukemias. Leukemia 25, 1147-1152. 10.1038/leu.2011.71.
52. Cluzeau, T., Robert, G., Mounier, N., Karsenti, J. M., Dufies, M., Puissant, A., Jacquel, A., Renneville, A., Preudhomme, C., Cassuto, J. P., et al. (2012). BCL2L10 is a predictive factor for resistance to azacitidine in MDS and AML patients. Oncotarget 3, 490-501. 10.18632/oncotarget.481.
53. Ettou, S., Audureau, E., Humbrecht, C., Benet, B., Jammes, H., Clozel, T., Bardet, V., Lacombe, C., Dreyfus, F., Mayeux, P., et al. (2012). Fas expression at diagnosis as a biomarker of azacitidine activity in high-risk MDS and secondary AML. Leukemia 26, 2297-2299. 10.1038/leu.2012.152.
54. Bogenberger, J. M., Kornblau, S. M., Pierceall, W. E., Lena, R., Chow, D., Shi, C. X., Mantei, J., Ahmann, G., Gonzales, I. M., Choudhary, A., et al. (2014). BCL-2 family proteins as 5-Azacytidine-sensitizing targets and determinants of response in myeloid malignancies. Leukemia 28, 1657-1665. 10.1038/leu.2014.44.
55. Cluzeau, T., Dubois, A., Jacquel, A., Luciano, F., Renneville, A., Preudhomme, C., Karsenti, J. M., Mounier, N., Rohrlich, P., Raynaud, S., et al. (2014). Phenotypic and genotypic characterization of azacitidine-sensitive and resistant SKM1 myeloid cell lines. Oncotarget 5, 4384-4391. 10.18632/oncotarget.2024.
56. Bejar, R., Lord, A., Stevenson, K., Bar-Natan, M., Perez-Ladaga, A., Zaneveld, J., Wang, H., Caughey, B., Stojanov, P., Getz, G., et al. (2014). TET2 mutations predict response to hypomethylating agents in myelodysplastic syndrome patients. Blood 124, 2705-2712. 10.1182/blood-2014-06-582809.
57. Vairy, S., and Tran, T. H. (2020). IKZF1 alterations in acute lymphoblastic leukemia: The good, the bad and the ugly. Blood Reviews 44, 100677. https://doi.org/10.1016fj.blre.2020.100677.
58. Eckardt, J.-N., Stasik, S., Röllig, C., Petzold, A., Sauer, T., Scholl, S., Hochhaus, A., Crysandt, M., Brummendorf, T. H., Naumann, R., et al. (2023). Mutated IKZF1 is an independent marker of adverse risk in acute myeloid leukemia. Leukemia 37, 2395-2403. 10.1038/s41375-023-02061-1.
59. Aubrey, B. J., Cutler, J. A., Bourgeois, W., Donovan, K. A., Gu, S., Hatton, C., Perlee, S., Perner, F., Rahnamoun, H., Theall, A. C. P., et al. (2022). IKAROS and MENIN coordinate therapeutically actionable leukemogenic gene expression in MLL-r acute myeloid leukemia. Nature cancer 3, 595-613. 10.1038/s43018-022-00366-1.
60. Dong, X., Ding, L., Thrasher, A., Wang, X., Liu, J., Pan, Q., Rash, J., Dhungana, Y., Yang, X., Risch, I., et al. (2023). NetBID2 provides comprehensive hidden driver analysis. Nature Communications 14, 2581. 10.1038/s41467-023-38335-6.
61. DepMap, B. (2024). DepMap 24Q4 Public. Figshare+. Dataset. https://depmap.org/portal.
62. Schneider, B. J., Shah, M. A., Klute, K., Ocean, A., Popa, E., Altorki, N., Lieberman, M., Schreiner, A., Yantiss, R., Christos, P. J., et al. (2017). Phase I Study of Epigenetic Priming with Azacitidine Prior to Standard Neoadjuvant Chemotherapy for Patients with Resectable Gastric and Esophageal Adenocarcinoma: Evidence of Tumor Hypomethylation as an Indicator of Major Histopathologic Response. Clinical Cancer Research 23, 2673-2680. 10.1158/1078-0432.CCR-16-1896.
63. Voso, M. T., Breccia, M., Lunghi, M., Poloni, A., Niscola, P., Finelli, C., Bari, A., Musto, P., Zambello, R., Fianchi, L., et al. (2013). Rapid loss of response after withdrawal of treatment with azacitidine: a case series in patients with higher-risk myelodysplastic syndromes or chronic myelomonocytic leukemia. Eur J Haematol 90, 345-348. 10.1111/ejh.12079.
64. Blackledge, N. P., Rose, N. R., and Klose, R. J. (2015). Targeting Polycomb systems to regulate gene expression: modifications to a complex story. Nature Reviews Molecular Cell Biology 16, 643-649. 10.1038/nrm4067.
65. Su, R., Dong, L., Li, C., Nachtergaele, S., Wunderlich, M., Qing, Y., Deng, X., Wang, Y., Weng, X., Hu, C., et al. (2018). R-2HG Exhibits Anti-tumor Activity by Targeting FTO/m(6)A/MYC/CEBPA Signaling. Cell 172, 90-105.e123. 10.1016/j.cell.2017.1 1.031.
66. Cerami, E., Gao, J., Dogrusoz, U., Gross, B. E., Sumer, S. O., Aksoy, B. A., Jacobsen, A., Byrne, C. J., Heuer, M. L., Larsson, E., et al. (2012). The cBio cancer genomics portal: an open platform for exploring multidimensional cancer genomics data. Cancer Discov 2, 401-404. 10.1158/2159-8290.Cd-12-0095.
67. Gao, J., Aksoy, B. A., Dogrusoz, U., Dresdner, G., Gross, B., Sumer, S. O., Sun, Y., Jacobsen, A., Sinha, R., Larsson, E., et al. (2013). Integrative analysis of complex cancer genomics and clinical profiles using the cBioPortal. Sci Signal 6, pl1. 10.1126/scisignal.2004088.
68. de Bruijn, I., Kundra, R., Mastrogiacomo, B., Tran, T. N., Sikina, L., Mazor, T., Li, X., Ochoa, A., Zhao, G., Lai, B., et al. (2023). Analysis and Visualization of Longitudinal Genomic and Clinical Data from the AACR Project GENIE Biopharma Collaborative in cBioPortal. Cancer Res 83, 3861-3867. 10.1158/0008-5472.Can-23-0816.
69. DepMap, B. (2024). DepMap 24Q4 Public. Figshare+. Dataset. https://depmap.org/portal.
70. Sondka, Z., Dhir, N.B., Carvalho-Silva, D., Jupe, S., Madhumita, McLaren, K., Starkey, M., Ward, S., Wilding, J., Ahmed, M., et al. (2024). COSMIC: a curated database of somatic variants and clinical data for cancer. Nucleic Acids Research 52, D1210-D1217. 10.1093/nar/gkad986.
71. Bairoch, A. (2018). The Cellosaurus, a Cell-Line Knowledge Resource. J Biomol Tech 29, 25-38. 10.7171/jbt.18-2902-002.
72. Blanco, S., Kurowski, A., Nichols, J., Watt, F. M., Benitah, S. A., and Frye, M. (2011). The RNA-methyltransferase Misu (NSun2) poises epidermal stem cells to differentiate. PLoS Genet 7, e1002403. 10.1371/journal.pgen.1002403.
73. Lee, H.-Y., Gao, X., Barrasa, M. I., Li, H., Elmes, R. R., Peters, L. L., and Lodish, H.F. (2015). PPAR-a and glucocorticoid receptor synergize to promote erythroid progenitor self-renewal. Nature 522, 474-477. 10.1038/nature14326.
74. Erythropoiesis: Methods and Protocols. (2018). (Springer New York). 10.1007/978-1-4939-7428-3.
75. Hong, J., Sui, P., Li, Y., Xu, K.Y., Lee, J.-H., Wang, J., Chen, S., Zhang, P., Wingate, N., Noor, A., et al. (2025). PSPC1 exerts an oncogenic role in AML by regulating a leukemic transcription program in cooperation with PU.1. Cell Stem Cell 32, 463-478.e466. 10.1016/j.stem.2025.01.010.
76. Liu, J., Dou, X., Chen, C., Chen, C., Liu, C., Xu, M. M., Zhao, S., Shen, B., Gao, Y., Han, D., and He, C. (2020). N6-methyladenosine of chromosome-associated regulatory RNA regulates chromatin state and transcription. Science 367, 580-586. 10.1126/science.aay6018.
77. van der Meer, P., van den Heuvel, D., and Luijsterburg, M. S. (2023). Unscheduled DNA Synthesis at Sites of Local UV-induced DNA Damage to Quantify Global Genome Nucleotide Excision Repair Activity in Human Cells. Bio-protocol 13, e4609. 10.21769/BioProtoc.4609.
78. Zou, Z., Dou, X., Li, Y., Zhang, Z., Wang, J., Gao, B., Xiao, Y., Wang, Y., Zhao, L., Sun, C., et al. (2024). RNA m5C oxidation by TET2 regulates chromatin state and leukemogenesis. Nature 634, 986-994. 10.1038/s41586-024-07969-x.
79. Martin, M. (2011). Cutadapt removes adapter sequences from high-throughput sequencing reads. 2011 17, 3. 10.14806/ej.17.1.200.
80. Kim, D., Paggi, J. M., Park, C., Bennett, C., and Salzberg, S. L. (2019). Graph-based genome alignment and genotyping with HISAT2 and HISAT-genotype. Nature Biotechnology 37, 907-915. 10.1038/s41587-019-0201-4.
81. Liao, Y., Smyth, G. K., and Shi, W. (2014). featureCounts: an efficient general purpose program for assigning sequence reads to genomic features. Bioinformatics 30, 923-930. 10.1093/bioinformatics/btt656.
82. Love, M. I., Huber, W., and Anders, S. (2014). Moderated estimation of fold change and dispersion for RNA-seq data with DESeq2. Genome Biology 15, 550. 10.1186/s13059-014-0550-8.
83. Risso, D., Ngai, J., Speed, T. P., and Dudoit, S. (2014). Normalization of RNA-seq data using factor analysis of control genes or samples. Nature Biotechnology 32, 896-902. 10.1038/nbt.2931.
84. Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: A knowledge-based approach for interpreting genome-wide expression profiles. Proceedings of the National Academy of Sciences 102, 15545-15550. 10.1073/pnas.0506580102.
85. Mootha, V. K., Lindgren, C. M., Eriksson, K.-F., Subramanian, A., Sihag, S., Lehar, J., Puigserver, P., Carlsson, E., Ridderstrile, M., Laurila, E., et al. (2003). PGC-1α-responsive genes involved in oxidative phosphorylation are coordinately downregulated in human diabetes. Nature Genetics 34, 267-273. 10.1038/ng1180.
86. Langmead, B., and Salzberg, S. L. (2012). Fast gapped-read alignment with Bowtie 2. Nature Methods 9, 357-359. 10.1038/nmeth.1923.
87. Criscione, S.W., Zhang, Y., Thompson, W., Sedivy, J. M., and Neretti, N. (2014). Transcriptional landscape of repetitive elements in normal and cancer human cells. BMC Genomics 15, 583. 10.1186/1471-2164-15-583.
88. Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based Analysis of ChIP-Seq (MACS). Genome Biology 9, R137. 10.1186/gb-2008-9-9-r137.
89. Ramírez, F., Ryan, D. P., Graning, B., Bhardwaj, V., Kilpert, F., Richter, A.S., Heyne, S., Dündar, F., and Manke, T. (2016). deepTools2: a next generation web server for deep-sequencing data analysis. Nucleic Acids Research 44, W160-W165. 10.1093/nar/gkw257.
90. Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589. 10.1016/j.molcel.2010.05.004.
91. Picard toolkit (2019). (Broad Institute).
92. Khatamian, A., Paull, E. O., Califano, A., and Yu, J. (2019). SJARACNe: a scalable software tool for gene network reverse engineering from big data. Bioinformatics 35, 2165-2166. 10.1093/bioinformatics/bty907.
93. Dong, X., Ding, L., Thrasher, A., Wang, X., Liu, J., Pan, Q., Rash, J., Dhungana, Y., Yang, X., Risch, I., et al. (2023). NetBID2 provides comprehensive hidden driver analysis. Nature Communications 14, 2581. 10.1038/s41467-023-38335-6.

1. A method of treating a disease in an individual, comprising the step of administering one or more inhibitors of Ikaros family zinc finger protein 1 (IKZF1), Enhancer of zeste homolog 2 (EZH2), and/or nucleophosmin 1 (NPM1), to an individual in need thereof.

2-4. (canceled)

5. The method of claim 1, wherein the disease comprises cancer of the blood, lung, brain, breast, skin, pancreas, liver, colon, head and neck, kidney, thyroid, stomach, spleen, gallbladder, bone, ovary, testes, endometrium, prostate, rectum, anus, and/or cervix; and/or the disease comprises clonal hematopoiesis of indeterminate potential (CHIP); and/or the disease is characterized as pro-inflammatory.

6-9. (canceled)

10. The method of claim 5, wherein the cancer comprises a leukemia, myeloid malignancy, or glioma; and/or the disease comprises clonal hematopoiesis of indeterminate potential (CHIP) and the disease is characterized by atherosclerosis, myocardial fibrosis, and/or heart failure.

11. The method of claim 10, wherein the myeloid malignancy comprises acute myeloid leukemia, chronic myelomonocytic leukemia, or myelodysplastic syndrome and/or the glioma comprises glioblastoma.

12-16. (canceled)

17. The method of claim 1, wherein the disease is associated with diseased cells comprising:
(a) one or more mutations in one or more genes encoding a ten-eleven translocation (tet) methylcytosine dioxygenase 2 (TET2), ASXL transcriptional regulator 1 (ASXL1), isocitrate dehydrogenase 1 (IDH1), isocitrate dehydrogenase 2 (IDH2), tumor protein p53 (p53), DNA (cytosine-5-)-methyltransferase 3A (DNMT3A), Janus kinase 2 (JAK2), Protein Phosphatase Mn2+/Mg2+-Dependent 1D (PPM1D), Spliceosome Factor 3b1 (SF3B1), and/or Serine and Arginine Rich Splicing Factor 2 (SRSF2); and/or
(b) a rearrangement of the lysine methyltransferase 2A (KMT2A)/mixed-lineage leukemia (MLL) gene; and/or
(c) one or more mutations in one or more genes encoding components of a canonical and/or non-canonical Polycomb Repressive Complex (PRC); and/or
(d) one or more mutations in one or more genes encoding Polycomb Repressive-Deubiquitinase (PR-DUB) complex associated components O-linked N-acetylglucosamine Transferase (OGT), Lysine Demethylase 1B (KDM1B), Forkhead Box K1 (FOXK1), Forkhead Box K2 (FOXK2), BRCA1 Associated Protein 1 (BAP1), ASXL Transcriptional Regulator 1 (ASXL1), ASXL Transcriptional Regulator 2 (ASXL2), ASXL Transcription Regulator 3 (ASXL3), and/or Host Cell Factor C1 (HCFC1); and/or
(e) one or more mutations in a TET2 encoding gene; and/or
(f) one or more mutations in one or more genes encoding SRSF2, ASXL1, p53, DNMT3A, JAK2, PPM1D, and/or SF3B1; and/or
(g) one or more mutations in IKZF1, EZH2, NPM1, TET2, ASXL1, IDH1, IDH2, p53, DNMT3A, JAK2, PPM1D, SF3B1, and/or SRSF2 encoding genes; and/or
(h) an open chromatin state relative to non-diseased cells of the same developmental lineage.

18-19. (canceled)

20. The method of claim 17, wherein:
(a) the one or more mutations in one or more genes encoding components of PRC comprises one or more loss of function mutations and/or one or more mutations in E3 Ubiquitin Ligase RING1A/B, Polycomb Group Ring Finger 1 (PCGF1), Polycomb Group Ring Finger 2 (PCGF2), Polycomb Group Ring Finger 3 (PCGF3), Polycomb Group Ring Finger 4 (PCGF4), Polycomb Group Ring Finger 5 (PCGF5), and/or Polycomb Group Ring Finger 6 (PCGF6); and/or
(b) the one or more mutations in PR-DUB complex associated components OGT, KDM1B, FOXK1, FOXK2, BAP1, ASXL1, ASXL2, ASXL3, and/or HCFC1 comprises one or more gain of function mutations; and/or
(c) the one or more mutations in a TET2 encoding gene comprises one or more loss of function mutations.

21-26. (canceled)

27. The method of claim 1, wherein the individual is administered an additional therapy.

28. The method of claim 27, wherein the additional therapy comprises administration of 5-AzaC, surgery, radiation, chemotherapy, hormone therapy, immunotherapy, administration of an inhibitor of TET2, administration of an inhibitor of NSUN1 and/or NSUN2; and/or administration of an inhibitor of MBD6.

29-30. (canceled)

31. The method of claim 28, wherein the inhibitor of TET2 comprises C35 and/or TETi76 and/or the inhibitor of NSUN1/NSUN2 comprises a compound of claim 94.

32-37. (canceled)

38. The method of claim 1, further comprising a step of diagnosing the disease in the individual.

39. (canceled)

40. The method of claim 1, wherein administering the one or more IKZF1, EZH2, and/or NPM1 inhibitors results in one or more of:
(a) promotion of a closed chromatin state in one or more diseased cells in the individual,
(b) inhibition of diseased cell proliferation,
(c) reduced proliferation of a cancer and/or pre-cancerous cell
(d) a decrease in m5C levels in one or more chromatin associated RNA (caRNA) and/or a decrease in association of one or more caRNA with a PR-DUB complex in one or more diseased cells in the individual,
(e) an increase in oxidation of m5C in the one or more caRNA and/or inhibition of installation of m5C in one or more caRNA in one or more diseased cells in the individual,
(f) an increase in histone ubiquitination in one or more diseased cells,
(g) modified histone methylation in one or more diseased cells in the individual.

41-45. (canceled)

46. The method of claim 40, wherein the histone ubiquitination comprises or consists of ubiquitination on H2A; the histone ubiquitination occurs at a site comprising or consisting essentially of H2AK119: a histone that comprises modified methylation comprises or consists essentially of H3; a histone that comprises modified methylation comprises modified methylation on a methylation site comprising or consisting essentially of H3K27me3; and/or a histone that comprises modified methylation is localized near or at a genetic locus overlapping with a histone that comprises modified histone ubiquitination.

47-51. (canceled)

52. The method of claim 1, wherein:
(a) the one or more inhibitors of IKZF1, EZH2, and/or NPM1 comprise one or more polynucleotides at least partially complementary to a gene encoding IKZF1, EZH2, and/or NPM1 and/or a proteolysis targeting chimera;
(b) the one or more inhibitors of EZH2 comprises Tazemetostat, CPI-1205, GSK2816126, PF-06821497, SHR2554, and/or DS3201b; and/or
(c) the one or more inhibitors of IKZF1 comprises CC-92480.

53. The method of claim 52, wherein the one or more polynucleotides comprise a short hairpin RNA and/or small interfering RNA: comprise a sequence at least 80% complementary to at least 15, 20, 25, or more than 25 contiguous nucleotides of any one or more of SEO ID NOs: 2-3, 5-6, 8-9, 11-12, 14-15, or 17-19; comprise modified RNA phosphoramidites: comprise RNA with one or more 2′-O-Methyl (2′-OMe) or 2′-O-Methoxyethyl (2′-MOE) modifications; and/or comprise RNA with one or more phosphorothioate bonds.

54-58. (canceled)

59. The method of claim 52, wherein the on or more polynucleotides are comprised within a lentiviral particle and/or nanoparticle; and/or the one or more polynucleotides comprise RNA with every nucleotide comprising a 2′-MOE modification.

60-69. (canceled)

70. A method of treating a disease in an individual, comprising determining the gene expression levels and/or protein activity levels of one or more of IKZF1, EZH2, and/or NPM1 in diseased cells in the individual, and administering 5-AzaC to the individual if the expression levels and/or protein activity levels of IKZF1, EZH2, and/or NPM1 are reduced relative to expression levels in a non-diseased cell of the same developmental lineage in the individual.

71. A method of treating a disease in an individual, comprising administering one or more inhibitors of NSUN2 to the individual in need thereof, wherein the one or more inhibitors of NSUN2 comprise a compound of claim 94.

72-93. (canceled)

94. A compound of Formula (I) or pharmaceutical appropriate salt thereof:
Ar-L-B (I), wherein:
Ar is a heteroaryl moiety or an aryl moiety substituted with a heteroaryl substituent, wherein Ar is optionally substituted with 1 to 3 R_{y} substituents, each R_{y} independently selected from halogen, cyano, nitro, alkyl, haloalkyl, alkoxy, haloalkoxy, alkoxycarbonyl, carboxy(C1-C6)alkoxy, aryl, heteroaryl, aryloxy, and aralkoxy, wherein the heteroaryl moiety or the heteroaryl substituent comprises at least one ring heteroatom selected from nitrogen, oxygen, and sulfur, and when the heteroaryl moiety or the heteroaryl substituent comprises a ring nitrogen atom, the ring nitrogen atom is unsubstituted or substituted with R_{N}, wherein R_{N} is selected from SO2R_{X}, C(═O)R_{X}, and C(═O)OR_{X}, and wherein R_{X} is selected from substituted or unsubstituted C1-C6 alkyl, substituted or unsubstituted aryl, substituted or unsubstituted heteroaryl, and substituted or unsubstituted heterocyclyl;
L is —CH═, wherein the carbon atom of L is singly bonded to Ar and doubly bonded to a carbon atom of B; and
B is selected from a barbiturate moiety, a thiobarbiturate moiety, a Meldrum's acid moiety, a thiazolidinedione moiety, a pyrazolone moiety, an indane-1,3-dione moiety, a cyanoacrylic acid moiety, a cyanoacrylate moiety, and an aryl-substituted acrylonitrile moiety.

95-123. (canceled)

124. The compound of claim 94, wherein the compound comprises a structure selected from: 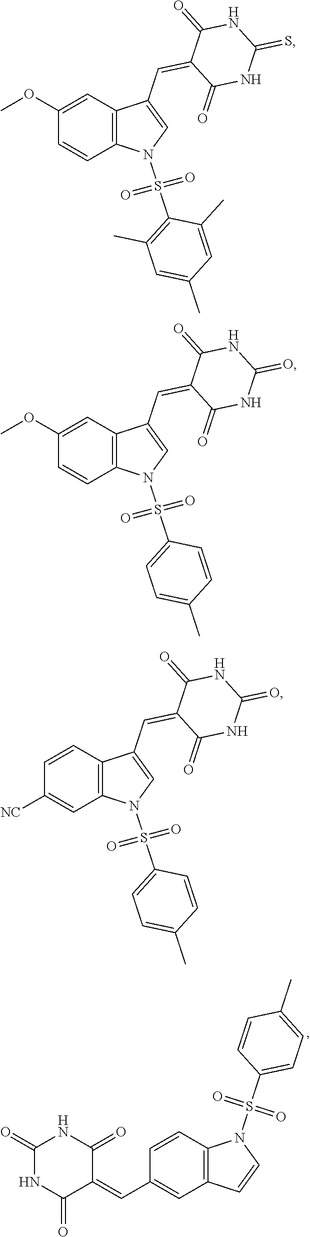 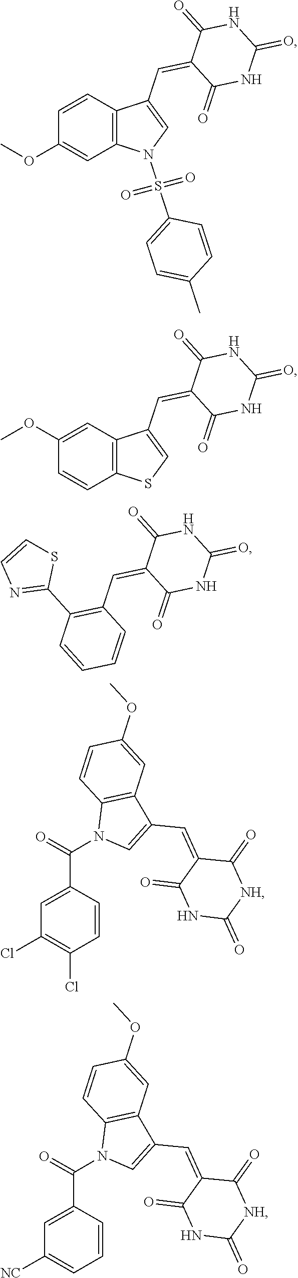 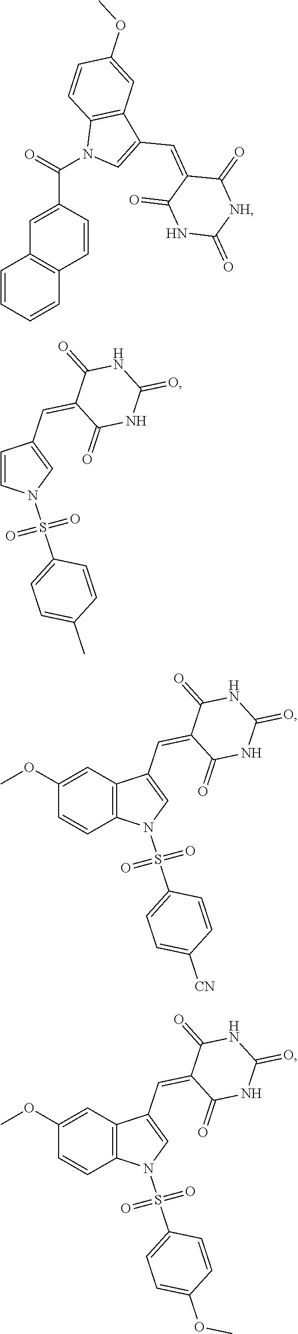 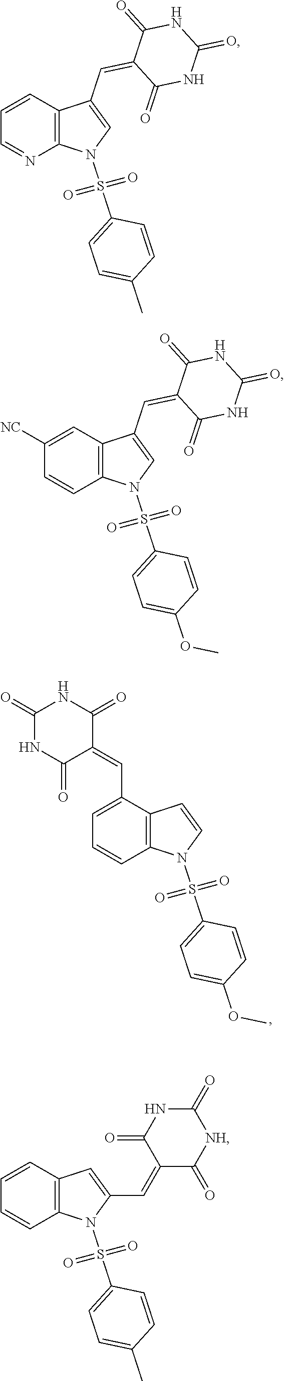 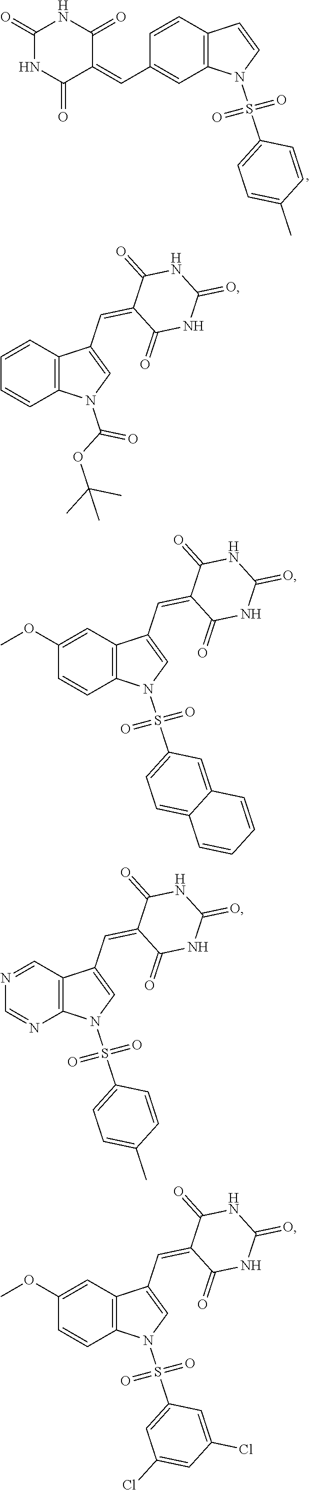 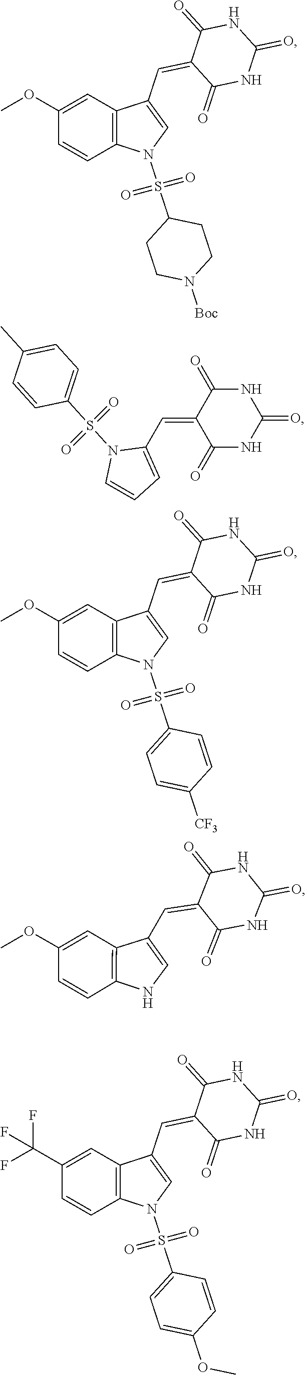 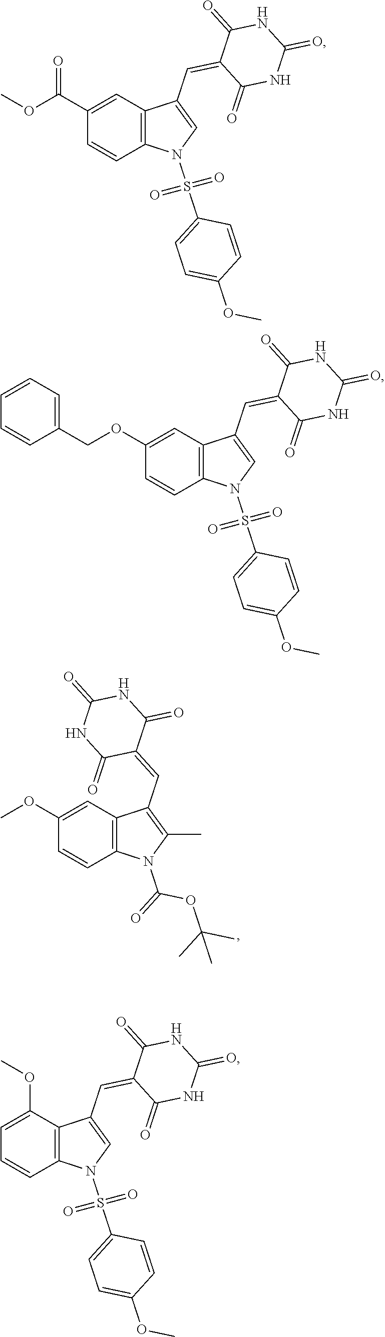 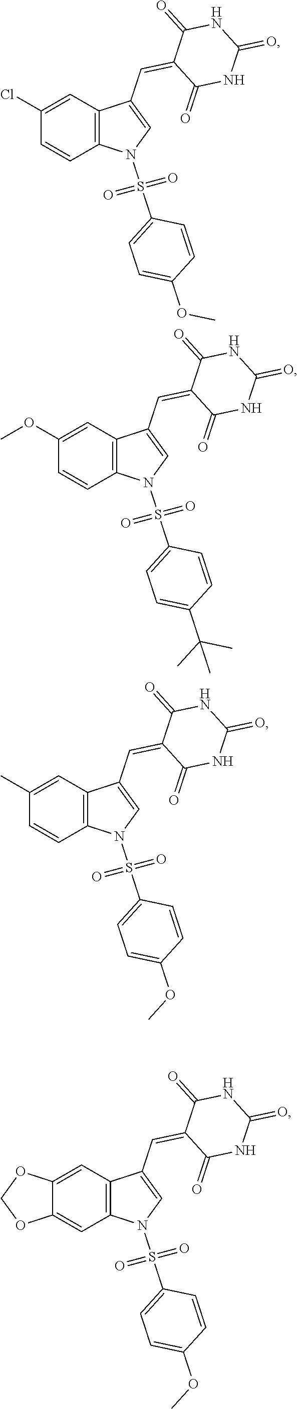 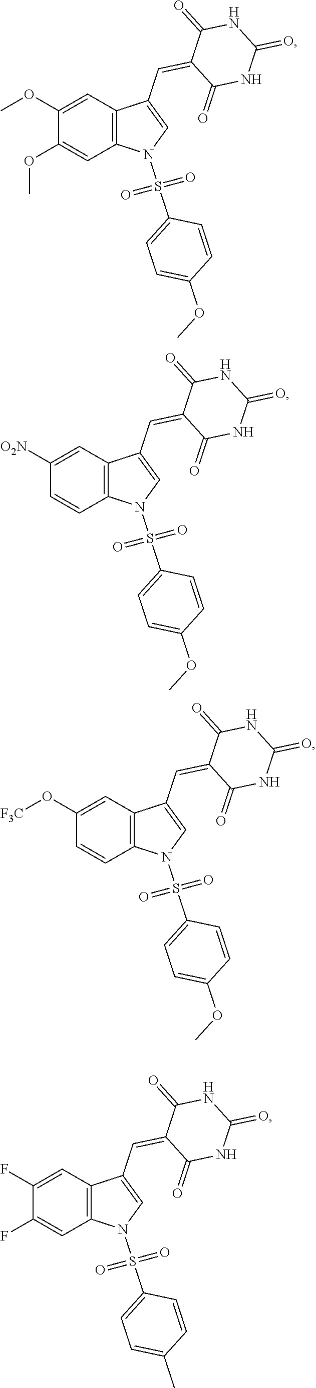 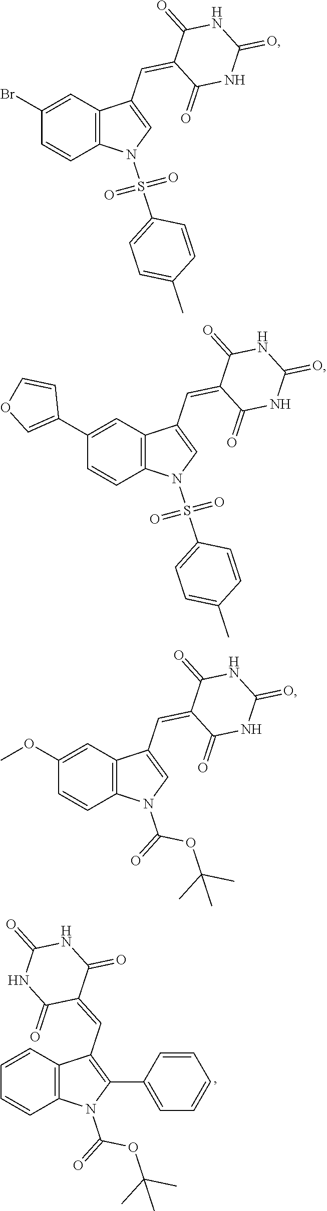 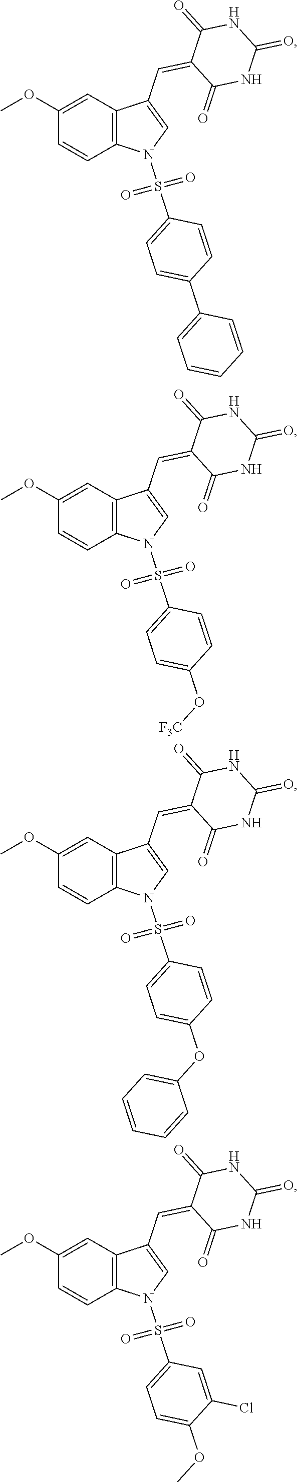 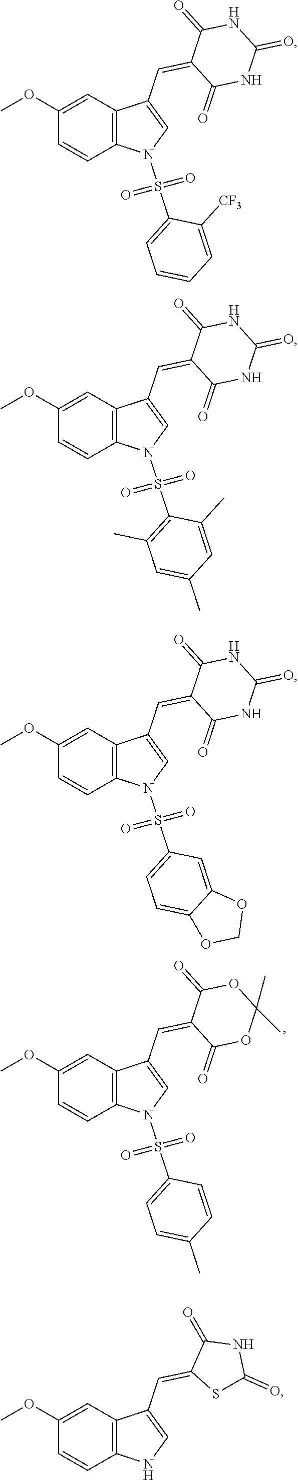 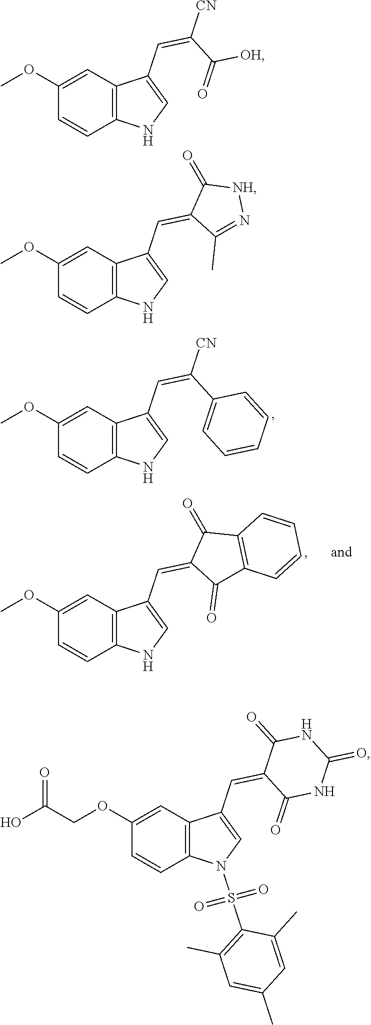 [SMILES: COc1ccc2[nH]cc(/C=C(/C#N)C(=O)O)c2c1 ; COc1ccc2[nH]cc(/C=C3/C(=O)NN=C3C)c2c1 ; COc1ccc2[nH]cc(/C=C(/C#N)c3ccccc3)c2c1 ; COc1ccc2[nH]cc(C=C3C(=O)c4ccccc4C3=O)c2c1 ; Cc1cc(C)c(S(=O)(=O)n2cc(C=C3C(=O)NC(=O)NC3=O)c3cc(OCC(=O)O)ccc32)c(C)c1]
or a pharmaceutically appropriate salt thereof.

125-126. (canceled)

127. A pharmaceutical composition comprising a compound of claim 94 and at least one pharmaceutically appropriate carrier or excipient.
